(19) United States
(12) Patent Application Publication
(10) Pub. No.: US 2023/0295651 A1
(43) Pub. Date: Sep. 21, 2023

(54) RECOMBINANT NUCLEIC ACID CONSTRUCT AND USE THEREOF

(71) Applicant: Pantherna Therapeutics GmbH, Hennigsdorf (DE)

(72) Inventors: Joerg KAUFMANN, Berlin (DE); Oliver KEIL, Berlin (DE); Daniel TONDERA, Berlin (DE)

(21) Appl. No.: 17/885,088

(22) Filed: Aug. 10, 2022

Related U.S. Application Data
(63) Continuation of application No. PCT/EP2021/053370, filed on Feb. 11, 2021.

(30) Foreign Application Priority Data
Feb. 11, 2020 (EP) 20000064.4
Feb. 12, 2020 (EP) 20156987.8
Feb. 10, 2021 (EP) 21156423.2

(51) Int. Cl. C12N 15/85; A61K 9/51; C07K 14/47; C12N 9/12

(52) U.S. Cl. CPC C12N 15/85; A61K 9/5123; C07K 14/4705; C12N 9/12; A61K 48/00; C12N 2800/107; C12N 2830/50; C12N 2840/203

(57) ABSTRACT

The present invention is related to a recombinant nucleic acid construct comprising in 5′-> 3′ direction
a 5′ UTR,
a coding region coding for an effector molecule, and
a 3′ UTR,

wherein the 5′ UTR is selected from the group consisting of a 5′ UTR of a gene coding for MCP-1 or a derivative thereof having a nucleotide identity of at least 85%, a 5′ UTR of a gene coding for RPL12s.c. or a derivative thereof having a nucleotide identity of at least 85%, a 5′ UTR of a gene coding for Ang-2 or a derivative thereof having a nucleotide identity of at least 85%, a 5′ UTR of a gene coding for HSP70 or a derivative thereof having a nucleotide identity of at least 85%, a 5′ UTR of a gene coding for H3.3. or a derivative thereof having a nucleotide identity of at least 85%, a 5′ UTR of a gene coding for Galectin-9 or a derivative thereof having a nucleotide identity of at least 85%, a 5′ UTR of a gene coding for GADD34 or a derivative thereof having a nucleotide identity of at least 85%, a 5′ UTR of a gene coding for EDN1 or a derivative thereof having a nucleotide identity of at least 85%, a 5′ UTR of a gene coding for HSP70m5 or a derivative thereof having a nucleotide identity of at least 85%, a 5′ UTR of a gene coding for E-selectin or a derivative thereof having a nucleotide identity of at least 85% a 5′ UTR of a gene coding for ICAM-1 or a derivative thereof having a nucleotide identity of at least 85%, a 5′ UTR of a gene coding for IL-6 or a derivative thereof having a nucleotide identity of at least 85% and a 5′ UTR of a gene coding for vWF or a derivative thereof having a nucleotide identity of at least 85 %;
wherein 3′ UTR is selected from the group consisting of a 3′ UTR of a gene coding for vWF or a derivative thereof having a nucleotide identity of at least 85%, a 3′ UTR of a gene coding for MCP-1 or a derivative thereof having a nucleotide identity of at least 85%,, a 3′ UTR of a gene coding for RPL12s.c. or a derivative thereof having a nucleotide identity of at least 85%, a 3′ UTR of a gene coding for HSP70 or a derivative thereof having a nucleotide identity of at least 85%, a 3′ UTR of a gene coding for H3.3. or a derivative thereof having a nucleotide identity of at least 85%, a 3′ UTR of a gene coding for GADD34 or a derivative thereof having a nucleotide identity of at least 85%, a 3′ UTR of a gene coding for EDN1 or a derivative thereof having a nucleotide identity of at least 85%, and a 3′ UTR of a gene coding for IL-6 or a derivative thereof having a nucleotide identity of at least 85%,
wherein the effector molecule is effective in restoring a cellular function of a cell or is effective in exercising a therapeutic effect in or on a cell, and
wherein the recombinant nucleic acid construct is different from a wild type mRNA coding for the effector molecule.

This application is a continuation of PCT/EP2021/053370, filed Feb. 11, 2021, and further claims priority to EP application nos. EP20000064.4, filed Feb. 11, 2020, EP20156987.8, filed Feb. 12, 2020, and EP21156423.2, filed Feb. 10, 2021, the contents of each of which are hereby incorporated by reference in their entireties.

The instant application contains a Sequence Listing which has been submitted electronically in ST.26 (XML) format and is hereby incorporated by reference in its entirety. Said Sequence List, created on Mar. 2, 2023, is named Sequence_Listing_28022023 and is 356 kilobytes in size.

The present invention is related to a recombinant nucleic acid construct, a vector, preferably an expression vector, comprising a recombinant nucleic acid construct, a cell comprising the recombinant nucleic acid construct and/or the vector, a delivery vehicle comprising the recombinant nucleic acid construct, a pharmaceutical composition comprising the recombinant nucleic acid construct, the recombinant nucleic acid construct for use in a method for the treatment and/or prevention of a disease, the recombinant nucleic acid construct for use in a method for restoring a cellular function of a cell, a method for the treatment and/or prevention of a disease and a method for restoring a cellular function of a cell.

Nucleic acid molecules serve various purposes in biological systems. One out of several purposes is to store genetic information, another one is to convey genetic information from deoxyribonucleic acid to the ribosome where such genetic information is translated into an amino acid sequence. The latter step involves the use of messenger ribonucleic acid (mRNA).

mRNA shows a very basic design in eukaryotic cells and typically comprises, in 5′ -> 3′ direction, a Cap structure, a 5′ untranslated region (5′ UTR), a coding sequence typically starting with a AUG codon attached to a coding sequence (CDS) terminating with a stop codon, a 3′ untranslated region (3′ UTR) and a poly-A-tail.

mRNA based therapy have been proposed as therapeutics since the beginning of the biotech. In principle, the administered mRNA sequence can cause a cell to make a protein, which in turn could directly treat a disease or could function as a vaccine; more indirectly the protein could interfere with an element of a pathway in such way that the pathway is either inhibited or stimulated, thereby treating or ameliorating a disease.

The problem underlying the present invention is the provision of a recombinant nucleic acid construct, preferably of an mRNA, whereby such recombinant nucleic acid construct allows for high expression of the coding sequence of the recombinant nucleic acid construct.

Another problem underlying the present invention is the provision of a recombinant nucleic acid construct, preferably of an mRNA, whereby such recombinant nucleic acid construct allows for the expression of the coding sequence of the recombinant nucleic acid construct in an endothelial cell.

These and other problems underlying the present invention are solved by the subject matter of the attached independent claims; preferred embodiments may be taken from the attached dependent claims.

Additionally, and more specifically, the problem underlying the present invention is solved in a first aspect, which is also the first embodiment of the first aspect, by a recombinant nucleic acid construct comprising in 5′ -> 3′ direction
a 5′ UTR,
a coding region coding for an effector molecule, and
a 3′ UTR,

wherein the 5′ UTR is selected from the group comprising a 5′ UTR of a gene coding for MCP-1 or a derivative thereof having a nucleotide identity of at least 85%, a 5′ UTR of a gene coding for RPL12s.c. or a derivative thereof having a nucleotide identity of at least 85%, a 5′ UTR of a gene coding for Ang-2 or a derivative thereof having a nucleotide identity of at least 85%, a 5′ UTR of a gene coding for HSP70 or a derivative thereof having a nucleotide identity of at least 85%, a 5′ UTR of a gene coding for H3.3. or a derivative thereof having a nucleotide identity of at least 85%, a 5′ UTR of a gene coding for Galectin-9 or a derivative thereof having a nucleotide identity of at least 85%, a 5′ UTR of a gene coding for GADD34 or a derivative thereof having a nucleotide identity of at least 85%, a 5′ UTR of a gene coding for EDN1 or a derivative thereof having a nucleotide identity of at least 85%, a 5′ UTR of a gene coding for HSP70m5 or a derivative thereof having a nucleotide identity of at least 85%, a 5′ UTR of a gene coding for E-selectin or a derivative thereof having a nucleotide identity of at least 85 % a 5′ UTR of a gene coding for ICAM-1 or a derivative thereof having a nucleotide identity of at least 85%, a 5′ UTR of a gene coding for IL-6 or a derivative thereof having a nucleotide identity of at least 85% and a 5′ UTR of a gene coding for vWF or a derivative thereof having a nucleotide identity of at least 85%;
wherein 3′ UTR is selected from the group comprising a 3′ UTR of a gene coding for vWF or a derivative thereof having a nucleotide identity of at least 85%, a 3′ UTR of a gene coding for MCP-1 or a derivative thereof having a nucleotide identity of at least 85%, a 3′ UTR of a gene coding for RPL12s.c. or a derivative thereof having a nucleotide identity of at least 85 %, a 3′ UTR of a gene coding for HSP70 or a derivative thereof having a nucleotide identity of at least 85%, a 3′ UTR of a gene coding for H3.3. or a derivative thereof having a nucleotide identity of at least 85%, a 3′ UTR of a gene coding for GADD34 or a derivative thereof having a nucleotide identity of at least 85%, a 3′ UTR of a gene coding for EDN1 or a derivative thereof having a nucleotide identity of at least 85%, and a 3′ UTR of a gene coding for IL-6 or a derivative thereof having a nucleotide identity of at least 85%,
wherein the effector molecule is effective in restoring a cellular function of a cell or is effective in exercising a therapeutic effect in or on a cell,
wherein the effector molecule is a mutant effector molecule, wherein the mutant effector molecule is a gain of function mutant effector molecule and/or a hypermorphic mutant effector molecule, and
wherein the recombinant nucleic acid construct is different from a wild type mRNA coding for the effector molecule.

In a second embodiment of the first aspect, which is also an embodiment of the first embodiment of the first aspect, the 5′ UTR and the 3′ UTR of the recombinant construct are of different origin, preferably are from different endogenous species.

In a third embodiment of the first aspect, which is also an embodiment of the first and second embodiment of the first aspect, the 5′ UTR and the 3′ UTR of the recombinant construct are of the same origin, preferably are from the same endogenous genes species.

In a fourth embodiment of the first aspect, which is also an embodiment of the first, second and third embodiment of the first aspect,
a) the 5′ UTR is a 5′ UTR of a gene coding for MCP-1 or a derivative thereof having a nucleotide identity of at least 85%, and wherein the 3′ UTR is selected from the group comprising a 3′ UTR of a gene coding for vWF or a derivative thereof having a nucleotide identity of at least 85%, a 3′ UTR of a gene coding for HSP70 or a derivative thereof having a nucleotide identity of at least 85%, a 3′ UTR of a gene coding for RPL12s.c. or a derivative thereof having a nucleotide identity of at least 85%, and a 3′ UTR of a gene coding for MCP-1 or a derivative thereof having a nucleotide identity of at least 85%.
b) the 5′ UTR is a 5′ UTR of a gene coding for RPL12s.c. or a derivative thereof having a nucleotide identity of at least 85%, and wherein the 3′ UTR is selected from the group comprising a 3′ UTR of a gene coding for vWF or a derivative thereof having a nucleotide identity of at least 85%, a 3′ UTR of a gene coding for HSP70 or a derivative thereof having a nucleotide identity of at least 85%, a 3′ UTR of a gene coding for RPL12s.c. or a derivative thereof having a nucleotide identity of at least 85% and a 3′ UTR of a gene coding for MCP-1 or a derivative thereof having a nucleotide identity of at least 85%,
c) the 5′ UTR is a 5′ UTR of a gene coding for ANG-2 or a derivative thereof having a nucleotide identity of at least 85%, and wherein the 3′ UTR is selected from the group comprising a 3′ UTR of a gene coding for HSP70 or a derivative thereof having a nucleotide identity of at least 85%, a 3′ UTR of a gene coding for RPL12s. c. or a derivative thereof having a nucleotide identity of at least 85%, a 3′ UTR of a gene coding for vWF or a derivative thereof having a nucleotide identity of at least 85% and a 3′ UTR of a gene coding for MCP-1 or a derivative thereof having a nucleotide identity of at least 85%, or
d) the 5′ UTR is a 5′ UTR of a gene coding for HSP70 or a derivative thereof having a nucleotide identity of at least 85%, and wherein the 3′ UTR is selected from the group comprising a 3′ UTR of a gene coding for HSP70 or a derivative thereof having a nucleotide identity of at least 85%, a 3′ UTR of a gene coding for RPL12s. c. or a derivative thereof having a nucleotide identity of at least 85%, a 3′ UTR of a gene coding for vWF or a derivative thereof having a nucleotide identity of at least 85% and a 3′ UTR of a gene coding for MCP-1 or a derivative thereof having a nucleotide identity of at least 85%.

In a fifth embodiment of the first aspect, which is also an embodiment of the first, second and third embodiment of the first aspect,
a) the 3′UTR is a 3′ UTR of a gene coding for vWF or a derivative thereof having a nucleotide identity of at least 85% and the 5′ UTR is selected from the group comprising a 5′ UTR of a gene coding for MCP-1 or a derivative thereof having a nucleotide identity of at least 85%, a 5′ UTR of a gene coding for RPL12s.c. or a derivative thereof having a nucleotide identity of at least 85%, a 5′ UTR of a gene coding for HSP70 or a derivative thereof having a nucleotide identity of at least 85%, a 5′ UTR of a gene coding for ANG-2 of a derivative thereof having a nucleotide identity of at least 85%,
b) the 3′UTR is a 3′ UTR of a gene coding for HSP70 or a derivative thereof having a nucleotide identity of at least 85% and the 5′ UTR is selected from the group comprising a 5′ UTR of a gene coding for HSP70 or a derivative thereof having a nucleotide identity of at least 85% a 5′ UTR of a gene coding for RPL12s.c. or a derivative thereof having a nucleotide identity of at least 85%, a 5′ UTR of a gene coding for ANG-2 of a derivative thereof having a nucleotide identity of at least 85%, and a 5′ UTR of a gene coding for MCP-1 or a derivative thereof having a nucleotide identity of at least 85%,
c) the 3′UTR is a 3′ UTR of a gene coding for RPL12s.c. or a derivative thereof having a nucleotide identity of at least 85% and the 5′ UTR is selected from the group comprising a 5′ UTR of a gene coding for HSP70 or a derivative thereof having a nucleotide identity of at least 85% a 5′ UTR of a gene coding for RPL12s.c. or a derivative thereof having a nucleotide identity of at least 85%, a 5′ UTR of a gene coding for ANG-2 of a derivative thereof having a nucleotide identity of at least 85%, and a 5′ UTR of a gene coding for MCP-1 or a derivative thereof having a nucleotide identity of at least 85%, or
d) the 3′UTR is a 3′ UTR of a gene coding for MCP-1 or a derivative thereof having a nucleotide identity of at least 85% and the 5′ UTR is selected from the group comprising a 5′ UTR of a gene coding for HSP70 or a derivative thereof having a nucleotide identity of at least 85%, a 5′ UTR of a gene coding for RPL12s.c. or a derivative thereof having a nucleotide identity of at least 85 %, a 5′ UTR of a gene coding for ANG-2 of a derivative thereof having a nucleotide identity of at least 85%, and a 5′ UTR of a gene coding for MCP-1 or a derivative thereof having a nucleotide identity of at least 85%.

In a sixth embodiment of the first aspect, which is also an embodiment of the first, second, third, fourth and fifth embodiment of the first aspect, the construct is one selected from the group comprising
a construct, wherein the 5′ UTR is a 5′ UTR of a gene coding for MCP-1 or a derivative thereof having a nucleotide identity of at least 85%, and the 3′ UTR is a 3′ UTR of a gene coding for vWF or a derivative thereof having a nucleotide identity of at least 85%,
a construct, wherein the 5′ UTR is a 5′ UTR of a gene coding for RPL12s.c. or a derivative thereof having a nucleotide identity of at least 85%, and the 3′ UTR is a 3′ UTR of a gene coding for vWF or a derivative thereof having a nucleotide identity of at least 85%,
a construct, wherein the 5′ UTR is a 5′ UTR of a gene coding for MCP-1 or a derivative thereof having a nucleotide identity of at least 85%, and the 3′ UTR is a 3′ UTR of a gene coding for MCP-1 or a derivative thereof having a nucleotide identity of at least 85%,
a construct, wherein the 5′ UTR is a 5′ UTR of a gene coding for ANG-2 or a derivative thereof having a nucleotide identity of at least 85%, and the 3′ UTR is a 3′ UTR of a gene coding for vWF or a derivative thereof having a nucleotide identity of at least 85%, a construct, wherein the 5′ UTR is a 5′ UTR of a gene coding for RPL12s.c. or a derivative thereof having a nucleotide identity of at least 85%, and the 3′ UTR is a 3′ UTR of a gene coding for RPL12s.c. or a derivative thereof having a nucleotide identity of at least 85%,
a construct, wherein the 5′ UTR is a 5′ UTR of a gene coding for HSP70 or a derivative thereof having a nucleotide identity of at least 85%, and the 3′ UTR is a 3′ UTR of a gene coding for HSP70 or a derivative thereof having a nucleotide identity of at least 85%,
a construct, wherein the 5′ UTR is a 5′ UTR of a gene coding for H3.3. or a derivative thereof having a nucleotide identity of at least 85%, and the 3′ UTR is a 3′ UTR of a gene coding for H3.3. or a derivative thereof having a nucleotide identity of at least 85%, and
a construct, wherein the 5′ UTR is a 5′ UTR of a gene coding for Galectin 9 or a derivative thereof having a nucleotide identity of at least 85%, and the 3′ UTR is a 3′ UTR of a gene coding for Galectin 9 or a derivative thereof having a nucleotide identity of at least 85%.

In a seventh embodiment of the first aspect, which is also an embodiment of the first, second, third, fourth, fifth and sixth embodiment of the first aspect, the construct comprises a poly-A tail, preferably at the 3′ terminal end of the recombinant nucleic acid construct.

In an eighth embodiment of the first aspect, which is also an embodiment of the first, second, third, fourth, fifth, sixth and seventh embodiment of the first aspect, the construct comprises a CAP structure, preferably at the 5′ terminal end of the recombinant nucleic acid construct.

In a ninth embodiment of the first aspect, which is also an embodiment of the first, second, third, fourth, fifth, sixth, seventh and eighth embodiment of the first aspect, the construct comprises a IRES (internal ribosomal entry site) sequence, preferably at the 5′ terminal end of the recombinant nucleic acid construct.

In a tenth embodiment of the first aspect, which is also an embodiment of the first, second, third, fourth, fifth, sixth, seventh, eighth and ninth embodiment of the first aspect, the construct comprises nucleic acid sequence coding for a signal peptide, preferably the signal peptide is in-frame with nucleic acid sequence coding for a signal peptide and is arranged between the 5′ UTR and the coding region coding for an effector molecule.

In an eleventh embodiment of the first aspect, which is also an embodiment of the tenth embodiment of the first aspect, the signal peptide allows secretion of the effector molecule.

In a 12^{th} embodiment of the first aspect, which is also an embodiment of the tenth and the eleventh embodiment of the first aspect, the nucleotide sequence coding for a signal peptide is selected from the group comprising a nucleotide sequence coding for a signal peptide of MCP-1 or a derivative thereof having a nucleotide identity of at least 85%, nucleotide sequence coding for a signal peptide of IL-6 or a derivative thereof having a nucleotide identity of at least 85%, a nucleotide sequence coding for a signal peptide of Ang-2 or a derivative thereof having a nucleotide identity of at least 85%, and a nucleotide sequence coding for a signal peptide of Ang-1 or a derivative thereof having a nucleotide identity of at least 85%.

In a13^{th} embodiment of the first aspect, which is also an embodiment of the first, second, third, fourth, fifth, sixth, seventh, eighth, ninth, tenth, eleventh and 12^{th} embodiment of the first aspect, the cell a cellular function of which is restored and/or the cell in or on which a therapeutic effect is exercised is an endothelial cell, preferably a vascular endothelial cell, more preferably the vascular endothelial cell is a microvascular endothelial cell.

In 14^{th} embodiment of the first aspect, which is also an embodiment of the first, second, third, fourth, fifth, sixth, seventh, eighth, ninth, tenth, eleventh, 12^{th} and 13^{th} embodiment of the first aspect, the cellular function is one which can be restored by an effector molecule having anti-permeability effect of endothelial cells, an anti-vascular leakage effect, an anti-apoptotic effect of endothelial cells or an anti-inflammatory effect of endothelial cells or an anti-stress response effect, preferably the effect is linked to or associated with the Tie-2 signalling pathway, VEGF-receptor pathway, NOTCH signalling pathway, PI3-kinase pathway, eNOS signalling pathway, sirtuin-dependent metabolic and energy homeostasis pathway, oxidative stress pathway, shear stress response pathway, ET-1 signal transduction pathway, NO-mediated signal transduction pathway, and mechanochemical transduction pathway.

More specifically, the problem underlying the present invention is solved in a second aspect, which is also a first embodiment of the second aspect, by a recombinant nucleic acid construct comprising in 5′ -> 3′ direction
a 5′ non-translated region,
a coding region coding for an effector molecule, and
a 3′ non-translated region,
wherein the effector molecule is effective in restoring a cellular function of a cell and wherein the effector molecule is a mutant effector molecule, wherein the mutant effector molecule is a gain of function mutant effector molecule and/or a hypermorphic mutant effector molecule, for use in a method for the treatment and/or prevention of a disease, wherein the effector molecule is expressed by an endothelial cell. In an embodiment and as preferably used herein restoring a cellular function comprises and, respectively, encompasses killing of a cell, more preferably killing of tumor endothelial cells and/or tumor cells; in another embodiment and as preferably used herein, restoring cellular function comprises and, respectively, encompasses neoangiogenesis, more preferably neoangiogenesis in case of vascular diseases.

In a second embodiment of the second aspect which is also an embodiment of the first embodiment of the second aspect, the treatment and/or prevention of a disease involves restoration of the cellular function of a cell.

More specifically, the problem underlying the present invention is solved in a third aspect, which is also a first embodiment of the third aspect, by a recombinant nucleic acid construct comprising in 5′ -> 3′ direction
a 5′ non-translated region,
a coding region coding for an effector molecule, and
a 3′ non-translated region,
wherein the effector molecule is effective in restoring a cellular function of a cell and wherein the effector molecule is a mutant effector molecule, wherein the mutant effector molecule is a gain of function mutant effector molecule and/or a hypermorphic mutant effector molecule, for use in a method for restoring the cellular function of a cell, wherein the effector molecule is expressed by an endothelial cell.

More specifically, the problem underlying the present invention is solved in a fourth aspect, which is also a first embodiment of the fourth aspect, by a recombinant nucleic acid construct comprising in 5′ -> 3′ direction
a 5′ non -translated region,
a coding region coding for an effector molecule, and
a 3′ non-translated region,
wherein the effector molecule is effective in exercising a therapeutic effect in or on a cell and wherein the effector molecule is a mutant effector molecule, wherein the mutant effector molecule is a gain of function mutant effector molecule and/or a hypermorphic mutant effector molecule, for use in a method for the treatment and/or prevention of a disease, wherein the effector molecule is expressed by an endothelial cell.

In a second embodiment of the fourth aspect which is also an embodiment of the first embodiment of the fourth aspect, the treatment and/or prevention of a disease involves restoration of a cellular function of a cell.

More specifically, the problem underlying the present invention is solved in a fifth aspect, which is also a first embodiment of the fifth aspect, by a recombinant nucleic acid construct comprising in 5′ -> 3′ direction
a 5′ non -translated region,
a coding region coding for an effector molecule, and
a 3′ non-translated region,
wherein the effector molecule is effective in exercising a therapeutic effect in or on a cell and wherein the effector molecule is a mutant effector molecule, wherein the mutant effector molecule is a gain of function mutant effector molecule and/or a hypermorphic mutant effector molecule, for use of in a method for restoring a cellular function of a cell, wherein the effector molecule is expressed by an endothelial cell.

In a third embodiment of the second aspect which is also an embodiment of the first and second embodiment of the second aspect, in a second embodiment of the third aspect which is also an embodiment of the first embodiment of the third aspect, in a third embodiment of the fourth aspect which is also an embodiment of the first and second embodiment of the fourth aspect, and in a second embodiment of the fifth aspect which is also an embodiment of the first embodiment of the fifth aspect, the effector molecule is expressed in the method by an endothelial cell.

In a fourth embodiment of the second aspect which is also an embodiment of the first, second and third embodiment of the second aspect, in a third embodiment of the third aspect which is also an embodiment of the first and the second embodiment of the third aspect, in a fourth embodiment of the fourth aspect which is also an embodiment of the first, second and third embodiment of the fourth aspect, and in a third embodiment of the fifth aspect which is also an embodiment of the first and second embodiment of the fifth aspect, the endothelial cell is a vascular endothelial cell, preferably the vascular endothelial cell is a microvascular endothelial cell.

In a fifth embodiment of the second aspect which is also an embodiment of the first, second, third and fourth embodiment of the second aspect, in a fourth embodiment of the third aspect which is also an embodiment of the first, second and third embodiment of the third aspect, in a fifth embodiment of the fourth aspect which is also an embodiment of the first, second, third and fourth embodiment of the fourth aspect, and in a fourth embodiment of the fifth aspect which is also an embodiment of the first, second and third embodiment of the fifth aspect, the endothelial cell is a non-dividing endothelial cell, a non-proliferating endothelial cell or a resting endothelial cell. In an embodiment thereof and as preferably used herein the endothelial is a vascular endothelial cell, a lymphatic endothelial cells, an endothelial stem cell (ESC) or an endothelial progenitor cells (EPC).

In a sixth embodiment of the second aspect which is also an embodiment of the first, second, third, fourth and fifth embodiment of the second aspect, in a fifth embodiment of the third aspect which is also an embodiment of the first, second, third and fourth embodiment of the third aspect, in a sixth embodiment of the fourth aspect which is also an embodiment of the first, second, third, fourth and fifth embodiment of the fourth aspect, and in a fifth embodiment of the fifth aspect which is also an embodiment of the first, second, third and fourth embodiment of the fifth aspect, the endothelial cell is a senescent endothelial cell.

In a seventh embodiment of the second aspect which is also an embodiment of the first, second, third, fourth, fifth and sixth embodiment of the second aspect, in a sixth embodiment of the third aspect which is also an embodiment of the first, second, third, fourth and fifth embodiment of the third aspect, in a seventh embodiment of the fourth aspect which is also an embodiment of the first, second, third, fourth, fifth and sixth embodiment of the fourth aspect, and in a sixth embodiment of the fifth aspect which is also an embodiment of the first, second, third, fourth and fifth embodiment of the fifth aspect, the endothelial cell is impaired by age and/or showing stress-related defects.

In an eighth embodiment of the second aspect which is also an embodiment of the first, second, third, fourth, fifth, sixth and seventh embodiment of the second aspect, in a seventh embodiment of the third aspect which is also an embodiment of the first, second, third, fourth, fifth and sixth embodiment of the third aspect, in an eighth embodiment of the fourth aspect which is also an embodiment of the first, second, third, fourth, fifth, sixth and seventh embodiment of the fourth aspect, and in a seventh embodiment of the fifth aspect which is also an embodiment of the first, second, third, fourth, fifth and sixth embodiment of the fifth aspect, the recombinant nucleic acid construct additionally comprises one element selected from the group comprising a cap structure, an IRES sequence, a further start codon providing sequence, a nucleic acid sequence coding for a signal peptide and a poly-A tail.

In a ninth embodiment of the second aspect which is also an embodiment of the first, second, third, fourth, fifth, sixth, seventh and eighth embodiment of the second aspect, in an eighth embodiment of the third aspect which is also an embodiment of the first, second, third, fourth, fifth, sixth and seventh embodiment of the third aspect, in a ninth embodiment of the fourth aspect which is also an embodiment of the first, second, third, fourth, fifth, sixth, seventh and eighth embodiment of the fourth aspect, and in an eighth embodiment of the fifth aspect which is also an embodiment of the first, second, third, fourth, fifth, sixth and seventh embodiment of the fifth aspect,

wherein the recombinant nucleic acid construct is one selected from the group comprising in 5′ -> 3′ direction
a) - a 5′ non -translated region,
   a coding region coding for an effector molecule,
   a 3′ non-translated region, and
   a poly-A tail;
b) - a 5′ non -translated region,
   a further start codon providing sequence,
   a coding region coding for an effector molecule,
   a 3′ non-translated region, and
   a poly-A tail;
c) - a cap structure,
   a 5′ non -translated region,
   a coding region coding for an effector molecule, and
   a 3′ non-translated region;
d) - a cap structure,
   a 5′ non -translated region,
   a further start codon providing sequence,
   a coding region coding for an effector molecule, and
   a 3′ non-translated region;
e) - an IRES sequence,
   a 5′ non -translated region,
   a coding region coding for an effector molecule, and
   a 3′ non-translated region;
f) - an IRES sequence,
   a 5′ non -translated region,
   a further start codon providing sequence,
   a coding region coding for an effector molecule, and
   a 3′ non-translated region;
g) - a cap structure,
   a 5′ non -translated region,
   a coding region coding for an effector molecule,
   a 3′ non-translated region, and
   a poly-A tail;
h) - a cap structure,
   a 5′ non -translated region,
   a further start codon providing sequence,
   a coding region coding for an effector molecule,
   a 3′ non-translated region, and
   a poly-A tail;
i) - an IRES sequence,
   a 5′ non -translated region,
   a coding region coding for an effector molecule,
   a 3′ non-translated region, and
   a poly-A tail;
j) - an IRES sequence,
   a 5′ non -translated region,
   a further start codon providing sequence,
   a coding region coding for an effector molecule,
   a 3′ non-translated region, and
   a poly-A tail;
k) - a 5′ non -translated region,
   a nucleic acid sequence coding for a signal peptide,
   a coding region coding for an effector molecule,
   a 3′ non-translated region, and
   a poly-A tail;
l) - a 5′ non -translated region,
   a further start codon providing sequence,
   a nucleic acid sequence coding for a signal peptide,
   a coding region coding for an effector molecule,
   a 3′ non-translated region, and
   a poly-A tail;
m) - a cap structure,
   a 5′ non -translated region,
   a nucleic acid sequence coding for a signal peptide,
   a coding region coding for an effector molecule, and
   a 3′ non-translated region;
   a cap structure
   a 5′ non -translated region,
   a further start codon providing sequence,
   a nucleic acid sequence coding for a signal peptide,
   a coding region coding for an effector molecule, and
   a 3′ non-translated region;
o) - an IRES sequence,
   a 5′ non -translated region,
   a nucleic acid sequence coding for a signal peptide, - a coding region coding for an effector molecule, and
   a 3′ non-translated region;
p) - an IRES sequence,
   a 5′ non -translated region,
   a further start codon providing sequence,
   a nucleic acid sequence coding for a signal peptide,
   a coding region coding for an effector molecule, and
   a 3′ non-translated region;
q) - a cap structure,
   a 5′ non -translated region,
   a nucleic acid sequence coding for a signal peptide,
   a coding region coding for an effector molecule,
   a 3′ non-translated region, and
   a poly-A tail;
r) - a cap structure,
   a 5′ non -translated region,
   a further start codon providing sequence,
   a nucleic acid sequence coding for a signal peptide,
   a coding region coding for an effector molecule,
   a 3′ non-translated region, and
   a poly-A tail;
s) - an IRES sequence,
   a 5′ non -translated region,
   a nucleic acid sequence coding for a signal peptide,
   a coding region coding for an effector molecule,
   a 3′ non-translated region, and
   a poly-A tail; and
t) - an IRES sequence,
   a 5′ non -translated region,
   a further start codon providing sequence,
   a nucleic acid sequence coding for a signal peptide,
   a coding region coding for an effector molecule,
   a 3′ non-translated region, and
   a poly-A tail.

In a tenth embodiment of the second aspect which is also an embodiment of the first, second, third, fourth, fifth, sixth, seventh, eighth and ninth embodiment of the second aspect, in an ninth embodiment of the third aspect which is also an embodiment of the first, second, third, fourth, fifth, sixth, seventh and eighth embodiment of the third aspect, in a tenth embodiment of the fourth aspect which is also an embodiment of the first, second, third, fourth, fifth, sixth, seventh, eighth and ninth embodiment of the fourth aspect, and in an ninth embodiment of the fifth aspect which is also an embodiment of the first, second, third, fourth, fifth, sixth, seventh and eighth embodiment of the fifth aspect, the recombinant nucleic acid comprises in 5′ -> 3′ direction
a cap structure,
a 5′ non -translated region,
a nucleic acid sequence coding for a signal peptide,
a coding region coding for an effector molecule,
a 3′ non-translated region, and
a poly-A tail.

In an eleventh embodiment of the second aspect which is also an embodiment of the first, second, third, fourth, fifth, sixth, seventh, eighth, ninth and tenth embodiment of the second aspect, in a tenth embodiment of the third aspect which is also an embodiment of the first, second, third, fourth, fifth, sixth, seventh, eighth and ninth embodiment of the third aspect, in an eleventh embodiment of the fourth aspect which is also an embodiment of the first, second, third, fourth, fifth, sixth, seventh, eighth, ninth and tenth embodiment of the fourth aspect, and in a tenth embodiment of the fifth aspect which is also an embodiment of the first, second, third, fourth, fifth, sixth, seventh, eighth and ninth embodiment of the fifth aspect, the recombinant nucleic acid comprises in 5′ -> 3′ direction
an IRES sequence,
a 5′ non -translated region,
a nucleic acid sequence coding for a signal peptide,
a coding region coding for an effector molecule,
a 3′ non-translated region, and
a poly-A tail.

In a 12th embodiment of the second aspect which is also an embodiment of the first, second, third, fourth, fifth, sixth, seventh, eighth, ninth, tenth and eleventh embodiment of the second aspect, in an eleventh embodiment of the third aspect which is also an embodiment of the first, second, third, fourth, fifth, sixth, seventh, eighth, ninth and tenth embodiment of the third aspect, in a 12^{th} embodiment of the fourth aspect which is also an embodiment of the first, second, third, fourth, fifth, sixth, seventh, eighth, ninth, tenth and eleventh embodiment of the fourth aspect, and in an eleventh embodiment of the fifth aspect which is also an embodiment of the first, second, third, fourth, fifth, sixth, seventh, eighth, ninth and tenth embodiment of the fifth aspect, recombinant nucleic acid comprises in 5′ -> 3′ direction
a cap structure,
a 5′ non -translated region,
a further start codon providing sequence,
a nucleic acid sequence coding for a signal peptide,
a coding region coding for an effector molecule,
a 3′ non-translated region, and
a poly-A tail.

In a 13^{th} embodiment of the second aspect which is also an embodiment of the first, second, third, fourth, fifth, sixth, seventh, eighth, ninth, tenth, eleventh and 12^{th} embodiment of the second aspect, in a 12^{th} embodiment of the third aspect which is also an embodiment of the first, second, third, fourth, fifth, sixth, seventh, eighth, ninth, tenth and eleventh embodiment of the third aspect, in a 13^{th} embodiment of the fourth aspect which is also an embodiment of the first, second, third, fourth, fifth, sixth, seventh, eighth, ninth, tenth, eleventh and 12^{th} embodiment of the fourth aspect, and in an 12^{th} embodiment of the fifth aspect which is also an embodiment of the first, second, third, fourth, fifth, sixth, seventh, eighth, ninth, tenth and eleventh embodiment of the fifth aspect, the recombinant nucleic acid comprises in 5′ -> 3′ direction
an IRES sequence,
a 5′ non -translated region,
a further start codon providing sequence,
a nucleic acid sequence coding for a signal peptide,
a coding region coding for an effector molecule,
a 3′ non-translated region, and
a poly-A tail.

In a 14^{th} embodiment of the second aspect which is also an embodiment of the first, second, third, fourth, fifth, sixth, seventh, eighth, ninth, tenth, eleventh, 12^{th} and 13^{th} embodiment of the second aspect, in a 13^{th} embodiment of the third aspect which is also an embodiment of the first, second, third, fourth, fifth, sixth, seventh, eighth, ninth, tenth, eleventh and 12^{th} embodiment of the third aspect, in a 14^{th} embodiment of the fourth aspect which is also an embodiment of the first, second, third, fourth, fifth, sixth, seventh, eighth, ninth, tenth, eleventh, 12^{th} and 13^{th} embodiment of the fourth aspect, and in an 13^{th} embodiment of the fifth aspect which is also an embodiment of the first, second, third, fourth, fifth, sixth, seventh, eighth, ninth, tenth, eleventh and 12^{th} embodiment of the fifth aspect, the further start codon providing sequence is in-frame with the coding region coding for an effector molecule.

In a 15^{th} embodiment of the second aspect which is also an embodiment of the first, second, third, fourth, fifth, sixth, seventh, eighth, ninth, tenth, eleventh, 12^{th}, 13^{th} and 14^{th} embodiment of the second aspect, in a 14^{th} embodiment of the third aspect which is also an embodiment of the first, second, third, fourth, fifth, sixth, seventh, eighth, ninth, tenth, eleventh, 12^{th} and 13^{th} embodiment of the third aspect, in a 15^{th} embodiment of the fourth aspect which is also an embodiment of the first, second, third, fourth, fifth, sixth, seventh, eighth, ninth, tenth, eleventh, 12^{th}, 13^{th} and 14^{th} embodiment of the fourth aspect, and in an 14^{th} embodiment of the fifth aspect which is also an embodiment of the first, second, third, fourth, fifth, sixth, seventh, eighth, ninth, tenth, eleventh 12^{th} and 13^{th} embodiment of the fifth aspect, the 5′ non -translated region is a 5′ UTR.

In a 16^{th} embodiment of the second aspect which is also an embodiment of the first, second, third, fourth, fifth, sixth, seventh, eighth, ninth, tenth, eleventh, 12^{th}, 13^{th}, 14^{th} and 15^{th} embodiment of the second aspect, in a 15^{th} embodiment of the third aspect which is also an embodiment of the first, second, third, fourth, fifth, sixth, seventh, eighth, ninth, tenth, eleventh, 12^{th}, 13^{th} and 14^{th} embodiment of the third aspect, in a 16^{th} embodiment of the fourth aspect which is also an embodiment of the first, second, third, fourth, fifth, sixth, seventh, eighth, ninth, tenth, eleventh, 12^{th}, 13^{th}, 14^{th} and 15^{th} embodiment of the fourth aspect, and in an 15^{th} embodiment of the fifth aspect which is also an embodiment of the first, second, third, fourth, fifth, sixth, seventh, eighth, ninth, tenth, eleventh 12^{th}, 13^{th} and 14^{th} embodiment of the fifth aspect, the 5′ non-translated region is from a eukaryotic organism, preferably a mammalian organism, more preferably a mammalian organism selected from the group comprising human, non-human primate such as cynomolgus, chimpanzee rhesus monkey, rat and mouse. In an alternative embodiment the 5′ non-translated region is from a viral gene, preferably the virus is a virus capable of infecting a eukaryotic organism.

In a 17^{th} embodiment of the second aspect which is also an embodiment of the first, second, third, fourth, fifth, sixth, seventh, eighth, ninth, tenth, eleventh, 12^{th}, 13^{th}, 14^{th}, 15^{th} and 16^{th} embodiment of the second aspect, in a 16^{th} embodiment of the third aspect which is also an embodiment of the first, second, third, fourth, fifth, sixth, seventh, eighth, ninth, tenth, eleventh, 12^{th}, 13^{th}, 14^{th} and 15^{th} embodiment of the third aspect, in a 17^{th} embodiment of the fourth aspect which is also an embodiment of the first, second, third, fourth, fifth, sixth, seventh, eighth, ninth, tenth, eleventh, 12^{th}, 13^{th}, 14^{th}, 15^{th} and 16^{th} embodiment of the fourth aspect, and in an 16^{th} embodiment of the fifth aspect which is also an embodiment of the first, second, third, fourth, fifth, sixth, seventh, eighth, ninth, tenth, eleventh 12^{th}, 13^{th}, 14^{th} and 15^{th} embodiment of the fifth aspect, the 5′ UTR is selected from the group comprising a 5′ UTR of a gene coding for MCP-1 or a derivative thereof having a nucleotide identity of at least 85%, a 5′ UTR of a gene coding for RPL12s.c. or a derivative thereof having a nucleotide identity of at least 85%, a 5′ UTR of a gene coding for Ang-2 or a derivative thereof having a nucleotide identity of at least 85%, a 5′ UTR of a gene coding for HSP70 or a derivative thereof having a nucleotide identity of at least 85%, a 5′ UTR of a gene coding for H3.3. or a derivative thereof having a nucleotide identity of at least 85%, and a 5′ UTR of a gene coding for Galectin-9 or a derivative thereof having a nucleotide identity of at least 85%.

In an 18^{th} embodiment of the second aspect which is also an embodiment of the first, second, third, fourth, fifth, sixth, seventh, eighth, ninth, tenth, eleventh, 12^{th}, 13^{th}, 14^{th}, 15^{th}, 16^{th} and 17^{th} embodiment of the second aspect, in a 17^{th} embodiment of the third aspect which is also an embodiment of the first, second, third, fourth, fifth, sixth, seventh, eighth, ninth, tenth, eleventh, 12^{th}, 13^{th}, 14^{th}, 15^{th} and 16^{th} embodiment of the third aspect, in an 18^{th} embodiment of the fourth aspect which is also an embodiment of the first, second, third, fourth, fifth, sixth, seventh, eighth, ninth, tenth, eleventh, 12^{th}, 13^{th}, 14^{th}, 15^{th}, 16^{th} and 17^{th} embodiment of the fourth aspect, and in an 17^{th} embodiment of the fifth aspect which is also an embodiment of the first, second, third, fourth, fifth, sixth, seventh, eighth, ninth, tenth, eleventh 12^{th}, 13^{th}, 14^{th} and 15^{th}, 16^{th} embodiment of the fifth aspect, the 3′ non-translated region is a 3′-UTR.

In a 19^{th} embodiment of the second aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th} and 18^{th} embodiment of the second aspect, in an 18^{th} embodiment of the third aspect which is also an embodiment of the first, second, third, fourth, fifth, sixth, seventh, eighth, ninth, tenth, eleventh, 12^{th}, 13^{th}, 14^{th}, 15^{th}, 16^{th} and 17^{th} embodiment of the third aspect, in a 19^{th} embodiment of the fourth aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th} and 18^{th} embodiment of the fourth aspect, and in an 18^{th} embodiment of the fifth aspect which is also an embodiment of the first, second, third, fourth, fifth, sixth, seventh, eighth, ninth, tenth, eleventh 12^{th}, 13^{th}, 14^{th} and 15^{th}, 16^{th}, 17^{th} embodiment of the fifth aspect, the 3′ non-translated region is from a eukaryotic organism, preferably a mammalian organism, more preferably a mammalian organism selected from the group comprising human, non-human primate such as cynomolgus, chimpanzee rhesus monkey, rat and mouse. In an alternative embodiment the 5′ non-translated region is from a viral gene, preferably the virus is a virus capable of infecting a eukaryotic organism.

In a 20^{th} embodiment of the second aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th} and 19^{th} embodiment of the second aspect, in a 19^{th} embodiment of the third aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th} and 18^{th} embodiment of the third aspect, in a 20^{th} embodiment of the fourth aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th} and 19^{th} embodiment of the fourth aspect, and in a 19^{th} embodiment of the fifth aspect which is also an embodiment of the first, second, third, fourth, fifth, sixth, seventh, eighth, ninth, tenth, eleventh 12^{th}, 13^{th}, 14^{th}, 15^{th}, 16^{th}, 17^{th} and 18^{th} embodiment of the fifth aspect, the 3′ UTR is selected from the group comprising a 3′ UTR of a gene coding for vWF or a derivative thereof having a nucleotide identity of at least 85%, a 3′ UTR of a gene coding for MCP-1 or a derivative thereof having a nucleotide identity of at least 85%, a 3′ UTR of a gene coding for RPL12s.c. or a derivative thereof having a nucleotide identity of at least 85%, a 3′ UTR of a gene coding for HSP70 or a derivative thereof having a nucleotide identity of at least 85%, and a 3′ UTR of a gene coding for H3.3. or a derivative thereof having a nucleotide identity of at least 85%.

In a 21^{st} embodiment of the second aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th} and 20^{th} embodiment of the second aspect, in a 20^{th} embodiment of the third aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th} and 19^{th} embodiment of the third aspect, in a 21^{st} embodiment of the fourth aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th} 19^{th} and 20^{th} embodiment of the fourth aspect, and in a 20^{th} embodiment of the fifth aspect which is also an embodiment of the first, second, third, fourth, fifth, sixth, seventh, eighth, ninth, tenth, eleventh 12^{th}, 13^{th}, 14^{th} 15^{th}, 16^{th}, 17^{th}, 18^{th} and 19^{th} embodiment of the fifth aspect, the 5′ UTR and 3′ UTR of the recombinant nucleic acid construct are of different origin, preferably are from different species.

In a 22^{nd} embodiment of the second aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th} and 21^{st} embodiment of the second aspect, in a 21^{st} embodiment of the third aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th} and 20^{th} embodiment of the third aspect, in a 22^{nd} embodiment of the fourth aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th} 19^{th}, 20^{th} and 21^{st} embodiment of the fourth aspect, and in a 21^{St} embodiment of the fifth aspect which is also an embodiment of the first, second, third, fourth, fifth, sixth, seventh, eighth, ninth, tenth, eleventh 12^{th}, 13^{th}, 14^{th} 15^{th}, 16^{th}, 17^{th}, 18^{th}, 19^{th} and 20^{th} embodiment of the fifth aspect, the 5′ UTR and 3′ UTR of the recombinant nucleic acid construct are of the same origin, preferably are from the same species.

In a 23^{rd} embodiment of the second aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}and 22^{nd}embodiment of the second aspect, in a 22^{nd} embodiment of the third aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th} 20^{th} and 21^{st} embodiment of the third aspect, in a 23^{rd} embodiment of the fourth aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th} 19^{th} and 20^{th}, 21^{st} and 22^{nd} embodiment of the fourth aspect, and in a 22^{nd} embodiment of the fifth aspect which is also an embodiment of the first, second, third, fourth, fifth, sixth, seventh, eighth, ninth, tenth, eleventh 12^{th}, 13^{th}, 14^{th} 15^{th}, 16^{th}, 17^{th}, 18^{th}, 19^{th}, 20^{th} and 21^{st} embodiment of the fifth aspect,
a) the 5′ UTR is a 5′ UTR of a gene coding for MCP-1 or a derivative thereof having a nucleotide identity of at least 85%, and wherein the 3′ UTR is selected from the group comprising a 3′ UTR of a gene coding for vWF or a derivative thereof having a nucleotide identity of at least 85%, a 3′ UTR of a gene coding for HSP70 or a derivative thereof having a nucleotide identity of at least 85%, a 3′ UTR of a gene coding for RPL12s.c. or a derivative thereof having a nucleotide identity of at least 85%, and a 3′ UTR of a gene coding for MCP-1 or a derivative thereof having a nucleotide identity of at least 85%.
b) the 5′ UTR is a 5′ UTR of a gene coding for RPL12s.c. or a derivative thereof having a nucleotide identity of at least 85%, and wherein the 3′ UTR is selected from the group comprising a 3′ UTR of a gene coding for vWF or a derivative thereof having a nucleotide identity of at least 85%, a 3′ UTR of a gene coding for HSP70 or a derivative thereof having a nucleotide identity of at least 85%, a 3′ UTR of a gene coding for RPL12s.c. or a derivative thereof having a nucleotide identity of at least 85%, and a 3′ UTR of a gene coding for MCP-1 or a derivative thereof having a nucleotide identity of at least 85%,
c) the 5′ UTR is a 5′ UTR of a gene coding for HSP70 or a derivative thereof having a nucleotide identity of at least 85%, and wherein the 3′ UTR is selected from the group comprising a 3′ UTR of a gene coding for HSP70 or a derivative thereof having a nucleotide identity of at least 85%, a 3′ UTR of a gene coding for RPL12s. c. or a derivative thereof having a nucleotide identity of at least 85%, a 3′ UTR of a gene coding for vWF or a derivative thereof having a nucleotide identity of at least 85% and a 3′ UTR of a gene coding for MCP-1 or a derivative thereof having a nucleotide identity of at least 85%, or
d) the 5′ UTR is a 5′ UTR of a gene coding for ANG-2 or a derivative thereof having a nucleotide identity of at least 85%, and wherein the 3′ UTR is selected from the group comprising a 3′ UTR of a gene coding for HSP70 or a derivative thereof having a nucleotide identity of at least 85%, a 3′ UTR of a gene coding for RPL12s. c. or a derivative thereof having a nucleotide identity of at least 85%, a 3′ UTR of a gene coding for vWF or a derivative thereof having a nucleotide identity of at least 85% and a 3′ UTR of a gene coding for MCP-1 or a derivative thereof having a nucleotide identity of at least 85%.

In a 24^{th} embodiment of the second aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd} and 23^{rd} embodiment of the second aspect, in a 23^{rd} embodiment of the third aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st} and 22^{nd} embodiment of the third aspect, in a 24^{th} embodiment of the fourth aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd} and 23^{rd} embodiment of the fourth aspect, and in a 23^{rd} embodiment of the fifth aspect which is also an embodiment of the first, second, third, fourth, fifth, sixth, seventh, eighth, ninth, tenth, eleventh 12^{th}, 13^{th}, 14^{th} 15^{th}, 16^{th}, 17^{th}, 18^{th}, 19^{th}, 20^{th}, 21^{st} and 22^{nd} embodiment of the fifth aspect,
a) 3′UTR is a 3′ UTR of a gene coding for vWF or a derivative thereof having a nucleotide identity of at least 85% and the 5′ UTR is selected from the group comprising a 5′ UTR of a gene coding for MCP-1 or a derivative thereof having a nucleotide identity of at least 85%, a 5′ UTR of a gene coding for RPL12s.c. or a derivative thereof having a nucleotide identity of at least 85%, a 5′ UTR of a gene coding for HSP70 or a derivative thereof having a nucleotide identity of at least 85 %, and a 5′ UTR of a gene coding for ANG-2 of a derivative thereof having a nucleotide identity of at least 85%, and,
b) 3′UTR is a 3′ UTR of a gene coding for HSP70 or a derivative thereof having a nucleotide identity of at least 85% and the 5′ UTR is selected from the group comprising a 5′ UTR of a gene coding for HSP70 or a derivative thereof having a nucleotide identity of at least 85% a 5′ UTR of a gene coding for RPL12s.c. or a derivative thereof having a nucleotide identity of at least 85%, a 5′ UTR of a gene coding for ANG-2 of a derivative thereof having a nucleotide identity of at least 85%, and a 5′ UTR of a gene coding for MCP-1 or a derivative thereof having a nucleotide identity of at least 85%, c) 3′UTR is a 3′ UTR of a gene coding for RPL12s.c. or a derivative thereof having a nucleotide identity of at least 85% and the 5′ UTR is selected from the group comprising a 5′ UTR of a gene coding for HSP70 or a derivative thereof having a nucleotide identity of at least 85% a 5′ UTR of a gene coding for RPL12s.c. or a derivative thereof having a nucleotide identity of at least 85%, a 5′ UTR of a gene coding for ANG-2 of a derivative thereof having a nucleotide identity of at least 85%, and a 5′ UTR of a gene coding for MCP-1 or a derivative thereof having a nucleotide identity of at least 85%, or
d) 3′UTR is a 3′ UTR of a gene coding for MCP-1 or a derivative thereof having a nucleotide identity of at least 85% and the 5′ UTR is selected from the group comprising a 5′ UTR of a gene coding for HSP70 or a derivative thereof having a nucleotide identity of at least 85%, a 5′ UTR of a gene coding for RPL12s.c. or a derivative thereof having a nucleotide identity of at least 85%, a 5′ UTR of a gene coding for ANG-2 of a derivative thereof having a nucleotide identity of at least 85%, and a 5′ UTR of a gene coding for MCP-1 or a derivative thereof having a nucleotide identity of at least 85%.

In a 25^{th} embodiment of the second aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd}, 23^{rd} and 24^{th} embodiment of the second aspect, in a 24^{th} embodiment of the third aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}; 21^{st}, 22^{nd} and 23^{rd} embodiment of the third aspect, in a 25^{th} embodiment of the fourth aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd}, 23^{rd} and 24^{th} embodiment of the fourth aspect, and in a 24th embodiment of the fifth aspect which is also an embodiment of the first, second, third, fourth, fifth, sixth, seventh, eighth, ninth, tenth, eleventh 12^{th}, 13^{th}, 14^{th} 15^{th}, 16^{th}, 17^{th}, 18^{th}, 19^{th}, 20^{th}, 21^{st}, 22^{nd} and 23^{rd} embodiment of the fifth aspect, the construct is one selected from the group comprising
a construct, wherein the 5′ UTR is a 5′ UTR of a gene coding for MCP-1 or a derivative thereof having a nucleotide identity of at least 85%, and the 3′ UTR is a 3′ UTR of a gene coding for vWF or a derivative thereof having a nucleotide identity of at least 85%,
a construct, wherein the 5′ UTR is a 5′ UTR of a gene coding for RPL12s.c. or a derivative thereof having a nucleotide identity of at least 85%, and the 3′ UTR is a 3′ UTR of a gene coding for vWF or a derivative thereof having a nucleotide identity of at least 85%,
a construct, wherein the 5′ UTR is a 5′ UTR of a gene coding for MCP-1 or a derivative thereof having a nucleotide identity of at least 85%, and the 3′ UTR is a 3′ UTR of a gene coding for MCP-1 or a derivative thereof having a nucleotide identity of at least 85%,
a construct, wherein the 5′ UTR is a 5′ UTR of a gene coding for ANG-2 or a derivative thereof having a nucleotide identity of at least 85%, and the 3′ UTR is a 3′ UTR of a gene coding for vWF or a derivative thereof having a nucleotide identity of at least 85%,
a construct, wherein the 5′ UTR is a 5′ UTR of a gene coding for RPL12s.c. or a derivative thereof having a nucleotide identity of at least 85%, and the 3′ UTR is a 3′ UTR of a gene coding for RPL12s.c. or a derivative thereof having a nucleotide identity of at least 85%,
a construct, wherein the 5′ UTR is a 5′ UTR of a gene coding for HSP70 or a derivative thereof having a nucleotide identity of at least 85%, and the 3′ UTR is a 3′ UTR of a gene coding for HSP70 or a derivative thereof having a nucleotide identity of at least 85 %,
a construct, wherein the 5′ UTR is a 5′ UTR of a gene coding for H3.3. or a derivative thereof having a nucleotide identity of at least 85%, and the 3′ UTR is a 3′ UTR of a gene coding for H3.3. or a derivative thereof having a nucleotide identity of at least 85%, or
a construct, wherein the 5′ UTR is a 5′ UTR of a gene coding for Galectin 9 or a derivative thereof having a nucleotide identity of at least 85%, and the 3′ UTR is a 3′ UTR of a gene coding for Galectin 9 or a derivative thereof having a nucleotide identity of at least 85%.

In a 26^{th} embodiment of the second aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th} and 25^{th} embodiment of the second aspect, in a 25^{th} embodiment of the third aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd}, 23^{rd} and 24^{th} embodiment of the third aspect, in a 26^{th} embodiment of the fourth aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd}, and 23^{rd}, 24^{th} and 25^{th} embodiment of the fourth aspect, and in a 25^{th} embodiment of the fifth aspect which is also an embodiment of the first, second, third, fourth, fifth, sixth, seventh, eighth, ninth, tenth, eleventh 12^{th}, 13^{th}, 14^{th} 15^{th}, 16^{th}, 17^{th}, 18^{th}, 19^{th}, 20^{th}, 21^{st}, 22^{nd}, 23^{rd} and 24^{th} embodiment of the fifth aspect, the construct comprises a poly-A tail.

In a 27^{th} embodiment of the second aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th} and 26^{th} embodiment of the second aspect, in a 26^{th} embodiment of the third aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th} and 25^{th} embodiment of the third aspect, in a 27^{th} embodiment of the fourth aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th} and 26^{th} embodiment of the fourth aspect, and in a 26^{th} embodiment of the fifth aspect which is also an embodiment of the first, second, third, fourth, fifth, sixth, seventh, eighth, ninth, tenth, eleventh 12^{th}, 13^{th}, 14^{th} 15^{th}, 16^{th}, 17^{th}, 18^{th}, 19^{th}, 20^{th}, 21^{st}, 22^{nd}, 23^{rd}, 24^{th} and 25^{th} embodiment of the fifth aspect, the construct comprises CAP structure.

In a 28^{th} embodiment of the second aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd} and 23^{rd}, 24^{th}, 25^{th}, 26^{th} and 27^{th} embodiment of the second aspect, in a 27^{th} embodiment of the third aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th} and 26^{th} embodiment of the third aspect, in a 28th embodiment of the fourth aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{St}, 22^{nd}, 23^{rd}, 24^{th}, 25^{th}, 26^{th} and 27^{th} embodiment of the fourth aspect, and in a 27th embodiment of the fifth aspect which is also an embodiment of the first, second, third, fourth, fifth, sixth, seventh, eighth, ninth, tenth, eleventh 12^{th}, 13^{th}, 14^{th} 15^{th}, 16^{th}, 17^{th}, 18^{th}, 19^{th}, 20^{th}, 21^{st}, 22^{nd}, 23^{rd}, 24^{th}, 25^{th} and 26^{th} embodiment of the fifth aspect, the construct comprises one or more IRESs (internal ribosomal entry sites) sequences. In a preferred embodiment, the IRES sequences replaced the Cap structure.

In a 29^{th} embodiment of the second aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd} and 23^{rd}, 24^{th}, 25^{th}, 26^{th}, 27^{th} and 28^{th} embodiment of the second aspect, in a 28^{th} embodiment of the third aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th} and 27^{th} embodiment of the third aspect, in a 29^{th} embodiment of the fourth aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th} and 28^{th} embodiment of the fourth aspect, and in a 28^{th} embodiment of the fifth aspect which is also an embodiment of the first, second, third, fourth, fifth, sixth, seventh, eighth, ninth, tenth, eleventh 12^{th}, 13^{th}, 14^{th} 15^{th}, 16^{th}, 17^{th}, 18^{th}, 19^{th}, 20^{th}, 21^{st}, 22^{nd}, 23^{rd}, 24^{th}, 25^{th}, 26^{th} and 27^{th} embodiment of the fifth aspect, the construct comprises nucleic acid sequence coding for a signal peptide.

In a 30^{th} embodiment of the second aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd} and 23^{rd}, 24^{th}, 25^{th}, 26^{th}, 27^{th}, 28^{th} and 29^{th} embodiment of the second aspect, in a 29^{th} embodiment of the third aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27tz and 28^{th} embodiment of the third aspect, in a 30^{th} embodiment of the fourth aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th} 28^{th} and 29^{th} embodiment of the fourth aspect, and in a 29^{th} embodiment of the fifth aspect which is also an embodiment of the first, second, third, fourth, fifth, sixth, seventh, eighth, ninth, tenth, eleventh 12^{th}, 13^{th}, 14^{th} 15^{th}, 16^{th}, 17^{th}, 18^{th}, 19^{th}, 20^{th}, 21^{st}, 22^{nd}, 23^{rd}, 24^{th}, 25^{th} 26^{th}, 27^{th} and 28^{th} embodiment of the fifth aspect, the signal peptide allows secretion of the effector molecule.

In a 31^{st} embodiment of the second aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd} and 23^{rd}, 24^{th}, 25^{th}, 26^{th}, 27^{th}, 28^{th}, 29^{th} and 30^{th} embodiment of the second aspect, in a 30^{th} embodiment of the third aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th} and 29^{th} embodiment of the third aspect, in a 31^{st} embodiment of the fourth aspect which is also an embodiment of the first, second, third, fourth, fifth, sixth, seventh, eighth, ninth, tenth, eleventh, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th} and 30^{th} embodiment of the fourth aspect, and in a 30^{th} embodiment of the fifth aspect which is also an embodiment of the first, second, third, fourth, fifth, sixth, seventh, eighth, ninth, tenth, eleventh 12^{th}, 13^{th}, 14^{th} 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th} and 29^{th} embodiment of the fifth aspect, the nucleotide sequence coding for a signal peptide is selected from the group comprising a nucleotide sequence coding for a signal peptide of MCP-1 or a derivative thereof having a nucleotide identity of at least 85%, nucleotide sequence coding for a signal peptide of IL-6 or a derivative thereof having a nucleotide identity of at least 85%,, a nucleotide sequence coding for a signal peptide of Ang-2 or a derivative thereof having a nucleotide identity of at least 85%, and a nucleotide sequence coding for a signal peptide of Ang-1 or a derivative thereof having a nucleotide identity of at least 85%.

In an embodiment of the first, second, third, fourth and fifth aspect, including any embodiment thereof, the cell a cellular function of which is restored and/or the cell in or on which a therapeutic effect is exercised is an endothelial cell, preferably a vascular endothelial cell, more preferably the vascular endothelial cell is a microvascular endothelial cell.

In an embodiment of the first, second, third, fourth and fifth aspect, including any embodiment thereof, the endothelial cell is a non-dividing endothelial cell, a non-proliferating endothelial cell or a resting endothelial cell.

In an embodiment of the first, second, third, fourth and fifth aspect, including any embodiment thereof, the endothelial cell is an endothelial cell stimulated by an inflammatory stimulus.

In an embodiment of the first, second, third, fourth and fifth aspect, including any embodiment thereof, the endothelial cell is a senescent endothelial cell.

In an embodiment of the first, second, third, fourth and fifth aspect, including any embodiment thereof, the endothelial cell is impaired by age and/or showing stress-related defects.

In an embodiment of the first, second, third, fourth and fifth aspect, including any embodiment thereof, the cellular function is one which can be restored by an effector molecule having anti-permeability effect of endothelial cells, an anti-vascular leakage effect, an anti-apoptotic effect of endothelial cells or an anti-inflammatory effect of endothelial cells or an anti-stress response effect. In an embodiment thereof, the effector molecule stimulates vessel survival, inhibits regression, inhibits apoptosis, stimulates migration, stimulates remodelling, stimulates angiogenesis, stimulates tube-formation/invasion, stimulates proliferation, inhibits leucocyte adhesion, inhibits adhesion molecule expression, inhibits tissue factor expression, inhibits NFKappaB activity or promotes monolayer integrity.

In an embodiment of the first, second, third, fourth and fifth aspect, including any embodiment thereof, the effect is linked to or associated with the Tie-2 signalling pathway, VEGF-receptor pathway, NOTCH signalling pathway, PI3-kinase pathway, eNOS signalling pathway, sirtuin-dependent metabolic and energy homeostasis pathway, oxidative stress pathway, shear stress response pathway, ET-1 signal transduction pathway, NO-mediated signal transduction pathway, and mechanochemical transduction pathway, preferably the pathway is selected from the group comprising Ang/Tie-2 signalling pathway, VEGF/VEGF receptor pathway and Notch/Notch ligand pathway.

In an embodiment of the first, second, third, fourth and fifth aspect, including any embodiment thereof, the effector molecule is an element of a pathway, wherein the pathway is selected from the group comprising the Tie-2 signalling pathway, VEGF-Receptor pathway, NOTCH signalling pathway, PI3-kinase pathway, eNOS signalling pathway, sirtuin-dependent metabolic and energy homeostasis pathway, oxidative stress pathway, shear stress response pathway ET-1 signal transduction pathway, NO-mediated signal transduction pathway, and mechanochemical transduction pathway.

In an embodiment of the first, second, third, fourth and fifth aspect, including any embodiment thereof, the effector molecule is selected from the group comprising Ang-1, Ang-4, COMP-Ang-1, hCOMP-Ang-1, COMP-Ang-1, COMP-ANG-2, Tie-2 receptor, Tie-1 receptor, PI3-kinase, preferably constitutive active PI3-kinase, hyperactive Tie-2 receptor (e.g. R849W), VE-cadherin, GRB2, GRB7, GRB14, GRB7, IQGAP1, RAC1, RAP1, DOK2, ABIN1 and ABIN2, KLF2 (Krueppel-like factor 2), alpha5 beta1 integrin, CD73, Akt 1, Akt 2 and Akt 3.

In an embodiment of the first, second, third, fourth and fifth aspect, including any embodiment thereof, the cellular function is vascular leakage and, preferably, the pathway is the Tie-2 pathway.

In an embodiment of the first, second, third, fourth and fifth aspect, including any embodiment thereof, the effector molecule is selected from the group comprising VEGF, VEGF-A, VEGF-B, PDGF, bFGF, Sirtuin, eNOS, RAS and c-MYC.

In an embodiment of the first, second, third, fourth and fifth aspect, including any embodiment thereof, wherein the effector molecule restores vascular regeneration or provides for vascular regeneration, preferably vascular endothelial vascular regeneration

In an embodiment of the first, second, third, fourth and fifth aspect, including any embodiment thereof, the cell a cellular function of which is restored and/or the cell in or on which a therapeutic effect is exercised is a non-endothelial cell.

In an embodiment of the first, second, third, fourth and fifth aspect, including any embodiment thereof, the non-endothelial cell is selected from the group comprising epithelial cells, alveolar macrophages, Alveolar type I cells, alveolar type II cells, alveolar macrophages, Pneumocytes, lung epithelial cells, hematopoietic cells, bone marrow cells, bone cells, stem cells, mesenchymal cells, neural cells, glia cells, neuron cells, Astrocytes, cells of the peripheral nervous system, cardiac cells, adipocytes, vascular smooth muscle cells, cardiomyocytes, skeletal muscle cells, beta cells, pituitary cells, synovial lining cells, ovarian cells, testicular cells, B cells, T cells, reticulocytes, leukocytes, granulocytes, macrophages, neutrophils, antigen presenting cells (dendritic cells), fibroblasts, hepatocytes, tumor cells and combination thereof.

In an embodiment of the first, second, third, fourth and fifth aspect, including any embodiment thereof, the effector molecule is selected from the group comprising a tumor death ligand, a factor involved in hematopoiesis and blood clotting, a stem cell factor, a growth factor and a cytokine.

In an embodiment of the first, second, third, fourth and fifth aspect, including any embodiment thereof, the tumor death ligand is selected from the group comprising IL-12, members of the TNF gene superfamily of death ligand proteins such as Apo2L/TRAIL, IL-13, IL-10, IL-8, IL-2, interferon beta, secreted frizzled-related protein (SFRP) 1, SFRP 2, SFRP 3, SFRP 4 and SFRP5.

In an embodiment of the first, second, third, fourth and fifth aspect, including any embodiment thereof, wherein the factor involved in hematopoiesis and blood clotting is selected from the group comprising erythropoietin, Factor VII, Factor VIII, Factor IX and heparan-N-sulfatase.

In an embodiment of the first, second, third, fourth and fifth aspect, including any embodiment thereof, the stem cell factor is selected from the group comprising Oct4, Sox2, Klf4 and c-Myc.

In an embodiment of the first, second, third, fourth and fifth aspect, including any embodiment thereof, the growth factor and cytokine is selected from the group comprising adrenomedullin, angiopoietin family, autocrine motility factor, bone morphogenetic proteins, ciliary neurotrophic factor (CNTF), leukemia inhibitory factor (LIF), interleukin-6 (IL-6), macrophage colony-stimulating factor (m-CSF), granulocyte colony-stimulating factor (G-CSF), granulocyte macrophage colony-stimulating factor (GM-CSF), epidermal growth fac-tor (EGF), ephrins, erythropoietin (EPO), fibroblast growth factor family (FGF1-23), somatotropin, GDNF family of ligands (such as glial cell line-derived neurotrophic factor (GDNF), neurturin, persephin, and artemin), growth differentiation factor-9 (GDF9), hepatocyte growth factor (HGF), hepatoma-derived growth factor (HDGF), insulin, insulin-like growth factors -1 and-2 (IGF-1; IGF-2), keratinocyte growth factor (KGF), migration-stimulating factor (MSF), macrophage-stimulating protein (MSP), also known as hepatocyte growth factor-like protein (HGFLP), myostatin (GDF-8), neuregulins 1-4 (NRG1-4), neurotrophins (brain-derived neurotrophic factor (BDNF), nerve growth factor (NGF), neurotrophin-3 (NT-3), neurotrophin-4 (NT-4)), placental growth factor (PGF), platelet-derived growth factor (PDGF), renalase (RNLS) - anti-apoptotic survival factor, T-cell growth factor (TCGF), thrombopoietin (TPO), transforming growth factor alpha (TGF-α), transforming growth factor beta (TGF-β), tumor necrosis factor-alpha (TNF-α), vascular endothelial growth factor family (VEGF), Wnt Signaling Pathway signaling glycoproteins (WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11, WNT16), granulocyte colony-stimulating factor (G-CSF), granulocyte macrophage colony-stimulating factor (GM-CSF), interferon- alfa, interferon-beta, interleukin 2 (IL-2), Interleukin 11 (IL-11), Interferon-gamma, IL-1, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23,IL-24, IL-25, IL-26, IL-27, IL-28, IL-29, IL-30, IL-31, IL-32, IL-33, IL-35 and IL-36.

In an embodiment of the first, second, third, fourth and fifth aspect, including any embodiment thereof, the disease is characterized by or caused by vascular leakage, preferably leakage of vascular endothelial cells, more preferably leakage of micro vascular endothelial cells.

In an embodiment of the first, second, third, fourth and fifth aspect, including any embodiment thereof, a subject suffering from the disease shows inflammation in the lung.

In an embodiment of the first, second, third, fourth and fifth aspect, including any embodiment thereof, the disease wherein the disease is selected from the group comprising pneumonia, sepsis and trauma.

In an embodiment of the first, second, third, fourth and fifth aspect, including any embodiment thereof, a subject suffering from the disease shows acute respiratory distress syndrome (ARDS).

In an embodiment of the first, second, third, fourth and fifth aspect, including any embodiment thereof, disease is pneumonia, preferably pneumonia selected from the group comprising severe community-acquired pneumonia (sCAP), community acquired pneumonia (CAP), hospital-acquired pneumonia (HAP).

In an embodiment of the first, second, third, fourth and fifth aspect, including any embodiment thereof, the disease is characterized by or shows lung damage.

In an embodiment of the first, second, third, fourth and fifth aspect, including any embodiment thereof, lung damage is immediate lung damage.

In an embodiment of the first, second, third, fourth and fifth aspect, including any embodiment thereof, immediate lung damage results from inhalation of toxic gases, toxic lung edema, lung infection of a virus or bacterium, aspiration of stomach contents, aspiration of fresh water, aspiration of salt water, pulmonary contusion, fat embolism, amniotic fluid embolism and inhalation of hyperbaric oxygen.

In an embodiment of the first, second, third, fourth and fifth aspect, including any embodiment thereof, lung damage is indirect lung damage.

In an embodiment of the first, second, third, fourth and fifth aspect, including any embodiment thereof, indirect lung damage results from sepsis, bacteremia, endotoxinemia, severe trauma, polytrauma including shock, bums, pancreatitis, malaria tropica, drugs and immunosuppression, chronic alcohol abuse, chronic pulmonary diseases, low pH of serum.

In an embodiment of the first, second, third, fourth and fifth aspect, including any embodiment thereof, the disease or effect is vascular regeneration or longevity, wherein preferably the effector molecule is selected from the group comprising VEGF, VEGF-A, VEGF-B, PDGF, bFGF, Sirtuin (Sirt 1), PPAR-gamma, AMPK, eNOS, RAS, c-MYC, FOXO3, PI3 kinase *, Akt, Akt* (* indicates fusion or truncated versions of the proteins to generate hyperactive and constitutively active derivatives of the kinases), adenosine A1 receptor, adenosine A2A receptor, adenosine A2B receptor, telomerase, Yamanaka factors (namely Oct4, sox2, klf4, c-My).

In an embodiment of the first, second, third, fourth and fifth aspect, including any embodiment thereof, the effector molecule is an antiapoptotic factor, preferably protecting normal, i.e. non-diseased endothelium, wherein the antiapoptotic factor is preferably selected from the group comprising PTEN, survivin, IAP, cIAP2 and XIAP, neuroglobin (binds cytochrome c), prosurvival pro-teins Bcl-2, Bcl-xl, Bcl-w, mcl-1 A1, NR-13, BHRF1, LMW5-HL, ORF16, KS-Bcl-2, E1b-19K and P53.

In an embodiment of the first, second, third, fourth and fifth aspect, including any embodiment thereof, the effector molecule is an apoptotic factor, preferably targeting tumor endothelium, wherein the apoptotic factor is preferably selected from the group comprising PTEN, survivin, IAP, cIAP2 and XIAP, neuroglobin (binds cytochrome c), prosurvival pro-teins Bcl-2, Bcl-xl, Bcl-w, mcl-1 A1, NR-13, BHRF1, LMW5-HL, ORF16, KS-BCl-2, E1b-19K, P53.

In an embodiment of the first, second, third, fourth and fifth aspect, including any embodiment thereof, the effector molecule is a tumor death ligand, preferably the tumor death ligand is proapoptotic or anti-angiogenic, more preferably the tumor death ligand is selected from the group comprising Members of the TNF gene superfamily of death ligand proteins such as TNF-alpha, Fas-L, Apo2L/TRAIL, IL-12, IL-13, IL-10, IL-8, IL-2,, secreted frizzled-related protein (SFRP) family (SFRP1, 2, 3, 4, 5), ELTD1, TGF-β, TNF-a, IL-6, PTEN, p53 and other tumor suppressor genes with proapoptotic function, thrombospondin C, TIMP-1, TIMP-2, interferon-alpha, interferon-beta, interferon-gamma, Ang-1 and derivatives with Tie-2 agonistic function, cytochrome c, BH3-only proteins, pro-apoptotic Bcl-2 proteins Bad, Bid, Bax, Bak and Bim, Apaf1, procaspase-8, -9 -10, caspases 2, 3, 6, 7, 8, 9 and 10.

In an embodiment of the first, second, third, fourth and fifth aspect, including any embodiment thereof, the effector molecule is anti-inflammatory, preferably the effector molecule is selected from the group comprising IkappaB and derivative with mutated phosphorylation sites, PI3 kinase and Akt kinases and hyperactive forms.

In an embodiment of the first, second, third, fourth and fifth aspect, including any embodiment thereof, the effector molecule is active in blood coagulation and/or hematopoiesis, preferably the effector molecule is selected from the group comprising erythropoietin, factor VII, factor VIII, factor IX and heparan-N-sulfatase.

In an embodiment of the first, second, third, fourth and fifth aspect, including any embodiment thereof, the effector molecule is a gene editing nuclease such as Cas9.

In an embodiment of the first, second, third, fourth and fifth aspect, including any embodiment thereof, the effector molecule is a secreted protein

In an embodiment of the first, second, third, fourth and fifth aspect, including any embodiment thereof, the effector molecule is effective as an autocrine factor.

In an embodiment of the first, second, third, fourth and fifth aspect, including any embodiment thereof, the effector molecule is effective as a paracrine factor.

In an embodiment of the first, second, third, fourth and fifth aspect, including any embodiment thereof, the recombinant nucleic acid construct comprises, preferably consists of ribonucleotides.

In an embodiment of the first, second, third, fourth and fifth aspect, including any embodiment thereof, the recombinant nucleic acid construct comprises, preferably consists of deoxyribonucleotides.

In an embodiment of the first, second, third, fourth and fifth aspect, including any embodiment thereof, the recombinant nucleic acid construct comprises, preferably consists of ribonucleotides and deoxyribonucleotides.

More specifically, the problem underlying the present invention is solved in a sixth aspect which is also a first embodiment of the sixth aspect, by a vector comprising a recombinant nucleic acid construct of the first aspect, including any embodiment thereof, and/or a recombinant nucleic acid construct as described in connection with each and any aspect, including any embodiment thereof, preferably of the second, third, fourth and fifth aspect.

In a second embodiment of the sixth aspect which is also an embodiment of the first embodiment of the sixth aspect, the vector is an expression vector.

In a third embodiment of the sixth aspect which is also an embodiment of the second embodiment of the sixth aspect, the vector allows expression of the recombinant nucleic acid construct in a cell, preferably an endothelial cell, more preferably the vector is a plasmid or a virus.

More specifically, the problem underlying the present invention is solved in a seventh aspect which is also a first embodiment of the seventh aspect, by a cell comprising a recombinant nucleic acid construct according to the first, second, third, fourth and fifth aspect, including any embodiment thereof, and/or a vector according to the sixth aspect, including any embodiment thereof.

In a second embodiment of the seventh aspect which is also an embodiment of the first embodiment of the seventh aspect, the cell is an endothelial cell or a mesenchymal stem cell.

In a third embodiment of the seventh aspect which is also an embodiment of the first and second embodiment of the seventh aspect, the cell is a recombinant cell.

In a fourth embodiment of the seventh aspect which is also an embodiment of the first, second and third embodiment of the seventh aspect, the cell is an isolated cell.

More specifically, the problem underlying the present invention is solved in an eighth aspect which is also a first embodiment of the eighth aspect, by a delivery vehicle comprising a recombinant nucleic acid construct according to the first, second, third, fourth and fifth aspect, including any embodiment thereof, wherein the delivery vehicle is a cationic lipid delivery particle.

In a second embodiment of the eighth aspect which is also an embodiment of the first embodiment of the eighth aspect, the particle is a nanoparticle.

In a third embodiment of the eighth aspect which is also an embodiment of the first and second embodiment of the eighth aspect, the average size of the nanoparticle is from about 30 nm to about 200 nm, preferably from about 30 nm to about 140 nm and more preferably from about 30 nm to about 60 nm.

More specifically, the problem underlying the present invention is solved in a ninth aspect which is also a first embodiment of the ninth aspect, by a composition comprising a recombinant nucleic acid construct according to the first, second, third, fourth and fifth aspect, including any embodiment thereof, a vector according to the sixth aspect, including any embodiment thereof, a cell according to the seventh aspect, including any embodiment thereof, and/or a delivery vehicle according to the eight aspect, including any embodiment thereof. In a preferred embodiment, the composition is a pharmaceutical composition comprising a pharmaceutically acceptable excipient.

More specifically, the problem underlying the present invention is also solved in a tenth aspect by the use in the manufacture of a medicament of a recombinant nucleic acid construct according to the first, second, third, fourth and fifth aspect, including any embodiment thereof, a vector according to the sixth aspect, including any embodiment thereof, a cell according to the seventh aspect, including any embodiment thereof, a cell according to the seventh aspect, including any embodiment thereof, and/or a delivery vehicle according to the eight aspect, including any embodiment thereof. In an embodiment thereof, the medicament is for the treatment of a disease, preferably a disease described herein in connection with the second, third, fourth and fifth aspect, including any embodiment thereof.

More specifically, the problem underlying the present invention is also solved in an eleventh aspect by a method for treating a subject, preferably a human subject, wherein the method comprises administering to the subject a therapeutically effective amount of a recombinant nucleic acid construct according to the first, second, third, fourth and fifth aspect, including any embodiment thereof, a vector according to the sixth aspect, including any embodiment thereof, a cell according to the seventh aspect, including any embodiment thereof, a delivery vehicle according to the eight aspect, including any embodiment thereof and/or a composition according to the ninth aspect, including any embodiment thereof. In an embodiment thereof, the method is for the treatment of a disease, preferably a disease described herein in connection with the second, third, fourth and fifth aspect, including any embodiment thereof.

More specifically, the problem underlying the present invention is also solved in an twelfth aspect by a method for vaccinating a subject, preferably a human subject, wherein the method comprises administering to the subject an amount effective to elicit in a or the subject an immune response, preferably a desired immune response, of a recombinant nucleic acid construct according to the first, second, third, fourth and fifth aspect, including any embodiment thereof, a vector according to the sixth aspect, including any embodiment thereof, a cell according to the seventh aspect, including any embodiment thereof, a delivery vehicle according to the eight aspect, including any embodiment thereof and/or a composition according to the ninth aspect, including any embodiment thereof. In an embodiment thereof, the method is for the treatment of a disease, preferably a disease described herein in connection with the second, third, fourth and fifth aspect, including any embodiment thereof. In a preferred embodiment, the effector molecule is one which is capable of or suitable for eliciting such immune response.

According to the present invention, any embodiment of the second, third, fourth and fifth aspect is also an embodiment of the first aspect, including any embodiment thereof.

According to the present invention, any embodiment of the first aspect is also an embodiment of the second, third, fourth and fifth embodiment, including any embodiment thereof.

In an embodiment and as preferably used herein, a heterologous construct, heterologous nucleic acid construct, recombinant construct or recombinant nucleic acid construct is a construct, preferably a nucleic acid construct, which as such is not existing in a wild type biological system such as a virus, a cell, a tissue, an organ or an organism. More preferably, a heterologous construct, heterologous nucleic acid construct, a recombinant construct or recombinant nucleic acid construct is one, where at least one element contained in the construct is not combined with a coding region for the effector, wherein said element is selected from the group consisting of a 5′ non-translated region, a 3′ non-translated region, a cap structure, a signal sequence and a poly-A tail. Preferably, the 5′ non-translated region is a 5′ UTR and the 3′ non-translated region is a 3′ UTR.

In an embodiment and as preferably used herein, a therapeutic method or a method for the treatment and/or prevention of a disease is a therapy.

In an embodiment of each and any aspect of the present invention, the coding region coding for an effector molecule is expressed by an endothelial cell. In preferred embodiment thereof, the endothelial cell expressing the effector molecule is an endothelial cell the function of which is impaired. In an alternative preferred embodiment, the endothelial cell expressing the effector molecule is an endothelial cell the function of which is not impaired; in such case, the effector molecule is exported or secreted by the effector molecule producing endothelial cell and exerts its effect on any other cell, typically any other endothelial cell, whereby such other cell is one the function of which is impaired. In a preferred embodiment, an impaired function of a cell is function of such cell which is to be restored in according with the present invention in its various aspect.

As preferably used herein, a derivative of a sequence which displays the same function as the parent sequence; preferably the parent sequence is a or the wild type sequence. In a preferred embodiment, the sequence of the derivative and of the parent case have an identity of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. It is within the present invention that the derivative is a truncated version of the parent sequence. Such truncation may exist at the 5′ end, the 3′ end or both the 5′ end and the 3′ end. If reference is made to a parent sequence in terms of identity, such identity preferable refers to a stretch of subsequent nucleotides shared by both the derivative and the parent sequence with the sequence of the parent sequence serving as the reference sequence.

In an embodiment and as preferably used herein, a coding region coding for an effector molecule comprises or consists of a nucleic acid sequence coding to said effector molecule.

In an embodiment and as preferably used herein, a 5′ non-translated region is a 5′ non-translated sequence.

In an embodiment and as preferably used herein, a 3′ non-translated region is a 5′ non-translated sequence.

In an embodiment and as preferably used herein, the indication of any source of any one of the elements contained in the recombinant nucleic acid construct of the invention is to be understood as a substitute of or an alternative description of a nucleotide sequence. Such nucleotide sequence is preferably the one of the respective wild type sequence, more preferably the one of the respective human wild type sequence. It will be appreciated by a person skilled in the art that other species than man may provide such wild type sequence; in an embodiment of the present invention such wild type sequence for such other species than man may be used in the practicing of the present invention. It will also be appreciated by a person skilled in the art that such wild type sequence may be taken from publicly available data bases such as GenBank. The wild type sequence may be subject to changes over time, typically to correct sequencing errors. In an embodiment of the invention, the sequence is the one described in said publicly available data bases such as GenBank at the date of filing of the instant application. To the extent various respective wild type sequences exist, each and any such wild type sequence shall be encompassed by the present invention. In another embodiment, where there is more than one wild type sequences and the more than one wild type sequence is provided by different groups of people such as different geographically defined groups of people and/or different genetically defined groups of people, the wild type sequence prevailing in the most comprehensive group of said different groups of people may be regarded as the wild type sequence.

In an embodiment of the present invention and as preferably used herein, if reference is made to a derivative of a 5′ UTR of a gene indicating that the derivative has a nucleotide identity of at least 85%, identity is identity referring or relative to the 5′ UTR of the gene.

In an embodiment of the present invention and as preferably used herein, if reference is made to a derivative of a 3′ UTR of a gene indicating that the derivative has a nucleotide identity of at least 85%, identity is identity referring or relative to the 3′ UTR of the gene.

In an embodiment and as preferably used herein, a signal peptide is a peptide which mediates directly or indirectly the transfer or secretion of a polypeptide or a protein fused or attached to the signal peptide into the extracellular space, whereupon, preferably, the signal sequence is removed from the polypeptide and protein, respectively.

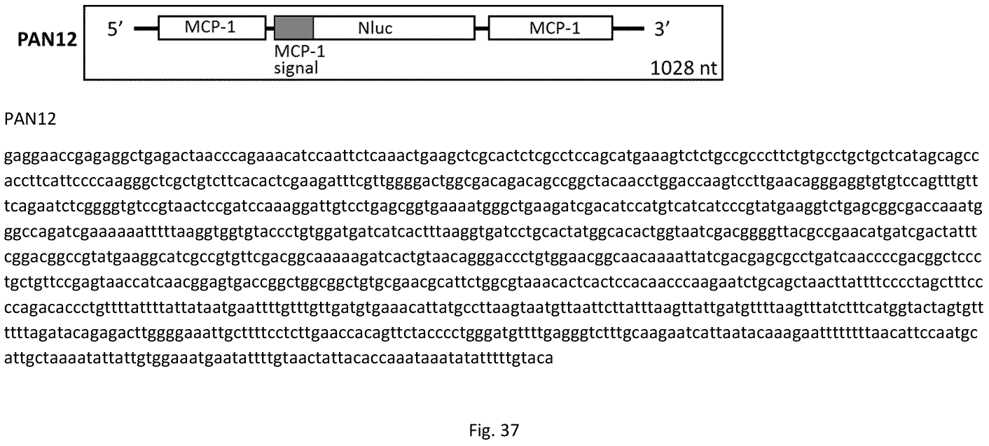

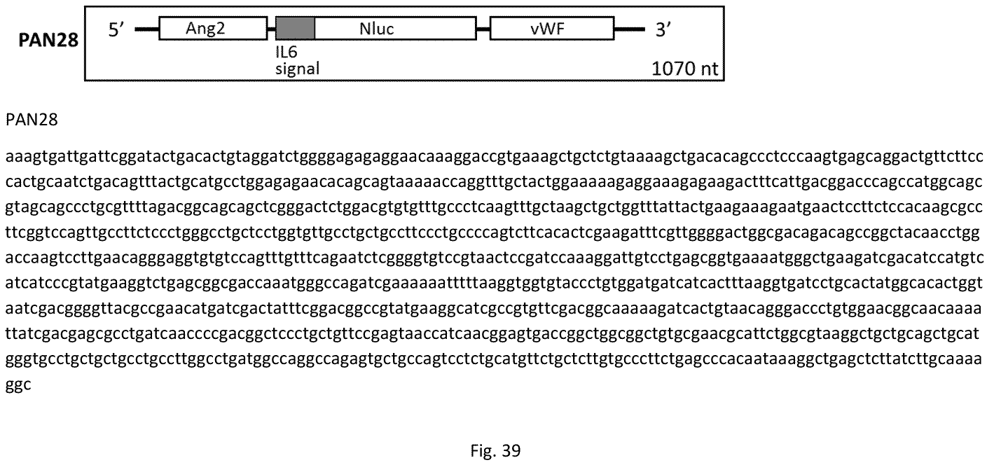

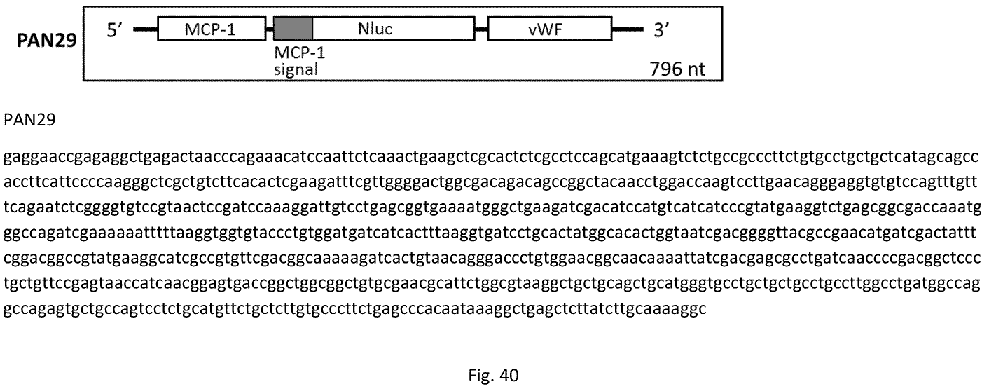

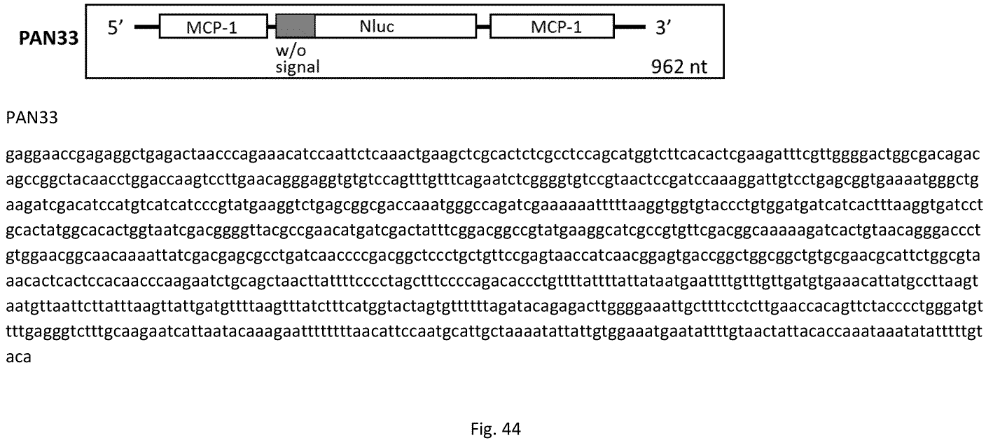

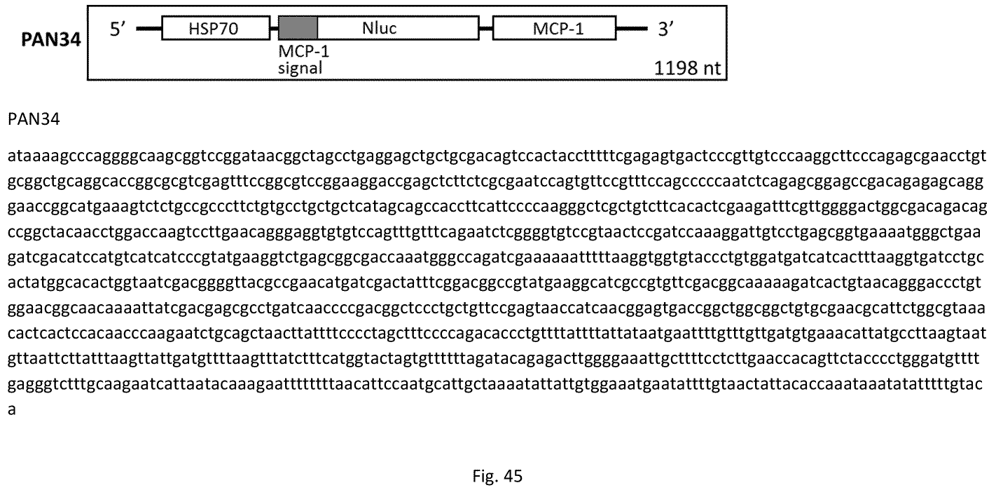

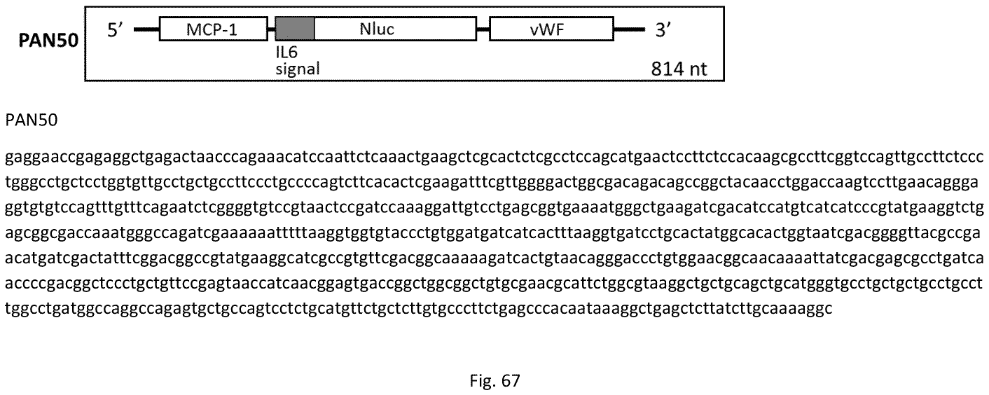

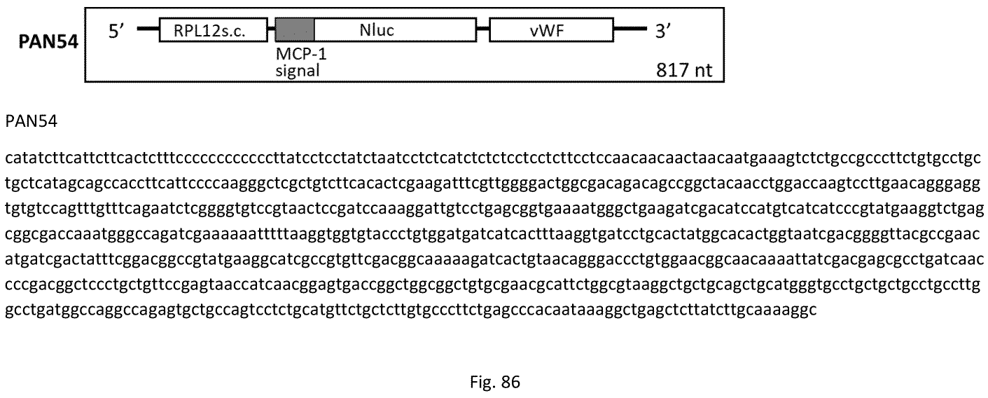

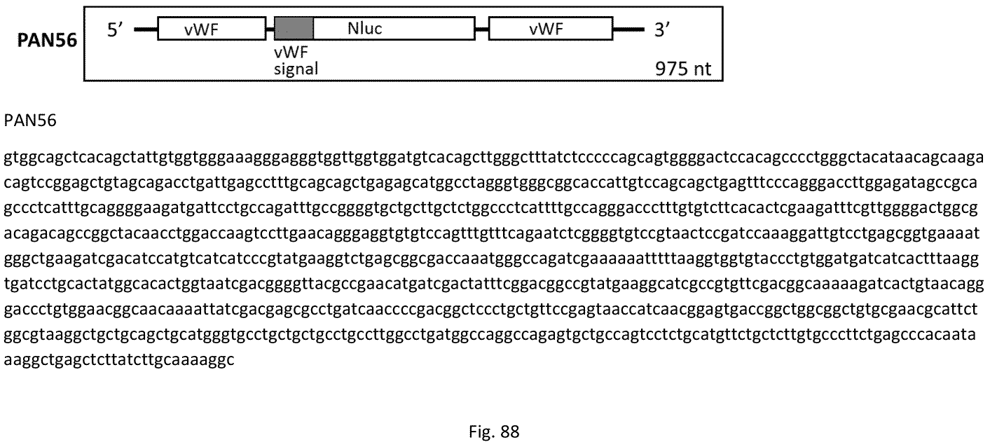

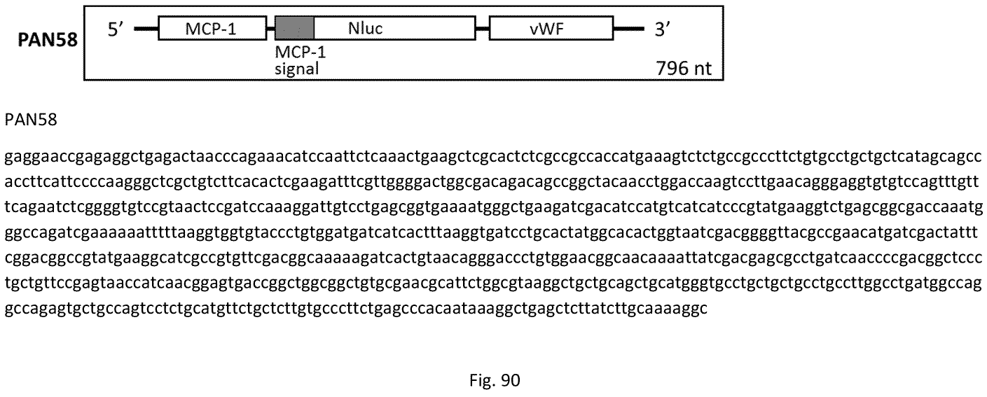

In an embodiment of the present invention, the recombinant nucleic acid construct is a recombinant nucleic acid construct disclosed in the Figs, more specifically a recombinant nucleic acid construct disclosed in FIG. 40 (PAN29), FIG. 90 (PAN58), FIG. 86 (PAN54), FIG. 67 (PAN50), FIG. 45 (PAN34), FIG. 44 (PAN33), FIG. 39 (PAN28), FIG. 37 (PAN12), FIG. 88 (PAN56).

In an embodiment of the present invention and as preferably used herein, a decoy is a mRNA encoded protein which binds a ligand and neutralizes the signaling activity.

In an embodiment and as preferably used herein, a poly-A tail is a nucleotide sequence comprising or consisting of a plurality of A’s covalently attached to each other, whereby such covalent linkage is preferably a phosphodiester linkage. In an embodiment thereof, the poly-A tail comprises about 20 to about 240 As (i.e. adenosine phosphates) nucleotides, preferably about 60 to 120 As, more preferable about 100 to 140 As and most preferably about 120 As.

The poly A tail is thought to stabilize natural messengers and synthetic sense RNA. Therefore, in one embodiment a long poly A tail can be added to an mRNA molecule thus rendering the RNA more stable. Poly A tails can be added using a variety of art-recognized techniques. For example, long poly A tails can be added to synthetic or in vitro transcribed RNA using poly A polymerase (Yokoe, et al. Nature Biotechnology. 1996; 14: 1252-1256). A transcription vector can also encode long poly A tails. In addition, poly A tails can be added by transcription directly from PCR products. Poly A may also be ligated to the 3′ end of a sense RNA with RNA ligase (see, e.g., Molecular Cloning A Laboratory Manual, 2nd Ed., ed. by Sambrook, Fritsch and Maniatis (Cold Spring Harbor Laboratory Press: 1991 edition)). In one embodiment, the length of the poly A tail is at least about 90, 200, 300, 400 at least 500 nucleotides. In one embodiment, the length of the poly A tail is adjusted to control the stability of a modified sense mRNA molecule of the invention and, thus, the transcription of protein. For example, since the length of the poly A tail can influence the half-life of a sense mRNA molecule, the length of the poly A tail can be adjusted to modify the level of resistance of the mRNA to nucleases and thereby control the time course of protein expression in a cell. In one embodiment, the stabilized nucleic acid molecules are sufficiently resistant to in vivo degradation (e.g., by nucleases), such that they may be delivered to the target cell without a transfer vehicle. In yet another embodiment the nucleic acid molecule can be purified by means of HPLC in order to remove double-stranded RNA contaminants and thus to reduce innate immune system activation (see e.g. Kariko et al., Nucleic Acids Res. 2011 Nov; 39(21):e142).

In an embodiment and as preferably used herein, the CAP structure is a nucleotide sequence forming a CAP structure which is known to a person skilled in the art. In eukaryotic cells the CAP-structure consists of a m7G(5′)ppp(5′)G 5′-5′-triphosphate linkage structure. The first two ribose sugars at the 5′-end of the mRNA can be methylated in the 2′-position which is known as CAP-1 (in case the first of the two ribose sugars is methylated) and CAP-2 structure (in case the second of the two ribose sugars is methylated). The CAP-structure can be incorporated during the mRNA transcription or post-transcriptionally by using capping and 2′-O-Methyltransferase enzymes and S-adenosylmethionine (SAM) as a methyl donor. In addition, a phosphatase treatment can be used in order to remove immune stimulatory 5′-phosphates.

In an embodiment and as preferably used herein, a gain of function mutation is a type of mutation in which the altered gene product possesses a new molecular function or a new pattern of gene expression. Gain of function mutations are almost always dominant or semi-dominant. In accordance therewith, a gain of function mutant effector molecule is an effector molecule possessing a new molecule function or a new pattern of expression.

In an embodiment and as preferably used herein, a hypermorphic mutation is a type of mutation in which the altered gene product possesses an increased level of activity. In accordance therewith, a hypermorphic mutant effector molecule is an effector molecule possessing an increased level of activity or an effector molecule which is expressed at an increased level. Preferably, such increase in expression level is caused by the mRNA coding the mutant effector molecule.

Hypermorphic mutations are, for example described in Muller, H. J. 1932. “Further studies on the nature and causes of gene mutations.” Proceedings of the 6th International Congress of Genetics, pp. 213-255, the disclosure of which is incorporated herein by reference.

In an embodiment and as preferably used herein, the terms dominant applied to the phenotypic effect of a particular allele in reference to another allele (usually the standard wild-type allele) with respect to a given trait. An allele “A” is said to be dominant with respect to the allele “a” if the A/A homozygote and the A/a heterozygote are phenotypically identical and different from the a/a homozygote.

In an embodiment and as preferably used herein, an allele “A” is said to be semi-dominant with respect to the allele “a” if the A/A homozygote has a mutant phenotype, the A/a heterozygote has a less severe phenotype, while the a/a homozygote is wild type.

It will be acknowledged and appreciated by a person skilled in the art that these dominant mutations identified in human genome-wide-analysis can be selected from groups with different biological consequences such as mRNA stabilization phenotypes (e.g. functional deletion of microRNA binding sites), protein stabilization phenotypes (e.g. mutation of PEST sequence), phenotypes with attenuation of inhibitory domains and interfering with binding sites for inhibitors, phenotypes with constitutively activation of catalytic domains such as kinase domains, G-coupled protein receptor domains, ligand binding and dimerization domains.

In an embodiment of the first, second, third, fourth, fifth, sixth, seventh, eighth, ninth, tenth, eleventh and twelfth aspect, including any embodiment thereof, and as preferably used herein, the non-mutant effector molecule is a or the wild type form of the effector molecule. In a preferred embodiment, the wild type form of the effector molecule is a wild type form of the effector molecule in human.

In an embodiment of the first, second, third, fourth, fifth, sixth, seventh, eighth, ninth, tenth, eleventh and twelfth aspect, including any embodiment thereof, the mutant effector molecule comprises an amino acid sequence and the non-mutant effector molecule comprises an amino acid sequence, wherein the mutant effector molecule differs from the non-mutant effector molecule at the level of the amino acid sequence and/or at the level of the nucleotide sequence of the mRNA level under the proviso that the mutant effector molecule is a gain of function mutant effector molecule and/or a hypermorphic mutant effector molecule compared to the non-mutant effector molecule.

In a preferred embodiment of the first, second, third, fourth, fifth, sixth, seventh, eighth, ninth, tenth, eleventh and twelfth aspect, including any embodiment thereof, the mutant effector molecule differs from the non-mutant molecule by one or more amino acid mutations; preferably, an amino acid mutation is a type of mutation, wherein the type of mutation is selected from the group comprising an amino acid substitution, an amino acid deletion and an amino acid insertion. In an embodiment of the first, second, third, fourth, fifth, sixth, seventh, eighth, ninth, tenth, eleventh and twelfth aspect, including any embodiment thereof, the mutant effector molecule comprises a mutation at one amino acid position defined by the non-mutant effector molecule. The mutation at the one amino acid position comprises one type of mutation; alternatively, the mutation at the one amino acid position comprises more than one type of mutation.

In a further embodiment of the first, second, third, fourth, fifth, sixth, seventh, eighth, ninth, tenth, eleventh and twelfth aspect, including any embodiment thereof, the mutant effector molecule comprises a mutation at two or more amino acid positions defined by the non-mutant effector molecule. At each and any of the two or more amino acid positions, the mutation comprises, individually and independently from any other of the two or more amino acid positions, one type of mutation; alternatively, the mutation at the two or more amino acid positions comprises individually and independently from any other of the two or more amino acid positions, more than one type of mutation.

In a further embodiment of the first, second, third, fourth, fifth, sixth, seventh, eighth, ninth, tenth, eleventh and twelfth aspect, including any embodiment thereof, the mutant effector molecule comprises an additional stop, which leads to a truncated version of the effector molecule. In a preferred embodiment thereof, such additional stop codon results in deletion of an inhibitors domain, such as, e.g., a terminal inhibitory domain and a C-terminal inhibitory domain.

In an embodiment of the first, second, third, fourth, fifth, sixth, seventh, eighth, ninth, tenth, eleventh and twelfth aspect, including any embodiment thereof, the mutant effector molecule is a mutant effector molecule of the Tie-2 pathway, the PI3 kinase pathway, the Akt pathway, the mTor pathway, the MAPK (mitogen activated protein kinase) pathway, the RAS pathway and the TCA (tricarboxylic acid) cylce. The recombinant nucleic acid construct according to the present invention comprising as the effector molecule this kind of mutant effector molecule are particularly effective in the treatment of vascular leakage and any diseases involving, going along with or being caused by such vascular leakage. A mutant effector molecule that is particularly preferred for use in the recombinant nucleic acid construct is a molecule that is responsible for or results in vascular malformation; preferably, vascular malfunction includes congenital vascular anomalies of veins, lymph vessels, both veins and lymph vessels, or both arteries and veins.

In a preferred embodiment of the first, second, third, fourth, fifth, sixth, seventh, eighth, ninth, tenth, eleventh and twelfth aspect, including any embodiment thereof, such mutant effector molecule of the Tie-2 pathway, the PI3 kinase pathway, the Akt pathway or the mTor pathway is contained as the effector molecule in the recombinant nucleic acid construct PAN29, PAN54 or PAN58, whereby in case of constructs PAN29, PAN54 or PAN58 replacing the Nano Luciferase as the effector molecule.

In an embodiment of the first, second, third, fourth, fifth, sixth, seventh, eighth, ninth, tenth, eleventh and twelfth aspect, including any embodiment thereof, the recombinant nucleic acid construct comprising such mutant effector molecule of the Tie-2 pathway, of the PI3 kinase pathway of the Akt pathway or of the mTor pathway, is for use in the treatment of a diseases, preferably the treatment of acute respiratory distress syndrome. Due to the mutant effector molecule the Tie-2 pathway is activated leading, among others, to the expression of activating ligand angiopoetin-1 and, as a result thereof, to reduced vascular leakage. Effector molecules of the Tie-2 pathway are Tie-2, AKT, FOXO1, ABIN2, IQGAP1, RAP1, RAC1 and VE-cadherin, effector molecules of the PI3 kinase pathway are PIK3CA, PDK1, AKT, XIAP, eNOS, RAC, RAS ERK and S6K, effector molecules of the Akt pathway are Akt1, eNOS, IKK, MEJ½, ERK and AP1, effector molecules of the mTor pathway are mTor, SGK1, PKCα, Akt, HIF-1, S6K, eIF4G, Lipin 1, SKAR, PGC-1α, PPARγ, PPARα, SREBP-1 and TFEB, effector molecules of the MAPK pathway are GNA11 and GNAQ, effector molecules of the RAS pathway are KRAS and NRAS, and an effector molecule of the TCA cycle is IDH1.

In an embodiment of the first, second, third, fourth, fifth, sixth, seventh, eighth, ninth, tenth, eleventh and twelfth aspect, including any embodiment thereof, the effector molecule is different from Tie-2 mutant (R849W).

In an embodiment of the first, second, third, fourth, fifth, sixth, seventh, eighth, ninth, tenth, eleventh and twelfth aspect, including any embodiment thereof, the effector molecule is different from a fusion or truncated version of PI3 kinase being hyperactive and/or being constitutively active.

In an embodiment of the first, second, third, fourth, fifth, sixth, seventh, eighth, ninth, tenth, eleventh and twelfth aspect, including any embodiment thereof, the effector molecule is different from a fusion or truncated version of Akt being hyperactive and/or being constitutively active.

In an embodiment of the first, second, third, fourth, fifth, sixth, seventh, eighth, ninth, tenth, eleventh and twelfth aspect, including any embodiment thereof, the effector molecule is a constitutively expressed Tie-2 receptor, preferably a constitutively expressed human Tie-2 receptor. The use of this embodiment allows restoration of vascular barrier function, preferably without the need for high concentrations of activating ligands to said Tie-2 receptor such as, e.g. Ang-1. The recombinant nucleic acid construct of this embodiment is particularly suitable for use in inflamed endothelium and for use in any diseases involving, going along with or being caused by inflamed endothelium, preferably inflamed endothelium showing decreased or downregulated expression of Tie-2 ligand such as SEPSIS, acute respiratory distress syndrom, vascular-leak-syndrome, cerebral malaria syndrome, Malaria, acute pancreatitis, clarkson disease, dengue virus infection, leakage associated infections.

In an embodiment of the first, second, third, fourth, fifth, sixth, seventh, eighth, ninth, tenth, eleventh and twelfth aspect, including any embodiment thereof, the effector molecule is a wild type Tie-2 receptor, wherein the recombinant nucleic acid construct coding for such wild type Tie-2 receptor additionally comprises a coding region for Ang-1 or wherein the recombinant nucleic acid construct coding for such wild type Tie-2 receptor is used together with or is for use together with a recombinant nucleic acid construct coding for Ang-1 as effector molecule.

In an embodiment of the first, second, third, fourth, fifth, sixth, seventh, eighth, ninth, tenth, eleventh and twelfth aspect, including any embodiment thereof, the effector molecule is a mutant human Tie-2, wherein the mutant human Tie-2 comprises one or more than one mutations selected from the group comprising A925S, K1100N, L914F, R915L, R915H, R915C, R918C, R918L, R918G, R918H, T1105N, T1106P, V919L, Y897N, Y897C, Y897H, Y897F and Y897S. In a preferred embodiment, the mutant human Tie-2 is selected from the group comprising L914F, R915L, R915C, T1105N, T 1106P, Y897H and Y897F.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 or a derivative thereof having a nucleotide identity of at least 85%, preferably the 5′ UTR comprises a nucleotide sequence of SEQ ID NO: 20, a 3′ UTR for a gene coding for von Willebrand factor (vWF) or a derivative thereof having a nucleotide identity of at least 85%, preferably the 3′ UTR comprises a nucleotide sequence of SEQ ID NO: 37, and a coding region coding for a mutant human Tie-2, wherein the mutant human Tie-2 comprises one or more than one mutations selected from the group comprising A925S, K1100N, L914F, R915L, R915H, R915C, R918C, R918L, R918G, R918H, T1105N, T1106P, V919L, Y897N, Y897C, Y897H, Y897F and Y897S, preferably one or more mutation selected from the group comprising L914F, R915L, R915C, T1105N, T 1106P, Y897H and Y897F; more preferably the mutant human Tie-2 comprises mutation L914F, the mutant human Tie-2 comprises mutations R849W and L914F, the mutant human Tie-2 comprises mutations Y897F and R915L, or the mutant human Tie-2 comprises mutations T1105N and T1106P.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human Tie-2 comprises mutation L914F, whereby the nucleotide sequence of the recombinant nucleic acid construct (PAN62) comprises SEQ ID NO: 128.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human Tie-2 comprises mutations R849W and L914F, whereby the nucleotide sequence of the recombinant nucleic acid construct (PAN63) comprises SEQ ID NO: 129.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human Tie-2 comprises mutations Y897F and R915L, whereby the nucleotide sequence of the recombinant nucleic acid construct (PAN64) comprises SEQ ID NO: 130.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human Tie-2 comprises mutations Y897F R915L T1105N T1106P, whereby the nucleotide sequence of the recombinant nucleic acid construct (PAN65) comprises SEQ ID NO: 131.

In an embodiment of the first, second, third, fourth, fifth, sixth, seventh, eighth, ninth, tenth, eleventh and twelfth aspect, including any embodiment thereof, the effector molecule is a mutant human PIK3CA (see, e.g., Tan FH et al.; Cells. 2019 Apr 9;8(4)) wherein the mutant human PIK3CA comprises one or more mutations selected from the group consisting of H1047R, E545K and E542K.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 or a derivative thereof having a nucleotide identity of at least 85%, preferably the 5′ UTR comprises a nucleotide sequence of SEQ ID NO: 20, a 3′ UTR for a gene coding for von Willebrand factor (vWF) or a derivative thereof having a nucleotide identity of at least 85%, preferably the 3′ UTR comprises a nucleotide sequence of SEQ ID NO: 37, and a coding region coding for a mutant human PIK3CA, wherein the mutant human PIK3CA comprises one or more than one mutations selected from the group comprising H1047R, E545K and E542K.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human PIK3CA comprises mutation E542K, whereby the nucleotide sequence of the recombinant nucleic acid construct (PAN67) comprises SEQ ID NO: 134.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human PIK3CA comprises mutation E545K, whereby the nucleotide sequence of the recombinant nucleic acid construct (PAN68) comprises SEQ ID NO: 135.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human PIK3CA comprises mutation H1047R, whereby the nucleotide sequence of the recombinant nucleic acid construct (PAN69) comprises SEQ ID NO: 136.

In an embodiment of the first, second, third, fourth, fifth, sixth, seventh, eighth, ninth, tenth, eleventh and twelfth aspect, including any embodiment thereof, the effector molecule is a mutant human PIK3CA, wherein the mutant human PIK3CA comprises one or more mutations selected from the group consisting of R88Q, N345K, C420R, E542K, E545A, E545D, E545G, E545K, Q546E, Q546K, Q546L, Q546R, M1043I, H1047L, H1047R, H1047Y and G1049R.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 or a derivative thereof having a nucleotide identity of at least 85%, preferably the 5′ UTR comprises a nucleotide sequence of SEQ ID NO: 20, a 3′ UTR for a gene coding for von Willebrand factor (vWF) or a derivative thereof having a nucleotide identity of at least 85%, preferably the 3′ UTR comprises a nucleotide sequence of SEQ ID NO: 37, and a coding region coding for a mutant human PIK3CA, wherein the mutant human PIK3CA comprises one or more than one mutations selected from the group comprising R88Q, N345K, C420R, E542K, E545A, E545D, E545G, E545K, Q546E, Q546K, Q546L, Q546R, M1043I, H1047L, H1047R, H1047Y and G1049R.

In an embodiment of the first, second, third, fourth, fifth, sixth, seventh, eighth, ninth, tenth, eleventh and twelfth aspect, including any embodiment thereof, the effector molecule is a mutant human Akt1. In a preferred embodiment, the mutant human Akt1 comprises one or more mutations, wherein the mutation is preferably E17K.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 or a derivative thereof having a nucleotide identity of at least 85%, preferably the 5′ UTR comprises a nucleotide sequence of SEQ ID NO: 20, a 3′ UTR for a gene coding for von Willebrand factor (vWF) or a derivative thereof having a nucleotide identity of at least 85%, preferably the 3′ UTR comprises a nucleotide sequence of SEQ ID NO: 37, and a coding region coding for a mutant human Akt1, wherein the mutant human Akt1 comprises one or more than one mutations, preferably the mutation is E17K.

In an embodiment of the first, second, third, fourth, fifth, sixth, seventh, eighth, ninth, tenth, eleventh and twelfth aspect, including any embodiment thereof, the effector molecule is a mutant human GNA11. In a preferred embodiment, the mutant human GNA11 comprises one or more mutations, wherein the mutation is preferably selected from the group comprising Q209H, R183C and Q209L.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 or a derivative thereof having a nucleotide identity of at least 85%, preferably the 5′ UTR comprises a nucleotide sequence of SEQ ID NO: 20, a 3′ UTR for a gene coding for von Willebrand factor (vWF) or a derivative thereof having a nucleotide identity of at least 85%, preferably the 3′ UTR comprises a nucleotide sequence of SEQ ID NO: 37, and a coding region coding for a mutant human GNA11, wherein the mutant human GNA11 comprises one or more than one mutations, preferably, the mutation is selected from the group comprising mutations Q209H, R183C and Q209L.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human GNA11 comprising mutation GNA11 Q 209H .

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human GNA11 comprising mutation GNA11 R183C.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human GNA11 comprising mutation GNA11 Q209L.

In an embodiment of the first, second, third, fourth, fifth, sixth, seventh, eighth, ninth, tenth, eleventh and twelfth aspect, including any embodiment thereof, the effector molecule is a mutant human GNAQ. In a preferred embodiment, the mutant human GNAQ comprises one or more mutations, wherein the mutation is preferably selected from the group comprising Q209H, R183Q and Q209L.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 or a derivative thereof having a nucleotide identity of at least 85%, preferably the 5′ UTR comprises a nucleotide sequence of SEQ ID NO: 20, a 3′ UTR for a gene coding for von Willebrand factor (vWF) or a derivative thereof having a nucleotide identity of at least 85%, preferably the 3′ UTR comprises a nucleotide sequence of SEQ ID NO: 37, and a coding region coding for a mutant human GNAQ, wherein the mutant human GNAQ comprises one or more than one mutations, preferably, the mutation is selected from the group comprising mutations Q209H, R183Q and Q209L.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human GNAQ comprising mutation GNAQ Q 209H.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human GNAQ comprising mutation GNAQ R183Q.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human GNAQ comprising mutation GNAQ Q209L.

In an embodiment of the first, second, third, fourth, fifth, sixth, seventh, eighth, ninth, tenth, eleventh and twelfth aspect, including any embodiment thereof, the effector molecule is a mutant human KRAS. In a preferred embodiment, the mutant human KRAS comprises one or more mutations, wherein the mutation is preferably selected from the group comprising Q22K, G12A, A146T, G13C and G12D.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 or a derivative thereof having a nucleotide identity of at least 85%, preferably the 5′ UTR comprises a nucleotide sequence of SEQ ID NO: 20, a 3′ UTR for a gene coding for von Willebrand factor (vWF) or a derivative thereof having a nucleotide identity of at least 85%, preferably the 3′ UTR comprises a nucleotide sequence of SEQ ID NO: 37, and a coding region coding for a mutant human KRAS, wherein the mutant human KRAS comprises one or more than one mutations, preferably, the mutation is selected from the group comprising mutations Q22K, G12A, A146T, G13C and G12D.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human KRAS comprising mutation KRAS Q22K.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human KRAS comprising mutation KRAS G12A.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human KRAS comprising mutation KRAS A146T.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human KRAS comprising mutation KRAS G13C.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human KRAS comprising mutation KRAS G12D.

In an embodiment of the first, second, third, fourth, fifth, sixth, seventh, eighth, ninth, tenth, eleventh and twelfth aspect, including any embodiment thereof, the effector molecule is a mutant human NRAS. In a preferred embodiment, the mutant human NRAS comprises one or more mutations, wherein the mutation is preferably mutation NRAS Q61R.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 or a derivative thereof having a nucleotide identity of at least 85%, preferably the 5′ UTR comprises a nucleotide sequence of SEQ ID NO: 20, a 3′ UTR for a gene coding for von Willebrand factor (vWF) or a derivative thereof having a nucleotide identity of at least 85%, preferably the 3′ UTR comprises a nucleotide sequence of SEQ ID NO: 37, and a coding region coding for a mutant human NRAS, wherein the mutant human NRAS comprises one or more than one mutations, preferably, the mutation is NRAS Q61R.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human NRAS comprising mutation NRAS Q61R.

In an embodiment of the first, second, third, fourth, fifth, sixth, seventh, eighth, ninth, tenth, eleventh and twelfth aspect, including any embodiment thereof, the effector molecule is a mutant human IDH1. In a preferred embodiment, the mutant human IDH1 comprises one or more mutations, wherein the mutation is preferably mutation IDH1 R132C.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 or a derivative thereof having a nucleotide identity of at least 85%, preferably the 5′ UTR comprises a nucleotide sequence of SEQ ID NO: 20, a 3′ UTR for a gene coding for von Willebrand factor (vWF) or a derivative thereof having a nucleotide identity of at least 85%, preferably the 3′ UTR comprises a nucleotide sequence of SEQ ID NO: 37, and a coding region coding for a mutant human IDH1, wherein the mutant human IDH1 comprises one or more than one mutations, preferably, the mutation is IDH1 R132C.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human IDH1 comprising mutation IDH1 R132C.

In an embodiment of the first, second, third, fourth, fifth, sixth, seventh, eighth, ninth, tenth, eleventh and twelfth aspect, including any embodiment thereof, the effector molecule is a mutant PIK3CA as disclosed herein, wherein the recombinant nucleic acid construct coding for such mutant PIK3CA additionally comprises a coding region for a mutant GNAQ as disclosed herein or wherein the recombinant nucleic acid construct coding for such mutant PIK3CA is used together with or is for use together with a recombing nucleic acid construct coding for a mutant GNAQ as disclosed herein as the effector molecule.

In an embodiment of the first, second, third, fourth, fifth, sixth, seventh, eighth, ninth, tenth, eleventh and twelfth aspect, including any embodiment thereof, the effector molecule is a mutant PIK3CA as disclosed herein, wherein the recombinant nucleic acid construct coding for such mutant PIK3CA additionally comprises a coding region for a mutant GNA11 as disclosed herein or wherein the recombinant nucleic acid construct coding for such mutant PIK3CA is used together with or is for use together with a recombing nucleic acid construct coding for a mutant GNA11 as disclosed herein as the effector molecule.

In an embodiment of the first, second, third, fourth, fifth, sixth, seventh, eighth, ninth, tenth, eleventh and twelfth aspect, including any embodiment thereof, the effector molecule is a mutant PIK3CA as disclosed herein, wherein the recombinant nucleic acid construct coding for such mutant PIK3CA additionally comprises a coding region for a mutant IDH1 as disclosed herein or wherein the recombinant nucleic acid construct coding for such mutant PIK3CA is used together with or is for use together with a recombing nucleic acid construct coding for a mutant IDH1 as disclosed herein as the effector molecule.

In an embodiment of the first, second, third, fourth, fifth, sixth, seventh, eighth, ninth, tenth, eleventh and twelfth aspect, including any embodiment thereof, the effector molecule is a mutant human PI 3-kinase which is referred to as myrP110*. Briefly, myrP110* is based on a constitutively active PI 3-kinase molecule p110* carrying a myristylation signal. P110* is a chimeric protein in which the iSH2 region of p85 is covalently linked to its binding site at the N-terminus of p110, using a flexible hinge region. The myristylation sequence is at the N-terminal end of the molecule. (see, for example “Constitutively active Phosphatidylinositol 3-Kinase And Uses Thereof.” Klippel et al. (1995); U.S. Pat. 6,043,062; and “Membrane Localization of Phosphatidylinositol 3-Kinase Is Sufficient To Activate Multiple Signal-Transducing Kinase Pathways.”, Klippel et al. (1996); Molecular and Cellular Biology; Vol. 16, No.8, p4117-4127.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 or a derivative thereof having a nucleotide identity of at least 85%, preferably the 5′ UTR comprises a nucleotide sequence of SEQ ID NO: 20, a 3′ UTR for a gene coding for von Willebrand factor (vWF) or a derivative thereof having a nucleotide identity of at least 85%, preferably the 3′ UTR comprises a nucleotide sequence of SEQ ID NO: 37, and a coding region coding for myrP110*.

In an embodiment of the first, second, third, fourth, fifth, sixth, seventh, eighth, ninth, tenth, eleventh and twelfth aspect, including any embodiment thereof, the recombinant nucleic acid construct comprises a 5′ UTR for a gene coding for MCP-1 (preferably of SEQ ID NO: 20), a 3′ UTR for a gene coding for von Willebrand factor (vWF) (preferably of SEQ ID NO: 37) and a coding region coding for the mutant human myrP110*, whereby the nucleotide sequence of the recombinant construct (PAN70) comprises SEQ ID NO: 151.

It will be acknowledged and appreciated by a person skilled in the art that the terms used herein preferably have the following meaning:
Tie-2: Tyrosine kinase with Ig and EGF homology domains-2 or Tyrosine-protein kinase receptor Tie-2
PIK3CA: Phosphatidylinositol-4,5-Bisphosphate 3-Kinase Catalytic Subunit Alpha
Akt: Protein kinase B (PKB
GNA11: G protein subunit alpha 11
GNAQ: G protein subunit alpha q
KRAS: Kirsten RAt Sarcoma virus
NRAS: Neuroblastoma RAS viral oncogene homolog
HRAS: Harvey murine sarcoma virus oncogene
IDH1: Isocitrat-Dehydrogenase 1
myrP110*: myristoylated form of the catalytic subunit p110alpha

In an embodiment of the first, second, third, fourth, fifth, sixth, seventh, eighth, ninth, tenth, eleventh and twelfth aspect, including any embodiment thereof, the effector molecule is a mutant of the EGFR gene (see, e.g., Lynch TJ et al. N Engl J Med. 2004 May 20;350(21):2129-39. Epub 2004 Apr 29).

In an embodiment of the first, second, third, fourth, fifth, sixth, seventh, eighth, ninth, tenth, eleventh and twelfth aspect, including any embodiment thereof, the effector molecule is a mutant of STAT3 and STAT1 (see, e.g. Fabre A et al.; J Allergy Clin Immunol Pract. 2019 Feb 27).

In an embodiment of the first, second, third, fourth, fifth, sixth, seventh, eighth, ninth, tenth, eleventh and twelfth aspect, including any embodiment thereof, the effector molecule is a mutant of KRAS, HRAS, NRAS (see, e.g., Ten Broek RW; Genes Chromosomes Cancer. 2019 Jan 24. doi: 10.1002/gcc.22739. [Epub ahead of print]).

In an embodiment of the first, second, third, fourth, fifth, sixth, seventh, eighth, ninth, tenth, eleventh and twelfth aspect, including any embodiment thereof, the effector molecule is a mutant of BRAF, KRAS, PIK3C, c-KIT, ABL, IDH1, IDH2 and JAK2 (see, e.g., Musaffe Tuna and Christopher I. Amos, “Activating Mutations and Targeted Therapy in Cancer” 2012 Oct 12. doi: 10.5772/48701.

In an embodiment of the first, second, third, fourth, fifth, sixth, seventh, eighth, ninth, tenth, eleventh and twelfth aspect, including any embodiment thereof, the effector molecule is a mutant of G-protein-coupled receptor genes (see, e.g., Fukami M et al. “Gain-of-function mutations in G-protein-coupled receptor genes associated with human endocrine disorders” Clin Endocrinol (Oxf). 2018 Mar; 88(3):351-359. doi: 0.1111/cen.13496. Epub 2017 Nov 7. Review)

In an embodiment of the first, second, third, fourth, fifth, sixth, seventh, eighth, ninth, tenth, eleventh and twelfth aspect, including any embodiment thereof, the effector molecule is a mutant of a receptor tyrosine kinase (RTK) (see, e.g., Du Z and Lovly CM; Mol Cancer. 2018 Feb 19;17(1)). In an embodiment, the receptor tyrosine kinase is oncogenically activated. In an alternative embodiment, a mutation is one in extracellular domain (ECD), transmembrane domain (TMD) or juxtamembrane domain (JMD) of RTKs. Three missense mutations within the EGFR ECD (P596L, G598 V, and A289V) were previously reported in glioblastoma (GBM) which are associated with increased expression of EGFR protein. In another embodiment, a mutation is a mutation within ECD of other RTKs such as RET (Rezeptor-Tyrosinkinase Ret) in thyroid cancer and KIT (Tyrosine kinase KIT) in gastrointestinal stromal tumor (GIST). HER2 G660D and V659E mutations within the TMD act as driver mutations in non-small cell lung cancer (NSCLC). HER2 V659 mutations are also found in a patient with Li-Fraumeni syndrome. These mutations disrupt specific protein-protein and protein-lipid interactions within the HER2 TMD that are essential for proper receptor dimerization. It has been shown that these two TMD mutations exhibit lower protein turnover than wild-type HER2. Finally, mutations within the JMD release autoinhibitory juxtamembrane interactions and subsequently hyperactivate these RTKs, such as KIT V560G and PDGFRA V561D mutation in GIST. In light thereof, it will be acknowledged and appreciated that mutations within the ECD, TMD and JM of RTKs adopt alternative activating mechanisms compared to mutations within the TKD.

Just for reason of clarity, as used herein, if a mutation of a protein such as an effector molecule is indicated herein, the amino acid residue refers to the amino acid residue of the respective human wild type protein, and commensurate with the usual practice in the field, the amino acid to the left represents the amino acid residue as present in the wild type protein, and the amino acid to the right represents the amino acid residue as present in the mutant protein. For example, Tie-2 R849W is a mutant based on the human wild type of Tie-2, preferably as disclosed herein, where amino acid residue R of the wild type sequence is replaced by amino acid residue W in the mutant Tie-2.

Also just for reason of clarity, to the extent the nucleotide sequence of a recombinant nucleic acid construct is indicated as a DNA sequence, any T will be an U in case the recombinant nucleic acid construct is present in the embodiment where the nucleotides are ribonucleotides (rather than deoxribonucleotides).

In an embodiment of the first, second, third, fourth, fifth, sixth, seventh, eighth, ninth, tenth, eleventh and twelfth aspect, including any embodiment thereof, the effector molecule is a mutant of an oncogene. Preferably, the mutant effector molecule us a constitutive active growth factor, a constitutive active receptor or an oncogenic mutation affecting cell proliferation (see, e.g., Lodish H, Berk A, Zipursky SL, et al.; Molecular Cell Biology; New York: W. H. Freeman; 2000).

Most tumors express constitutively active forms of one or more intracellular signal-transduction proteins, causing growth-promoting signaling in the absence of normal growth factors. Cancer can result from expression of mutant forms of growth factors, growth factor receptors, signal-transduction proteins, transcription factors, pro- or anti-apoptotic proteins, and DNA repair proteins. Mutations changing the structure or expression of proteins in these classes generally give rise to dominantly active oncogenes greatly increasing the probability that the mutant cells will become tumor cells. Virus-encoded proteins that activate growth-factor receptors also can induce cancer.

This oncogenic protein expression is permanent in nature due to genetic changes allowing for a sustained protein expression. The use of these mutations in the practicing of the present invention and the recombinant nucleic acid construct of the present invention is possible due to the highly transient nature of the oncogenic activity of these growth-related proteins making a permanent oncogenic transformation highly unlikely.

Oncogenes rarely arise from genes encoding growth factors. In fact, only one naturally occurring growth-factor oncogene — sis — has been discovered. The sis oncogene, which encodes a type of PDGF, can aberrantly autostimulate proliferation of cells that normally express the PDGF receptor. Several types of mutations leading to production of constitutively active receptors have been identified, which transmit growth signals in the absence of the normal ligands. In some cases, a point mutation changes a normal Receptor-Tyrosine-Kinase into one that dimerizes and is activated in the absence of ligand. For instance, a single point mutation converts the normal Her2 receptor into the Neu oncoprotein, which is found in certain mouse cancers. A mutation that alters a single amino acid (valine to glutamine) in the transmembrane region of the Her2 receptor causes dimerization of two receptor proteins in the absence of the normal EGF-related ligand, making the protein constitutively active as a kinase. Because of this, such mutant Her2 receptor constitutes an effector molecule of the recombinant nucleic acid construct according to the first, second, third, fourth, fifth, sixth, seventh, eighth, ninth, tenth, eleventh and twelfth aspect, including any embodiment thereof.

A deletion that causes loss of the extracellular ligand binding domain in the EGF receptor leads, for unknown reasons, to constitutive activation of the protein kinase, ErbB. Because of this, such mutant ErbB constitutes an effector molecule of the recombinant nucleic acid construct according to the first, second, third, fourth, fifth, sixth, seventh, eighth, ninth, tenth, eleventh and twelfth aspect, including any embodiment thereof. It will be appreciated and acknowledged by a person skilled in the art that ErbB is actually a protein family consisting of, namely ErbB-1, also named epidermal growth factor receptor (EGFR), ErbB-2, also named HER2 in humans and neu in rodents, ErbB-3, also named HER3, and ErbB-4, also named HER4.v-ErbBs are homologous to EGFR, but lack sequences within the ligand binding ectodomain.

Similarly, human tumors called multiple endocrine neoplasia type 2 have been found to express a constitutively active dimeric GDNF (Glial cell-derived neurotrophic factor) receptor that results from a point mutation in the extracellular domain. In other cases, deletion of much of the extracellular ligand-binding domain produces a constitutively active oncoprotein receptor. Because of this, such mutant GDNF receptor constitutes an effector molecule of the recombinant nucleic acid construct according to the first, second, third, fourth, fifth, sixth, seventh, eighth, ninth, tenth, eleventh and twelfth aspect, including any embodiment thereof.

A large number of oncogenes are derived from protooncogenes whose encoded proteins act as intracellular transducers, proteins that aid in transmitting signals from a receptor to a cellular target. In this class are the ras oncogenes, which were the first non-viral oncogenes to be recognized. E.g. a single point mutation in Ras, a key transducing protein in many signaling pathways, reduces its GTPase activity, thereby maintaining it in an activated state. One common mutation by which the ras proto-oncogene becomes an oncogene causes only one change in the protein, substitution of any amino acid for glycine at position 12 of the sequence. This simple mutation reduces the protein’s GTPase activity, thus linking GTP hydrolysis to the maintenance of normal, controlled Ras function. The oncogenic change causes only a slight alteration in the three-dimensional structure of Ras, but this is sufficient to change the normal protein into an oncoprotein. Constitutively active Ras oncoproteins are expressed by many types of human tumors including bladder, colon, mammary, skin, and lung carcinomas, neuroblastomas, and leukemias. Ras proteins are anchored to the inner side of the cell’s plasma membrane by a particular type of covalently attached fatty acid called a farnesyl group, this attachment is essential for either normal or oncogenic Ras proteins to function as signal transducers. Because of this, such mutant Ras constitutes an effector molecule of the recombinant nucleic acid construct according to the first, second, third, fourth, fifth, sixth, seventh, eighth, ninth, tenth, eleventh and twelfth aspect, including any embodiment thereof.

Oncogenes encoding other components of the RTK-Ras - MAP kinase pathway also have been identified. One example, found in certain transforming mouse retroviruses, encodes a constitutively activated Raf serine/threonine kinase, which is in the pathway between Ras and MAP kinase. Another is the crk oncogene found in avian sarcoma virus, which causes certain tumors when overexpressed. The Crk protein, which has no known biochemical activity, contains one SH2 and two SH3 domains, and is similar to the GRB2 adapter protein that also functions between an RTK and Ras. The SH2 and SH3 domains in GRB2 and other adapter proteins mediate formation of specific protein aggregates that normally serve as signaling units for cellular events. However, overexpression of Crk leads to formation of protein aggregates that inappropriately signal the growth and metastatic abilities characteristic of cancer cells. Because of this, such mutant Raf serine/threonine kinase and such mutant Crk protein constitute an effector molecule of the recombinant nucleic acid construct according to the first, second, third, fourth, fifth, sixth, seventh, eighth, ninth, tenth, eleventh and twelfth aspect, including any embodiment thereof.

Several oncogenes, some initially identified in human tumors, others in transforming retroviruses, encode nonreceptor protein-tyrosine kinases. One of these is generated by a chromosomal translocation that results in fusion of a portion of the bcr gene (whose function is unknown but whose N-terminal segment forms a coiled-coil domain that links several bcr polypeptides together) with part of the c-abl gene, which encodes a protein-tyrosine kinase whose normal substrates are not known. The chimeric polypeptides expressed from the resulting Bcr-Abl oncogene form a tetramer that exhibits constitutive Abl kinase activity. Although Abl is normally localized to the nucleus, addition of the Bcr segment causes the Bcr-Abl oncoprotein to be localized to the cytosol. Through the Abl SH2 and SH3 domains Bcr-Abl binds to many intracellular signal-transduction proteins and then phosporylates them, proteins that Abl would not normally activate. As a consequence, these signaling proteins, including JAK2 protein-tyrosine kinase, Stat5 transcription factor, and PI-3 kinase become activated in the absence of growth factors. Because of this, such oncogenes and mutant oncogenes, respectively, constitute an effector molecule of the recombinant nucleic acid construct according to the first, second, third, fourth, fifth, sixth, seventh, eighth, ninth, tenth, eleventh and twelfth aspect, including any embodiment thereof.

Another oncogene that encodes a constitutively active cytosolic protein-tyrosine kinase is src. As we noted previously, v-src from Rous sarcoma retrovirus was the first oncogene to be discovered. The regulation of Src activity and the alterations by which the c-src proto-oncogene becomes an oncogene is known. The normal Src kinase (c-Src) contains an SH2, an SH3, and a protein-tyrosine kinase domain. The kinase activity of this 60-kDa protein is normally inactivated by phosphorylation of the tyrosine residue at position 527, which is six residues from the C-terminus. Hydrolysis of phosphotyrosine 527 by a specific phosphatase enzyme normally activates c-Src. Tyrosine 527 is often missing or altered in Src oncoproteins that have constitutive kinase activity; that is, they do not require activation by a phosphatase. In Rous sarcoma virus, for instance, the src gene has suffered a deletion that eliminates the C-terminal 18 amino acids of c-Src; as a consequence, the v-Src kinase is constitutively active. Phosphorylation of target proteins by aberrant Src oncoproteins contributes to abnormal proliferation of many types of cells. Because of this, such aberrant or mutant Src oncoproteins, respectively, constitute an effector molecule of the recombinant nucleic acid construct according to the first, second, third, fourth, fifth, sixth, seventh, eighth, ninth, tenth, eleventh and twelfth aspect, including any embodiment thereof.

In an embodiment of the first, second, third, fourth, fifth, sixth, seventh, eighth, ninth, tenth, eleventh and twelfth aspect, including any embodiment thereof, any condition of disease can thus be addressed where more cellular growth is beneficial e.g. indications related to regeneration of damaged tissue in indications such as wound healing, stroke, heart failure, muscle and bone regeneration, cartilage regeneration, organ transplantation, induction of stem cell growth. More preferably, the effector molecule of the recombinant nucleic acid construct used for such purpose is a hypermorphic mutant effector molecule.

In an embodiment of the first, second, third, fourth, fifth, sixth, seventh, eighth, ninth, tenth, eleventh and twelfth aspect, including any embodiment thereof, the effector molecule is a mutant effector molecule and more specifically a mutant effector molecule which is hypermorphic. It will be appreciated and acknowledged by a person skilled in the art that such mutant effector molecule which is hypermorphic is transiently expressed by the recombinant nucleic acid construct of the present invention so as to avoid oncogenic transformation by excessive proliferation of cells and to prevent the induction of several diseases associated with uncontrolled proliferation, including cancer, atherosclerosis, vascular malformation, rheumatoid arthritis, psoriasis, liver fibrosis, idiopathic pulmonary fibrosis, scleroderma and cirrhosis of the liver. On the other hand, such mutant effector molecule which is hypermorphic can be used in any regenerative treatment. Preferably, such regenerative treatment includes, but is not limited to the treatment of heart failure, vascular disease, stroke, pulmonary disease including fibrosis, arthritis, burns, wound healing and skin disorders, Crohn’s disease, pediatric neurological disease, spinal cord injuries, Alzheimer, Parkinson, and frailty in the elderly.

Other conditions and diseases which can be treated by this kind of mutant effector molecule being contained in the recombinant nucleic acid construct of the present invention encompass any diseases in which an increased regenerative potential after tissue injury are potential fields of application. Humans are limited in their capacity for reparative regeneration, which occurs in response to injury. One of the most studied regenerative responses in humans is the hypertrophy of the liver following liver injury. Cardiac myocyte renewal has been found to occur in normal adult humans, and at a higher rate in adults following acute heart injury such as infarction. Even in adult myocardium following infarction, proliferation is only found in around 1% of myocytes around the area of injury, which is not enough to restore function of cardiac muscle. However, this is an important target for regenerative medicine as it implies that regeneration of cardiomyocytes, and consequently of myocardium, can be induced. When there is chronic or acute damage to tissue involving the immune system, as in inflammation or heart disease, the body loses the ability to repair itself.

In a preferred embodiment of the first, second, third, fourth, fifth, sixth, seventh, eighth, ninth, tenth, eleventh and twelfth aspect, including any embodiment thereof, the mutant effector molecule is phospholipase C-gamma (PLCG2) which is a functional hypermorphic mutant of PLCG2. A preferred mutant PLCG2 is PLCG2 P522R (see, e.g. Magno L et al., Alzheimers Res Ther. 2019 Feb 2;11(1):16). A recombinant nucleic acid construct of the present invention comprising as the effector molecule such mutant PLCG2 and PLCG2 P22R in particular, is useful in the treatment of Alzheimer’s disease (Magno L et al., supra).

In an embodiment of the first, second, third, fourth, fifth, sixth, seventh, eighth, ninth, tenth, eleventh and twelfth aspect, including any embodiment thereof, the effector molecule is a mutant Factor IX and more specifically a mutant Factor IX which is hypermorphic (VandenDriessche T and Chual MK; Molecular Therapy, Vol. 26, No. 1, January 2018, p. 18 ff). More preferably the mutant effector molecule is Factor IX R338L). A recombinant nucleic acid construct of the present invention comprising as the effector molecule such mutant Factor IX and Factor IX R338L in particular, is useful in the treatment of hemophilia (VandenDriessche T and Chual MK; supra).

In a preferred embodiment of the recombinant nucleic acid construct according to each and any aspect, including any embodiment thereof, the recombinant nucleic acid construct is an mRNA.

In an embodiment of each and any aspect of the present invention, including any embodiment thereof, a 5′ UTR of a gene coding for MCP-1 comprises a nucleotide sequence of SEQ ID NO: 20.

In an embodiment of each and any aspect of the present invention, including any embodiment thereof, a 3′ UTR of a gene coding for MCP-1 comprises a nucleotide sequence of SEQ ID NO: 23.

In an embodiment of each and any aspect of the present invention, including any embodiment thereof, a 5′ UTR of a gene coding for RPL12s.c. comprises a nucleotide sequence of SEQ ID NO: 24.

In an embodiment of each and any aspect of the present invention, including any embodiment thereof, a 3′ UTR of a gene coding for RPL12s.c. comprises a nucleotide sequence of SEQ ID NO: 25.

In an embodiment of each and any aspect of the present invention, including any embodiment thereof, a 5′ UTR of a gene coding for Ang-2 comprises a nucleotide sequence of SEQ ID NO: 26.

In an embodiment of each and any aspect of the present invention, including any embodiment thereof, a 3′ UTR of a gene coding for Ang-2 comprises a nucleotide sequence of SEQ ID NO: 29.

In an embodiment of each and any aspect of the present invention, including any embodiment thereof, a 5′ UTR of a gene coding for HSP70 comprises a nucleotide sequence of SEQ ID NO: 38 or 40.

In an embodiment of each and any aspect of the present invention, including any embodiment thereof, a 3′ UTR of a gene coding for HSP70 comprises a nucleotide sequence of SEQ ID NO: 39 or 43.

In an embodiment of each and any aspect of the present invention, including any embodiment thereof, a 5′ UTR of a gene coding for H3.3. comprises a nucleotide sequence of SEQ ID NO: 44.

In an embodiment of each and any aspect of the present invention, including any embodiment thereof, a 3′ UTR of a gene coding for H3.3. comprises a nucleotide sequence of SEQ ID NO: 45.

In an embodiment of each and any aspect of the present invention, including any embodiment thereof, a 5′ UTR of a gene coding for Galectin-9 (LHALS9) comprises a nucleotide sequence of SEQ ID NO: 46.

In an embodiment of each and any aspect of the present invention, including any embodiment thereof, a 3′ UTR of a gene coding for Galectin-9 (LHALS9) comprises a nucleotide sequence of SEQ ID NO: 47.

In an embodiment of each and any aspect of the present invention, including any embodiment thereof, a 5′ UTR of a gene coding for IL-6 comprises a nucleotide sequence of SEQ ID NO: 30.

In an embodiment of each and any aspect of the present invention, including any embodiment thereof, a 3′ UTR of a gene coding for IL-6 comprises a nucleotide sequence of SEQ ID NO: 33.

In an embodiment of each and any aspect of the present invention, including any embodiment thereof, a 5′ UTR of a gene coding for vWF comprises a nucleotide sequence of SEQ ID NO: 34.

In an embodiment of each and any aspect of the present invention, including any embodiment thereof, a 3′ UTR of a gene coding for vWF comprises a nucleotide sequence of SEQ ID NO: 37.

In an embodiment of each and any aspect of the present invention, including any embodiment thereof, a 5′ UTR of a gene coding for Ang-1 comprises a nucleotide sequence of SEQ ID NO: 83.

In an embodiment of each and any aspect of the present invention, including any embodiment thereof, a 3′ UTR of a gene coding for Ang-1 comprises a nucleotide sequence of SEQ ID NO: 88.

In an embodiment of each and any aspect of the present invention, including any embodiment thereof, a 5′ UTR of a gene coding for Ang-4 comprises a nucleotide sequence of SEQ ID NO: 89.

In an embodiment of each and any aspect of the present invention, including any embodiment thereof, a 3′ UTR of a gene coding for Ang-4 comprises a nucleotide sequence of SEQ ID NO: 94.

In an embodiment of each and any aspect of the present invention, including any embodiment thereof, a 5′ UTR of construct PAN57 comprises a nucleotide sequence of SEQ ID NO: 120.

In an embodiment of each and any aspect of the present invention, including any embodiment thereof, a 3′ UTR of construct PAN57 comprises a nucleotide sequence of SEQ ID NO: 121.

In an embodiment of each and any aspect of the present invention, including any embodiment thereof, the various combinations of a 5′ UTR and a 3′ UTR as realized in each and any of the specific constructs disclosed herein is a combination of 5′ UTR and a 3′ UTR which may be realized in recombinant nucleic acid construct irrespective of any other constituent and in particular irrespective of any coding region coding for an effector molecule contained in a recombinant nucleic acid construct of the invention.

In an embodiment of each and any aspect of the present in invention, including any embodiment thereof, and as preferably used herein, an endogenous gene is a gene contained in any cell described herein. In accordance therewith, a recombinant nucleic acid construct where the 5′ UTR and the 3′ UTR are from different endogenous genes, comprises a 5′ UTR from a first gene of a cell and a 3′ UTR from a second gene of a cell, for example the 5′ UTR is from a gene encoding MCP-1 or Ang-2, and the 3′ UTR is from a gene coding for vWF. Said cell may be the same cell or a different cell and/or said cell may be the same kind of cell or a different kind of cell. Preferably, the cell is of human origin.

In a preferred embodiment of the recombinant nucleic acid construct according to each and any aspect, including any embodiment thereof, the recombinant nucleic acid construct is a recombinant nucleic acid molecule.

It is within the present invention defined by each and any aspect, including any embodiment thereof, that the nucleotides forming the recombinant nucleic acid molecule may be modified. Suitable modifications include alterations in one or more nucleotides of a codon such that the codon encodes the same amino acid but is more stable than the codon found in the wild-type version of the nucleic acid. For example, an inverse relationship between the stability of RNA and a higher number cytidines (C’s) and/or uridines (U’s) residues has been demonstrated, and RNA devoid of C and U residues have been found to be stable to most RNases (Heidenreich, et al. J Biol Chem 269, 2131-8 (1994)). In some embodiments, the number of C and/or U residues in an mRNA sequence is reduced. In another embodiment, the number of C and/or U residues is reduced by substitution of one codon encoding a particular amino acid for another codon encoding the same or a related amino acid. Contemplated modifications to the mRNA nucleic acids of the present invention also include the incorporation of pseudouridine, N1-methyl-pseudouridine, 5-methoxyuridine and 5′-Methylcytidine. The incorporation of such modified nucleotides into the recombinant nucleic acid construct, preferably the mRNA of the present invention may enhance stability and translational capacity, as well as diminishing immunogenicity in vivo. (See, e.g., Kariko, K., et al., Molecular Therapy 16 (11): 1833-1840 (2008)). Substitutions and modifications to the recombinant nucleic acid construct of the present invention, particularly at the level of the individual nucleotide of the recombinant nucleic acid construct, may be performed by methods readily known to one of ordinary skill in the art.

The constraints on reducing the number of C and U residues in a sequence will likely be greater within the coding region of an mRNA, compared to an untranslated region, (i.e., it will likely not be possible to eliminate all of the C and U residues present in the message while still retaining the ability of the message to encode the desired amino acid sequence). The degeneracy of the genetic code, however presents an opportunity to allow the number of C and/or U residues that are present in the sequence to be reduced, while maintaining the same coding capacity (i.e., depending on which amino acid is encoded by a codon, several different possibilities for modification of RNA sequences may be possible). For example, the codons for Gly can be altered to GGA or GGG instead of GGU or GGC.

The term modification also includes, for example, the incorporation of non-nucleotide linkages or modified nucleotides into the nucleic acid sequences of the present invention (e.g., modifications to one or both the 3′ and 5′ ends of an mRNA molecule encoding a functional protein or enzyme). Such modifications include the addition of bases to a nucleic acid sequence (e.g., the inclusion of a poly A tail or a longer poly A tail), the alteration of the 3′ UTR or the 5′ UTR, complexing the nucleic acid with an agent (e.g., a protein or a complementary nucleic acid molecule), and inclusion of elements which change the structure of a nucleic acid molecule (e.g., which form secondary structures).

The delivery vehicle according to the eighth aspect is preferably based on a composition of one or more suitable lipids (e.g. liposomes) which in combination with the recombinant nucleic acid construct according to the present invention result in overall cationic-lipid-nucleic acid particles (cLNPs). Such cLNPs can be used for functionally transferring a nucleic acid, particularly an mRNA, into endothelial cells of the mammalian vasculature.

Lipid-nucleic acid nanoparticles (LNPs) formed by complexation of nucleic acids such as the recombinant nucleic acid construct of the present invention with cationic lipids in combination with other lipidic components, such as zwitterionic phospholipids, cholesterol and PEGylated lipids, have already been used to block degradation of RNAs in plasma and to facilitate the functional cellular uptake of the RNAs.

However, in a therapeutic context, particularly in view of parenteral therapeutic applications, the overall charge of the lipid-nucleic acid nanoparticles, characterized by the Zeta-potential, is essential in directing the organ distribution of the nanoparticles. Someone skilled in the art will appreciate that overall neutrally charged LNPs are almost exclusively taken up by organs of the reticuloendothelial system, particularly by the liver and spleen. On the other hand, it is also known to those skilled in the art, that overall positively charged lipid nanoparticles are particularly useful to functionally deliver a pharmaceutical payload into cells of the mammalian vasculature.

Those skilled in the art will also appreciate that free nucleic acids, particularly mRNAs, are immune stimulatory if applied intravenously.

Thus, in view of parenteral therapeutic applications a thorough and stable complexation of the nucleic acid inside the lipidic nanoparticle will be essential in order to prevent undesired immunogenic side reactions. Furthermore, it is also known to those skilled in the art that the size of the lipid-nucleic acid nanoparticle is crucial in suppressing the reticuloendothelial clearance of the particles.

It has recently been described that highly potent limit sized siRNA LNPs (~30 nm mean particle size) can be prepared using a microfluidic mixing process based on a staggered herringbone micromixer (SHM) device (Belliveau et al.; Molecular Therapy-Nucleic Acids (2012) 1(8)).

Due to the millisecond mixing of the lipids and the nucleic acid such as the recombinant nucleic acid construct of the invention on a nanoliter scale, the resulting particles are characterized by a very tight packaging of the nucleic acid inside a dense, solid particle core. On the contrary, those skilled in the art will appreciate that macrofluidic mixing processes using T-type connectors, generally result in larger nanoparticles with a more heterogeneous, multilamellar morphology.

In an embodiment, the present invention provides novel positively charged lipid-nucleic acid nanoparticles comprising a nucleic acid such as recombinant nucleic acid construct of the invention for the functional delivery of said recombinant nucleic acid construct of the invention, particularly mRNA, into cells of the mammalian vasculature, particularly into endothelial cells of the lung vasculature.

In an embodiment of the various aspects of the present invention, including any embodiment thereof, a cationic lipid selected from the group comprising β-(L-Arginyl)-L-2,3-diamino propionic acid-N-palmityl-N-oleyl-amide, L-Arginyl-β-alanine-N-palmityl-N-oleyl-amide, DOTAP (N-[1-(2,3-Dioleoyloxy)propyl]-N,N,N-trimethylammonium methyl-chloride), DOTMA (1,2-di-O-octadecenyl-3-trimethylammonium propane (chloride salt)) or DC-cholesterol is combined with neutral lipids and further shielding lipid components bearing a polyethylene glycol (PEG) modification. The neutral lipid can be either a zwitterionic phospholipid selected from a group comprising Diphytanoyl-PE (1,2-Diphytanoyl-sn-glycero-3-phosphoethanolamine), DOPE (1,2-Dioleoyl-sn-glycero-3-phosphoethanolamine), DLPE (1,2-Lauroyl-sn-glycero-3-phosphoethanolamine), DMPE, POPE, DSPE or an uncharged sterol lipid selected from a group comprising cholesterol and stigmasterol. The PEGylated lipid component is selected from a group comprising methoxyPEG-DSPE, methoxyPEG-DLG, methoxyPEG-DMG, methoxyPEG-DPG, methoxyPEG-DSG, methoxyPEG-c-DMA, methoxyPEG-C8-Ceramide, methoxyPEG-C16-Ceramide. In a particular embodiment the chain length of the PEG-chain corresponds to a molecular weight in the range of 750 Da to 5000 Da, preferably in a range of 1500 Da to 3000 Da.

In another embodiment of the present invention, the molar ratio of the lipids in the lipid mixture is in a range of 20 - 80 mol% cationic lipid, 10 - 70 mol% neutral lipid and 1 - 10 mol% PEGylated lipid, preferably in a range of 35-65 mol% cationic lipid, 35-65 mol% neutral lipid and 1-5 mol% PEGylated lipid (with the overall lipid content being set as 100%).

In a preferred embodiment, the cationic lipid is β-(L-Arginyl)-L-2,3-diamino propionic acid-N-palmityl-N-oleyl-amide or L-Arginyl-β-alanine-N-palmityl-N-oleyl-amide, the neutral lipid is the phospholipid Diphytanoyl-PE and the PEGylated lipid is methoxyPEG2000-DSPE. The molar ratio is 50 mol% cationic lipid, 49 mol% Diphytanoyl-PE and 1 mol% mPEG2000-DSPE (1,2-distearoyl-sn-glycero-3-phosphoethanolamine-N-[methoxy(polyethylene glycol)-2000] (sodium salt)).

In yet another embodiment, the mixture of above mentioned lipids is dissolved in a water miscible solvent selected from a group comprising ethanol, acetone, 1-butanol, 2-butanol, tert.-butanol, 3-methyl-1-butanol, 2-methyl-1-propanol, 1-propanol, 2-propanol, dimethylsulfoxide, preferably from a group comprising ethanol, tert.-butanol and 1-butanol.

The dissolved lipid mixture is subsequently rapidly mixed with a dissolution of a nucleic acid such as the recombinant nucleic acid construct of the invention in an aqueous solvent, resulting in the formation of lipid-nucleic acid nanoparticles (LNPs). In a preferred embodiment of the present invention, the mixing is performed using a microfluidic mixing device, particularly using a staggered herringbone-type mixing chamber, e.g. NanoAssemblr™ (Precision NanoSystems Inc.; Vancouver, Canada) device.

In yet another embodiment, the mass ratio of total lipids to nucleic acid nucleic acid such as the recombinant nucleic acid construct of the invention is in a range of 5 to 60, preferably in a range of 15 to 40 and the volumetric mixing ratio of aqueous nucleic acid solution to organic total lipid solution is in the range of 1:1 to 6:1, preferably in the range of 2:1 to 4:1. The mixing flow-rate of the solutions is in the range of 5 ml/min to 25 ml/min, preferably in a range of 10 ml/min to 20 ml/min.

In a certain embodiment of the invention the organic solvent of the resulting mixture is removed after the mixing step by dialysis or by tangential flow filtration using ultrafiltration membranes, whereby the ultrafiltration membranes can be either hollow fiber membranes or flat-screen membranes. The pore size of the ultrafiltration membrane corresponds to a molecular weight cutoff in the range of 1500 Da to 500.000 Da, preferably in a range of 1500 Da to 100.000 Da and even more preferably in a range of 1500 Da to 30.000 Da. Within the tangential flow filtration step it is also possible to concentrate the LNPs to a desired concentration.

The mean particle size of the resulting LNPs can be measured using dynamic light scattering (DLS) technologies and is preferably in the range of 15 nm to 400 nm, more preferably in a range of 25 nm to 200 nm and even more preferable the particles have a size in the range of 25 nm to 100 nm. The overall particle charge of the LNPs is positive and the Zeta-potential of the particles is preferably in a range between +5 mV to +60 mV, more preferably in a range of +25 mV to +60 mV.

In certain embodiments, the delivery vehicle of the invention is a liposomal transfer vehicle, e.g. a lipid nanoparticle or a lipidoid nanoparticle. In one embodiment, the transfer vehicle may be selected and/or prepared to optimize delivery of the recombinant nucleic acid construct of the invention to a target cell. For example, if the target cell is an endothelial cell, the properties of the transfer vehicle (e.g., size, charge and/or pH) may be optimized to effectively deliver such transfer vehicle to the target cell, reduce immune clearance and/or promote retention in that target cell. Alternatively, if the target cell is in the central nervous system (e.g., mRNA such as the recombinant nucleic acid construct of the invention administered for the treatment of neurodegenerative diseases may specifically target brain or spinal tissue), selection and preparation of the transfer vehicle must consider penetration of, and retention within, the blood brain barrier and/or the use of alternate means of directly delivering such transfer vehicle to such target cell. In one embodiment, the compositions of the present invention may be combined with agents that facilitate the transfer of exogenous mRNA (e.g., agents which disrupt or improve the permeability of the blood brain barrier and thereby enhance the transfer of exogenous mRNA to the target cells).

Liposomes (e.g., liposomal lipid nanoparticles) are known to be particularly suitable for their use as transfer vehicles of diagnostic or therapeutic compounds in vivo (Lasic, Trends Biotechnol, 16: 307-321, 1998; Drummond et al, Pharmacol. Rev., 51: 691-743, 1999) and are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol, 16: 307- 321, 1998). Bilayer membranes of the liposomes can also be formed by amphiphilic polymers and surfactants (e.g., polymerosomes, niosomes, etc.). Such liposomes may also be used as the lipid moiety in the delivery vehicle of the present invention comprising a recombinant nucleic acid construct of the present invention.

It is to be acknowledged that any feature related to the recombinant nucleic acid construct as disclosed in connection with a particular aspect of the present invention is equally an embodiment of the recombinant nucleic acid construct of each and any other aspect of the present invention, including any embodiment thereof. More specifically, any embodiment of the recombinant nucleic acid construct disclosed in connection with the first aspect is also an embodiment of the recombinant nucleic acid construct of the second, third and fourth aspect; similarly, any embodiment of the recombinant nucleic acid construct disclosed in connection with the second, third and fourth aspect is also an embodiment of the recombinant nucleic acid construct of the first aspect.

It is generally acknowledged in the art that the UTRs flanking a coding region do not interfere with the coding region and, more specifically, that the UTRs may, in principle, be exchanged without interfering with the expression of the coding region (see, e.g. WO 2017/100551 A1; Trepotec et al., Tissue Engineering Part A, Vol 25 No. 1-2 p. 69-79 (January 2019).

Without wishing to be bound by any theory, the present inventors have surprisingly found that the recombinant nucleic acid molecule of the present invention and more specifically the mutant effector molecule having a gain of function mutation or a hypermorphic mutation is suitable for increasing the level of protein expression of the effector molecule, for increasing the duration of the expression of the effector molecule and/or for increasing the functional activity of the effector molecule. The use of the mutant effector molecule leads to better therapeutic efficacy compared to the non-mutant effector molecule. Preferably, gain of function mutation can be derived by means of routine measures from sporadic somatic mutation of the non-mutant effector molecule or from inherited germline mutations of the non-mutant effector molecule causing the desired phenotype. Preferably, a mutant effector molecule is one verified by human genetic studies.

It will be appreciated by a person skilled in the art that due to the transient, i.e. timely restricted expression of the effector molecule from the recombinant nucleic acid construct of the present invention, no pathological phenotype will be caused despite the effector molecule in the mutant effector molecule in particular being potentially pathogenic. Rather, the transient increase in activity will provide for a better therapeutic response and/or will require less frequent dosing.

The present invention is further illustrated by the following Figs. and Examples form which further features, embodiments and advantages of the present invention may be taken.

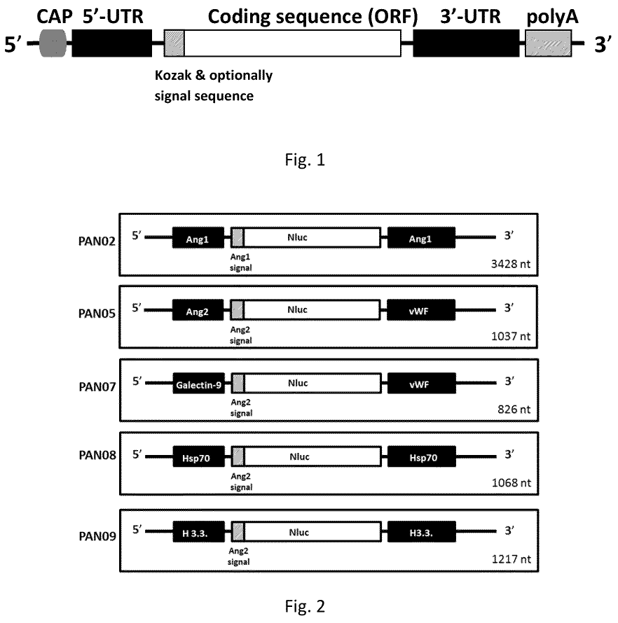

FIG. 1 is a schematic representation of conventional mRNA molecule structure.

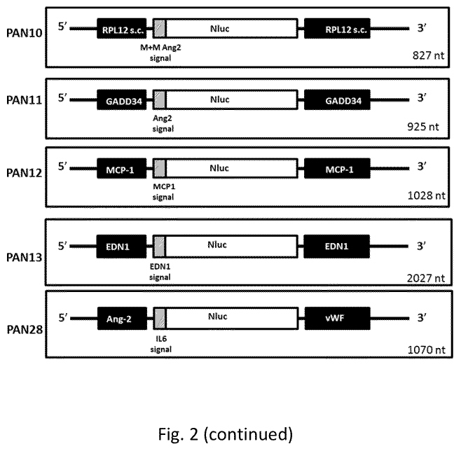

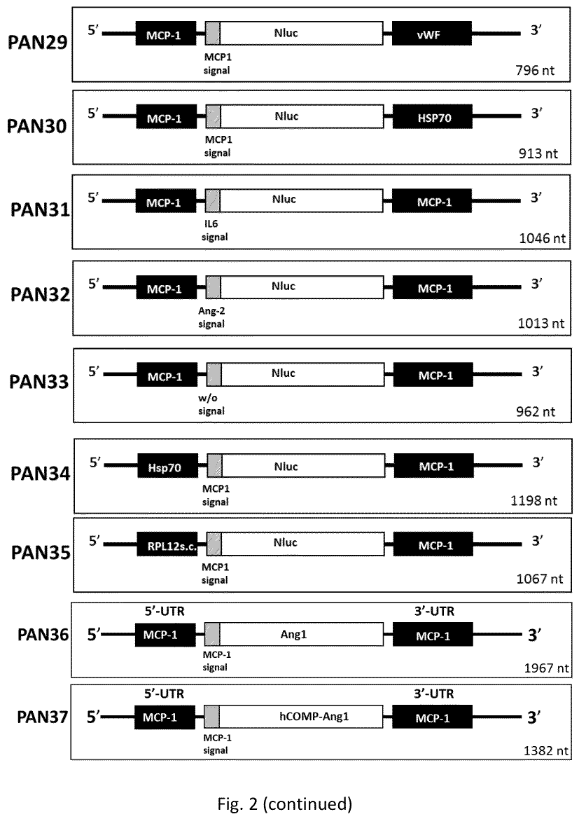

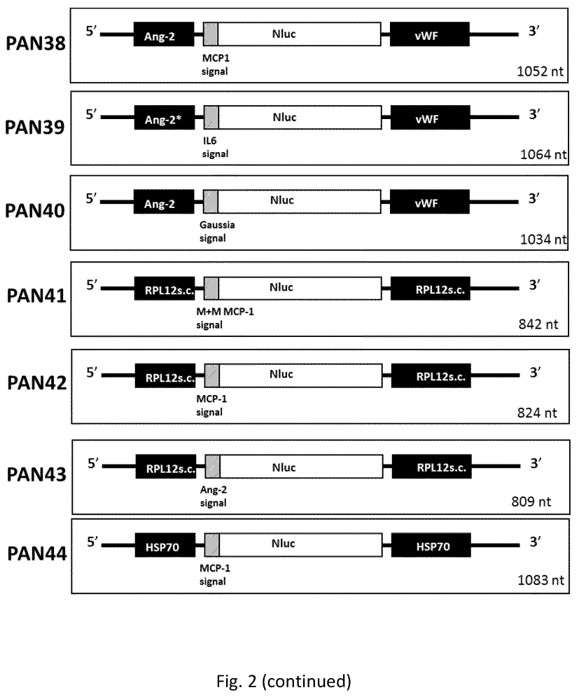

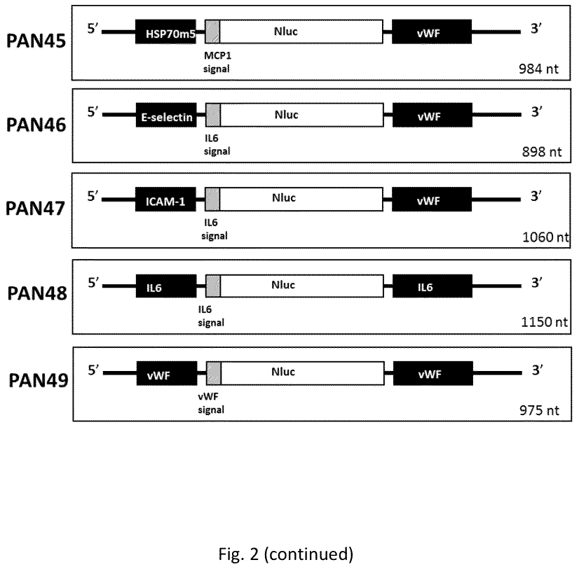

FIG. 2 is a schematic representation of selected examples for mRNA constructs with the coding sequence with an open reading frame being the one of Nano-luciferase, namely PAN02, PAN05, PAN07, PAN08, PAN09, PAN10, PAN11, PAN12, 13, PAN28, PAN29, PAN30, PAN31, PAN32, PAN33, PAN34, PAN35, PAN36, PAN37, PAN38, PAN39, PAN40, PAN41, PAN42, PAN43, PAN44, PAN45, PAN46, PAN47, PAN48 and PAN49.

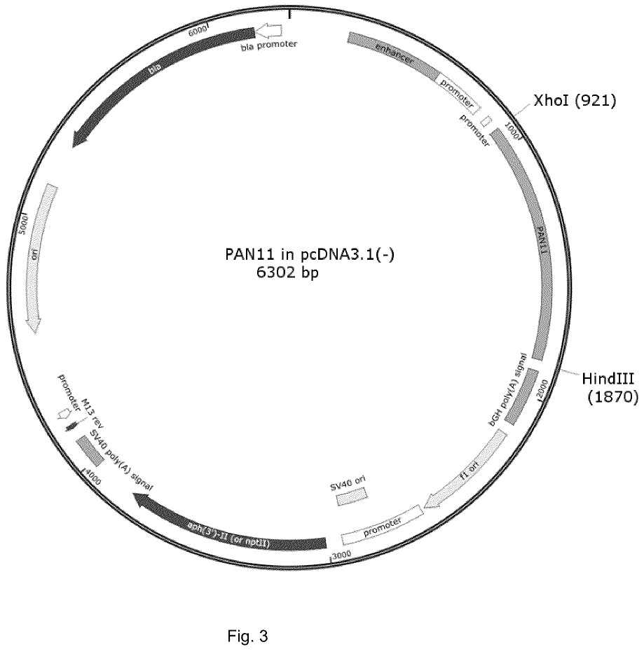

FIG. 3 is a restriction map of plasmid pcDNA3.1(-) containing construct PAN11.

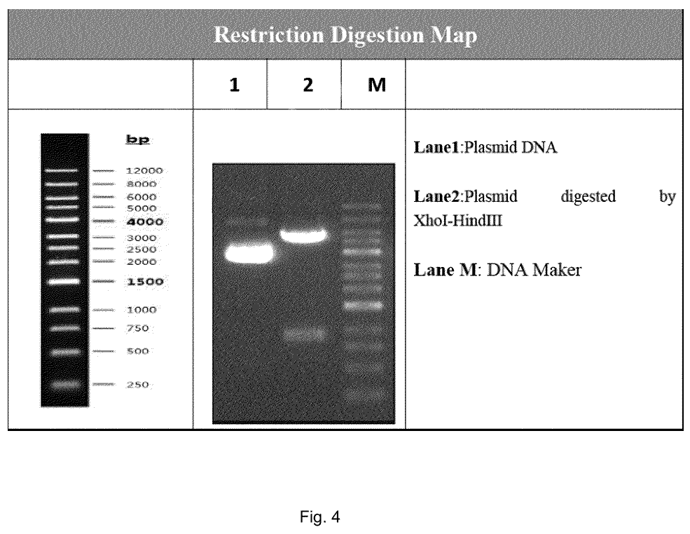

FIG. 4 is a restriction digest map of plasmid pdDNA3.1(-) of FIG. 3.

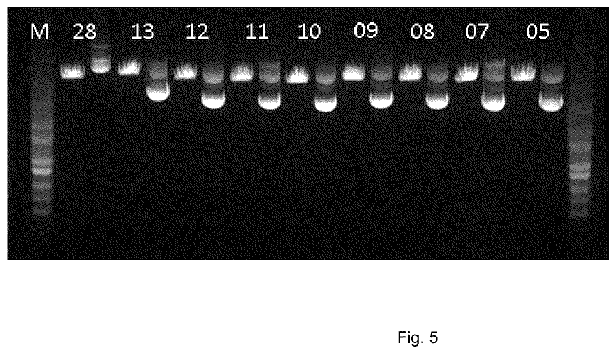

FIG. 5 is an 1% agarose gel stained with EtBr showing the non-linearized (uncut, supercoiled, right lanes) and linearization product (left lanes) of pcDNA3.1(-) plasmids containing constructs PAN28 (“28”), PAN13 (“13”), PAN12 (“12”), PAN11 (“11”), PAN10 (“10”), PAN09 (“09”), PAN08 (“08”), PAN07 (“07”) and PAN05 (“05”) upon BamHI restriction.

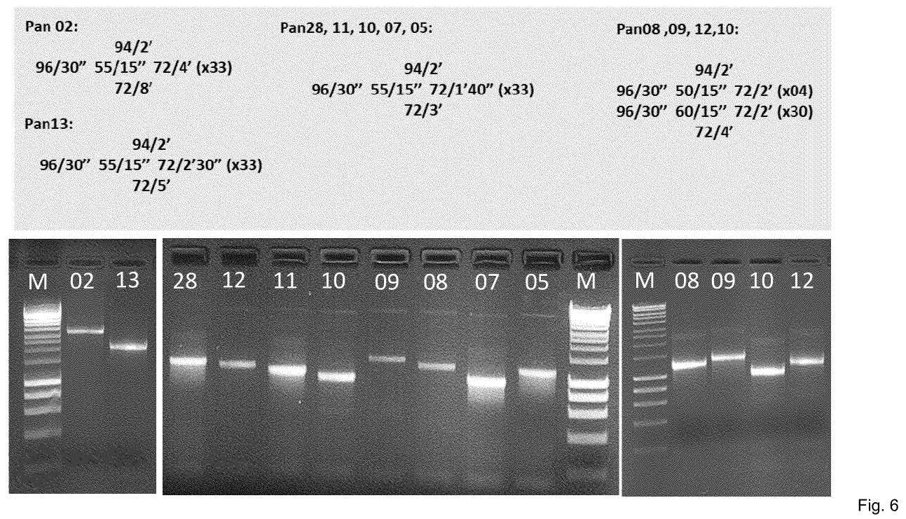

FIG. 6 shows 1% agarose gels stained with EtBr showing PCR products of Poly-A tailing PCRs for the addition of 120 nt of Poly-A tail.

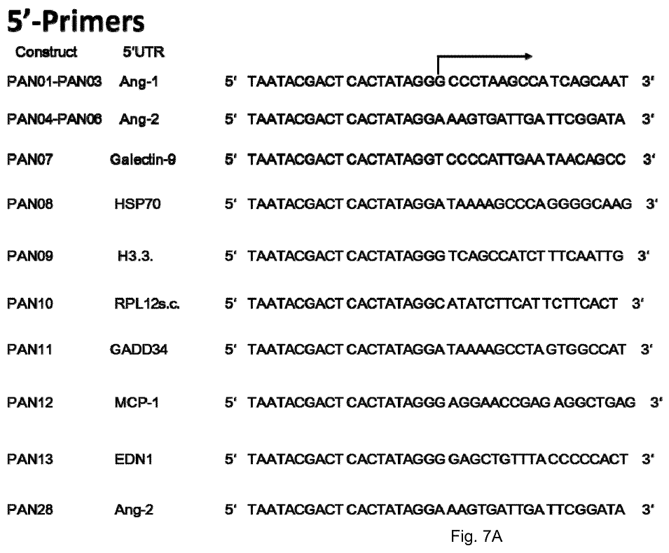

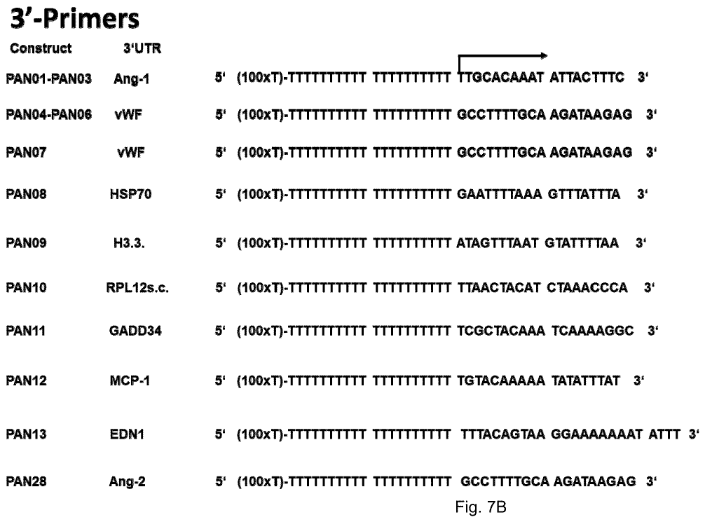

FIG. 7A shows the 5′ primers used for the 5′ UTRs of the indicated gene; the first column indicates in connection with which construct such primer is to be used, the second column indicates the origin of the 5′ UTR, and the third column indicates the position where the primer hybridizes to the nucleotide sequence.

FIG. 7B shows the 3′ primers used for the 3′ UTRs of the indicated gene; the first column indicates in connection with which construct such primer is to be used, the second column indicates the origin of the 3′ UTR, and the third column indicates the position where the primer hybridizes to the nucleotide sequence.

FIG. 8 is an image of a non-denaturing agarose gel after EtBr staining showing the in vitro mRNA transcripts of various construct.

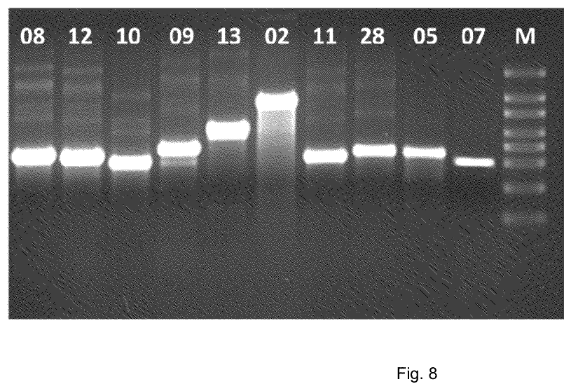

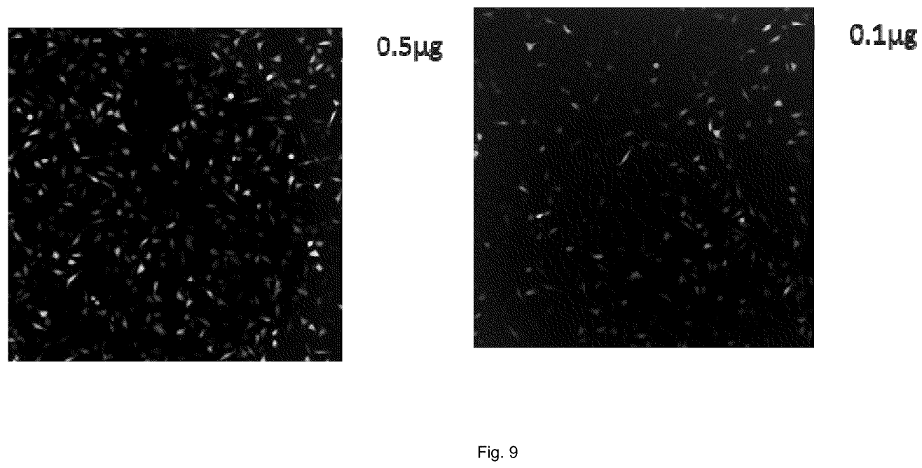

FIG. 9 are photographs of fluorescence microscopy 24 h post transfection of HeLa cells transfected with 0.5 µg (left) and 0.1 µg (right) of a commercially, enhanced mRNA version of the producing green fluorescent protein (TriLINK Biotechnology).

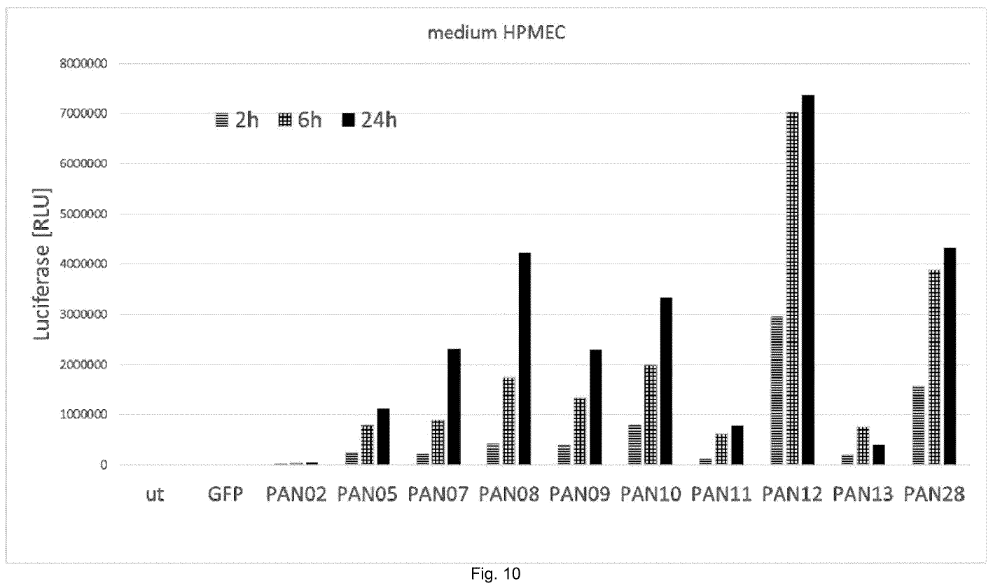

FIG. 10 is a bar diagram showing expression of luciferase, indicated as RLU (relative light units) of luciferase in medium, by the indicated recombinant nucleic acid constructs in HPMEC where luciferase is used as the effector molecule after 2 hours (left column), 6 hours (middle column) and 24 hours (right column) post transfection.

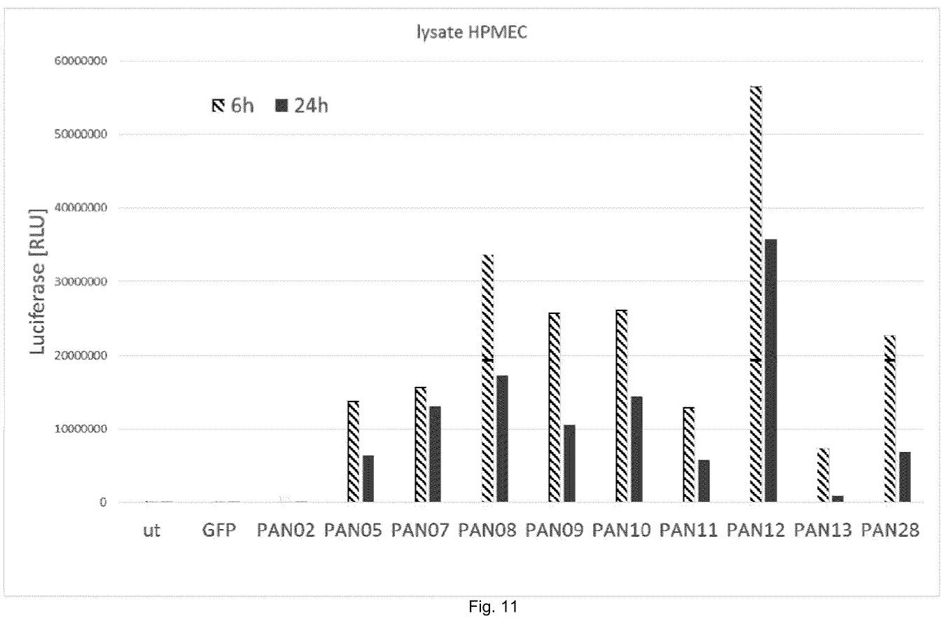

FIG. 11 is a bar diagram showing expression of luciferase, indicated as RLU (relative light units) of luciferase in whole cell lysates of HPMEC, by the indicated recombinant nucleic acid constructs in HPMEC, where luciferase is used as the effector molecule after, 6 hours (left column) and 24 hours (right column) post transfection.

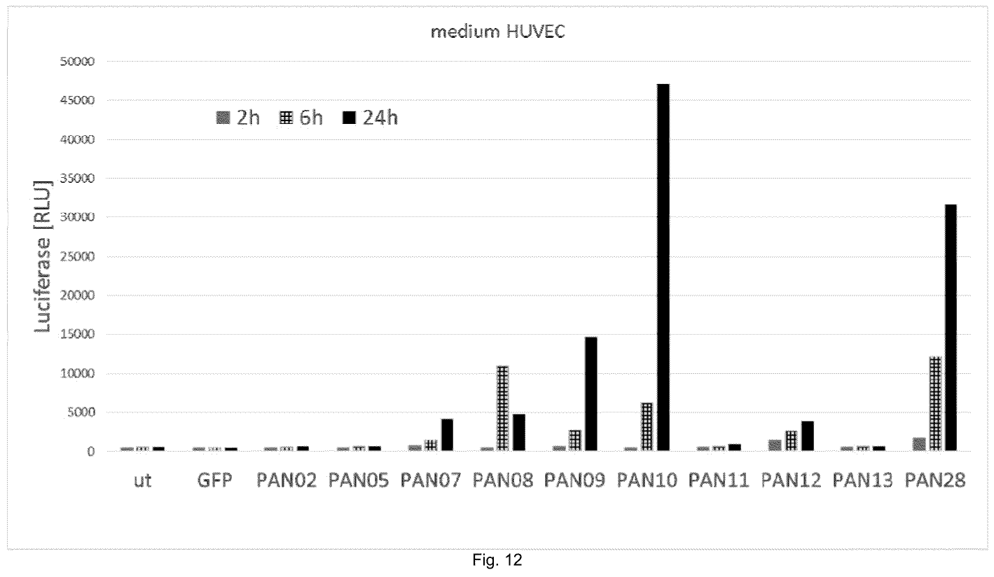

FIG. 12 is a bar diagram showing expression of luciferase, indicated as RLU (relative light units) of luciferase in medium, by the indicated recombinant nucleic acid constructs in HUVEC where luciferase is used as the effector molecule after 2 hours (left column), 6 hours (middle column) and 24 hours (right column) post transfection.

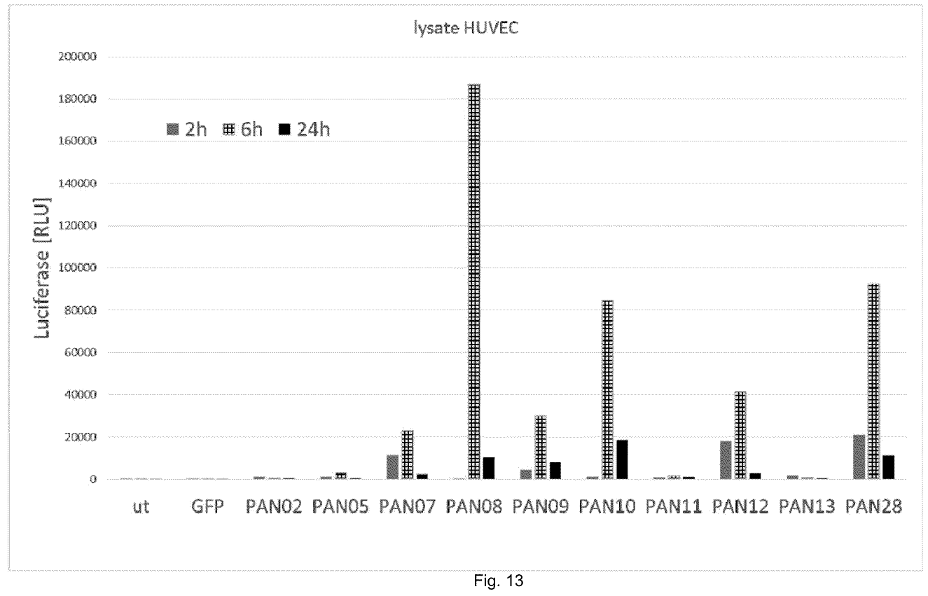

FIG. 13 is a bar diagram showing expression of luciferase, indicated as RLU (relative light units) of luciferase in whole cell lysates of HUVEC, by the indicated recombinant nucleic acid constructs after lysis of HUVEC, where luciferase is used as the effector molecule after 2 hours (left column), 6 hours (middle column) and 24 hours (right column) post transfection.

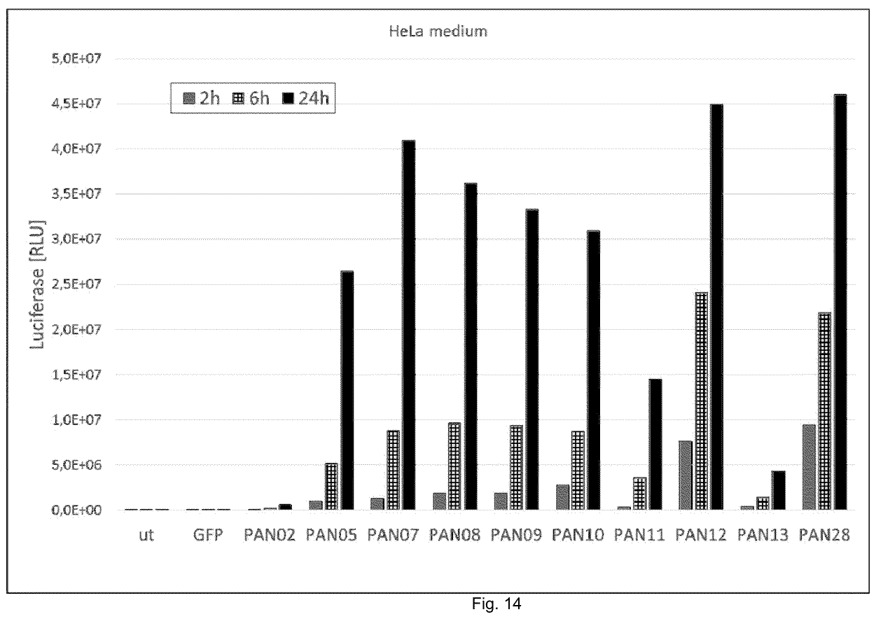

FIG. 14 is a bar diagram showing expression of luciferase, indicated as RLU [(relative light units) of luciferase in medium, by the indicated recombinant nucleic acid constructs in HeLa cells where luciferase is used as the effector molecule after 2 hours (left column), 6 hours (middle column) and 24 hours (right column) post transfection.

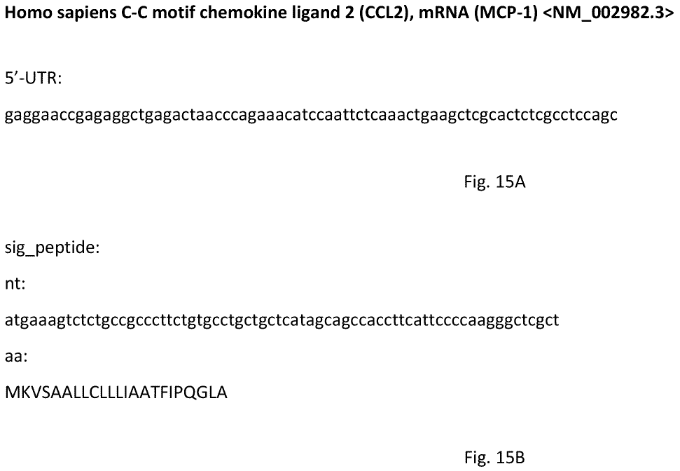

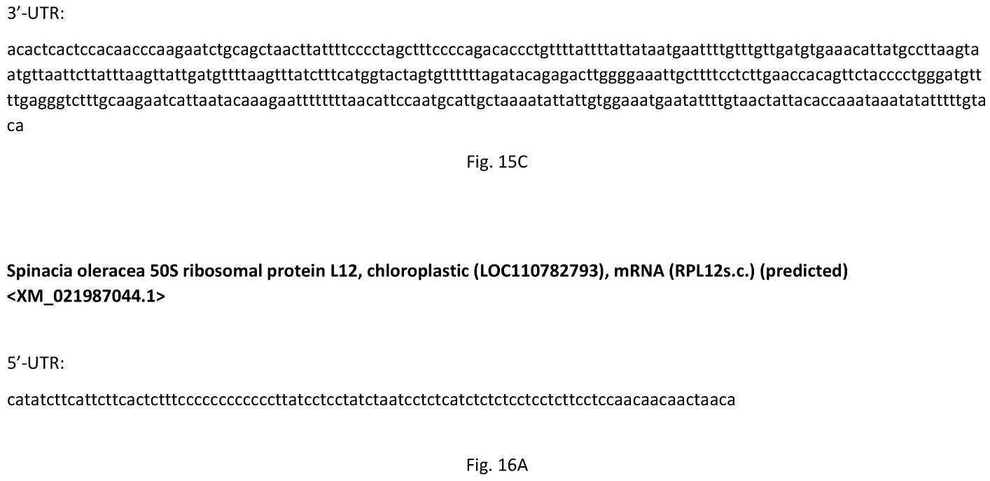

FIG. 15A shows the 5′ UTR nucleotide sequence of the mRNA of human MCP-1 which is also referred to as homo sapiens C-C motif chemokine ligand 2 (CCL2) (GenBank entry NM_002982.3).

FIG. 15B shows both the nucleotide sequence and the amino acid sequence of the signal peptide sequence of human MCP-1.

FIG. 15C shows the 3′ UTR nucleotide sequence of the mRNA of human MCP-1.

FIG. 16A shows the 5′ UTR nucleotide sequence of the mRNA of the 50S ribosomal protein L12, chloroplastic (LOC110782793), of Spinacia oleracea which is also referred to as RPL12s.c. (GenBank entry XM_021987044.1).

FIG. 16B shows the 3′ UTR from the mRNA of the 50S ribosomal protein L12, chloroplastic (LOC110782793), of Spinacia oleracea.

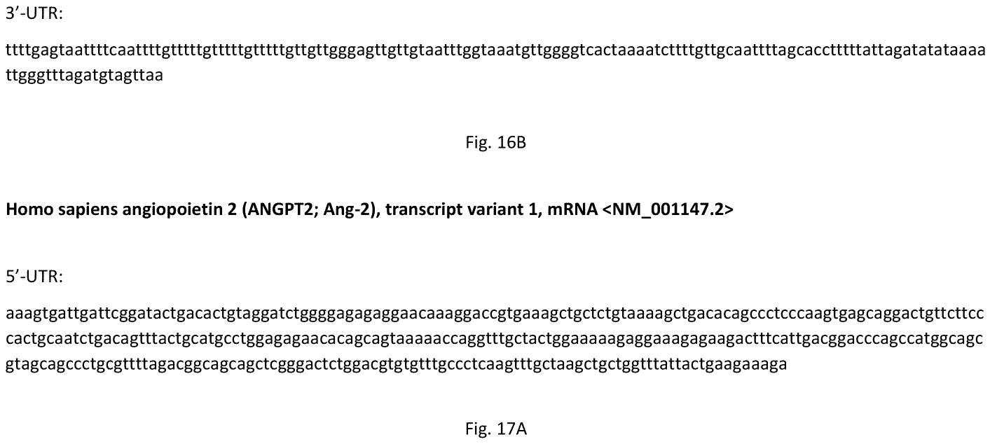

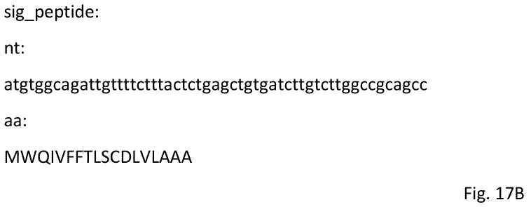

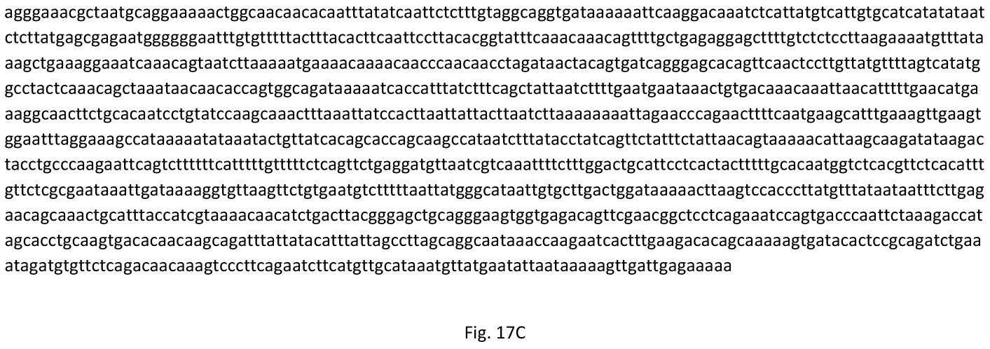

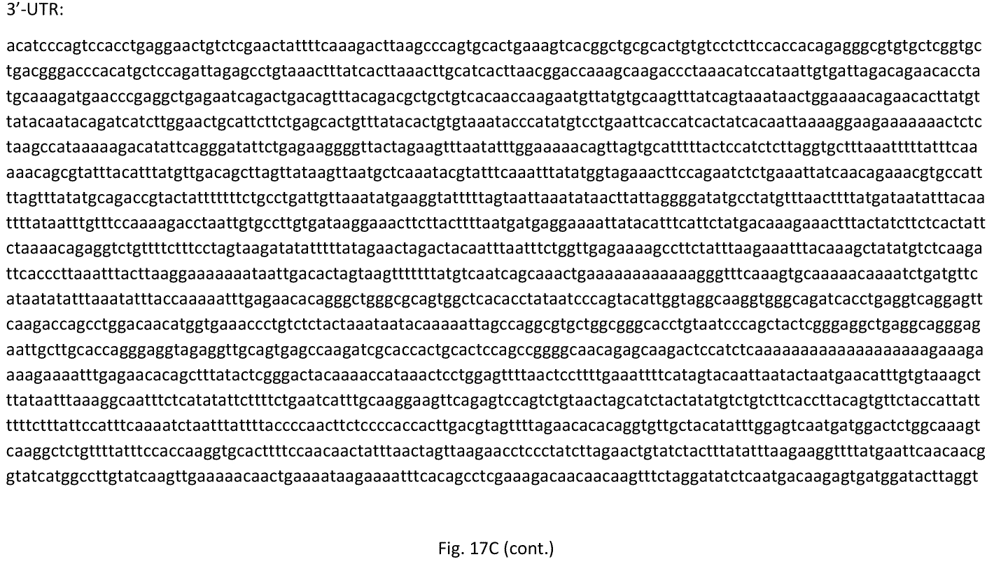

FIG. 17A shows the 5′ UTR nucleotide sequence of the mRNA of human angiopoietin 2 which is also referred to as ANGPT2 or Ang-2, transcript variant 1 (GenBank entry NM_001147.2).

FIG. 17B shows both the nucleotide sequence and the amino acid sequence of the signal sequence of the mRNA of human Ang-2.

FIG. 17C shows the 3′ UTR nucleotide sequence of the mRNA of human Ang-2.

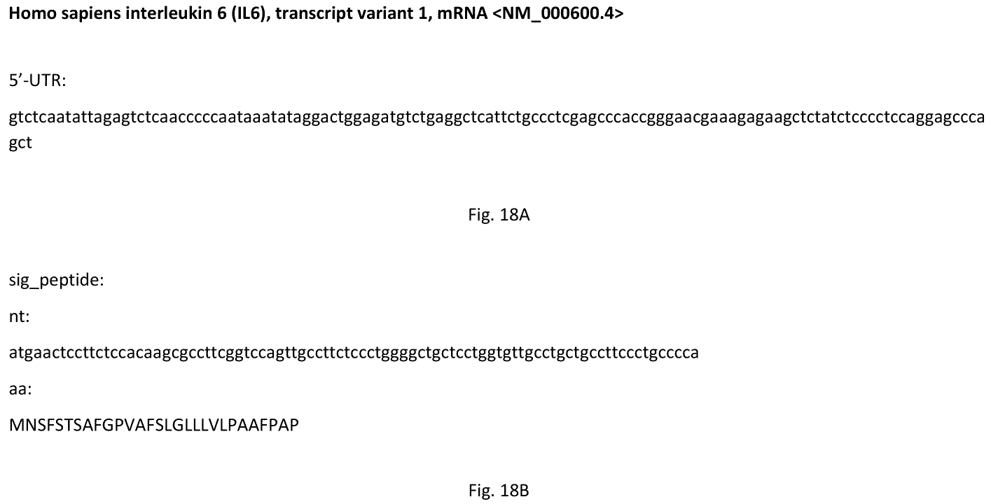

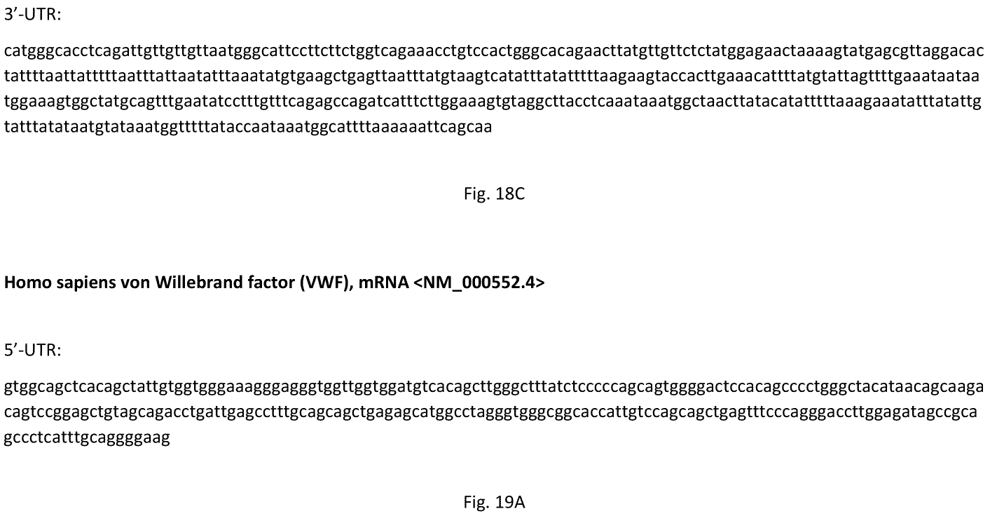

FIG. 18A shows the 5′ UTR nucleotide sequence of the mRNA of human interleukin 6 (IL-6), transcript variant 1 (GenBank entry NM_000600.4).

FIG. 18B shows both the nucleotide sequence and the amino acid sequence of the signal sequence of the mRNA of human IL-6.

FIG. 18C shows the 3′ UTR nucleotide sequence of the mRNA of human IL-6.

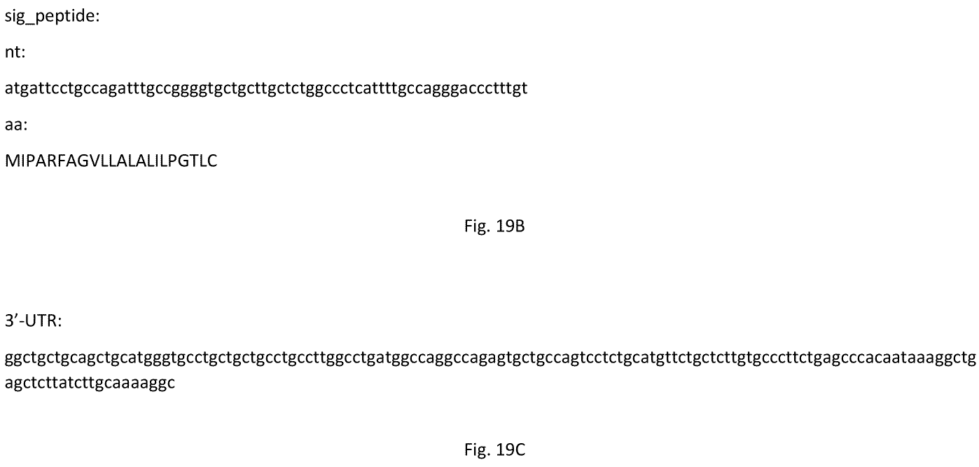

FIG. 19A shows the 5′ UTR nucleotide sequence of the mRNA of human von Willebrand factor (vWF) (GenBank entry NM_000552.4).

FIG. 19B shows both the nucleotide sequence and the amino acid sequence of the signal sequence of the mRNA of von Willebrand factor (vWF).

FIG. 19C shows the 3′ UTR nucleotide sequence of the mRNA of von Willebrand factor (vWF).

FIG. 20A shows the 5′ UTR nucleotide sequence of the mRNA of human heat shock protein family A (Hsp70) member 1A, also referred to as HSPA1A (GenBank entry NM_005345.5).

FIG. 20B shows the 3′ UTR nucleotide sequence of the mRNA of human HSPA1A.

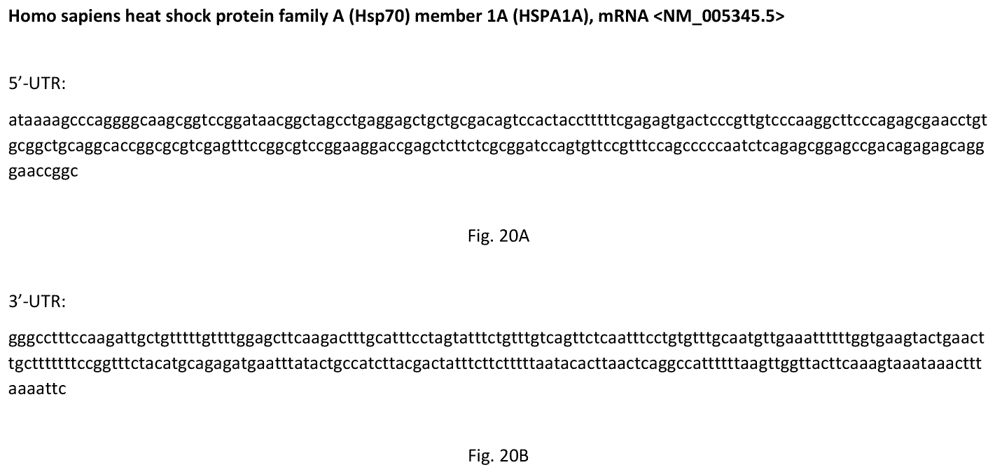

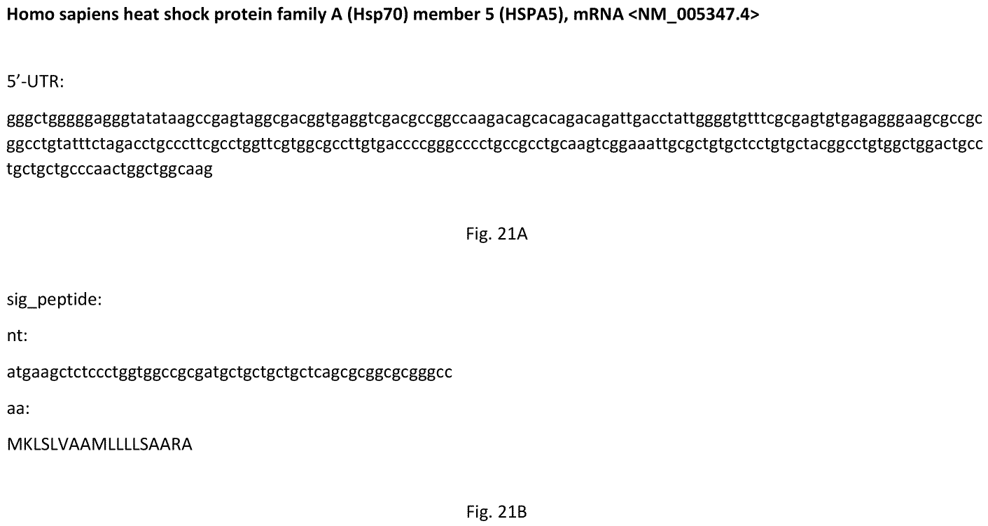

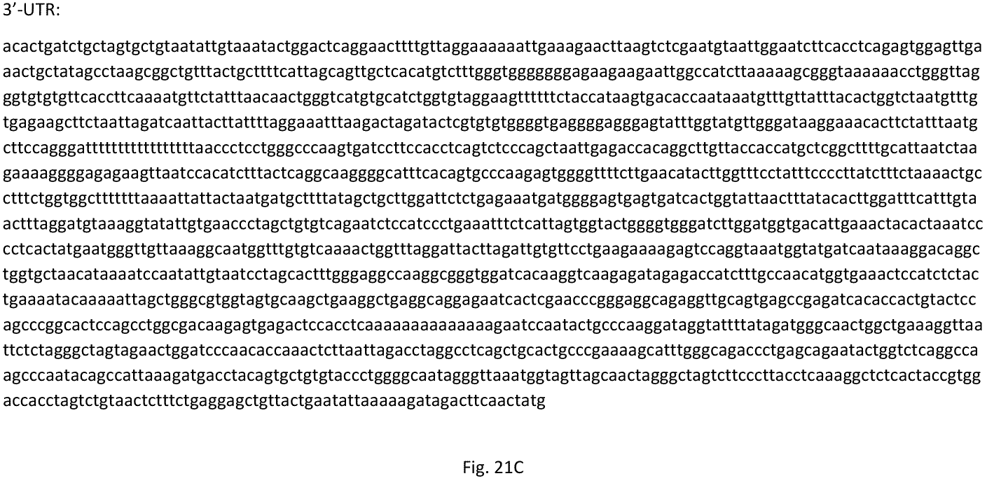

FIG. 21A shows the 5′ UTR nucleotide sequence of the mRNA of human heat shock protein family A (Hsp70) member 5, also referred to as HSPA5, (GenBank entry NM_005347.4).

FIG. 21B shows both the nucleotide sequence and the amino acid sequence of the signal sequence of the mRNA of human HSPA5.

FIG. 21B shows both the nucleotide sequence and the amino acid sequence of the signal sequence of the mRNA of human HSPA5.

FIG. 21C shows the 3′ UTR nucleotide sequence of the mRNA of human HSPA1A.

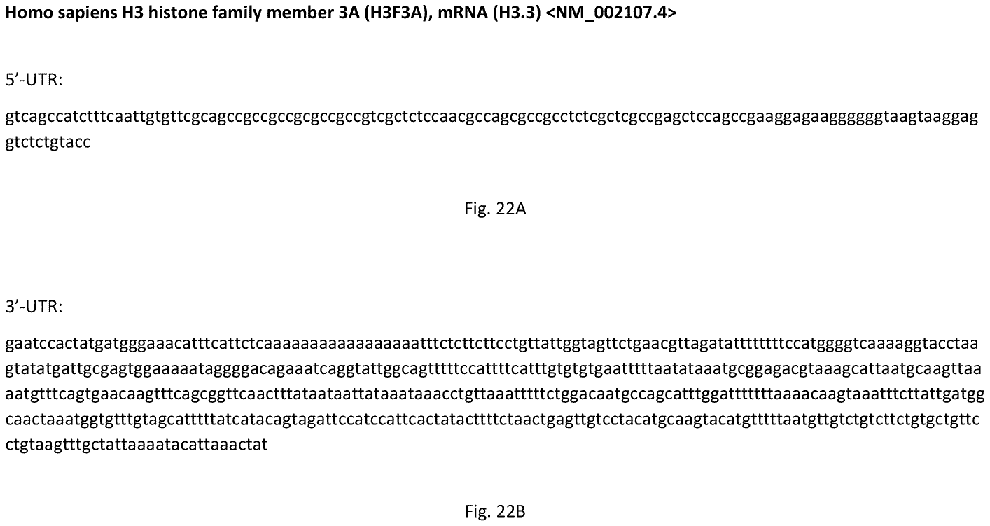

FIG. 22A shows the 5′ UTR nucleotide sequence of the mRNA of human H3 histone family member 3A (H3F3A), also referred to as H3.3, (GenBank entry NM_002107.4).

FIG. 22B shows the 3′ UTR nucleotide sequence of the mRNA of human H3.3.

FIG. 23A shows the 5′ UTR nucleotide sequence of the mRNA of human galectin-9 (LGALS9), transcript variant 1 (GenBank entry NM_009587.2).

FIG. 23B shows the 3′ UTR nucleotide sequence of the mRNA of human galectin-9.

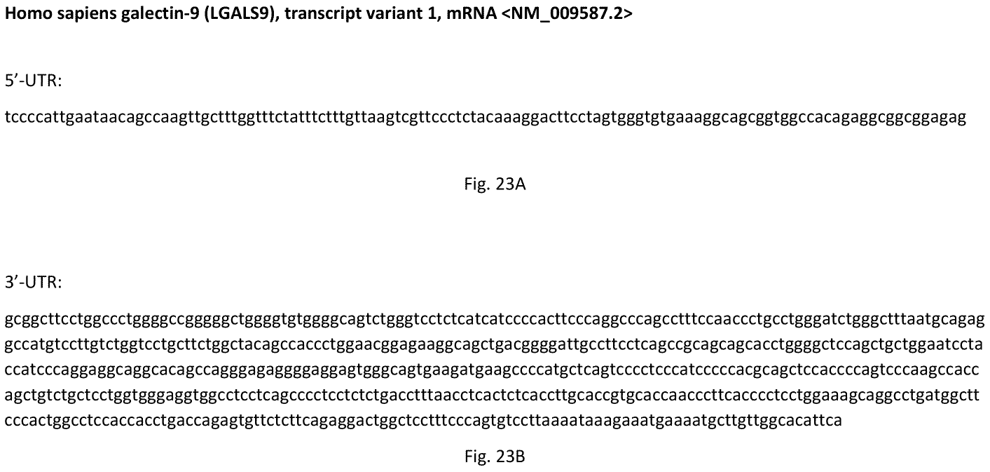

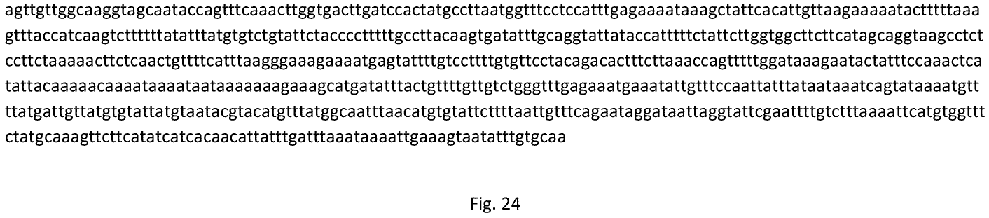

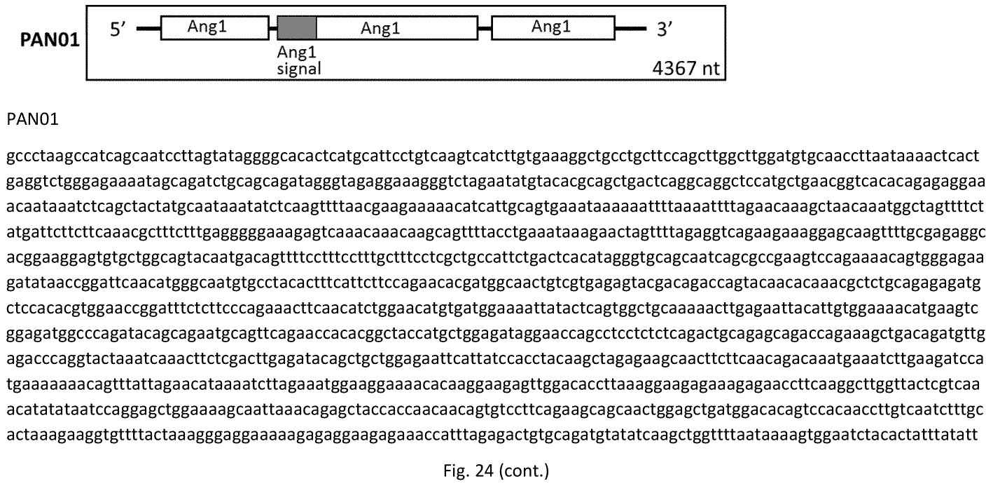

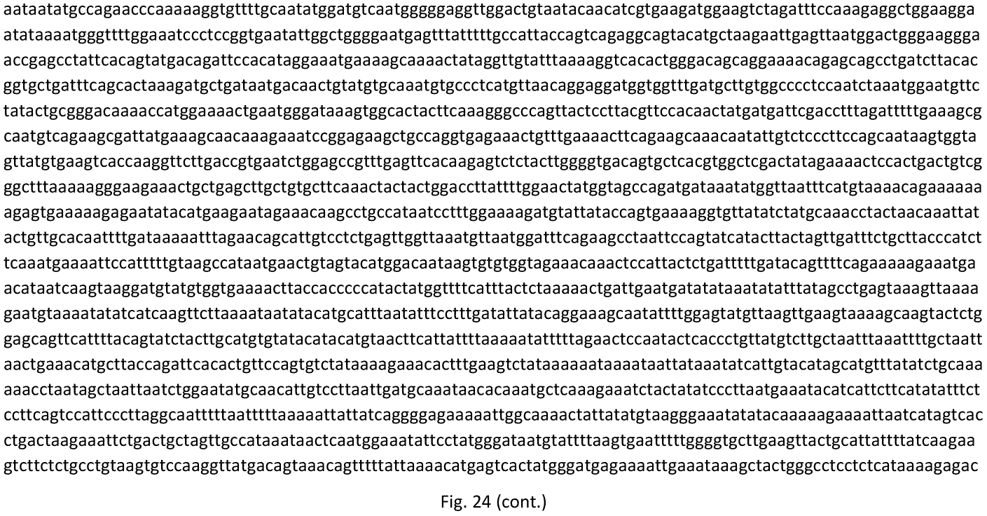

FIG. 24 shows the basic structure and nucleotide sequence of recombinant nucleic acid construct PAN01.

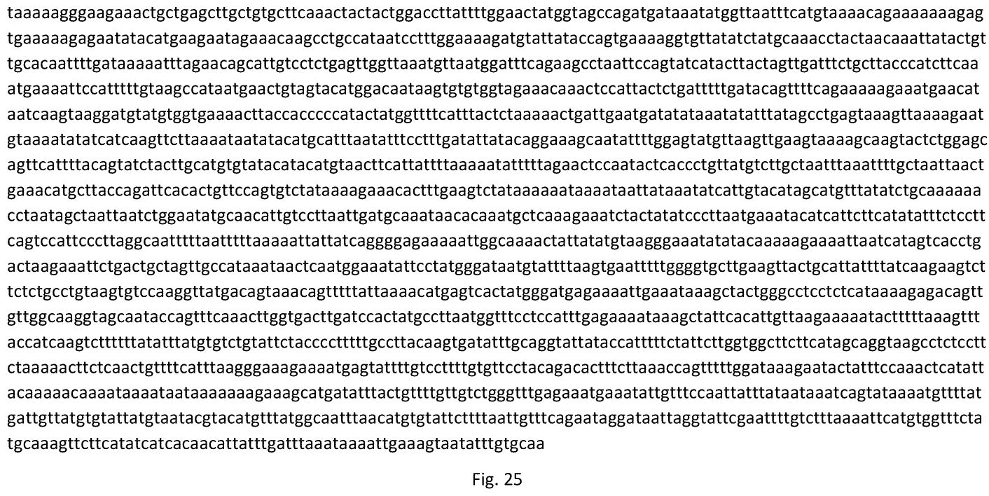

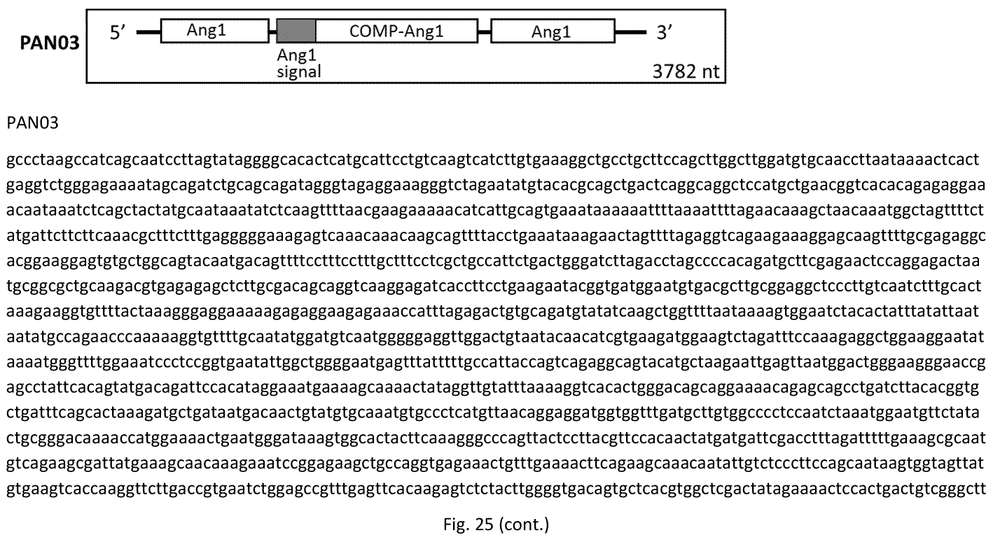

FIG. 25 shows the basic structure and nucleotide sequence of recombinant nucleic acid construct PAN03.

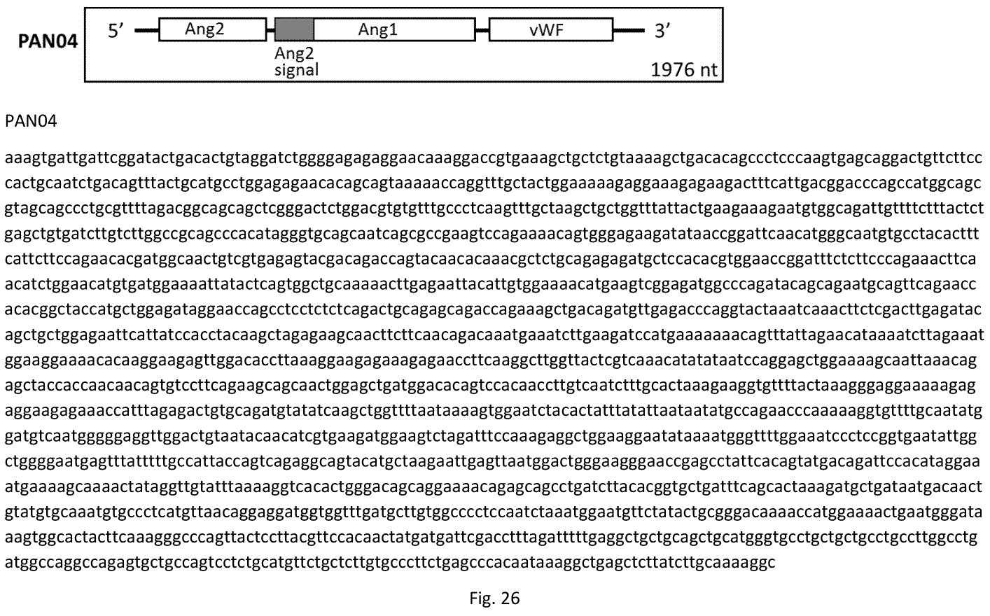

FIG. 26 shows the basic structure and nucleotide sequence of recombinant nucleic acid construct PAN04.

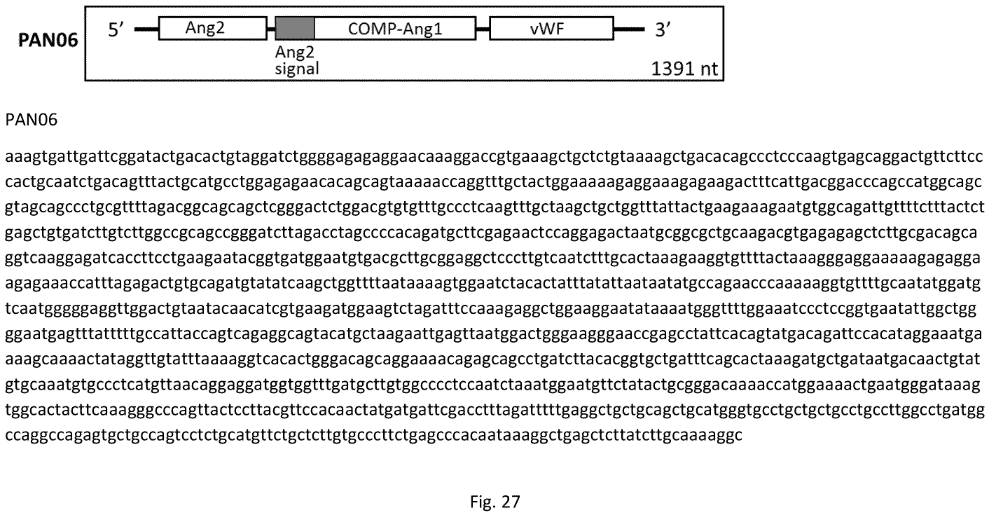

FIG. 27 shows the basic structure and nucleotide sequence of recombinant nucleic acid construct PAN06.

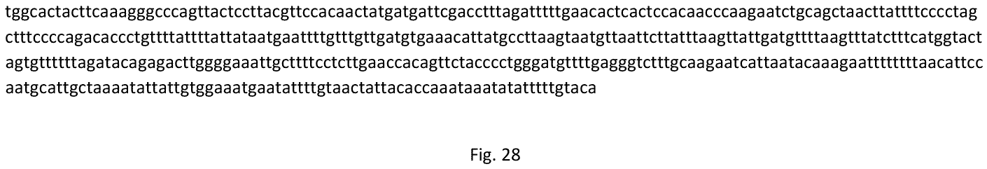

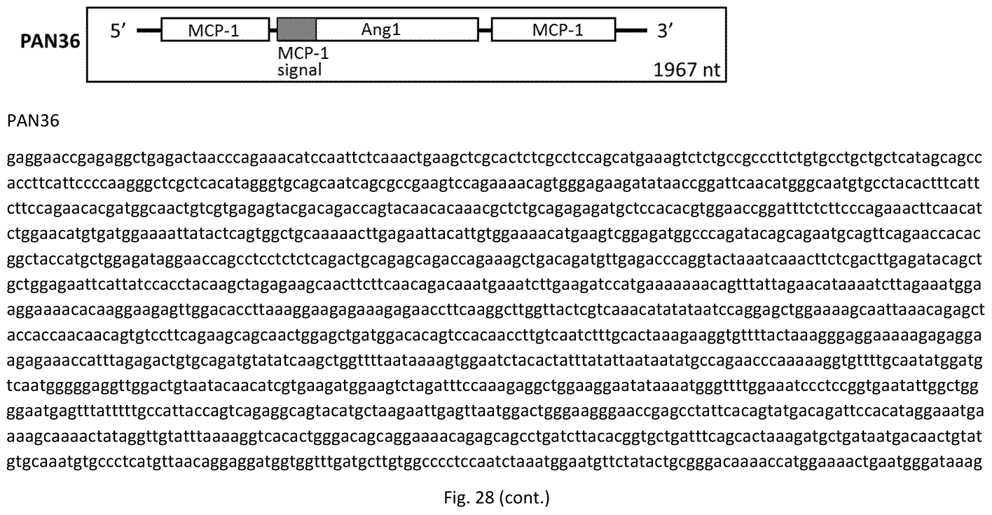

FIG. 28 shows the basic structure and nucleotide sequence of recombinant nucleic acid construct PAN36.

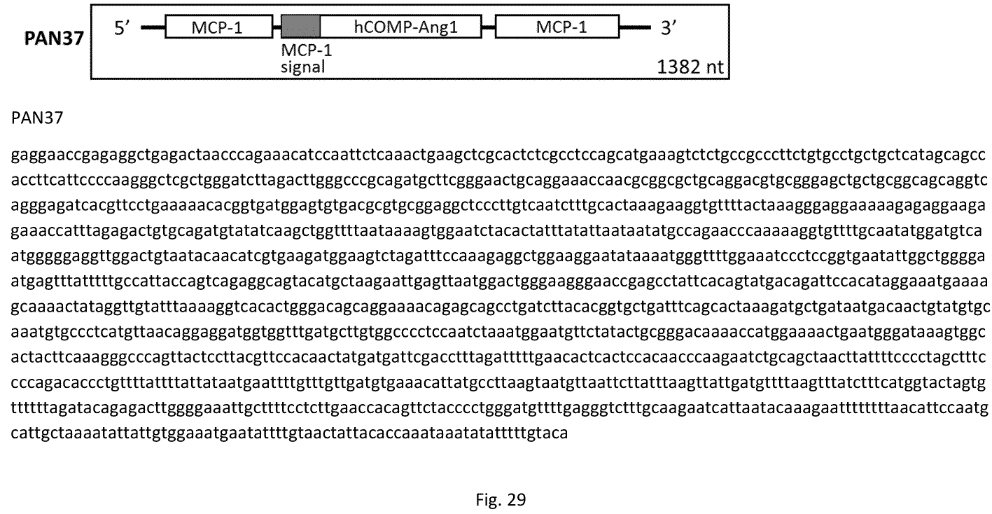

FIG. 29 shows the basic structure and nucleotide sequence of recombinant nucleic acid construct PAN37.

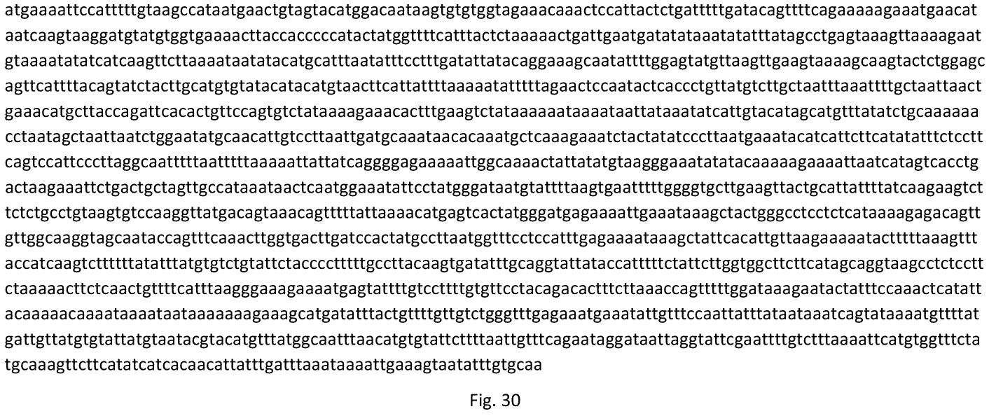

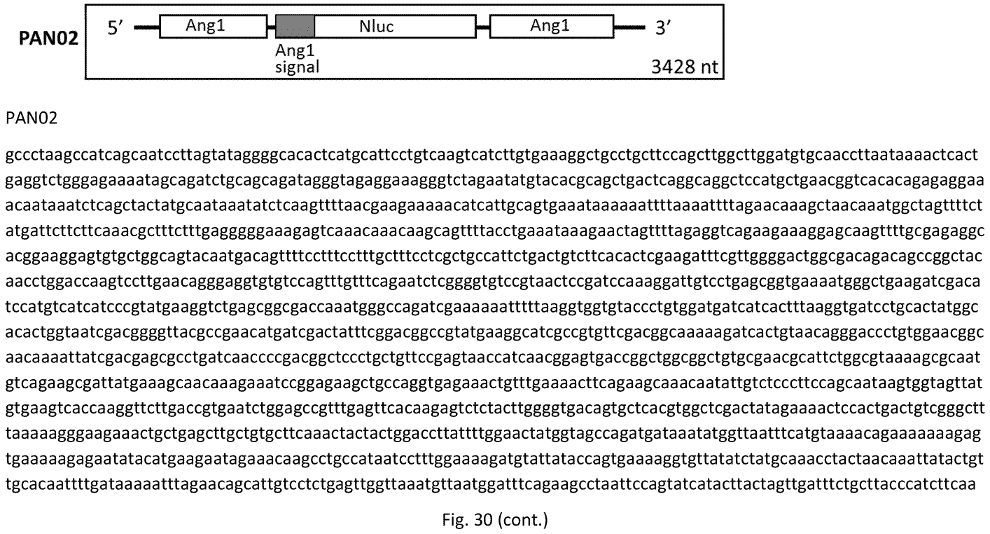

FIG. 30 shows the basic structure and nucleotide sequence of recombinant nucleic acid construct PAN02.

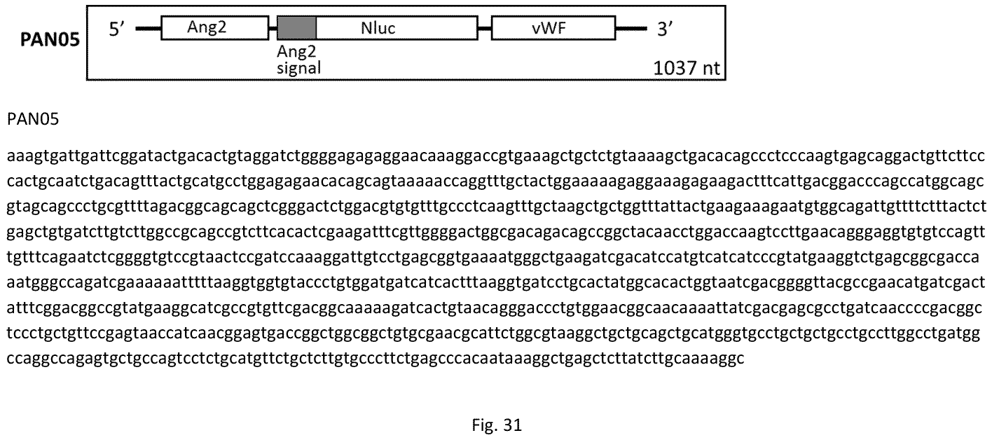

FIG. 31 shows the basic structure and nucleotide sequence of recombinant nucleic acid construct PAN05.

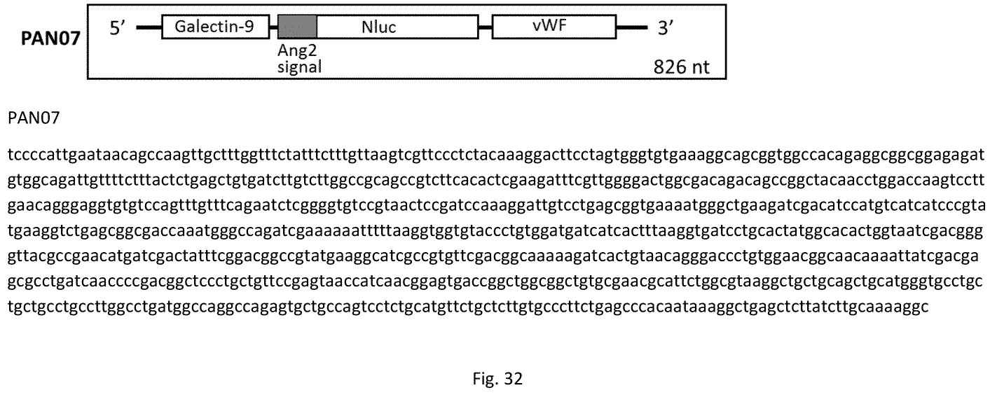

FIG. 32 shows the basic structure and nucleotide sequence of recombinant nucleic acid construct PAN07.

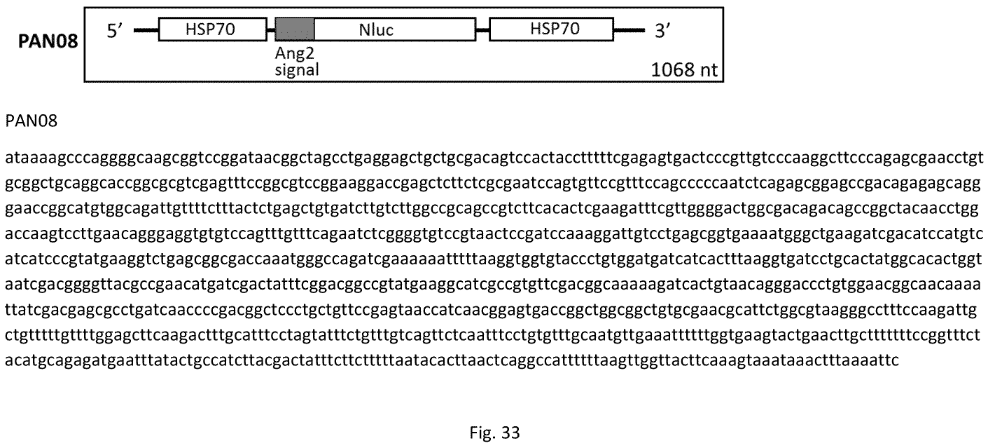

FIG. 33 shows the basic structure and nucleotide sequence of recombinant nucleic acid construct PAN08.

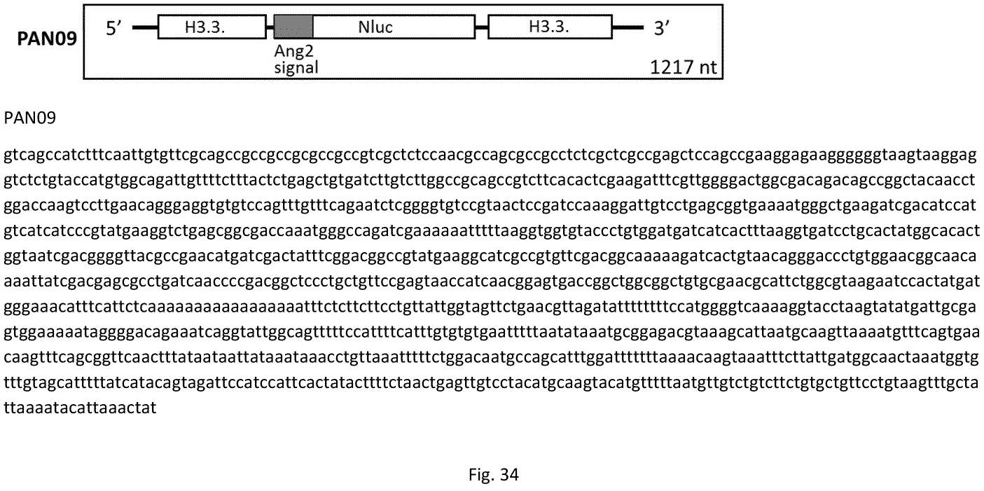

FIG. 34 shows the basic structure and nucleotide sequence of recombinant nucleic acid construct PAN09.

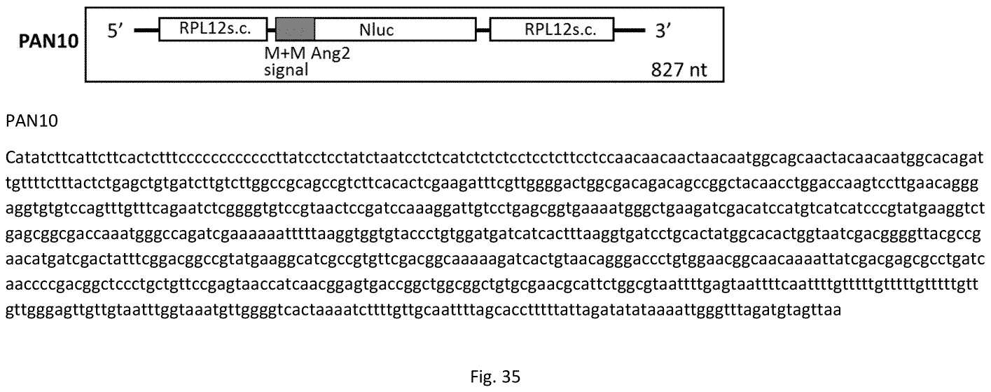

FIG. 35 shows the basic structure and nucleotide sequence of recombinant nucleic acid construct PAN10.

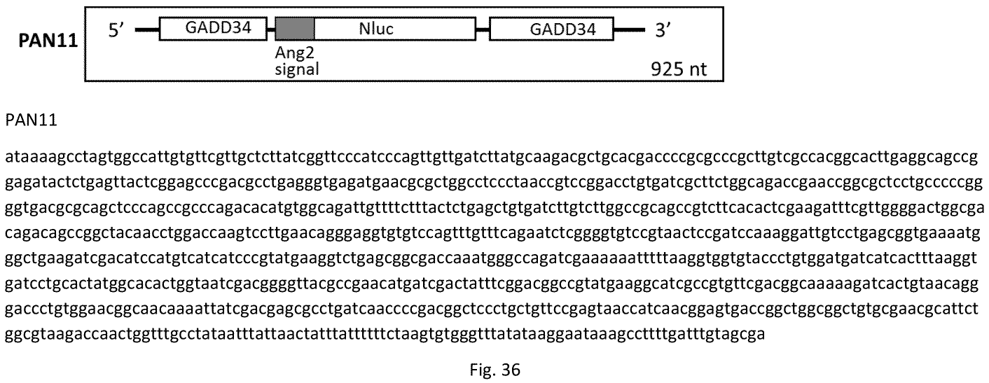

FIG. 36 shows the basic structure and nucleotide sequence of recombinant nucleic acid construct PAN11.

FIG. 37 shows the basic structure and nucleotide sequence of recombinant nucleic acid construct PAN12.

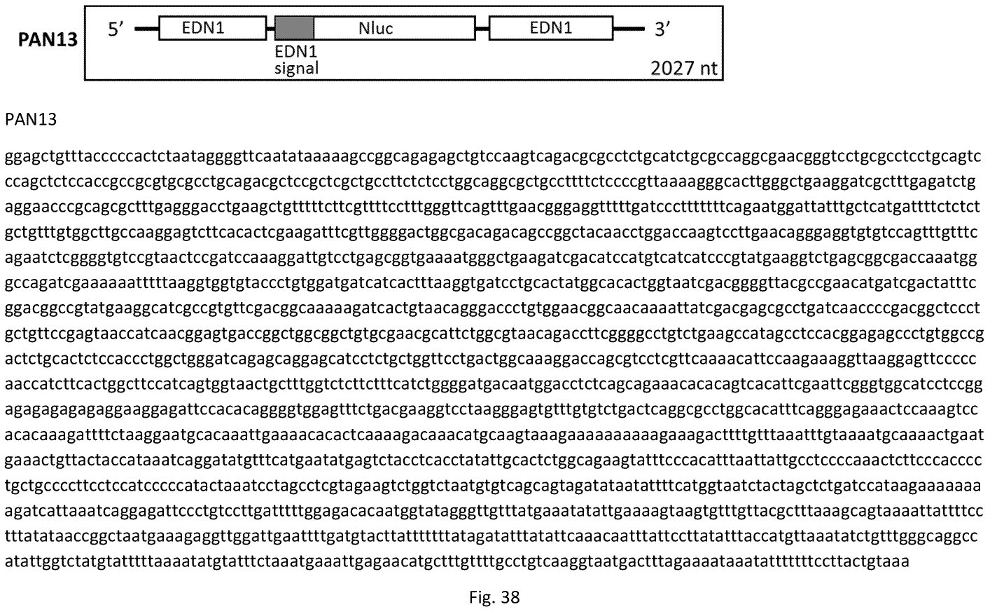

FIG. 38 shows the basic structure and nucleotide sequence of recombinant nucleic acid construct PAN13.

FIG. 39 shows the basic structure and nucleotide sequence of recombinant nucleic acid construct PAN28.

FIG. 40 shows the basic structure and nucleotide sequence of recombinant nucleic acid construct PAN29.

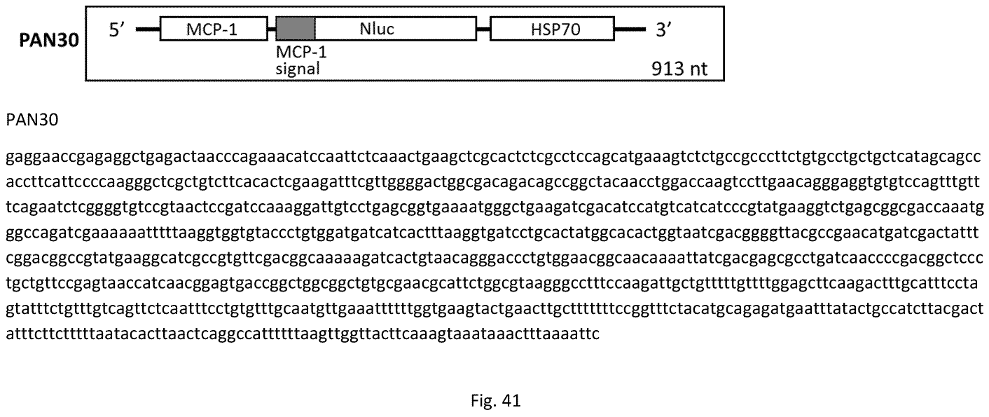

FIG. 41 shows the basic structure and nucleotide sequence of recombinant nucleic acid construct PAN30.

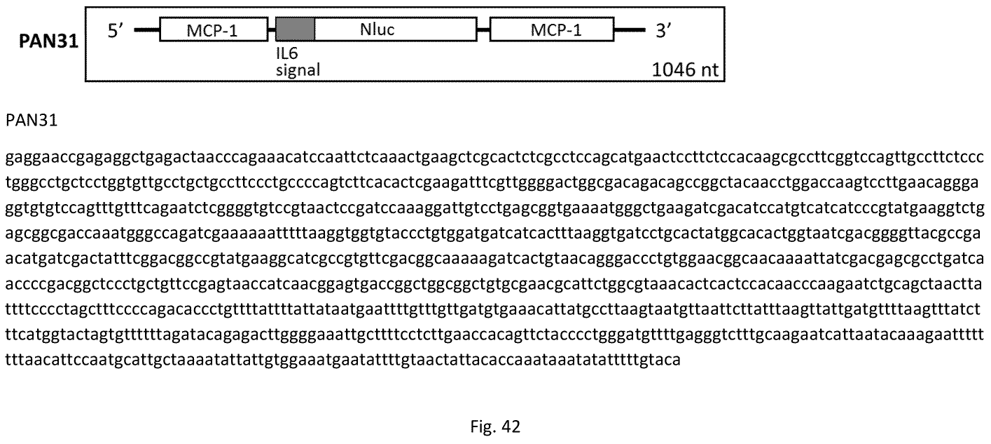

FIG. 42 shows the basic structure and nucleotide sequence of recombinant nucleic acid construct PAN31.

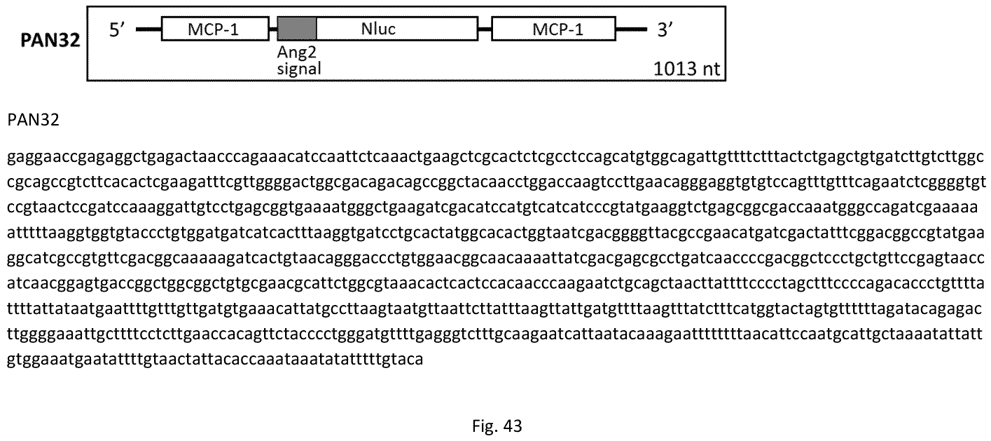

FIG. 43 shows the basic structure and nucleotide sequence of recombinant nucleic acid construct PAN32.

FIG. 44 shows the basic structure and nucleotide sequence of recombinant nucleic acid construct PAN33.

FIG. 45 shows the basic structure and nucleotide sequence of recombinant nucleic acid construct PAN34.

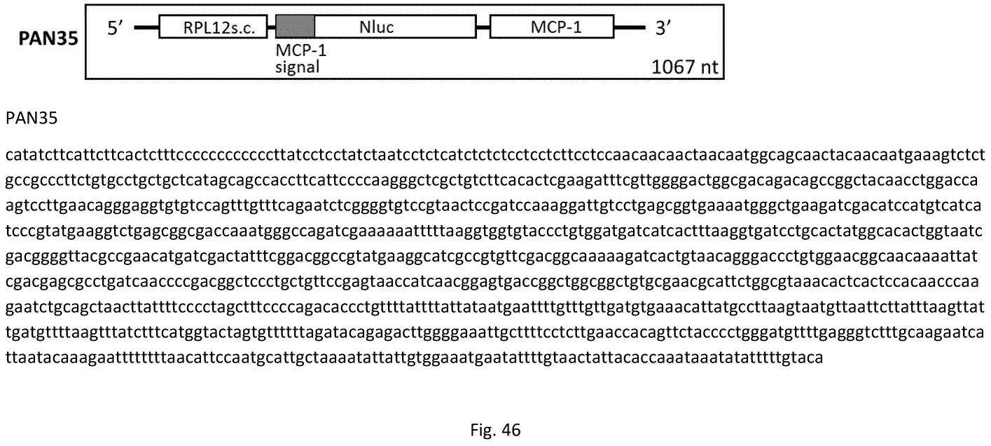

FIG. 46 shows the basic structure and nucleotide sequence of recombinant nucleic acid construct PAN35.

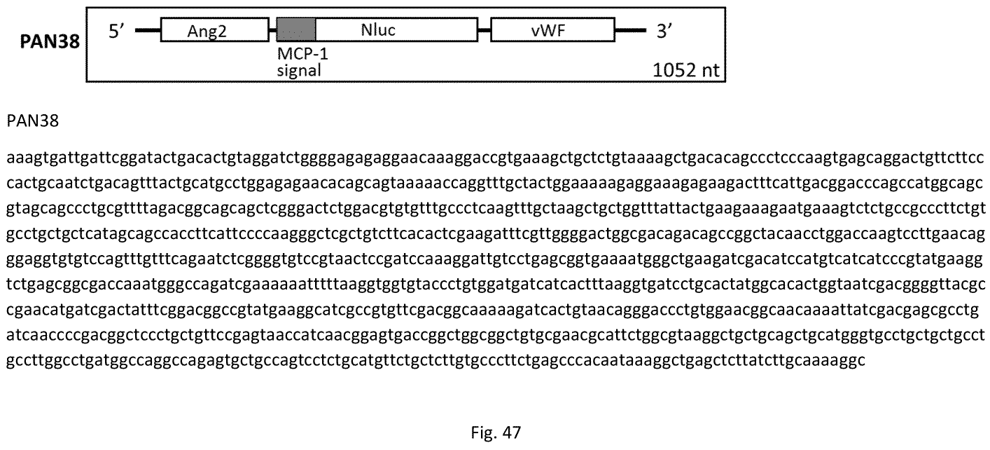

FIG. 47 shows the basic structure and nucleotide sequence of recombinant nucleic acid construct PAN38.

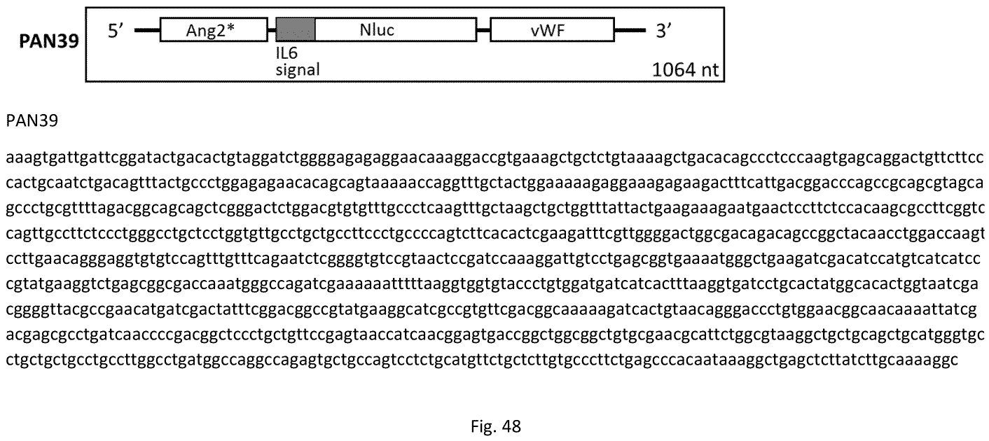

FIG. 48 shows the basic structure and nucleotide sequence of recombinant nucleic acid construct PAN39.

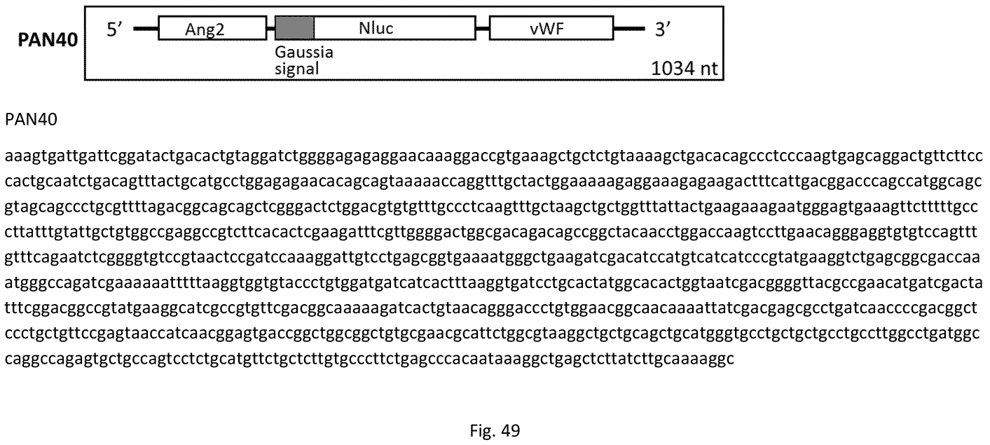

FIG. 49 shows the basic structure and nucleotide sequence of recombinant nucleic acid construct PAN40.

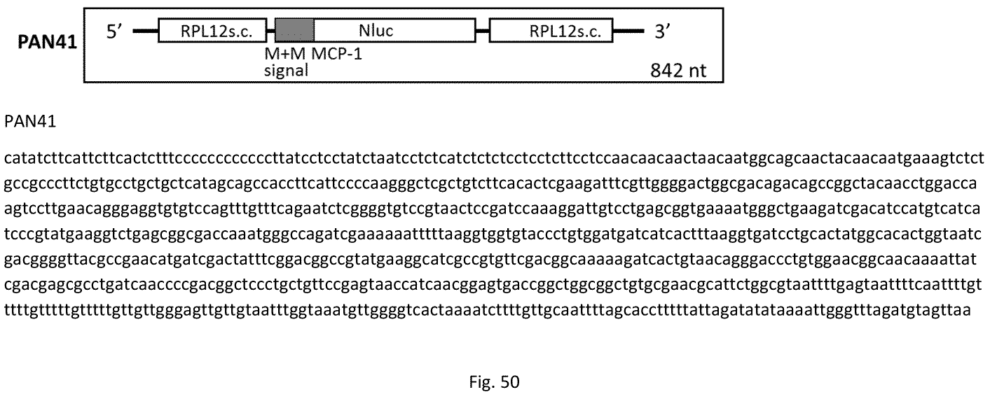

FIG. 50 shows the basic structure and nucleotide sequence of recombinant nucleic acid construct PAN41.

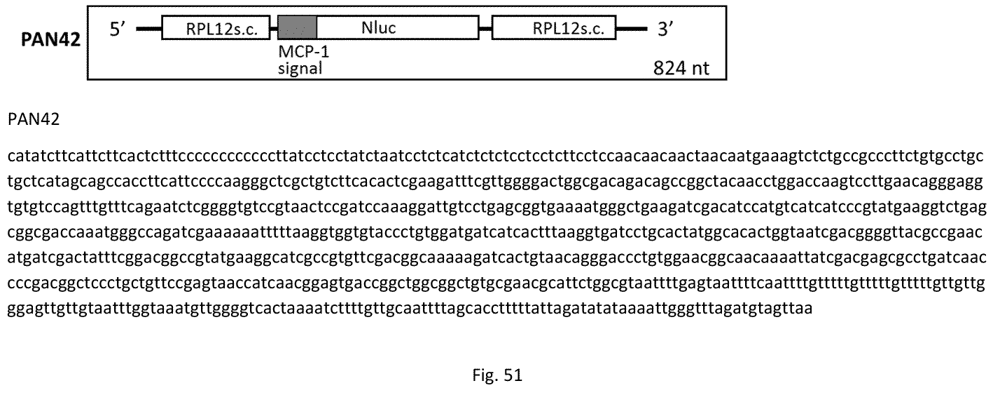

FIG. 51 shows the basic structure and nucleotide sequence of recombinant nucleic acid construct PAN42.

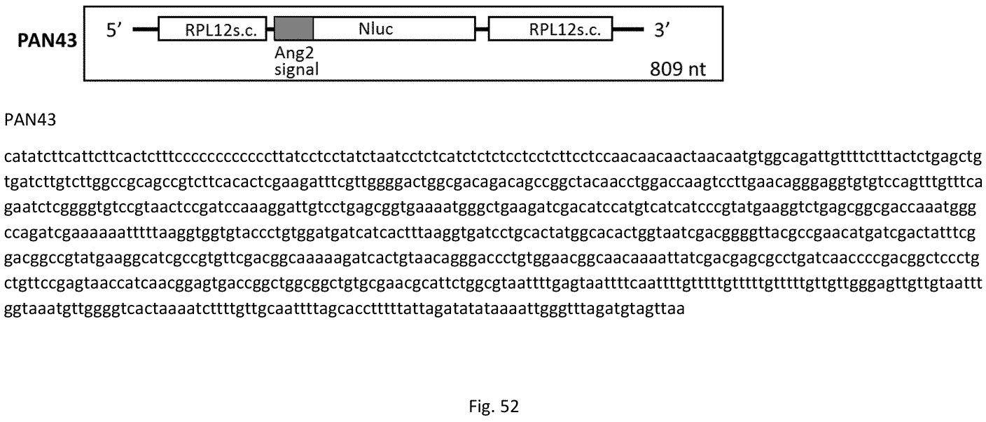

FIG. 52 shows the basic structure and nucleotide sequence of recombinant nucleic acid construct PAN43.

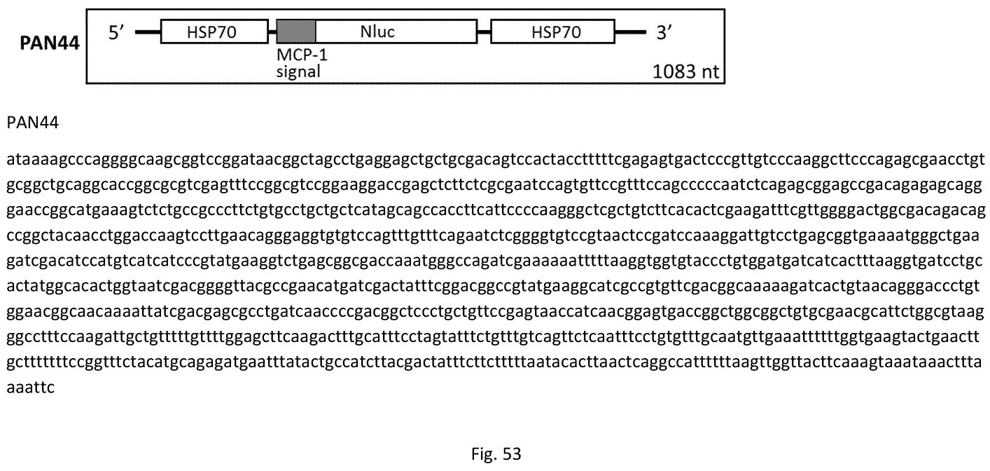

FIG. 53 shows the basic structure and nucleotide sequence of recombinant nucleic acid construct PAN44.

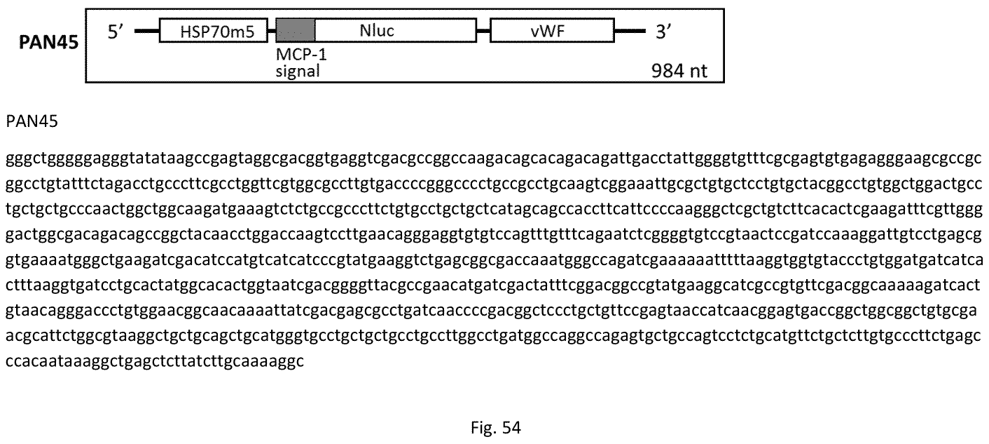

FIG. 54 shows the basic structure and nucleotide sequence of recombinant nucleic acid construct PAN45.

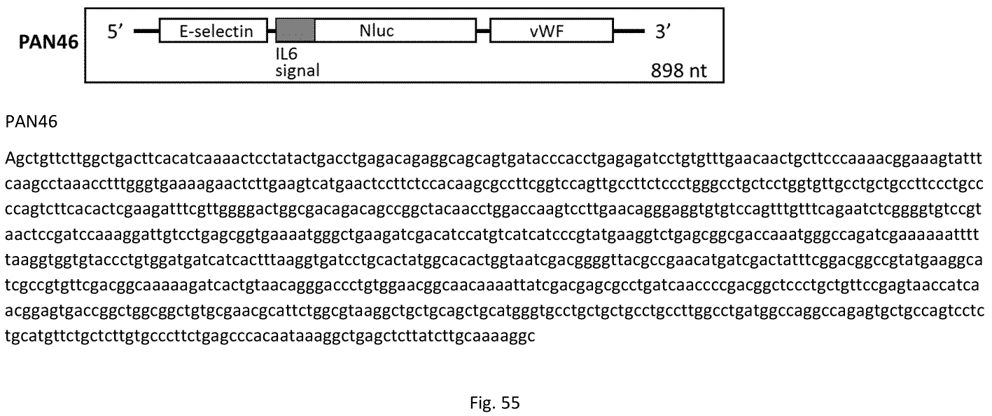

FIG. 55 shows the basic structure and nucleotide sequence of recombinant nucleic acid construct PAN46.

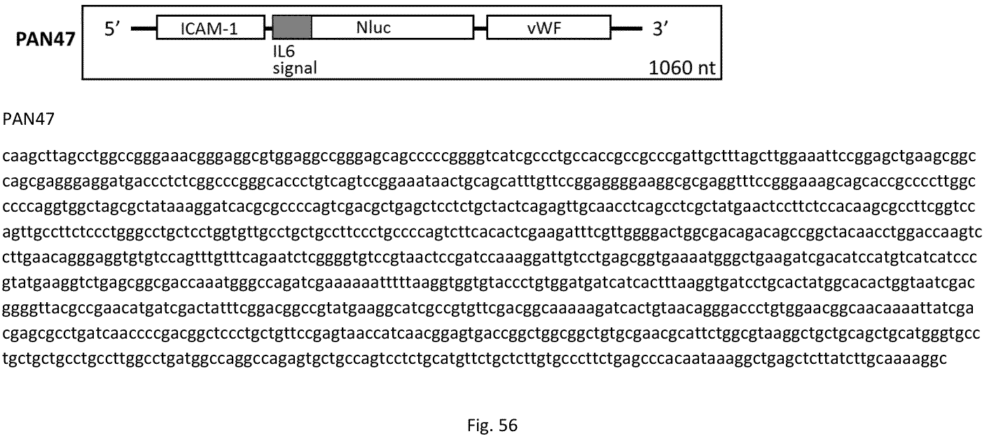

FIG. 56 shows the basic structure and nucleotide sequence of recombinant nucleic acid construct PAN47.

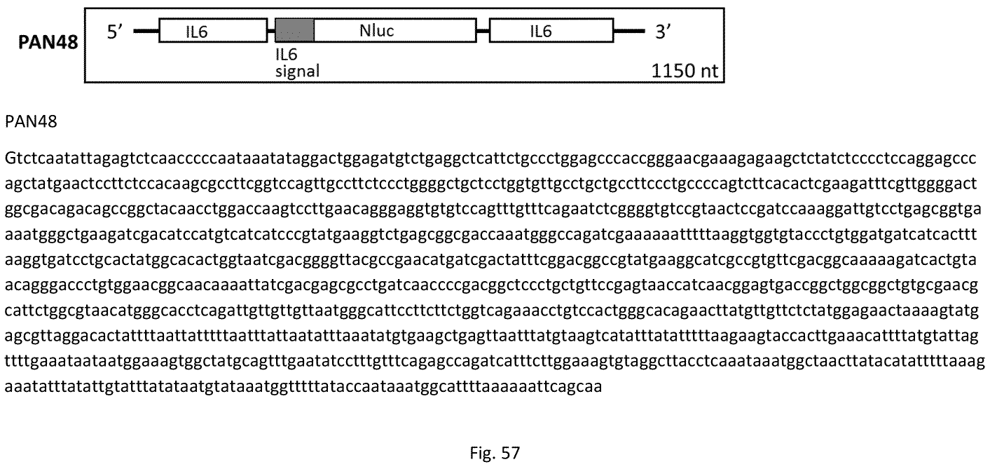

FIG. 57 shows the basic structure and nucleotide sequence of recombinant nucleic acid construct PAN48.

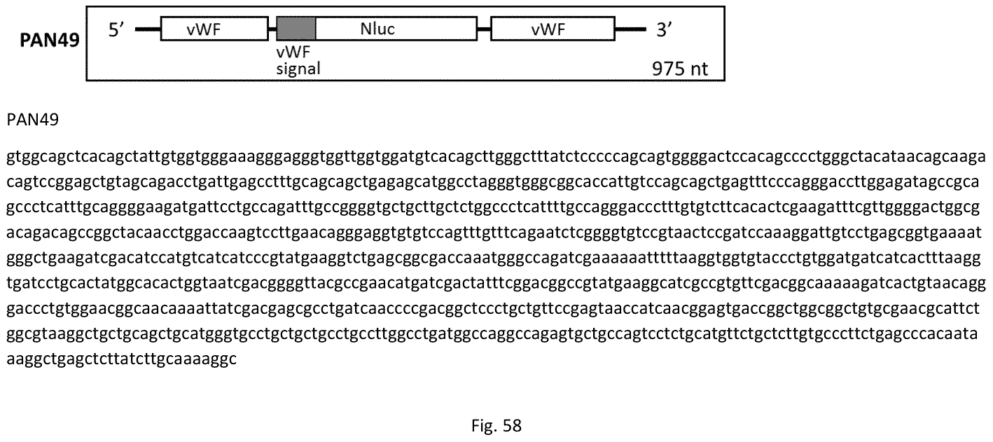

FIG. 58 shows the basic structure and nucleotide sequence of recombinant nucleic acid construct PAN49.

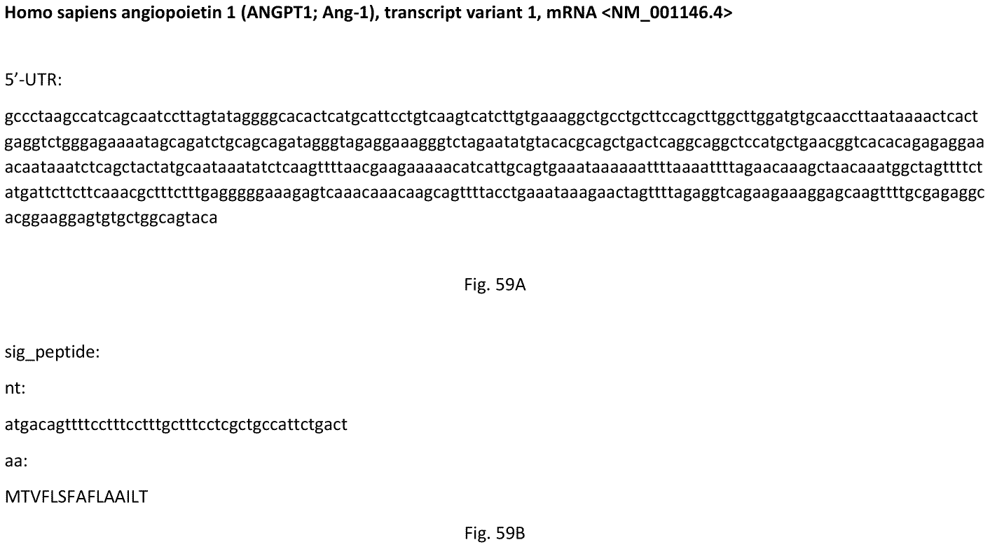

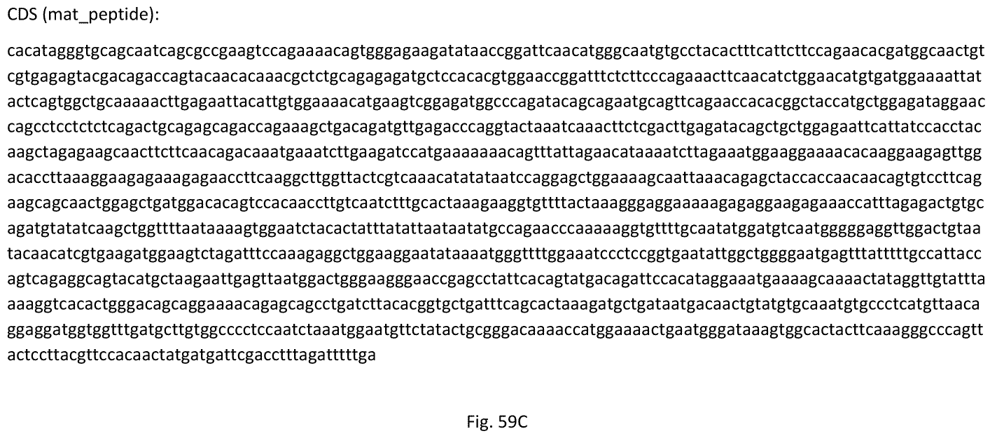

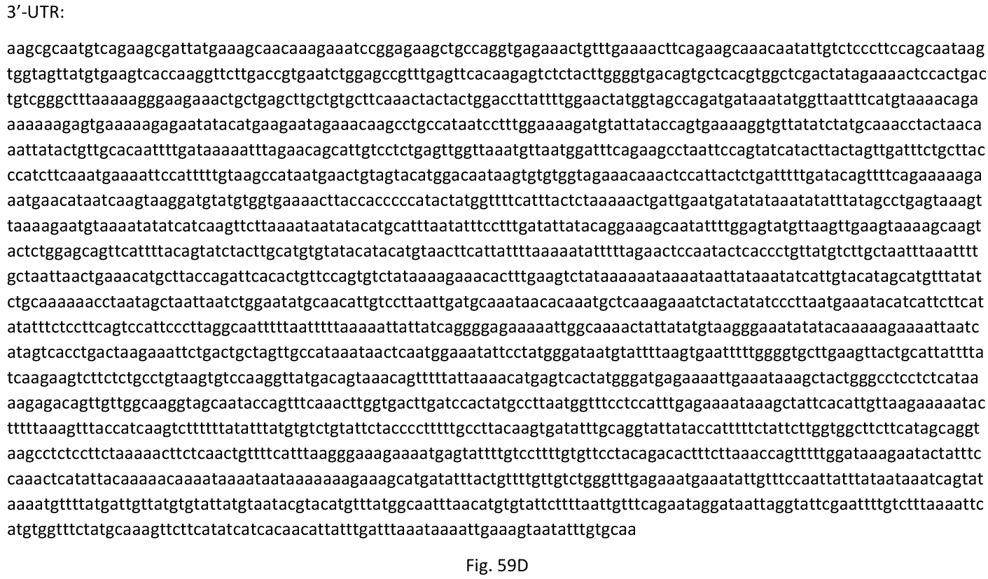

FIG. 59A shows the nucleotide sequence of mRNA of human angiopoietin 1, also referred to as ANGPT1 or Ang-1, transcript variant 1, GenBank entry NM_001146.4.

FIG. 59B shows both the nucleotide sequence and the amino acid sequence of the mRNA of human Ang-1.

FIG. 59C shows the coding sequence (CDS) of the mature peptide of mRNA of human Ang-1.

FIG. 59D shows the 3′ UTR nucleotide sequence of the mRNA of human Ang-1.

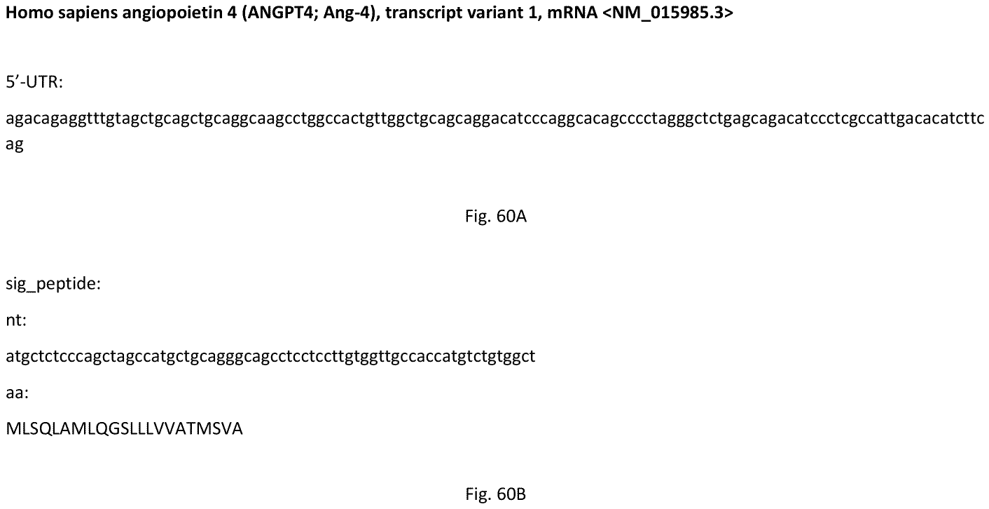

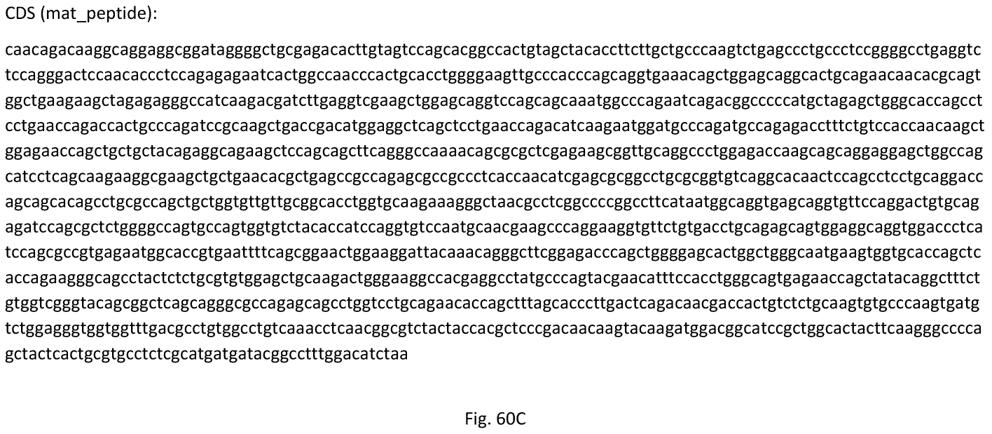

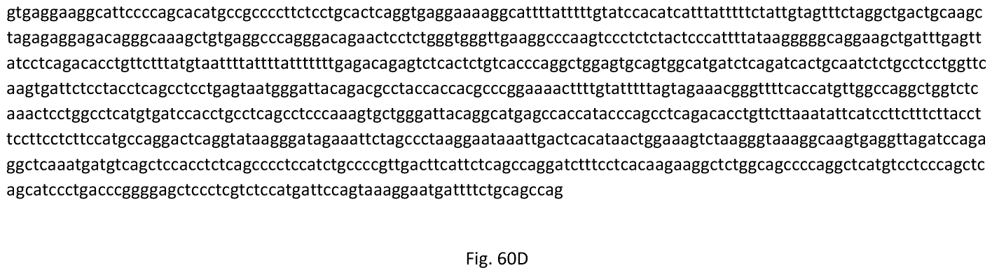

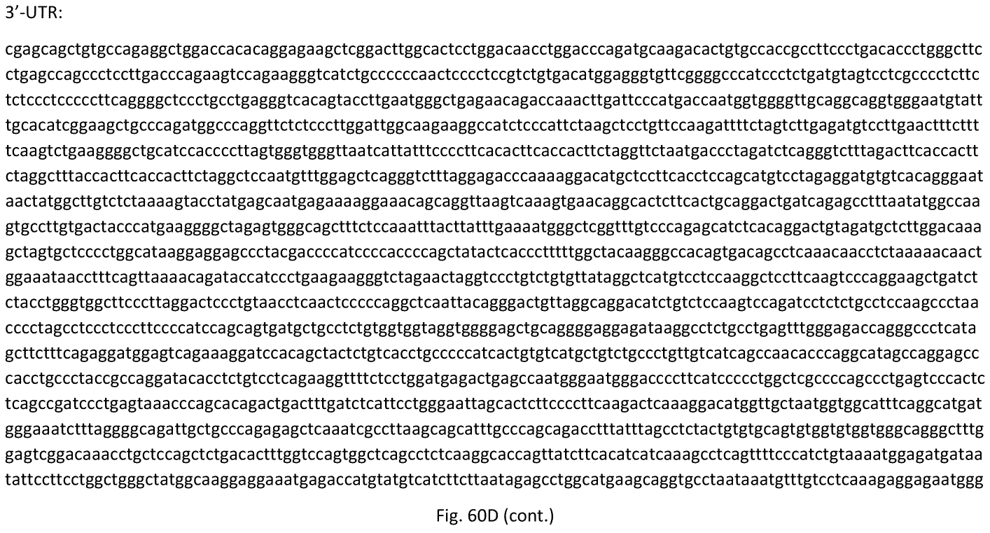

FIG. 60A shows the nucleotide sequence of mRNA of human angiopoietin 4, also referred to as ANGPT4 or Ang-4, transcript variant 1, GenBank entry NM_015985.3.

FIG. 60B shows both the nucleotide sequence and the amino acid sequence of the mRNA of human Ang-4.

FIG. 60C shows the coding sequence (CDS) of the mature peptide of mRNA of human Ang-4.

FIG. 60D shows the 3′ UTR nucleotide sequence of the mRNA of human Ang-4.

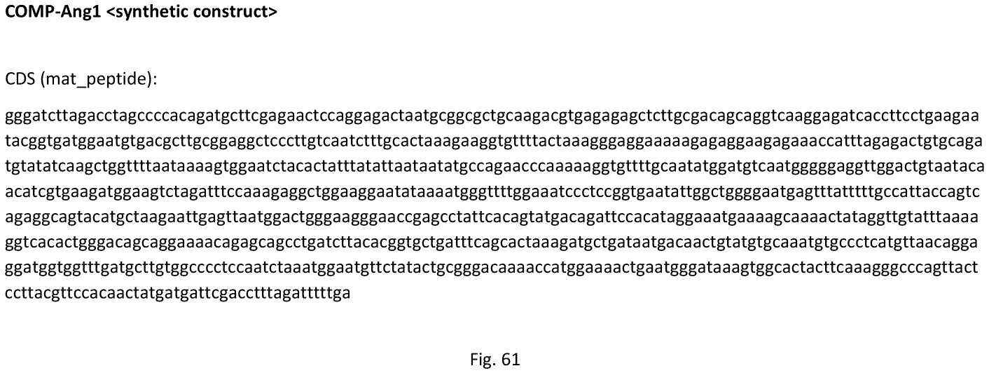

FIG. 61 shows the nucleotide sequence coding for COMP-Ang1 which is a synthetic construct.

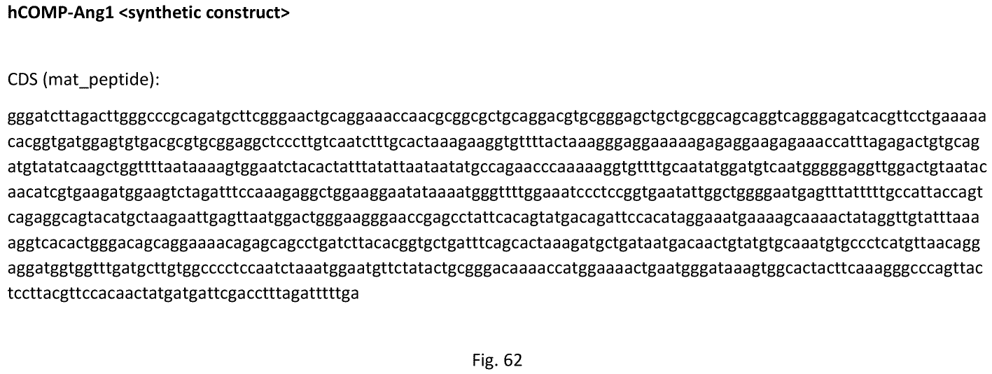

FIG. 62 shows the nucleotide sequence coding for hCOMP-Ang1 which is a synthetic construct.

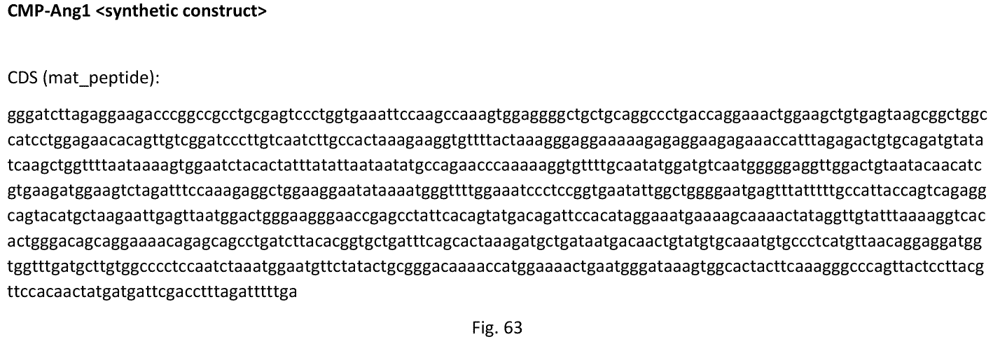

FIG. 63 shows the nucleotide sequence coding for CMP-Ang1 which is a synthetic construct.

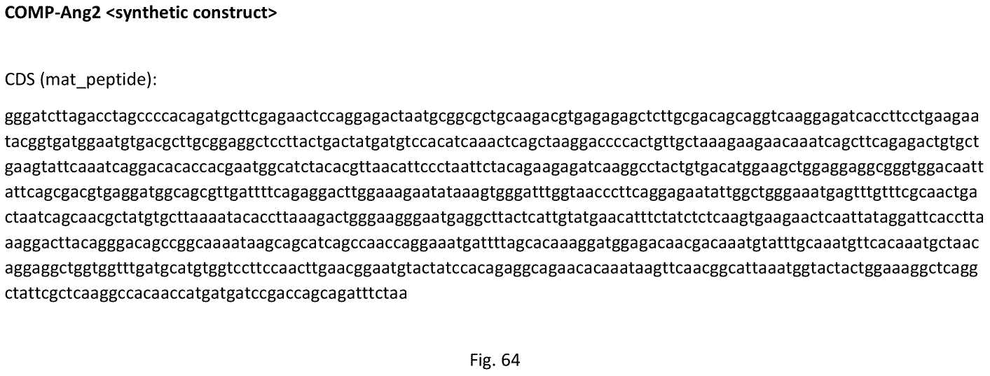

FIG. 64 shows the nucleotide sequence coding for COMP-Ang2 which is a synthetic construct.

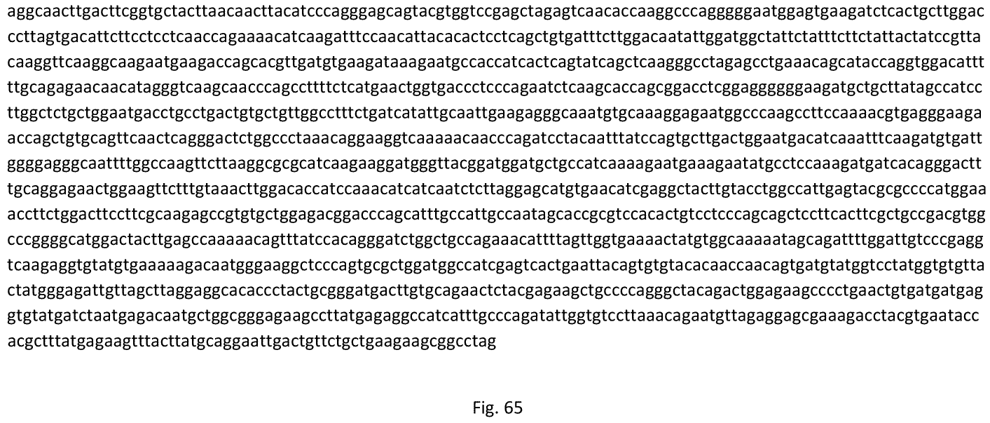

FIG. 65 shows the coding nucleotide sequence of mRNA of human TEK receptor tyrosine kinase (TEK), transcript variant 1, also referred to as Tie-2, GenBank entry.

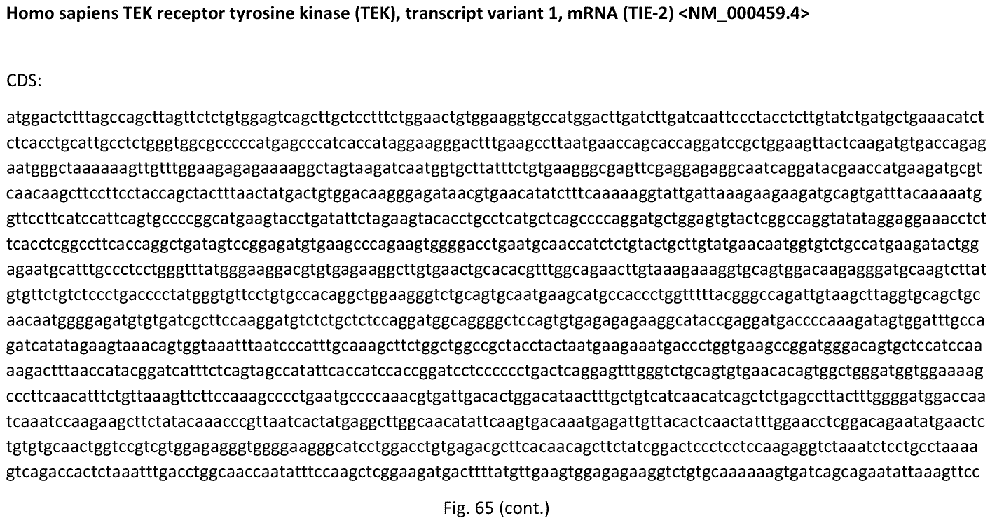

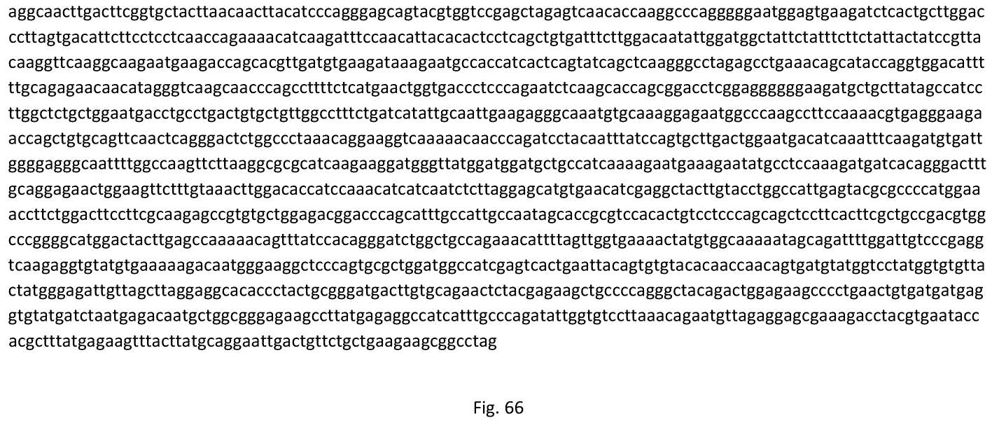

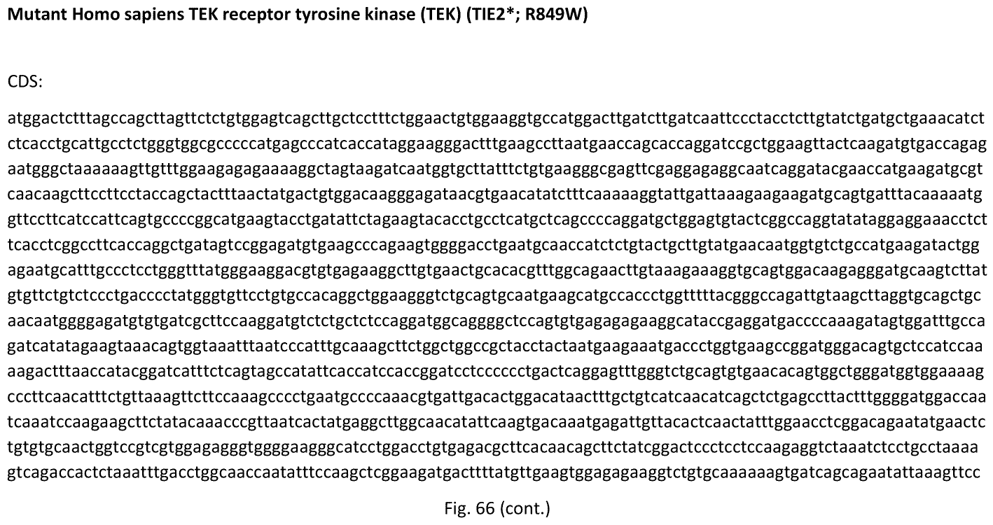

FIG. 66 shows the coding nucleotide sequence of mRNA of human mutant TEK receptor tyrosine kinase (TEK), which is also referred to as TIE* having a R849W mutation.

FIG. 67 shows the basic structure and nucleotide sequence of recombinant nucleic acid construct PAN50.

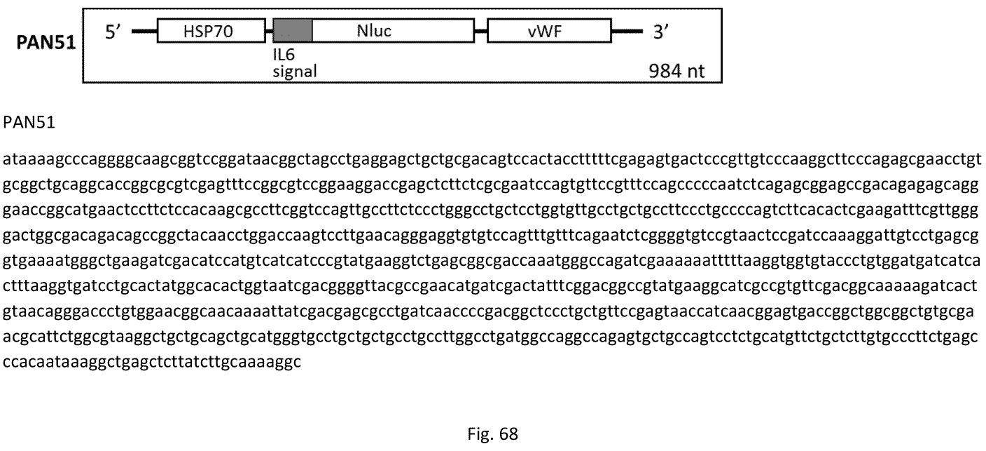

FIG. 68 shows the basic structure and nucleotide sequence of recombinant nucleic acid construct PAN51.

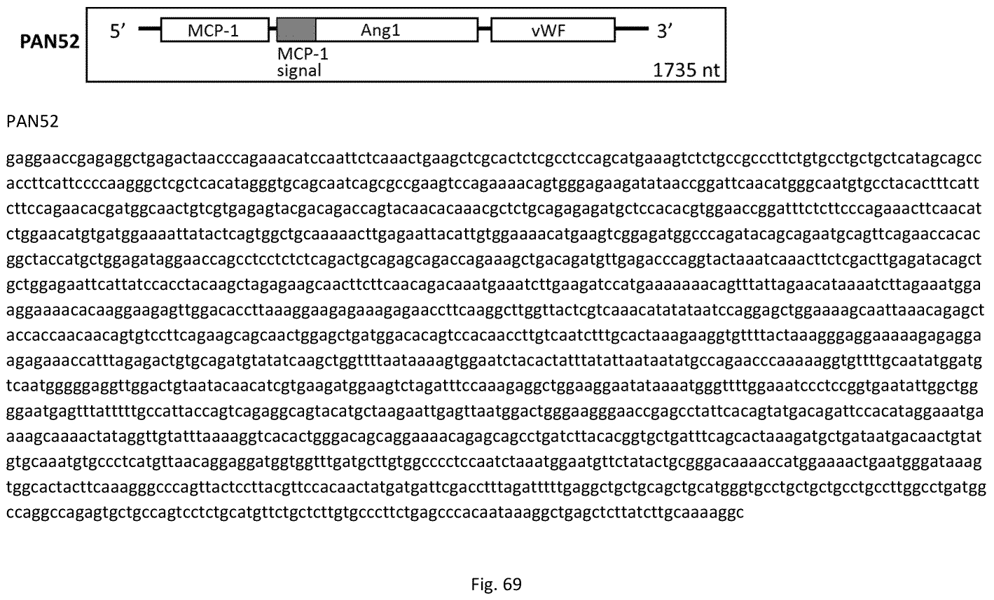

FIG. 69 shows the basic structure and nucleotide sequence of recombinant nucleic acid construct PAN52.

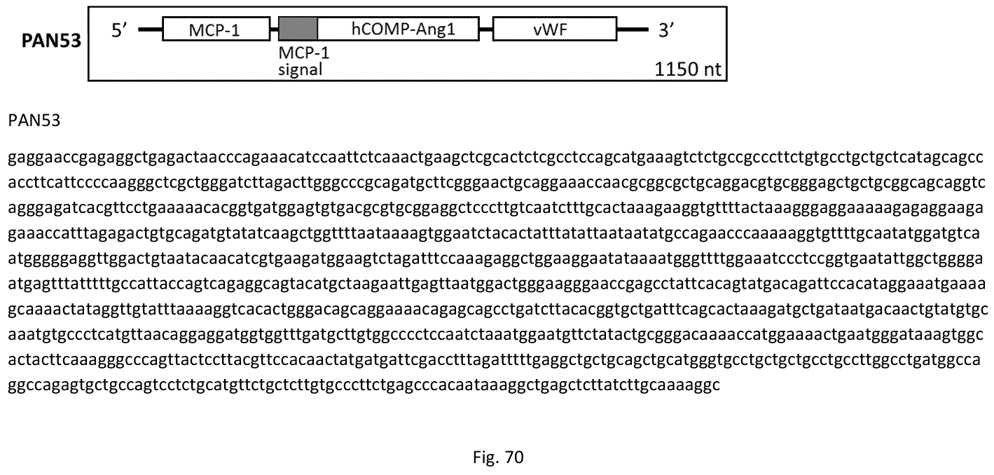

FIG. 70 shows the basic structure and nucleotide sequence of recombinant nucleic acid construct PAN53.

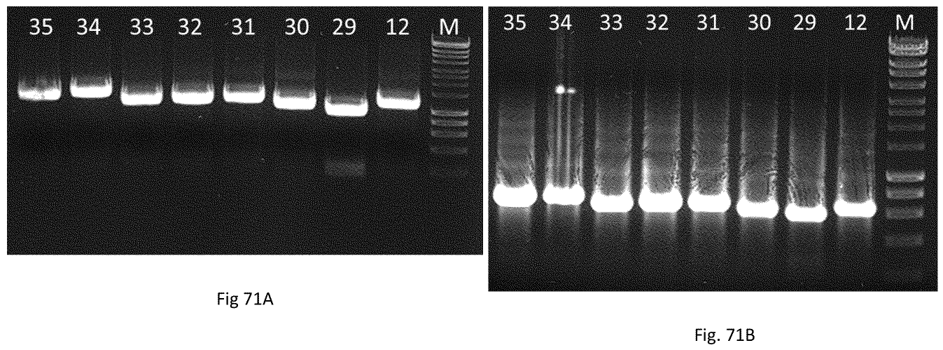

FIG. 71A is an image of a non-denaturing agarose gel after EtBr staining showing the tail-PCR products of various construct.

FIG. 71B is an image of a non-denaturing agarose gel after EtBr staining showing the in vitro mRNA transcripts of various construct.

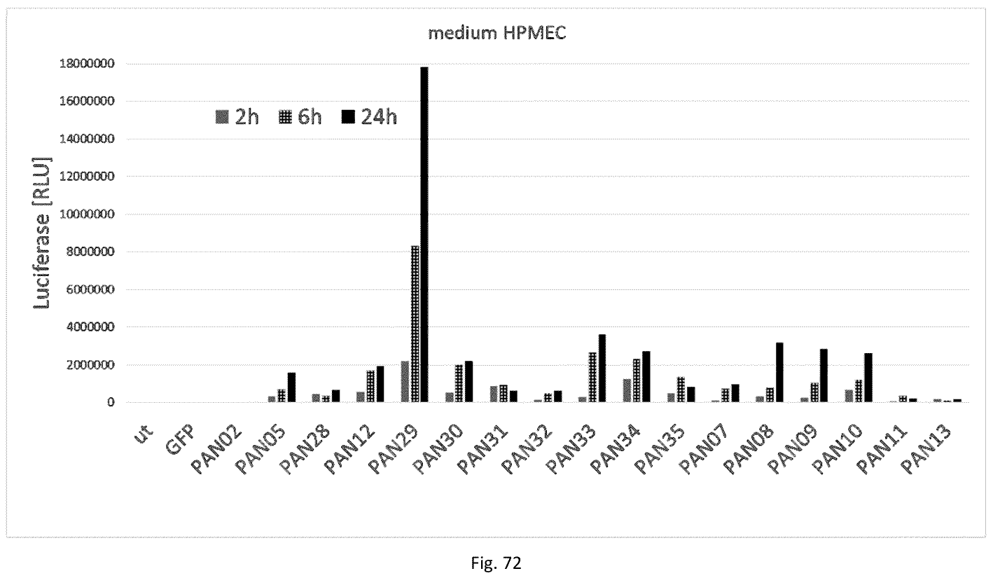

FIG. 72 is a bar diagram showing expression of luciferase, indicated as RLU (relative light units) of luciferase in medium, by the indicated recombinant nucleic acid constructs in HPMEC where luciferase is used as the effector molecule after 2 hours (left column), 6 hours (middle column) and 24 hours (right column) post transfection.

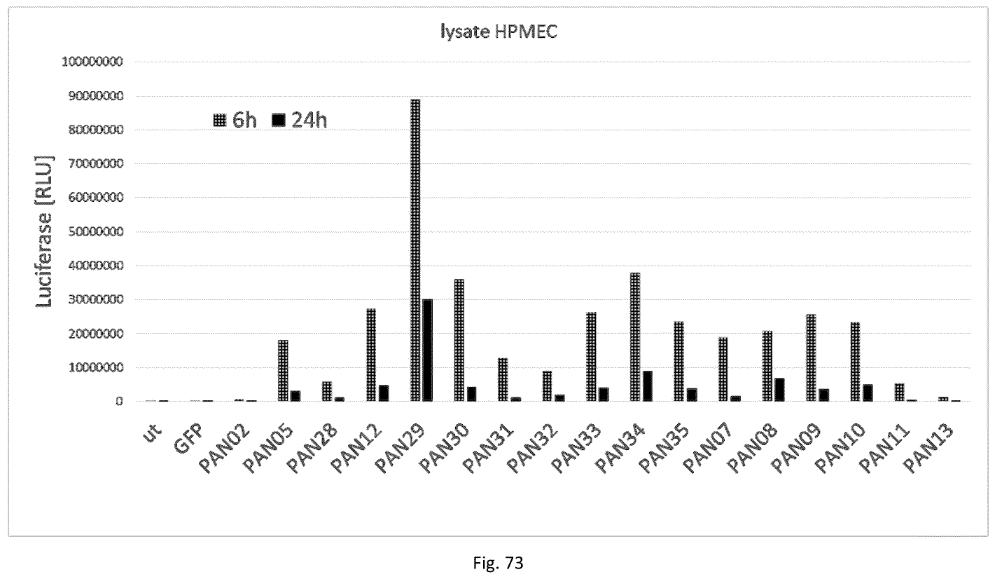

FIG. 73 is a bar diagram showing expression of luciferase, indicated as RLU (relative light units) of luciferase in whole cell lysates of HPMEC, by the indicated recombinant nucleic acid constructs in HPMEC, where luciferase is used as the effector molecule after 6 hours (left column) and 24 hours (right column) post transfection.

FIG. 74A (left) is a bar diagram showing expression of luciferase, indicated as RLU (relative light units) of luciferase in medium, by the indicated recombinant nucleic acid constructs in HPMEC (transfected with GFP mRNA, PAN12 mRNA and PAN12* mRNA) where luciferase is used as the effector molecule after 2 hours (left column), 6 hours (middle column) and 24 hours (right column) post transfection. In PAN12* 100% of uridine and 100% cytidine nucleotides are replaced by pseudo-uridine and 5-methyl-cytidine, respectively.

FIG. 74B (right) is a bar diagram showing expression of luciferase, indicated as RLU (relative light units) of luciferase in cell lysates, by the indicated recombinant nucleic acid constructs in HPMEC (transfected with GFP mRNA, PAN12 mRNA and PAN12* mRNA) where luciferase is used as the effector molecule after 6 hours (left column) and 24 hours (right column) post transfection. In PAN12* 100% of uridine and 100% cytidine nucleotides are replaced by pseudo-uridine and 5-methyl-cytidine, respectively.

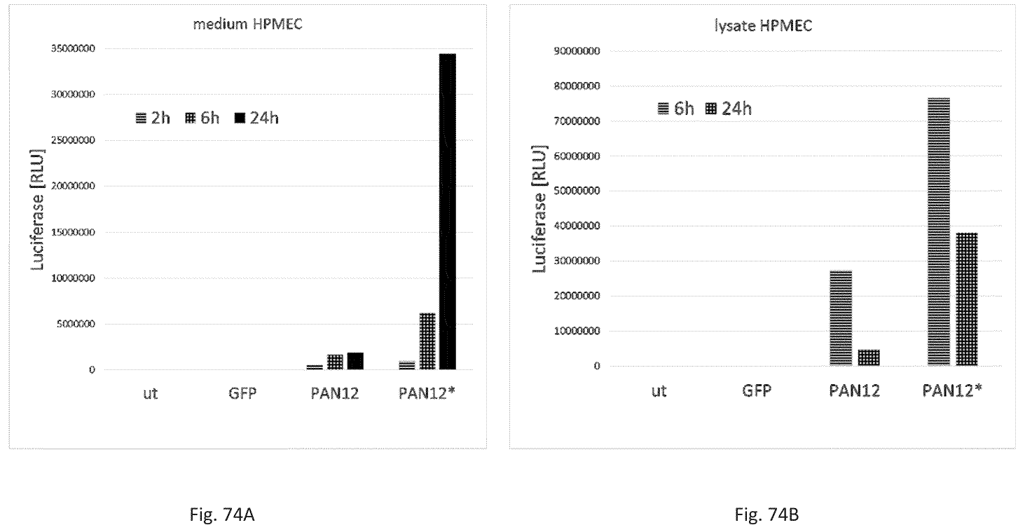

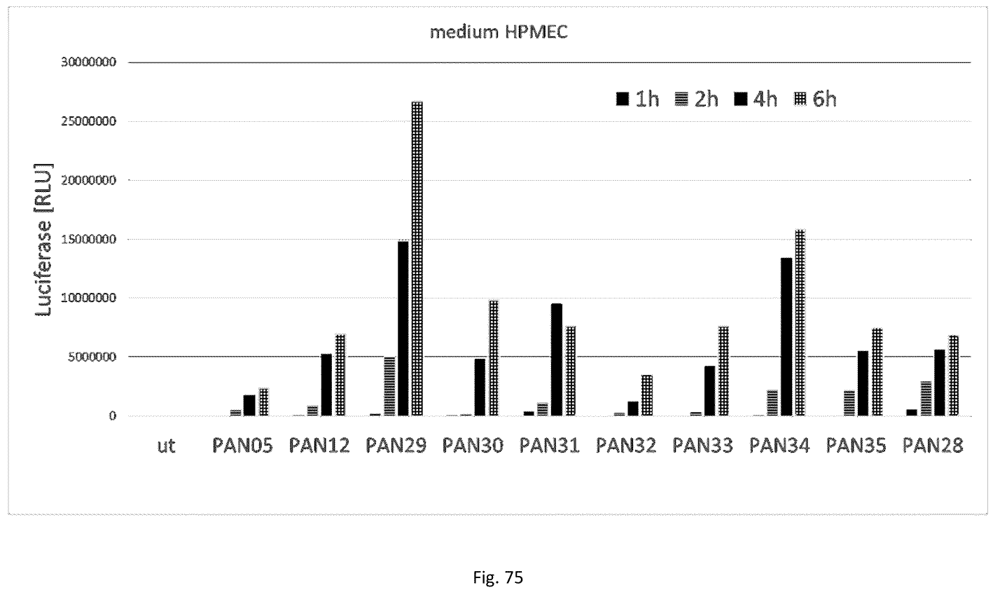

FIG. 75 is a bar diagram showing expression of luciferase, indicated as RLU (relative light units) of luciferase in medium, by the indicated recombinant nucleic acid constructs in HPMEC where luciferase is used as the effector molecule after 1 hour, 2 hours, 4 hours and 24 hours post transfection (from left to right, with 1 hour results being shown to the utmost left side and 24 hours result being shown to the utmost right side).

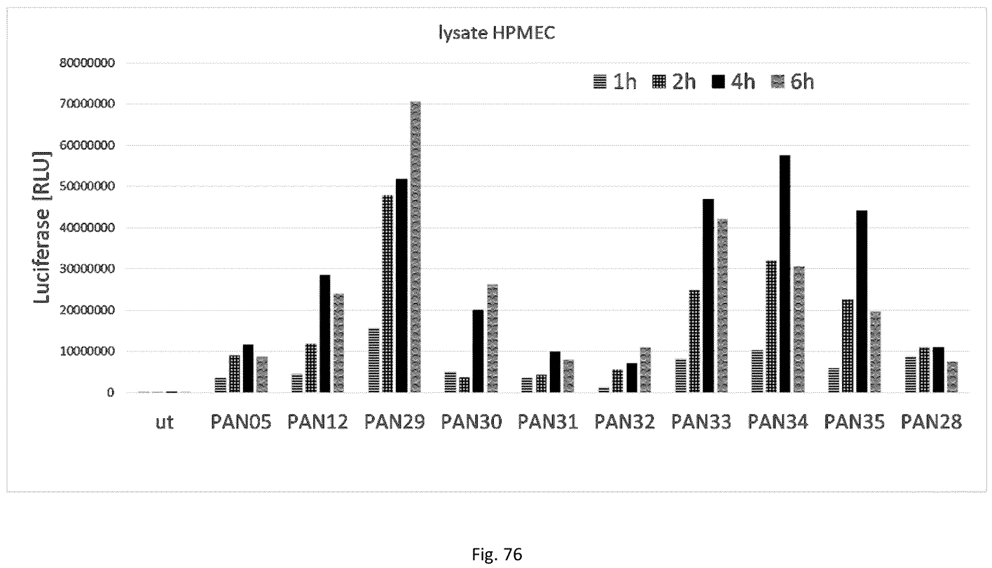

FIG. 76 is a bar diagram showing expression of luciferase, indicated as RLU (relative light units) of luciferase in whole cell lysates of HPMEC, by the indicated recombinant nucleic acid constructs in HPMEC, where luciferase is used as the effector molecule after 1 hour, 2 hours, 4 hours and 24 hours post transfection (from left to right, with 1 hour results being shown to the utmost left side and 24 hours result being shown to the utmost right side).

FIG. 77A (left) is a bar diagram showing expression of luciferase, indicated as RLU (relative light units) of luciferase in medium, by the indicated recombinant nucleic acid constructs in HPMEC (transfected with GFP mRNA, PAN12 mRNA and PAN12* mRNA) where luciferase is used as the effector molecule after 1 hour, 2 hours, 4 hours and 6 hours post transfection (from left to right, with 1 hour results being shown to the utmost left side and 6 hours result being shown to the utmost right side). In PAN12* 100% of uridine and 100% cytidine nucleotides are replaced by pseudo-uridine and 5-methyl-cytidine, respectively.

FIG. 77B (right) is a bar diagram showing expression of luciferase, indicated as RLU (relative light units) of luciferase in cell lysates, by the indicated recombinant nucleic acid constructs in HPMEC (transfected with GFP mRNA, PAN12 mRNA and PAN12* mRNA) where luciferase is used as the effector molecule after 1 hour, 2 hours, 4 hours and 6 hours post transfection (from left to right, with 1 hour results being shown to the utmost left side and 6 hours result being shown to the utmost right side). In PAN12* 100% of uridine and 100% cytidine nucleotides are replaced by pseudo-uridine and 5-methyl-cytidine, respectively.

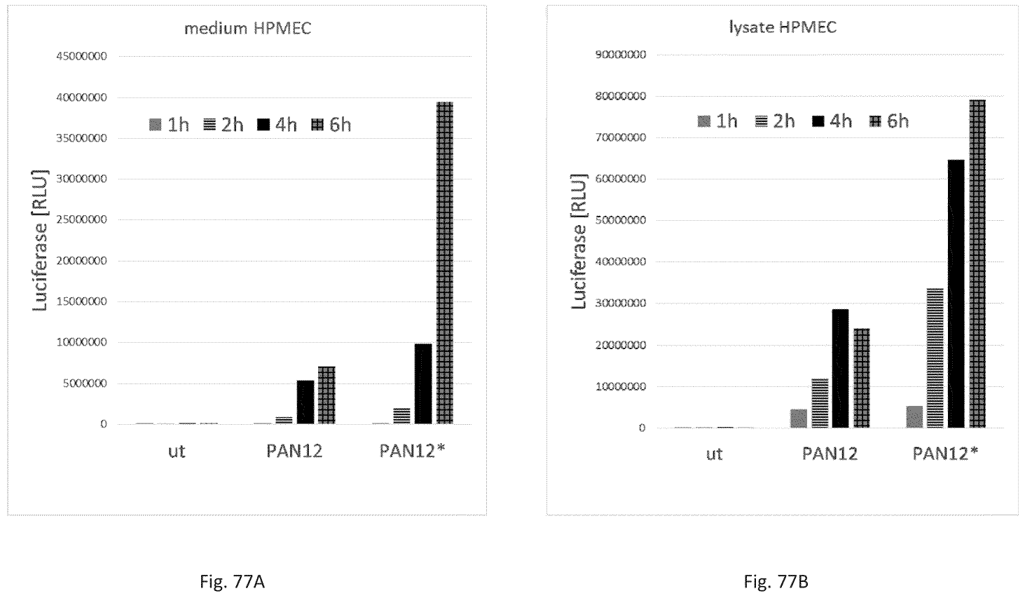

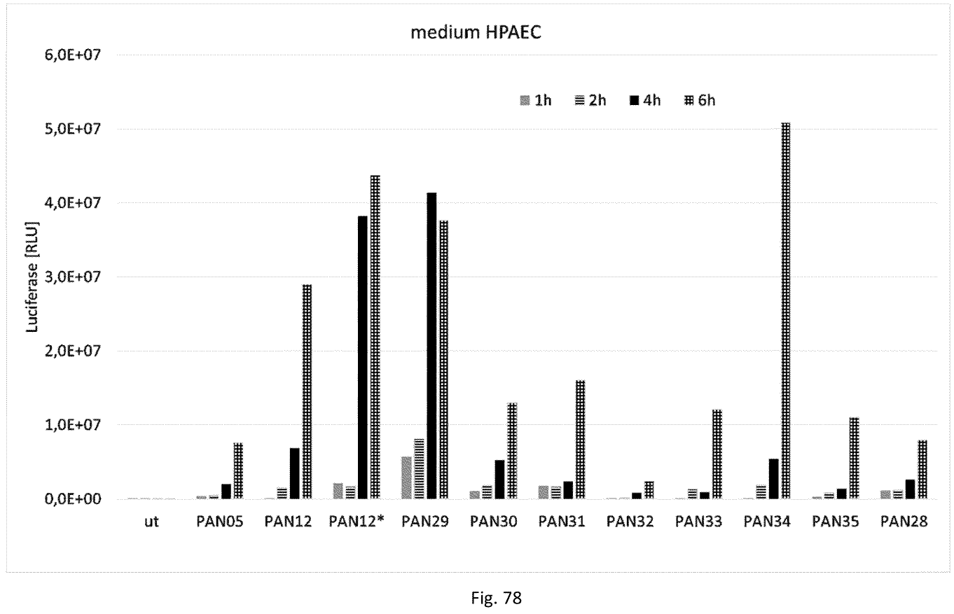

FIG. 78 is a bar diagram showing expression of luciferase, indicated as RLU (relative light units) of luciferase in medium, by the indicated recombinant nucleic acid constructs in HPAEC where luciferase is used as the effector molecule after 1 hour, 2 hours, 4 hours and 24 hours post transfection (from left to right, with 1 hour results being shown to the utmost left side and 24 hours result being shown to the utmost right side).

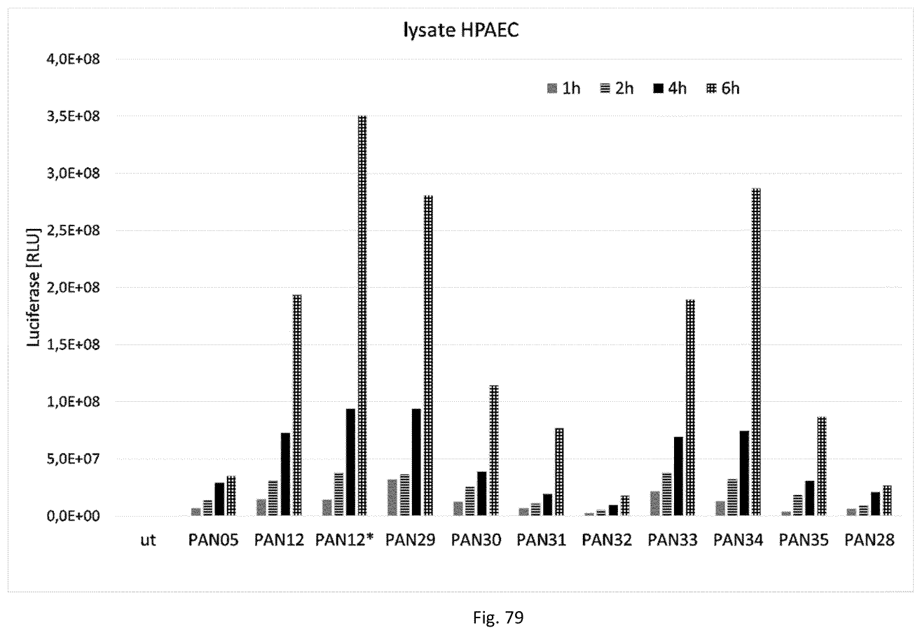

FIG. 79 is a bar diagram showing expression of luciferase, indicated as RLU (relative light units) of luciferase in whole cell lysates of HPAEC, by the indicated recombinant nucleic acid constructs in HPAEC, where luciferase is used as the effector molecule after 1 hour, 2 hours, 4 hours and 24 hours post transfection (from left to right, with 1 hour results being shown to the utmost left side and 24 hours result being shown to the utmost right side).

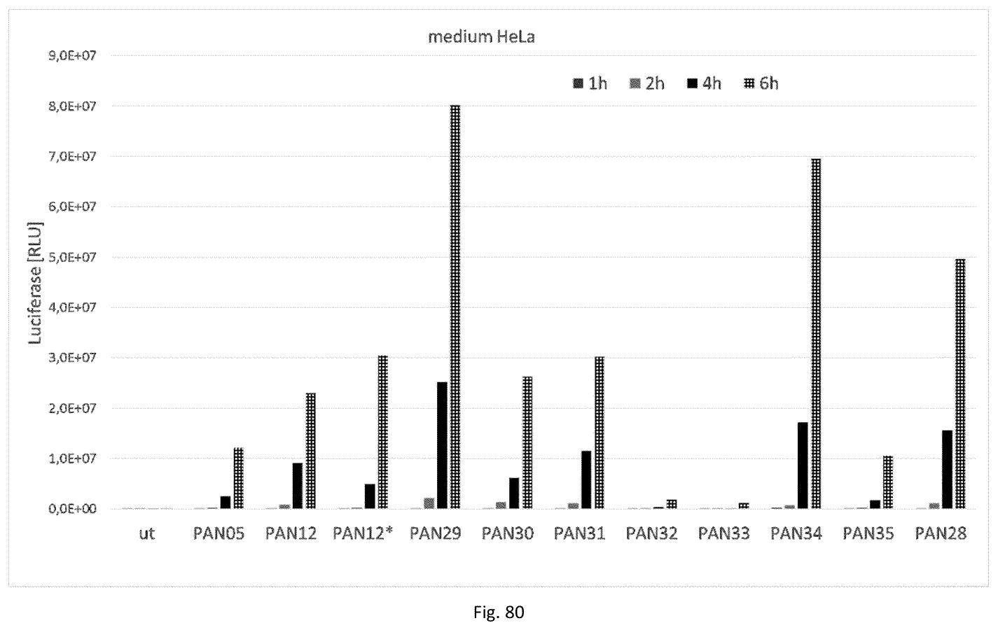

FIG. 80 is a bar diagram showing expression of luciferase, indicated as RLU (relative light units) of luciferase in medium, by the indicated recombinant nucleic acid constructs in HeLa cells where luciferase is used as the effector molecule after 1 hour, 2 hours, 4 hours and 24 hours post transfection (from left to right, with 1 hour results being shown to the utmost left side and 24 hours result being shown to the utmost right side).

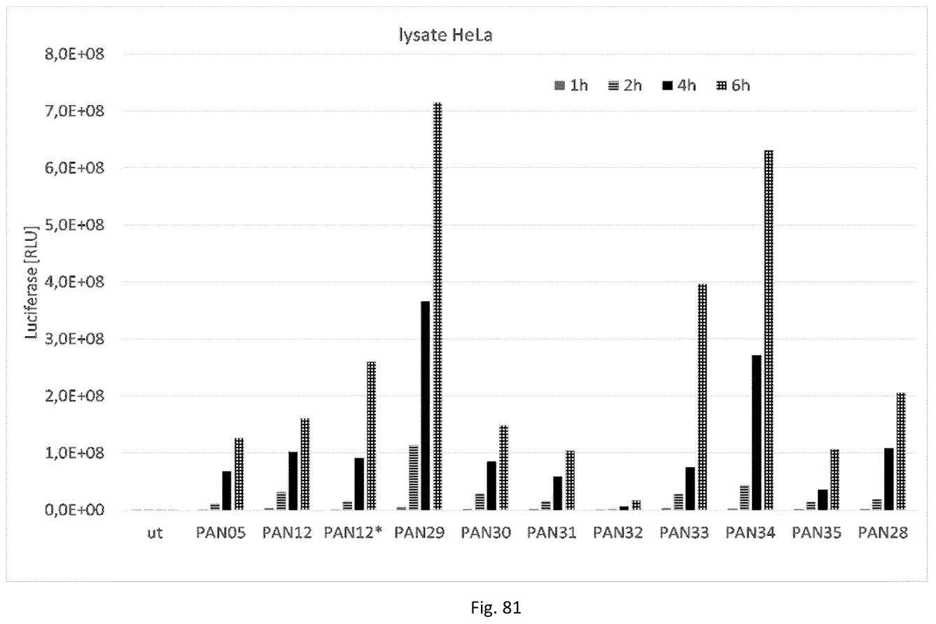

FIG. 81 is a bar diagram showing expression of luciferase, indicated as RLU (relative light units) of luciferase in whole cell lysates of HeLa cells, by the indicated recombinant nucleic acid constructs in HeLA cells, where luciferase is used as the effector molecule after 1 hour, 2 hours, 4 hours and 24 hours post transfection (from left to right, with 1 hour results being shown to the utmost left side and 24 hours result being shown to the utmost right side).

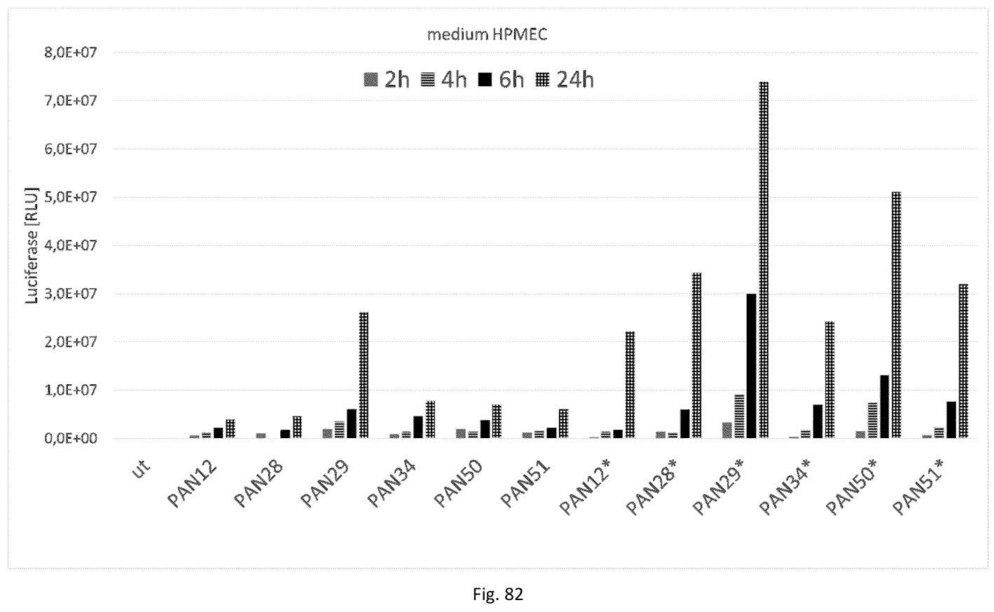

FIG. 82 is a bar diagram showing expression of luciferase, indicated as RLU (relative light units) of luciferase in medium, by the indicated recombinant nucleic acid constructs in HPMEC where luciferase is used as the effector molecule after 2 hours, 4 hours, 6 hours and 24 hours post transfection (from left to right, with 1 hour results being shown to the utmost left side and 24 hours result being shown to the utmost right side). In constructs PAN12*, PAN28*, PAN29*, PAN34*, PAN50* and PAN51*100% of uridine and 100% cytidine nucleotides are replaced by pseudo-uridine and 5-methyl-cytidine, respectively.

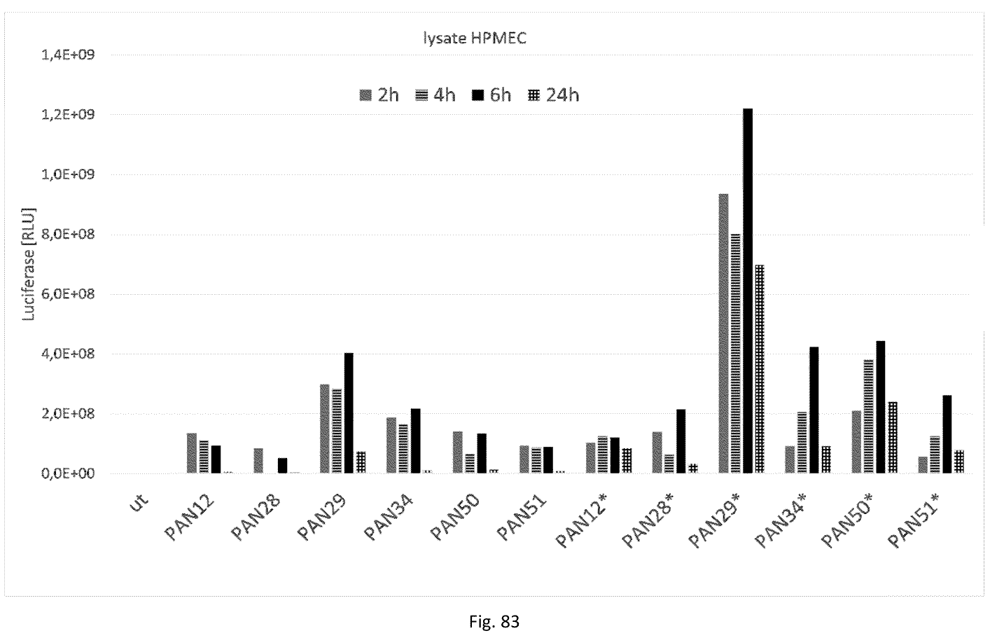

FIG. 83 is a bar diagram showing expression of luciferase, indicated as RLU (relative light units) of luciferase in whole cell lysates of HPMEC, by the indicated recombinant nucleic acid constructs in HPMEC, where luciferase is used as the effector molecule after 2 hours, 4 hours, 6 hours and 24 hours post transfection (from left to right, with 1 hour results being shown to the utmost left side and 24 hours result being shown to the utmost right side). In constructs PAN12*, PAN28*, PAN29*, PAN34*, PAN50* and PAN51*100% of uridine and 100% cytidine nucleotides are replaced by pseudo-uridine and 5-methyl-cytidine, respectively.

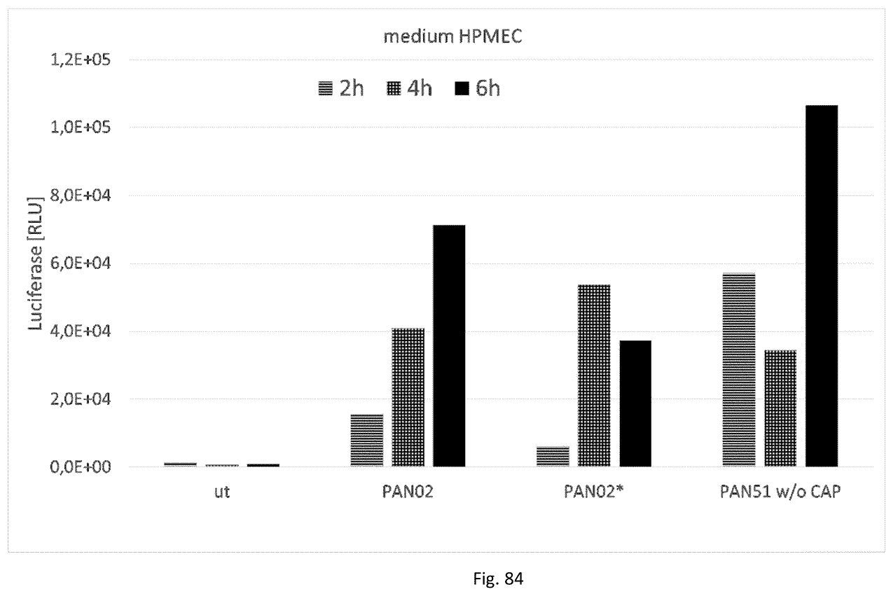

FIG. 84 is a bar diagram showing expression of luciferase, indicated as RLU (relative light units) of luciferase in medium, by the indicated recombinant nucleic acid constructs in HPMEC where luciferase is used as the effector molecule after 2 hours (left column), 4 hours (middle column) and 6 hours (right column) post transfection. In construct PAN02*, 100% of uridine and 100% cytidine nucleotides are replaced by pseudo-uridine and 5-methyl-cytidine, respectively; and in construct PAN51 w/o CAP the mRNA sequence is the one of construct PAN51, but without any 5′-capping structure.

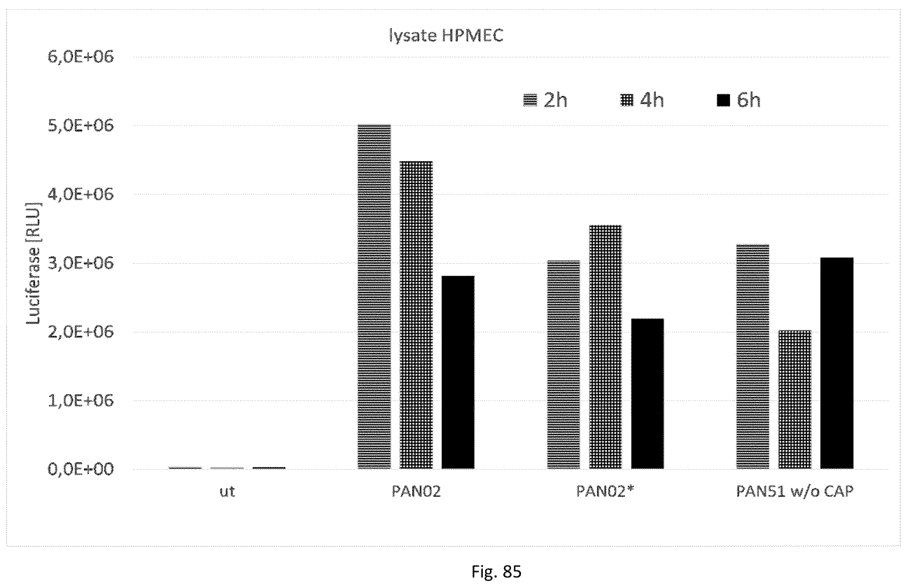

FIG. 85 is a bar diagram showing expression of luciferase, indicated as RLU (relative light units) of luciferase in whole cell lysates of HPMEC, by the indicated recombinant nucleic acid constructs in HPMEC, where luciferase is used as the effector molecule after 2 hours (left column) 4 hours (middle column) and 6 hours (right column) post transfection. In construct PAN02*, 100% of uridine and 100% cytidine nucleotides are replaced by pseudo-uridine and 5-methyl-cytidine, respectively; and in construct PAN51 w/o CAP the mRNA sequence is the one of construct PAN51, but without any 5′-capping structure.

FIG. 86 shows the basic structure and nucleotide sequence of recombinant nucleic acid construct PAN54.

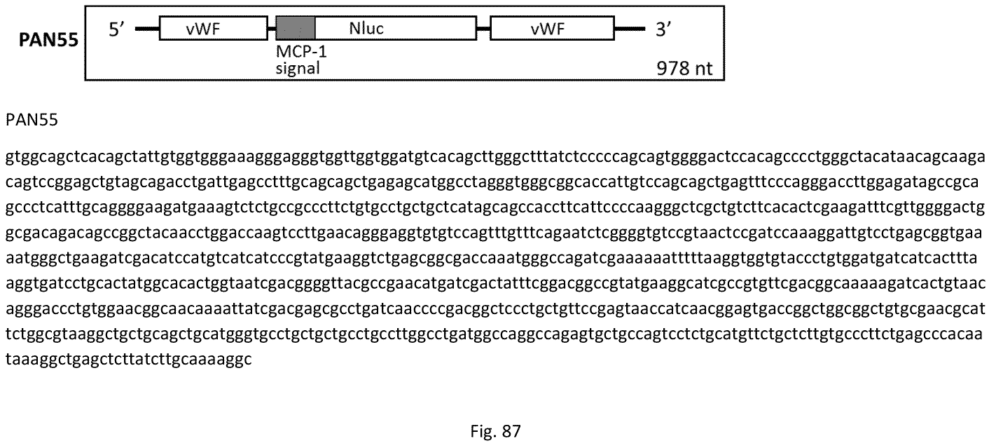

FIG. 87 shows the basic structure and nucleotide sequence of recombinant nucleic acid construct PAN55.

FIG. 88 shows the basic structure and nucleotide sequence of recombinant nucleic acid construct PAN56.

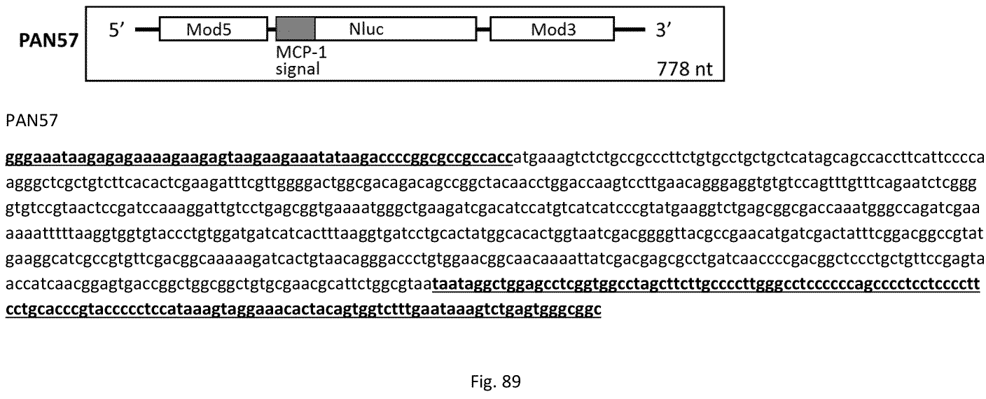

FIG. 89 shows the basic structure and nucleotide sequence of recombinant nucleic acid construct PAN57.

FIG. 90 shows the basic structure and nucleotide sequence of recombinant nucleic acid construct PAN58.

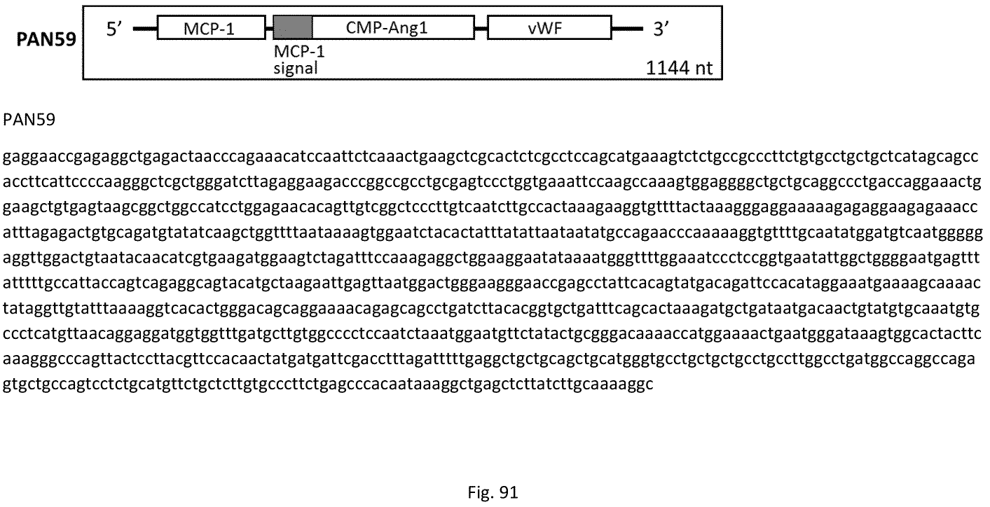

FIG. 91 shows the basic structure and nucleotide sequence of recombinant nucleic acid construct PAN59.

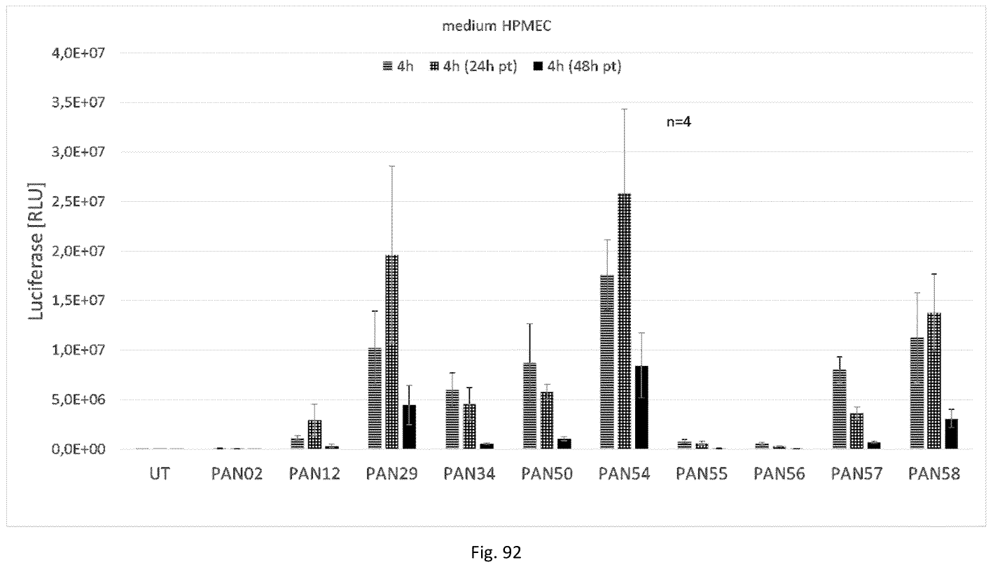

FIG. 92 is a bar diagram showing expression of luciferase, indicated as RLU (relative light units) of luciferase in medium, by the indicated recombinant nucleic acid constructs in HPMEC, where luciferase is used as the effector molecule after 4 hours (left column). Additional 4-hours luciferase activity values are assessed 24 hours (middle column) and 48 hours (right column) post transfection, after washing of the cells and supplementing the cells with fresh medium.

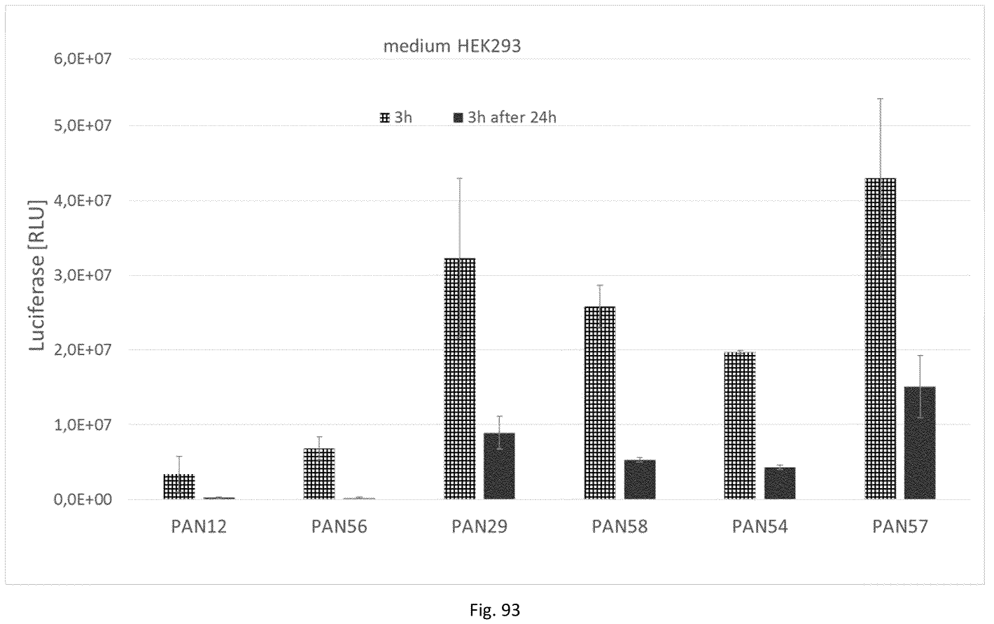

FIG. 93 is a bar diagram showing expression of luciferase, indicated as RLU (relative light units) of luciferase in medium, by the indicated recombinant nucleic acid constructs in HEK293 cells, where luciferase is used as the effector molecule after 3 hours (left column). An additional 3-hours luciferase activity value is assessed 24 hours (right column) post transfection, after washing of the cells and supplementing the cells with fresh medium.

The human embryonic kidney 293 cell line (HEK293) is grown in EMEM (EBSS) + 2 mM Glutamine + 1% Non-Essential Amino Acids (NEAA) + 10% FCS culture medium. Subculture Routine is to split sub-confluent cultures (70-80%) 1:2 to 1:6 i.e. seeding at 2-5×10,000 cells/cm² using 0.25% trypsin or trypsin/EDTA; 5% CO₂; 37° C. mRNA transfection are performed with the Lipofectamine™ MessengerMAX™ Transfection Reagent (Invitrogen™) according to the manufactures protocol.

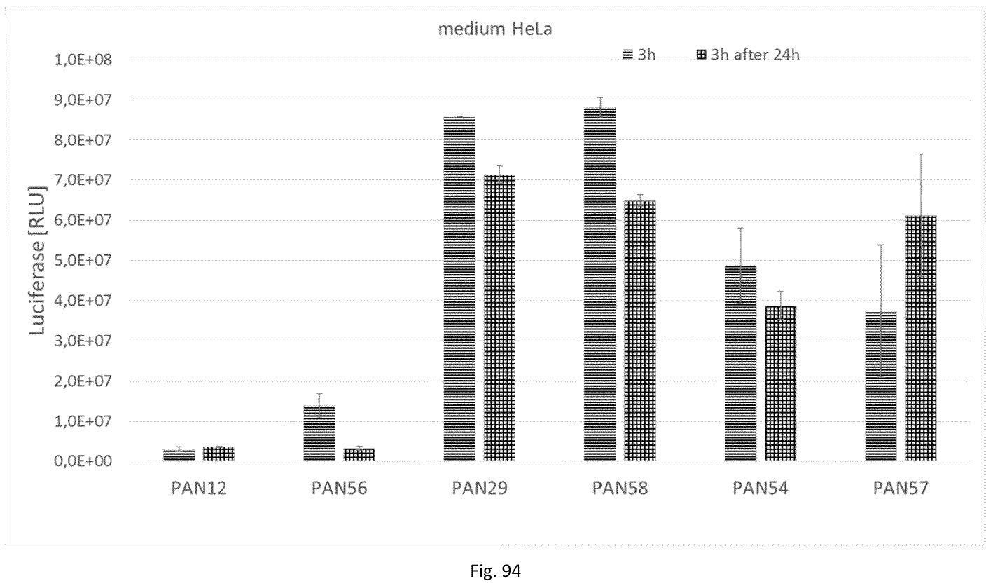

FIG. 94 is a bar diagram showing expression of luciferase, indicated as RLU (relative light units) of luciferase in medium, by the indicated recombinant nucleic acid constructs in HeLa cells, where luciferase is used as the effector molecule after 3 hours (left column). An additional 3-hours luciferase activity value is assessed 24 hours (right column) post transfection, after washing of the cells and supplementing the cells with fresh medium.

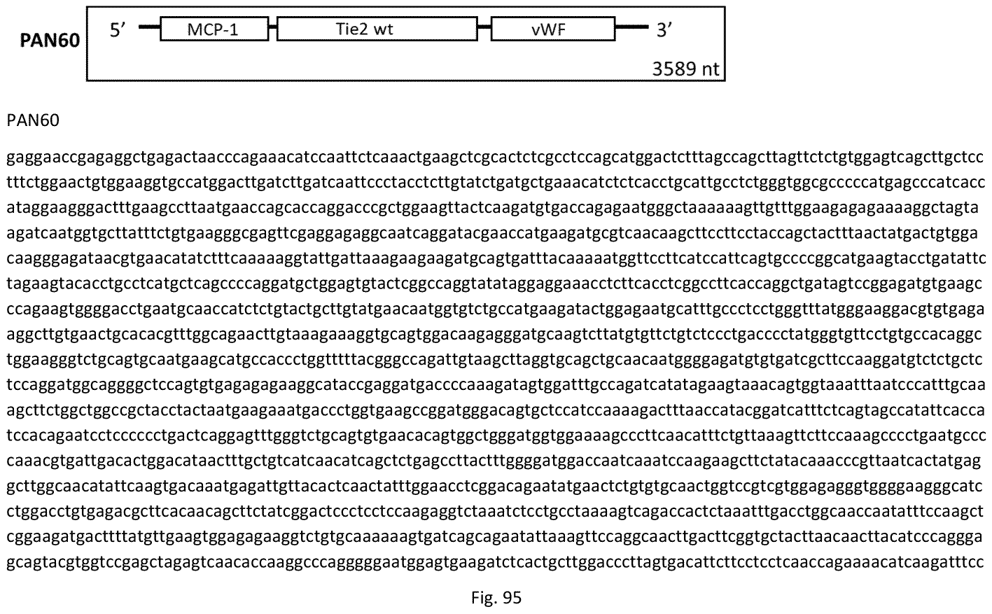

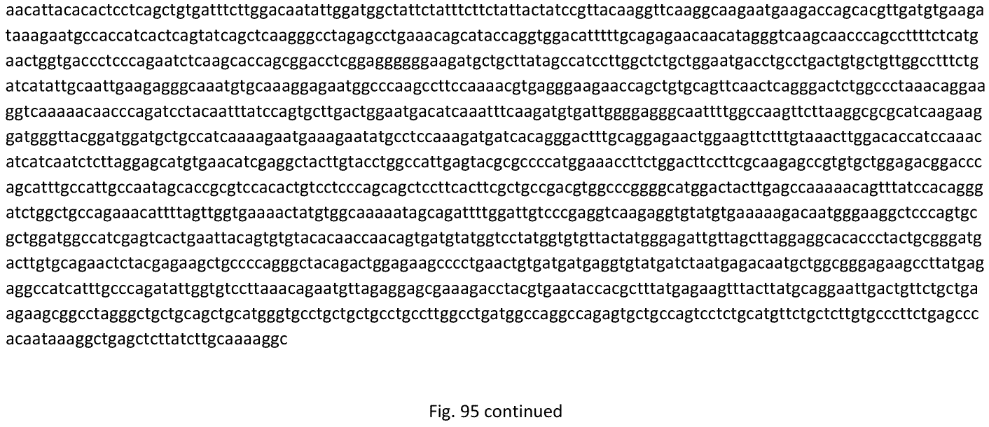

FIG. 95 shows the basic structure and nucleotide sequence of recombinant nucleic acid construct PAN60.

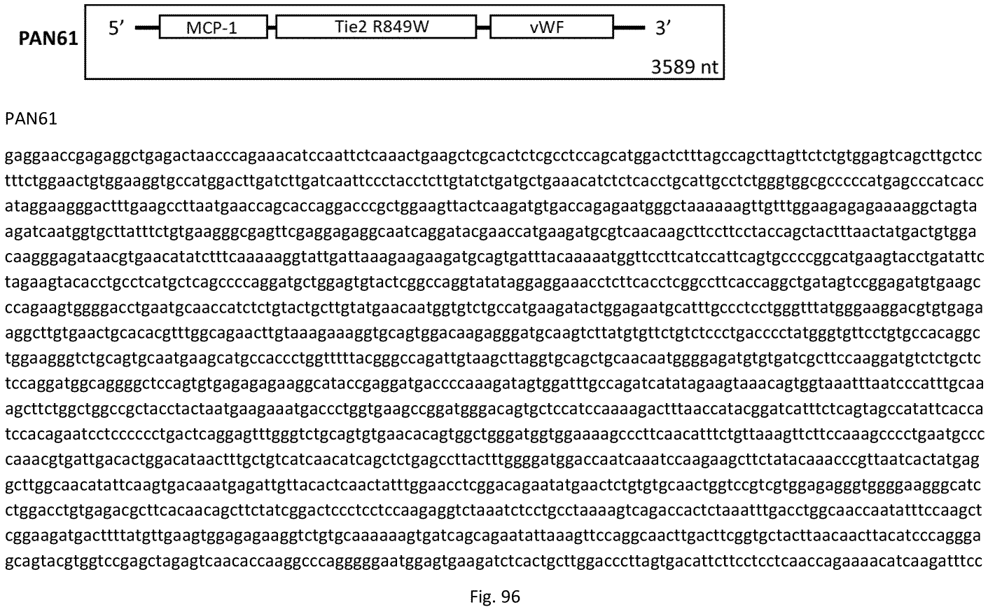

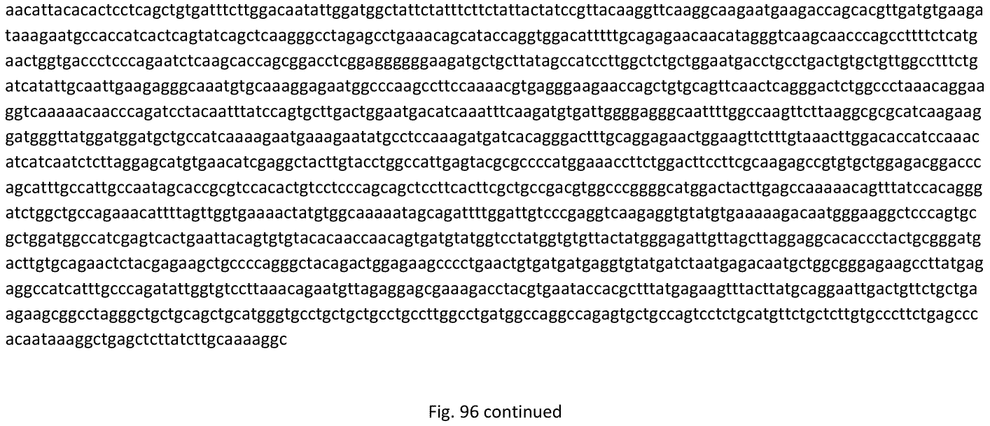

FIG. 96 shows the basic structure and nucleotide sequence of recombinant nucleic acid construct PAN61.

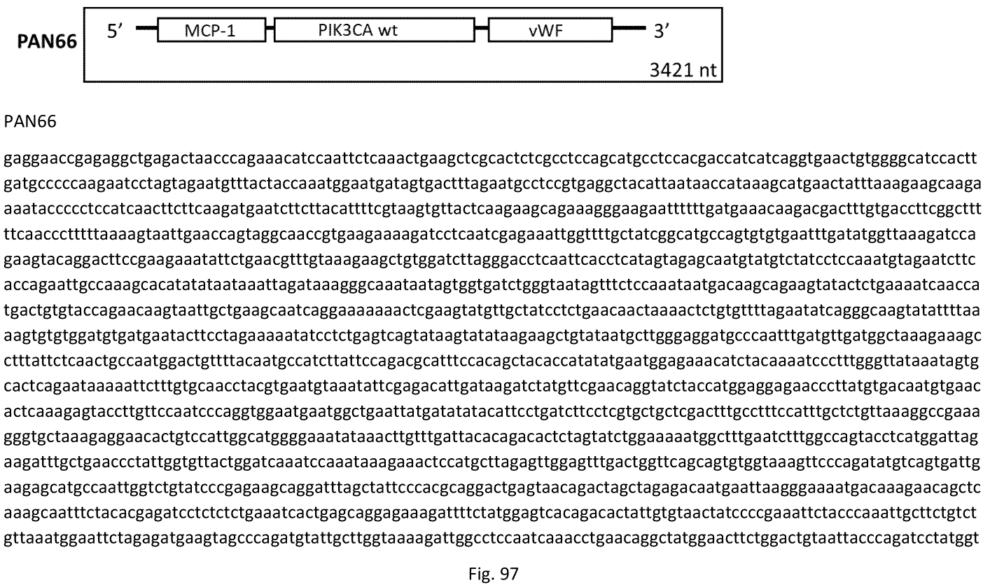

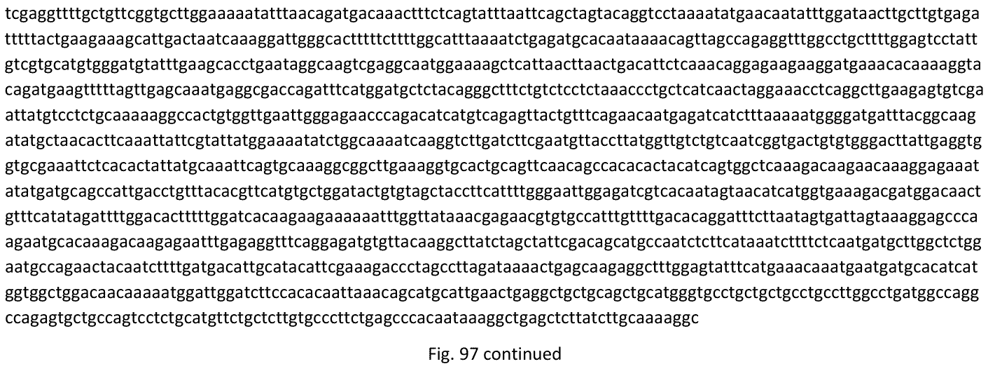

FIG. 97 shows the basic structure and nucleotide sequence of recombinant nucleic acid construct PAN66.

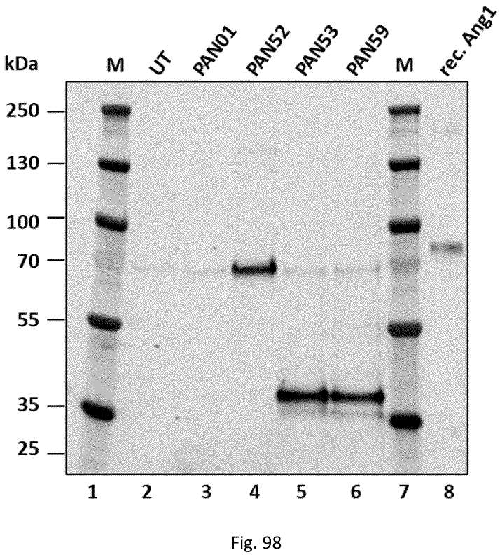

FIG. 98 shows Western Blot analysis of protein lysates from HPMEC cells transfected with indicated PAN mRNA constructs. Cells were transfected with 1 µg the mRNAs and harvested after 6 hours. Total cell lysates were separated by SDS-PAGE and analyzed by immunoblot using anti-Ang1 antibody. M, Marker; UT, untreated cells, rec. Ang1, recombinant hAng1.

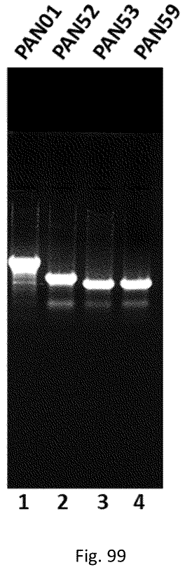

FIG. 99 shows Agarose gels of mRNAs after in vitro transcription. 2 µg of indicated mRNAs were separated by agarose gel electrophoresis and visualized by ethidium bromide staining and UV illumination.

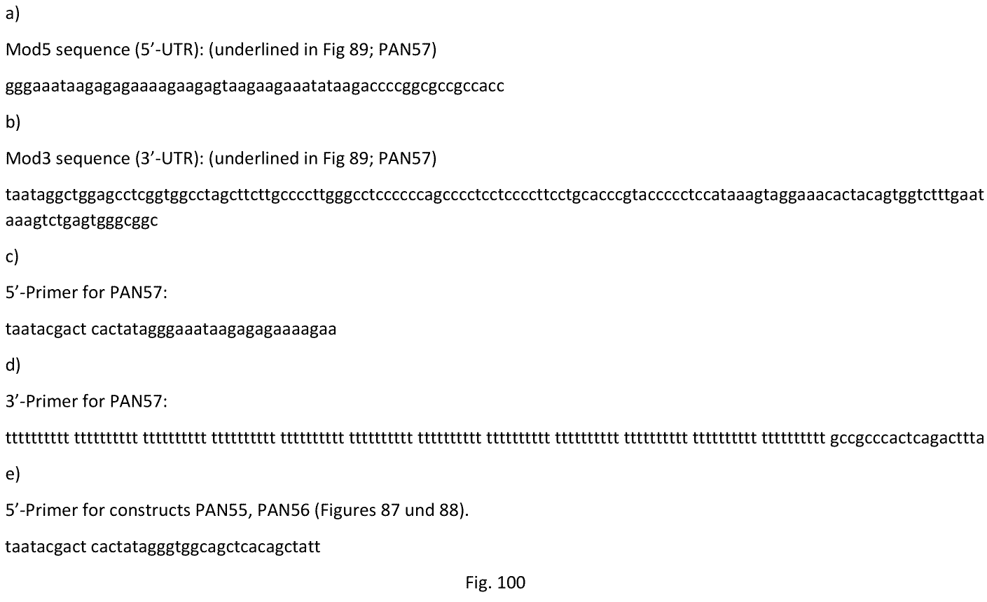

FIG. 100 shows the sequences of: a) the 5′UTR of PAN57; b) the 3′UTR of PAN57; c) the 5′PCR-Primer for PAN57; d) the 3′PCR-Primer for PAN57 and e) the 5′PCR-Primer for PAN55, PAN56.

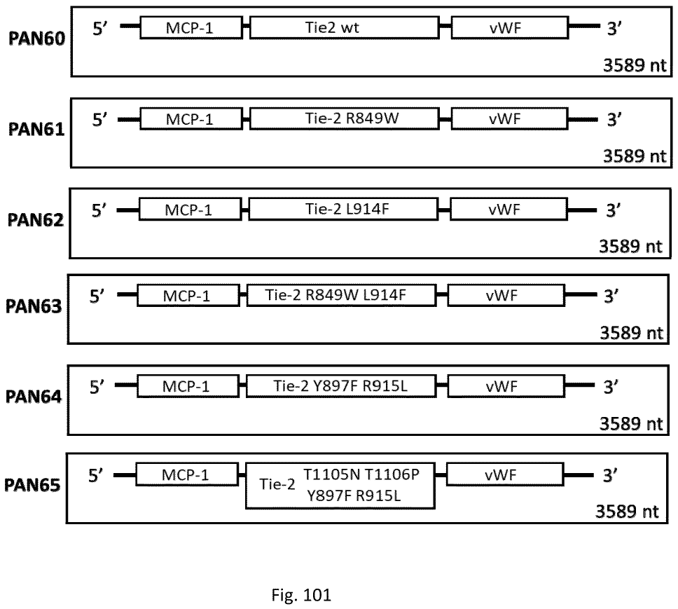

FIG. 101 shows the basic structure and length of recombinant nucleic acid constructs PAN60, PAN61, PAN62, PAN63, PAN64 and PAN65.

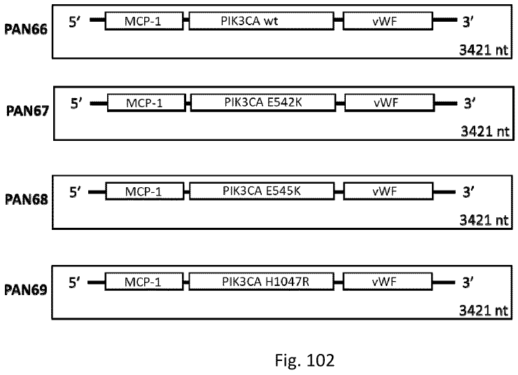

FIG. 102 shows the basic structure and length of recombinant nucleic acid constructs PAN66, PAN67, PAN68, and PAN69.

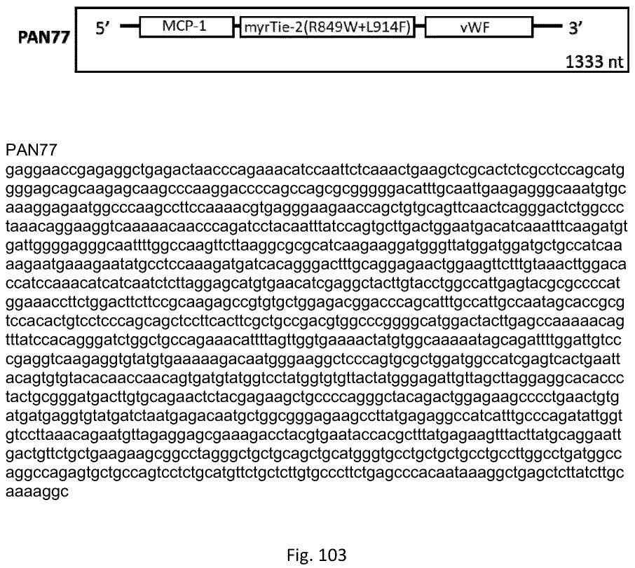

FIG. 103 shows the basic structure, length and nucleotide sequence of recombinant nucleic acid construct PAN77.

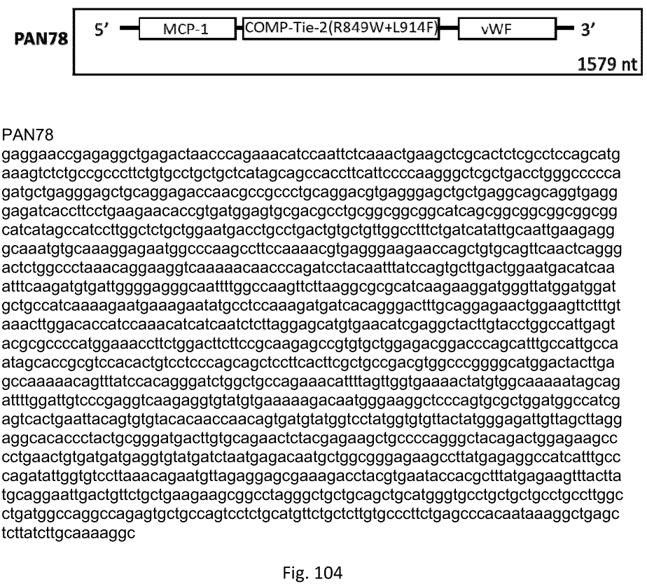

FIG. 104 shows the basic structure, length and nucleotide sequence of recombinant nucleic acid construct PAN78.

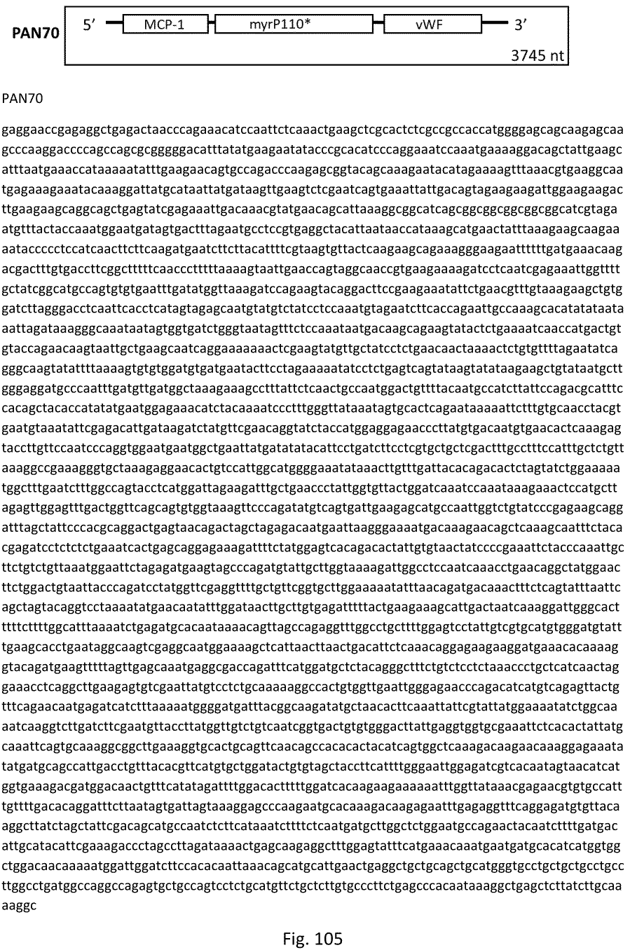

FIG. 105 shows the basic structure, the length and nucleotide sequence of recombinant nucleic acid and the nucleotide sequence of construct PAN70.

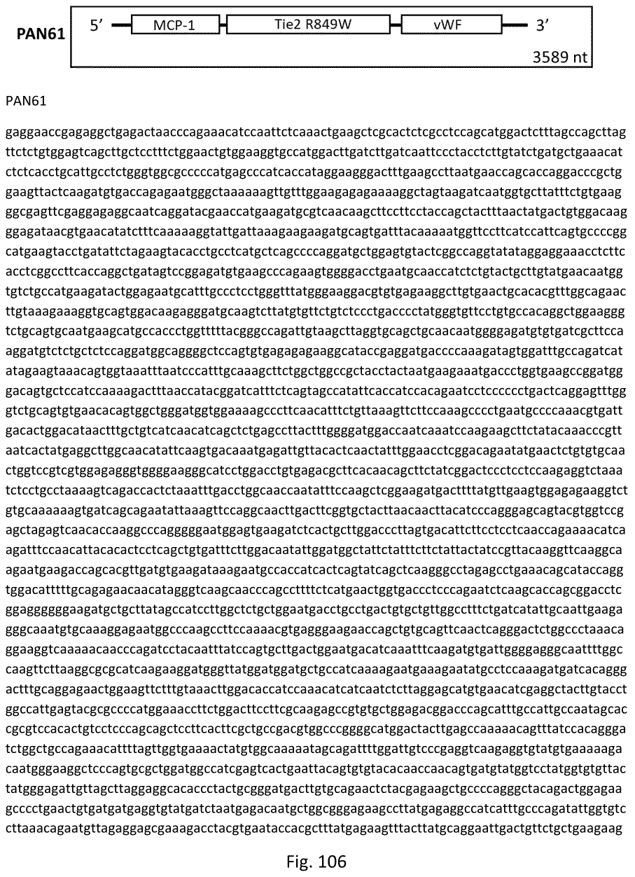

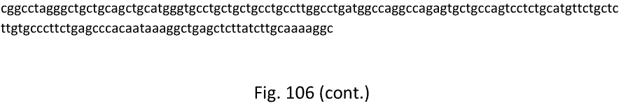

FIG. 106 shows the basic structure, length and nucleotide sequence of recombinant nucleic acid and the nucleotide sequence of construct PAN61.

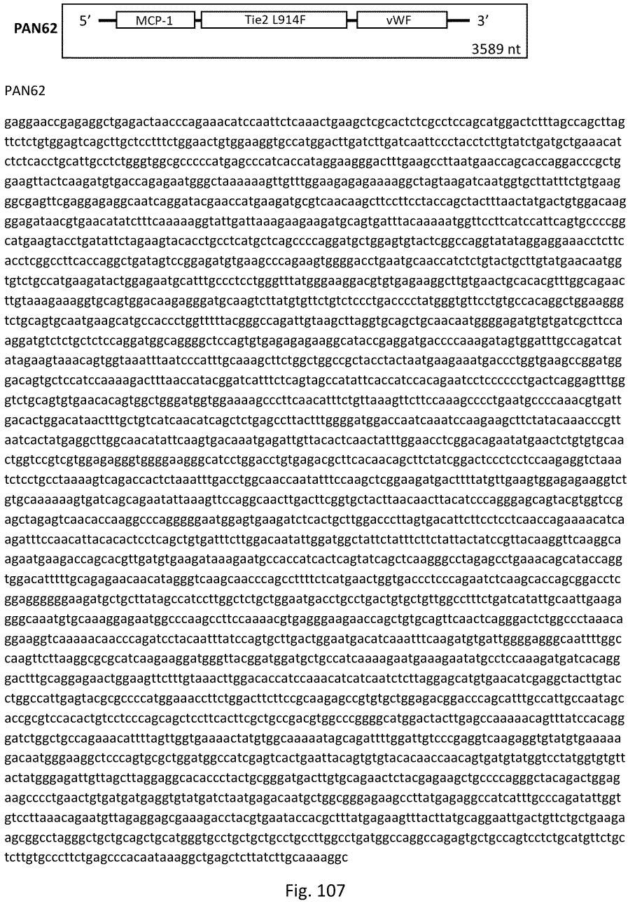

FIG. 107 shows the basic structure, length and nucleotide sequence of recombinant nucleic acid and the nucleotide sequence of construct PAN62.

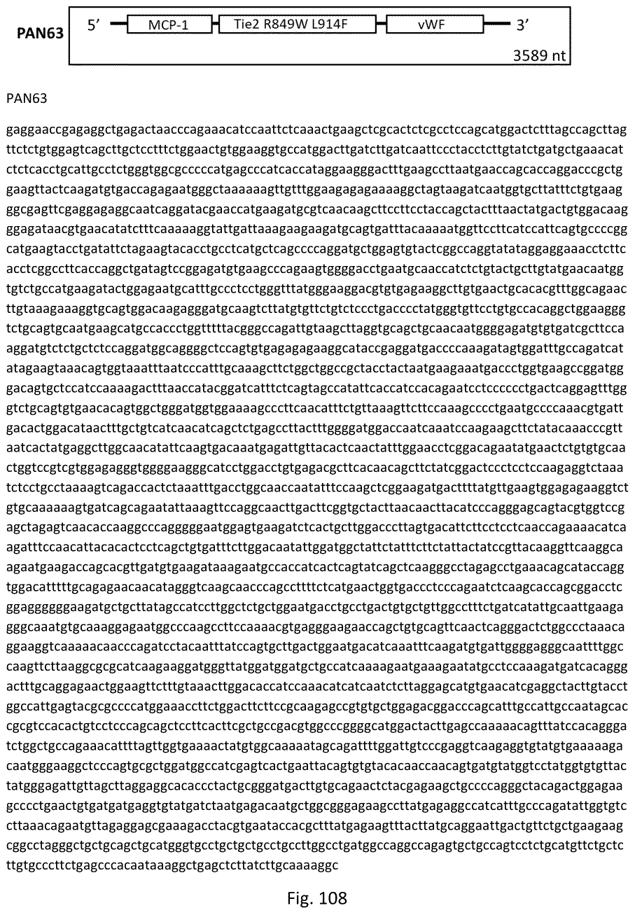

FIG. 108 shows the basic structure, length and nucleotide sequence of recombinant nucleic acid and the nucleotide sequence of construct PAN63.

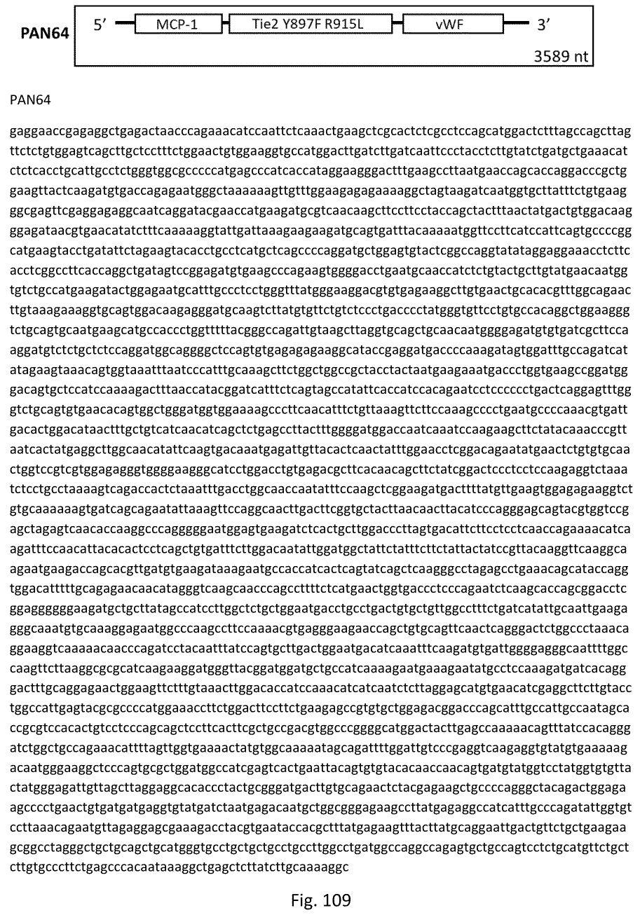

FIG. 109 shows the basic structure, length and nucleotide sequence of recombinant nucleic acid and the nucleotide sequence of construct PAN64.

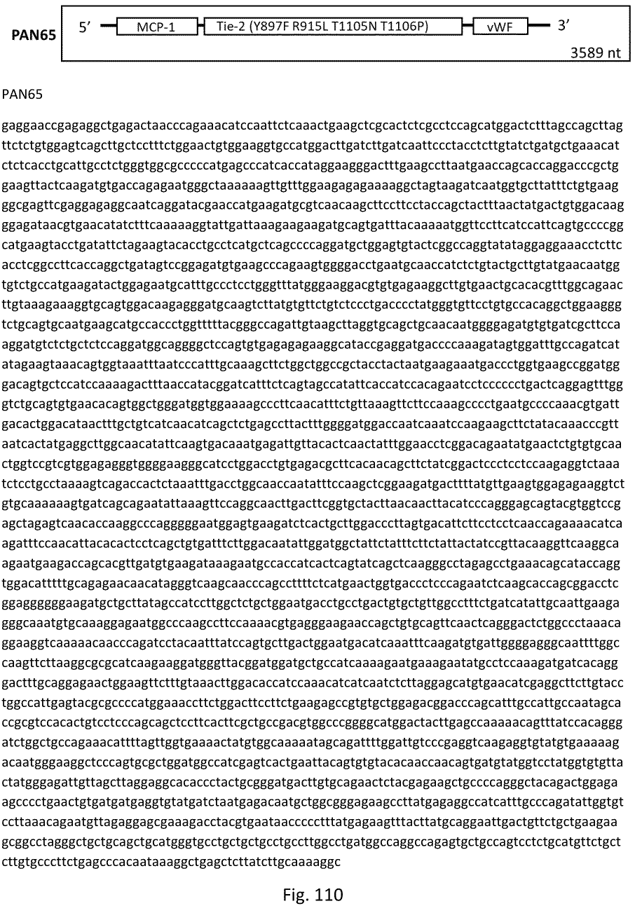

FIG. 110 shows the basic structure, length and nucleotide sequence of recombinant nucleic acid and the nucleotide sequence of construct PAN65.

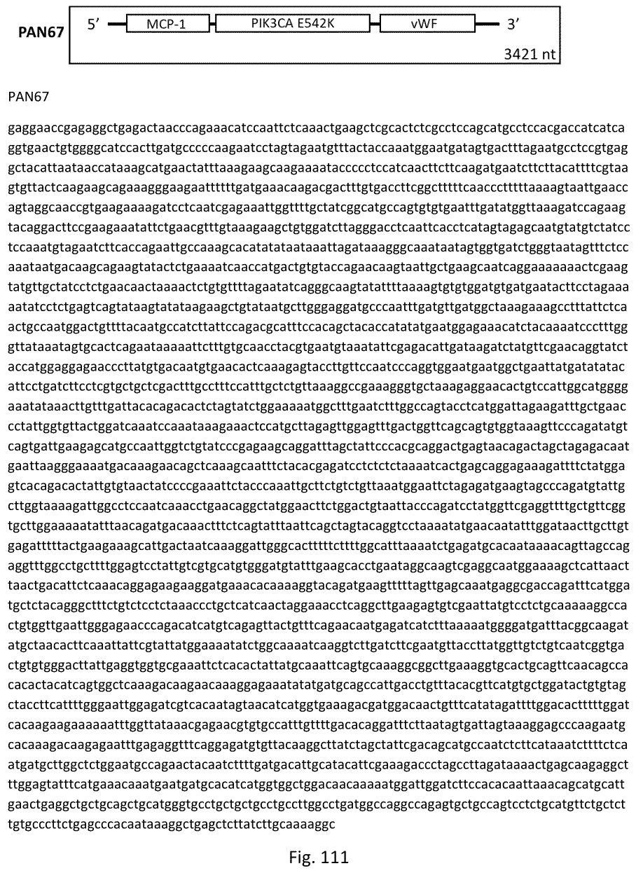

FIG. 111 shows the basic structure, length and nucleotide sequence of recombinant nucleic acid and the nucleotide sequence of construct PAN67.

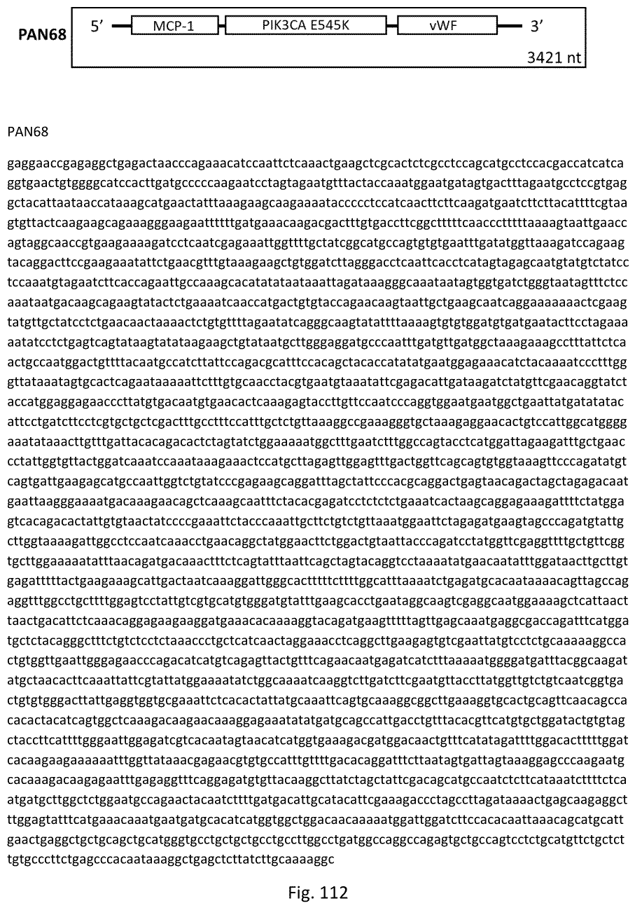

FIG. 112 shows the basic structure, length and nucleotide sequence of recombinant nucleic acid and the nucleotide sequence of construct PAN68.

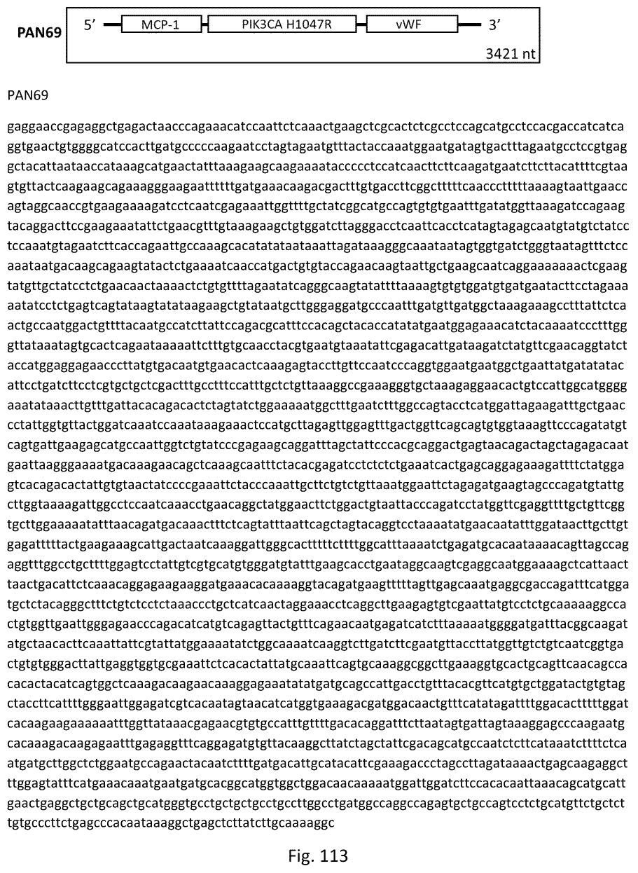

FIG. 113 shows the basic structure, length and nucleotide sequence of recombinant nucleic acid and the nucleotide sequence of construct PAN69.

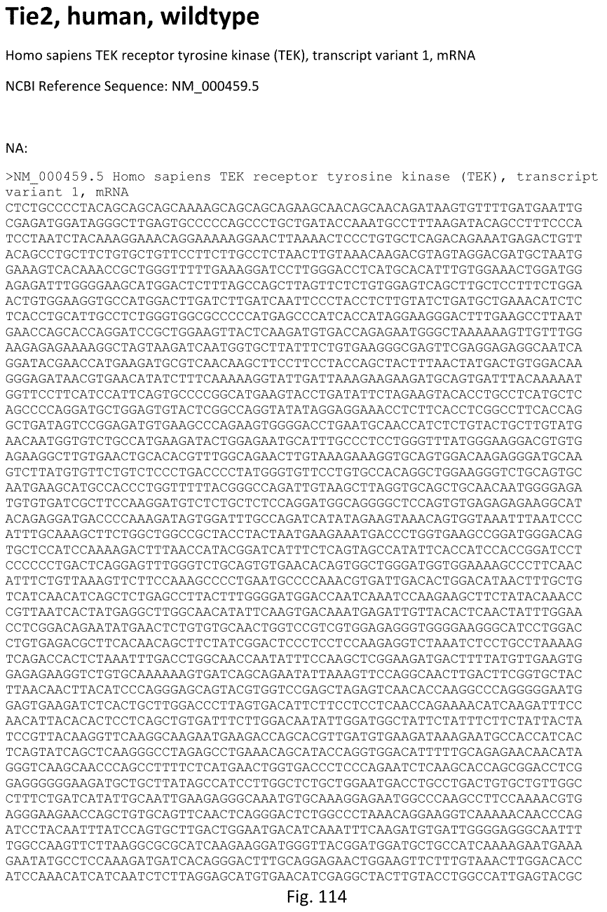

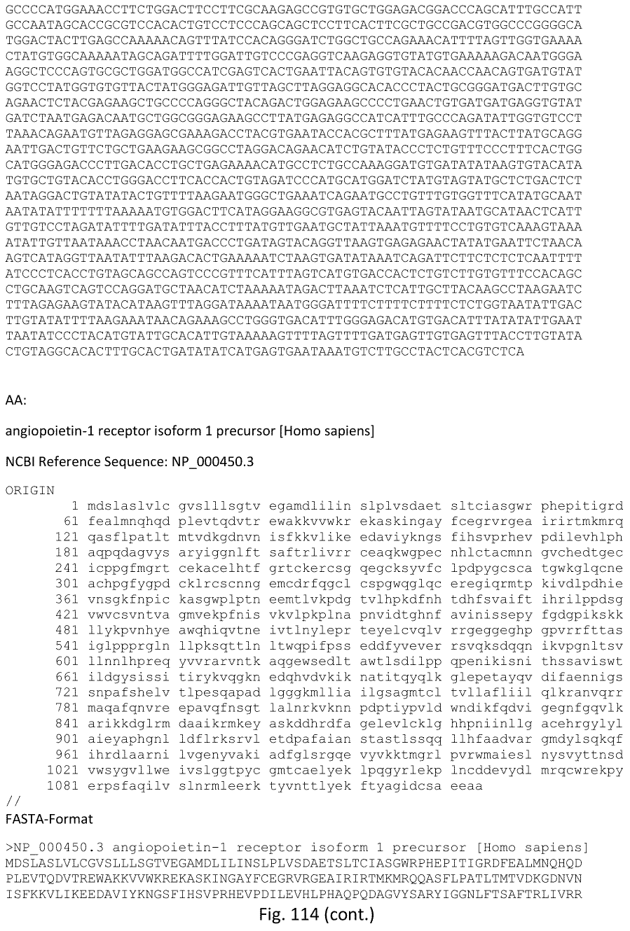

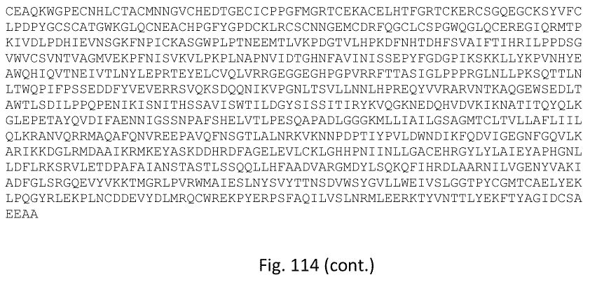

FIG. 114 shows the nucleotide sequence and amino acid sequence of human wild type Tie-2.

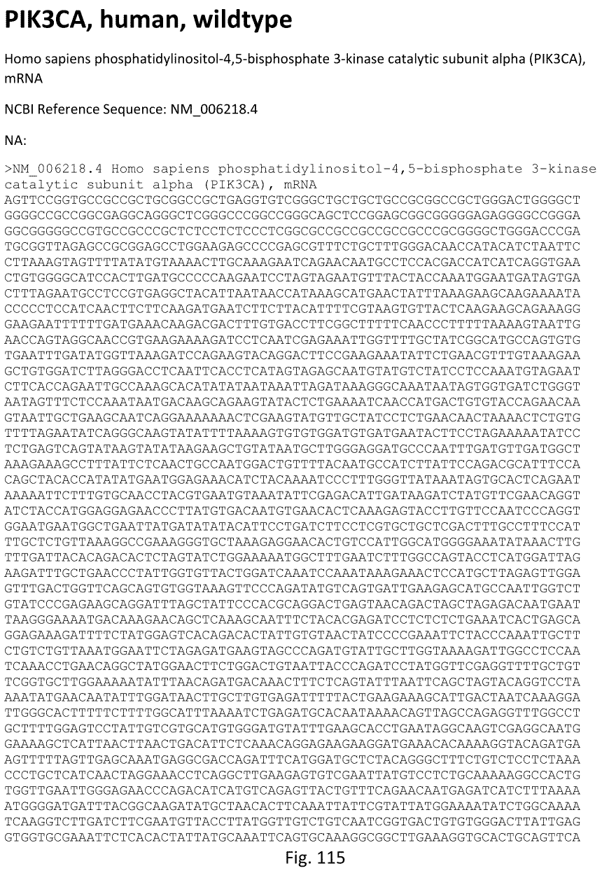

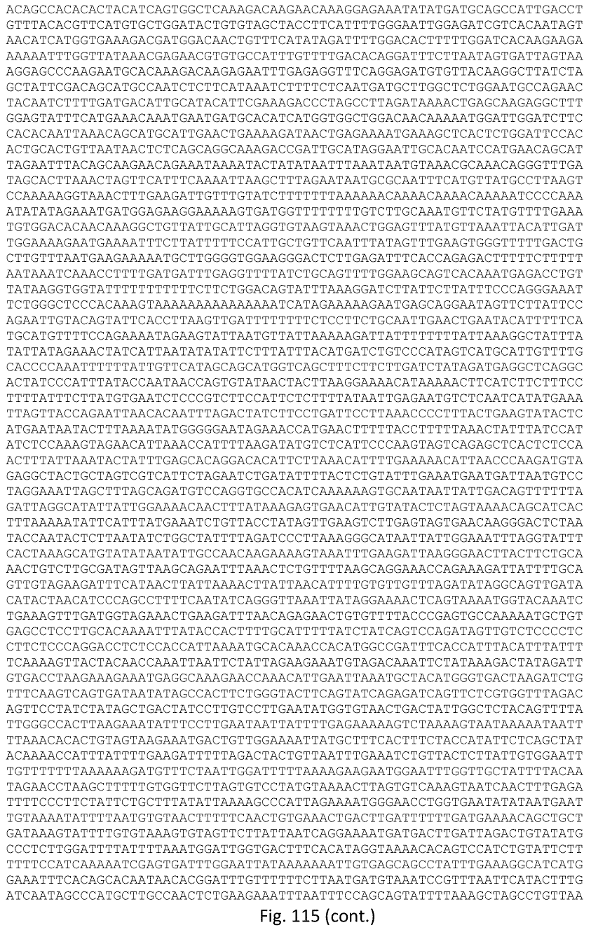

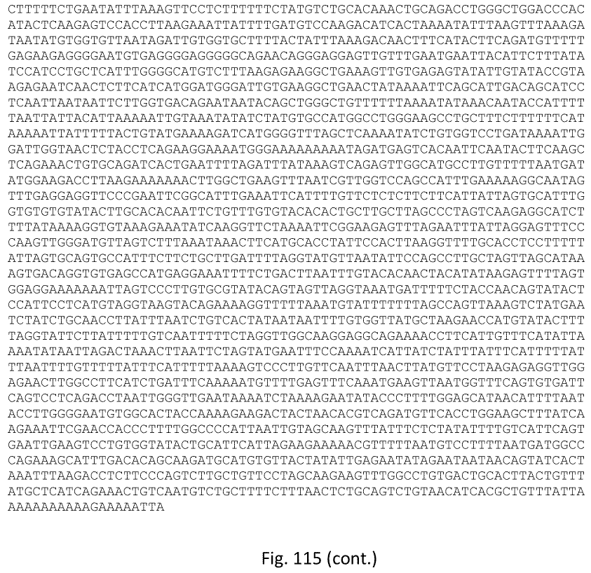

FIG. 115 shows the nucleotide sequence and amino acid sequence of human wild type PIK3CA.

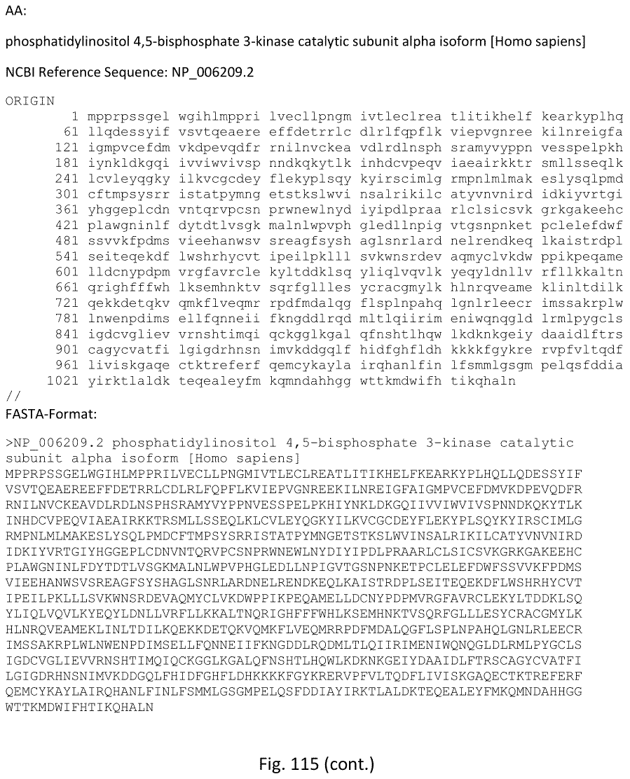

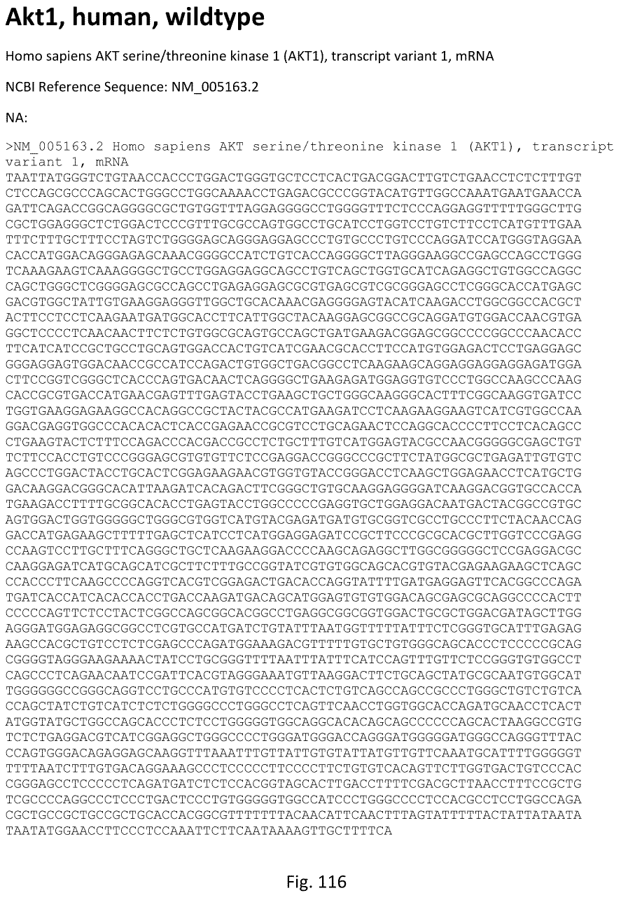

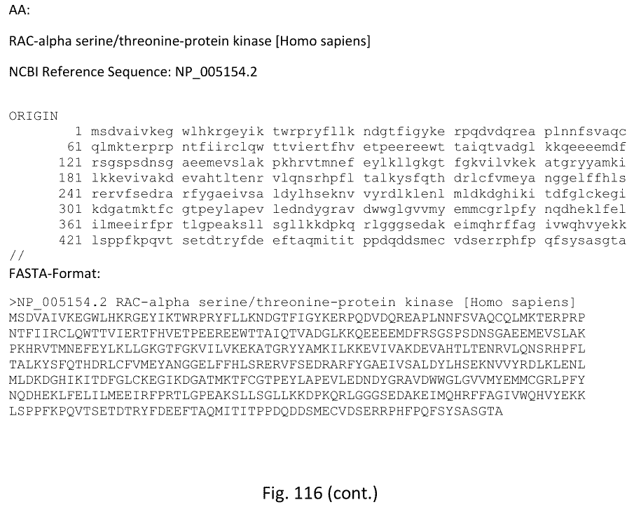

FIG. 116 shows the nucleotide sequence and amino acid sequence of human wild type Akt1.

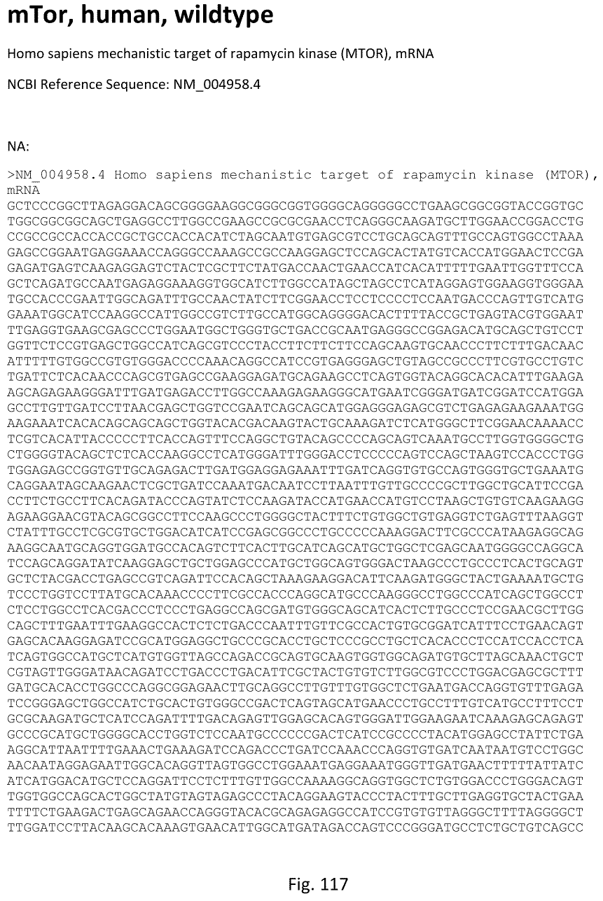

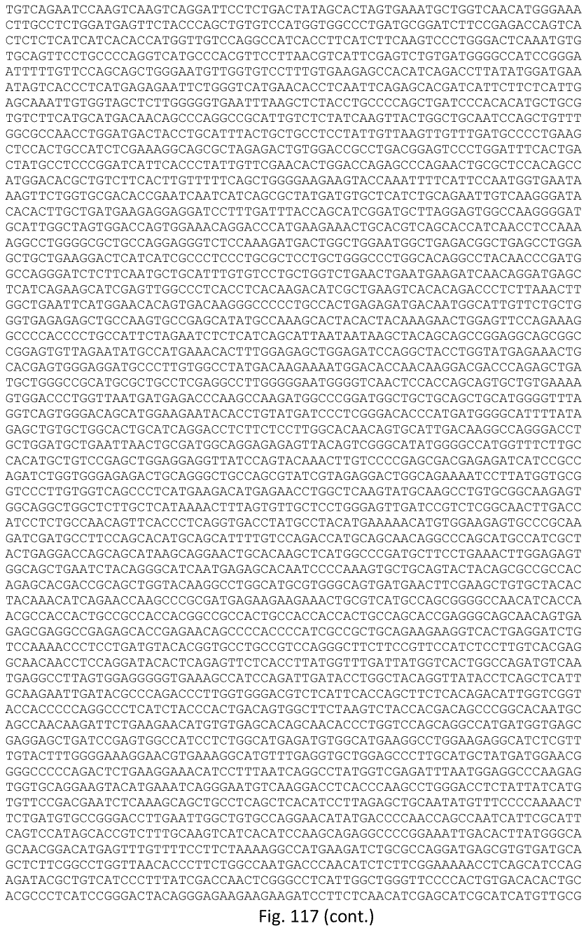

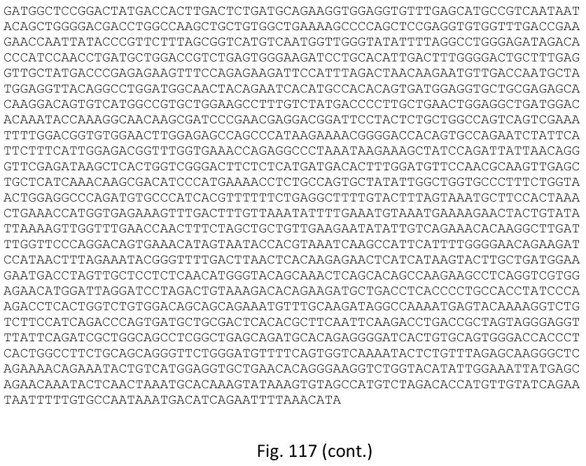

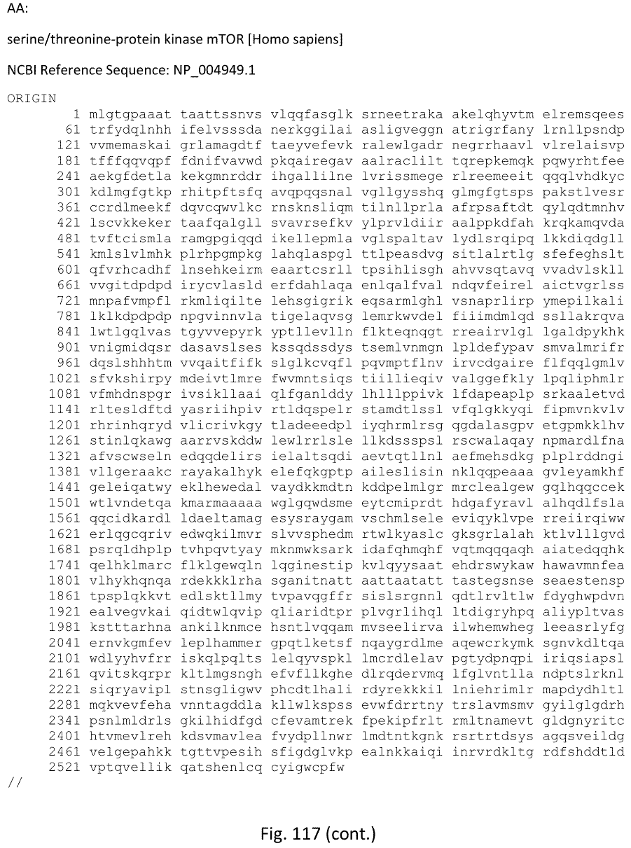

FIG. 117 shows the nucleotide sequence and amino acid sequence of human wild type mTOR.

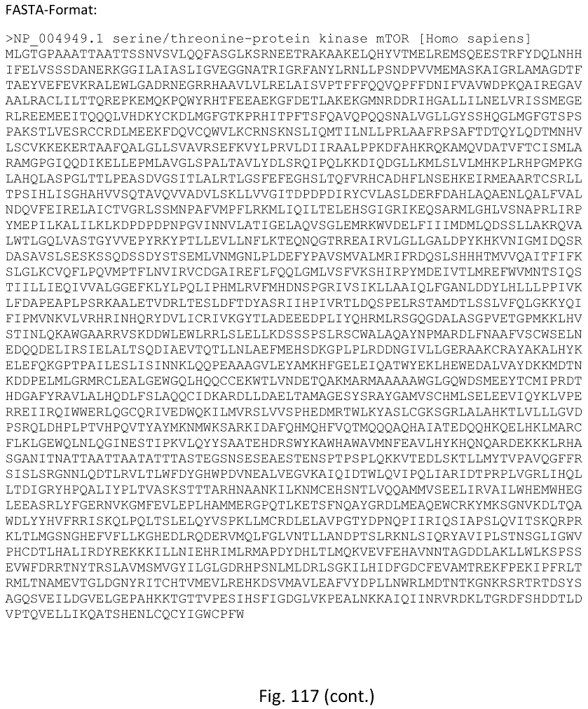

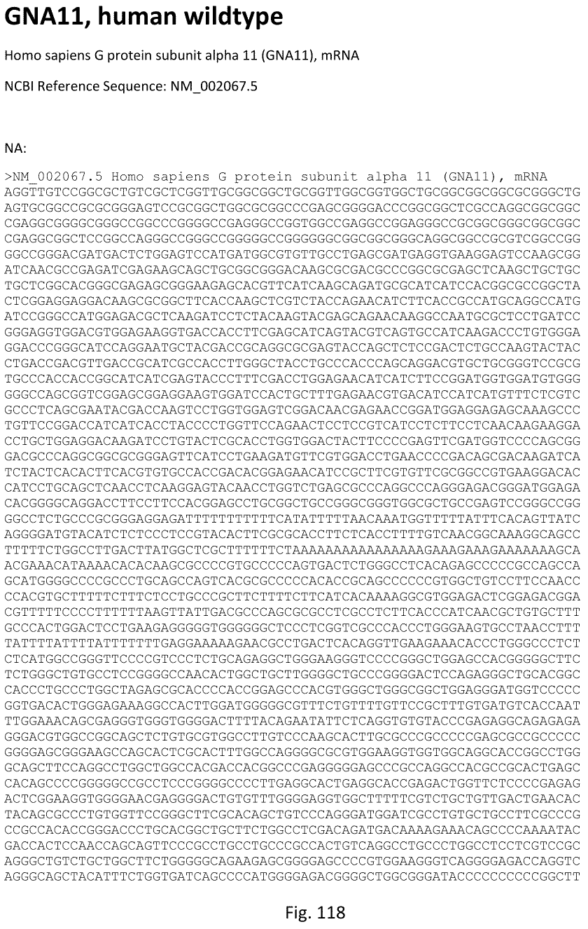

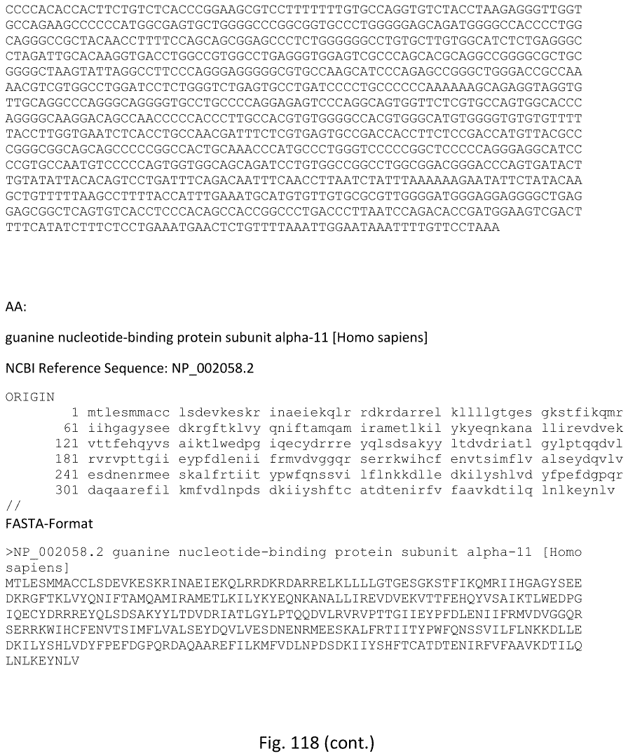

FIG. 118 shows the nucleotide sequence and amino acid sequence of human wild type GNA11.

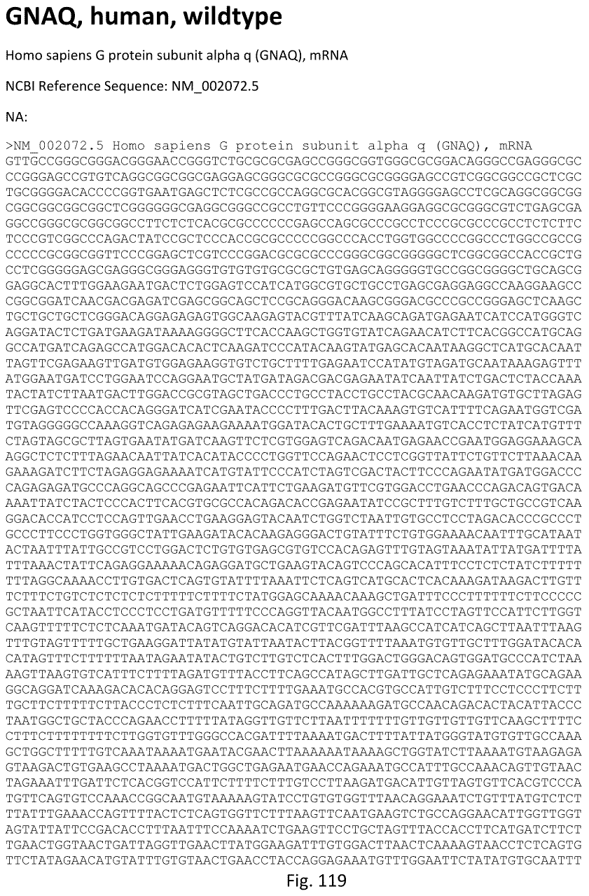

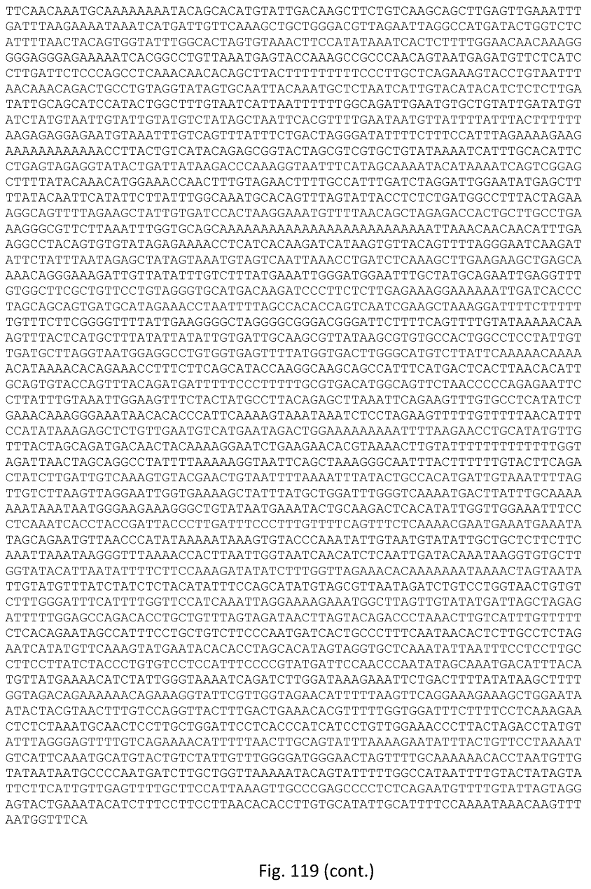

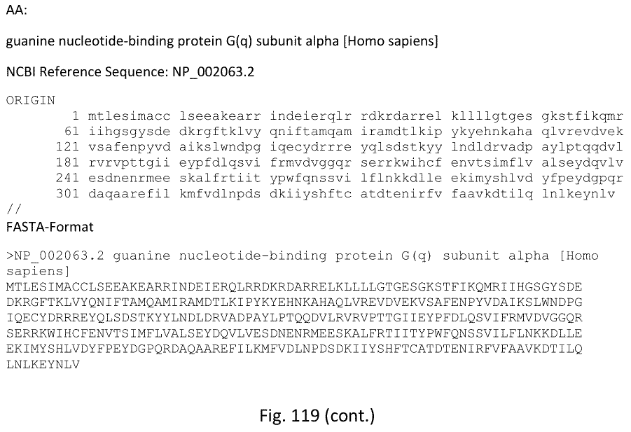

FIG. 119 shows the nucleotide sequence and amino acid sequence of human wild type GNAQ.

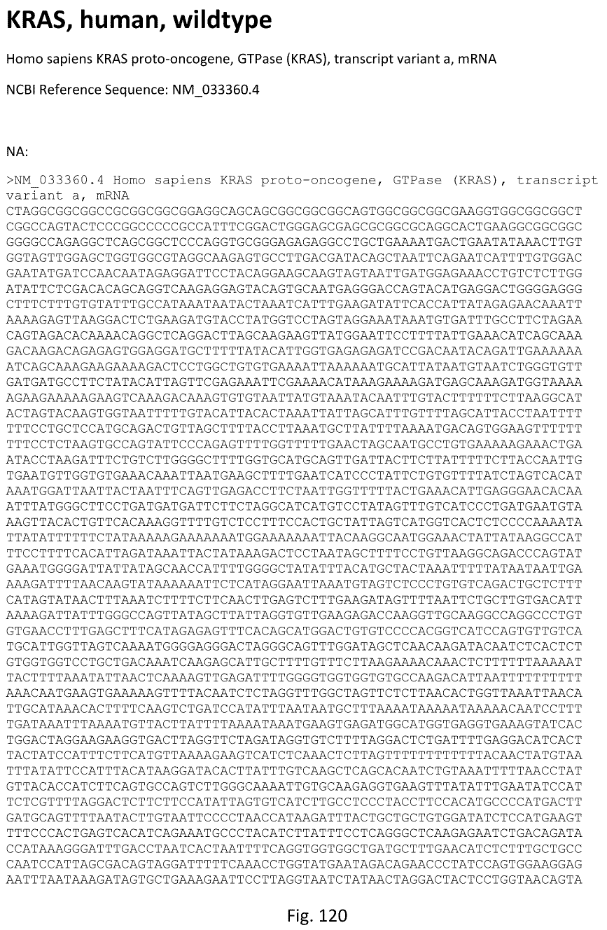

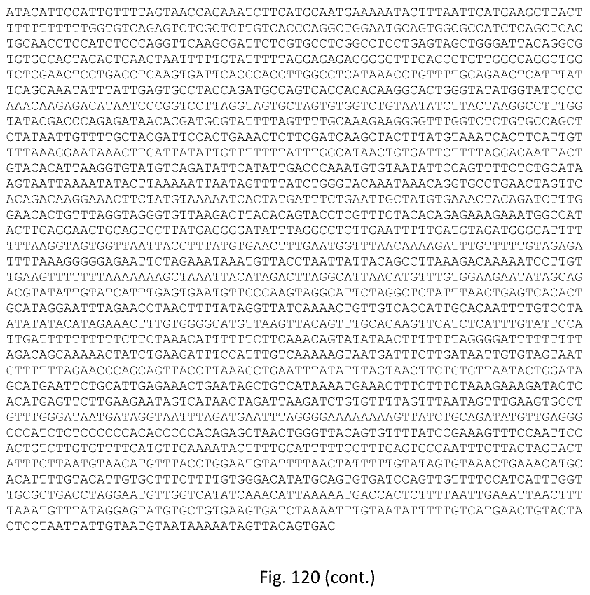

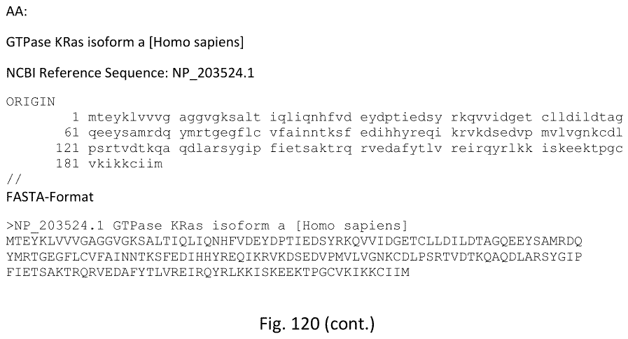

FIG. 120 shows the nucleotide sequence and amino acid sequence of human wild type KRAS.

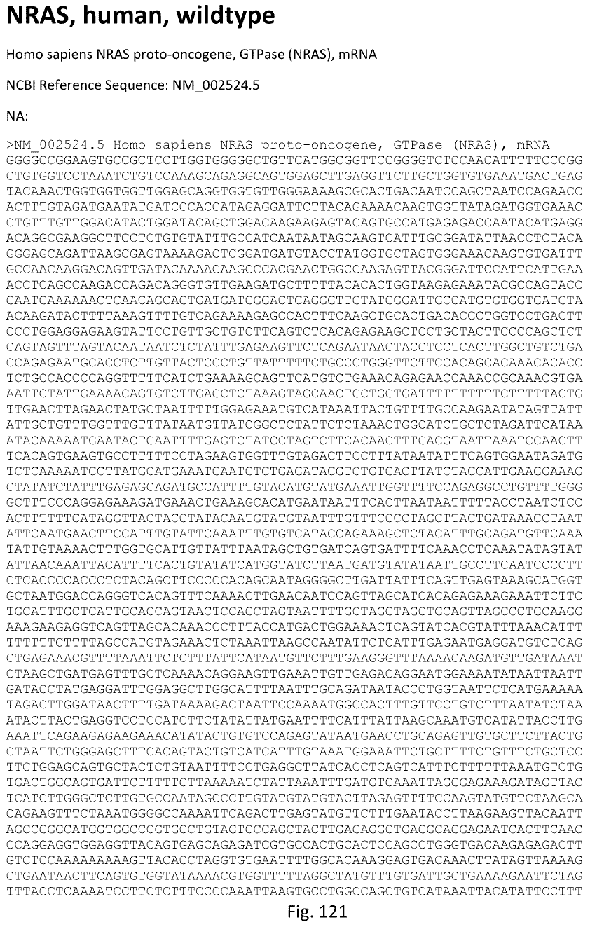

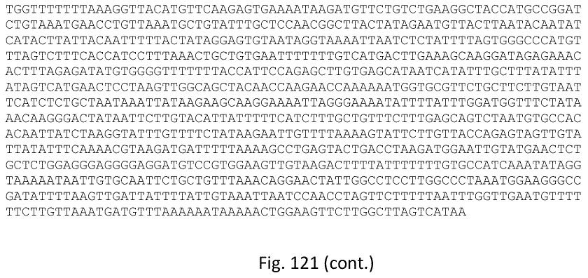

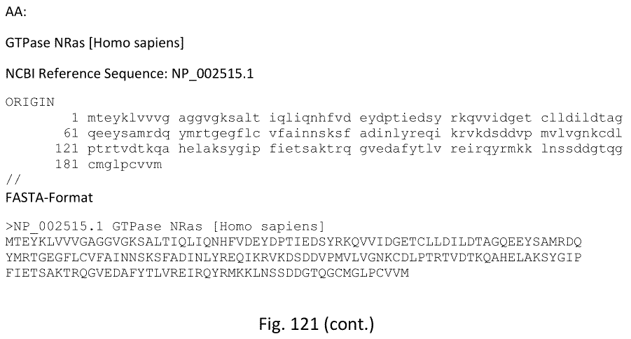

FIG. 121 shows the nucleotide sequence and amino acid sequence of human wild type NRAS.

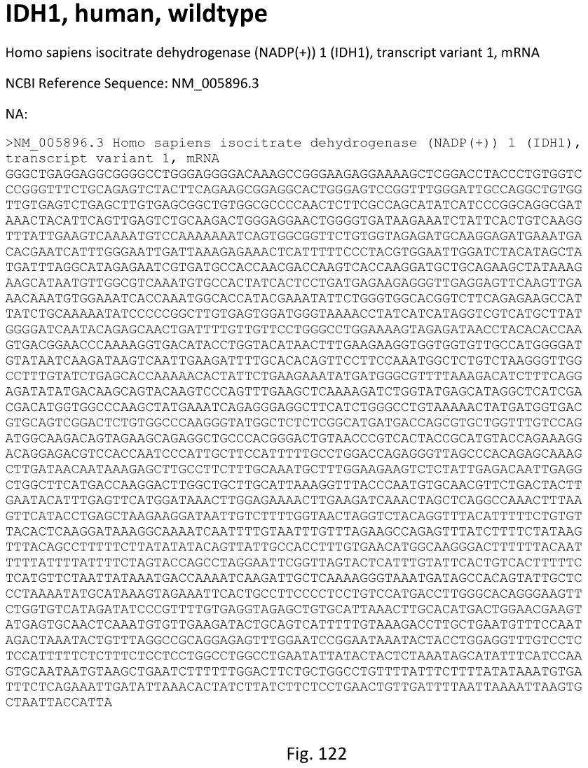

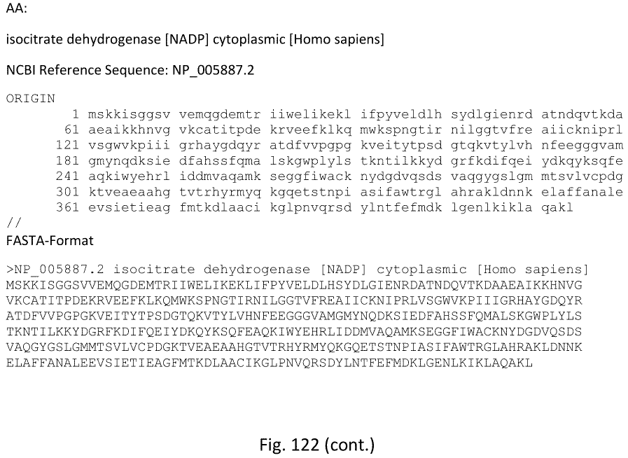

FIG. 122 shows the nucleotide sequence and amino acid sequence of human wild type IDH1.

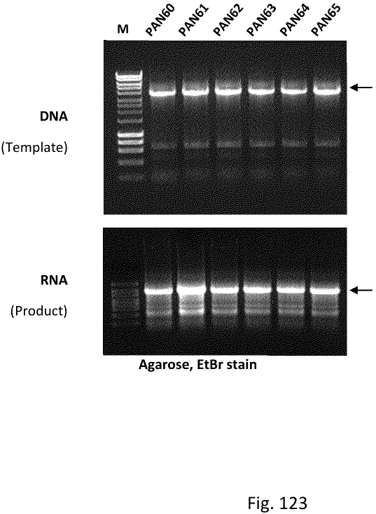

FIG. 123 shows 1% agarose gels stained with EtBr showing PCR products of Poly-A tailing PCRs.

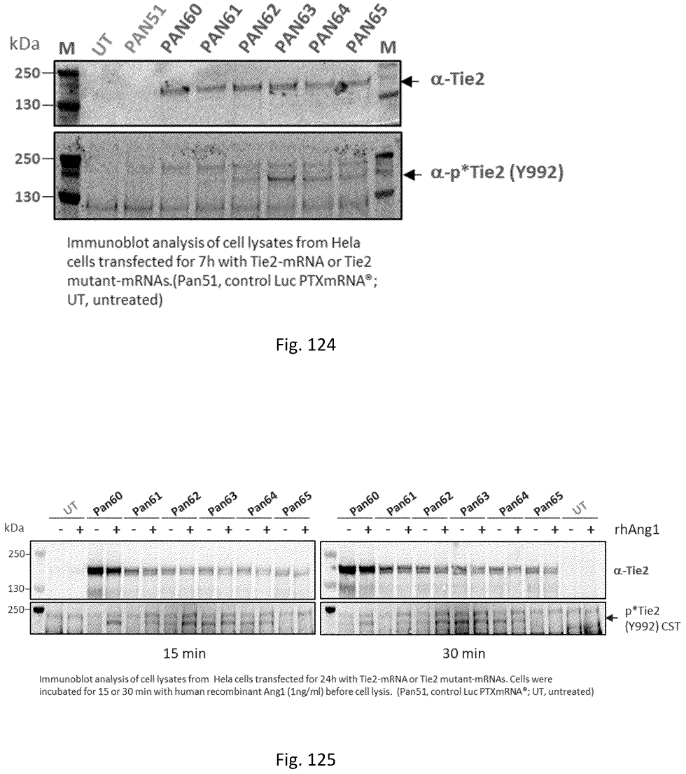

FIGS. 124 and 125 show the result of an immunoblot analysis of cell lysates from Hela cells transfected with Tie-2 mRNA or Tie-2 mutant mRNA.

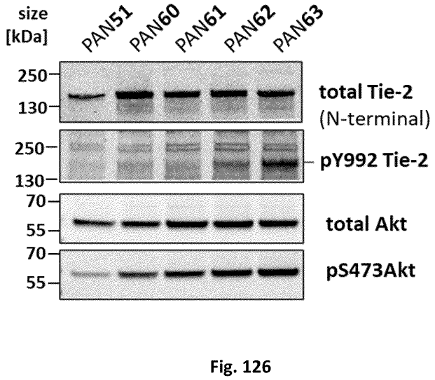

FIG. 126 shows the result of an immunoblot analysis of the effect of mRNAs encoding full-length wild type Tie-2 (construct PAN60) or mutant full-length Tie-2 constructs (constructs PAN61, PAN62 and PAN63) on Tie-2 pathway activation in human pulmonary microvascular endothelial cells (HPMEC) using total full-length Tie-2 (detected by an anti-Tie-2 antibody targeting the N-terminus of Tie-2), autophosphorylated full-length Tie-2 (detected by anti-phospho-Tie-2 antibody pY992 Tie-2 ), total Akt (detected by an anti-Akt antibody) and phosphorylated Akt (detected by anti-phospho-Akt antibody pS473Akt) as read-out.

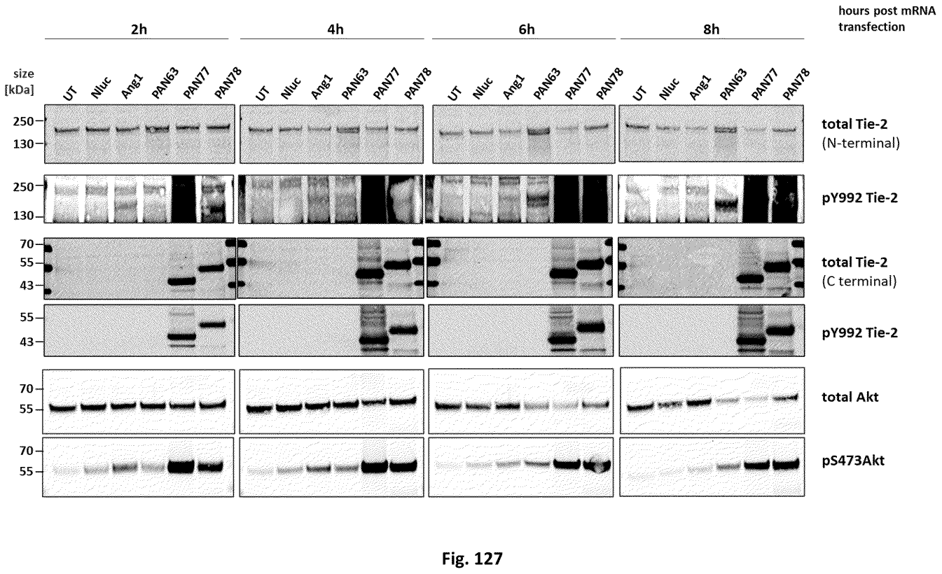

FIG. 127 shows the result of an immunoblot analysis of the effect over time of mRNAs encoding Nluc, Ang1, hypermorphic full-length Tie-2 (construct PAN63), and truncated hypermorphic Tie-2 variants with heterologous N-terminal domains (construct PAN77 and construct PAN78) on Tie-2 pathway activation in Tie-2-positive human pulmonary microvascular endothelial cells (HPMEC) using total full-length Tie-2 (detected by an anti-Tie-2 antibody targeting the N-terminus of Tie-2), phosphorylated full-length Tie-2 (detected by anti-phospho-Tie-2 antibody pY992 Tie-2) total truncated Tie-2 (detected by an anti-Tie-2 antibody targeting the C-terminus of Tie-2), phosphorylated truncated Tie-2 (detected by anti-phospho-Tie-2 antibody pY992 Tie-2) total Akt (detected by an anti-Akt antibody) and phosphorylated Akt (detected by anti-phospho-Akt antibody pS473Akt) as read-out. Total Akt and phosphorylated Akt was used to evaluate downstream Tie-2-pathway activation. mRNA encoding for Nluc (PAN29) was used as an unrelated control protein, whereas mRNA encoding for Ang1 was used for expression of the endogenous Tie-2-receptor agonist and to assess the endogenous Ang1 mediated Tie-2-receptor activation.

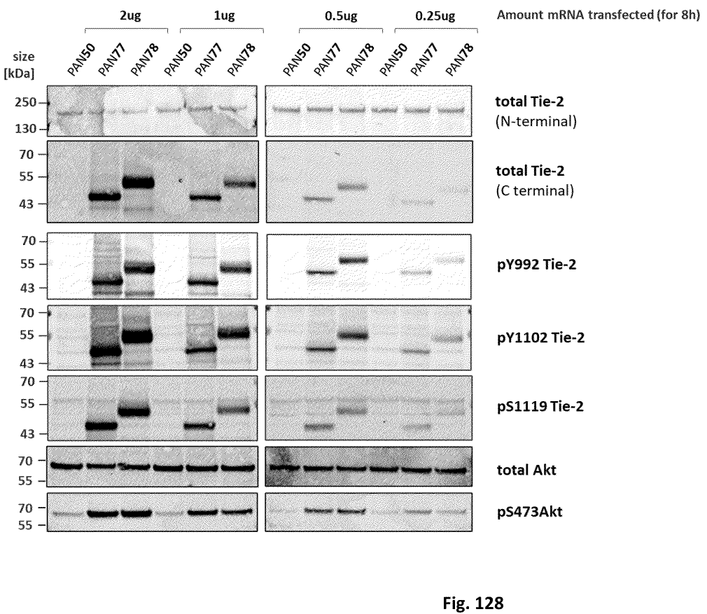

FIG. 128 shows the result of an immunoblot analysis of the dose-dependent expression of mRNAs encoding Nluc (PAN50) as an unrelated control and the truncated, hypermorphic Tie-2 variants with heterologous N-terminal domains (construct PAN77 and PAN78) in Tie-2-positive human pulmonary microvascular endothelial cells (HPMEC). An N-terminus-specific antibody was used to evaluate the amount of full-length total Tie-2, whereas a C-terminus-specific antibody was used to evaluate the amount of total truncated Tie-2 variants. The amount of phosphorylated truncated Tie-2 variants was assessed by using pY992, pY1102 and pS1119-specific antibodies. Concomitantly, the downstream Tie-2-pathway activation was evaluated using Akt-specific (Akt) and phosphorylated-Akt-specific (pS473Akt) antibodies.

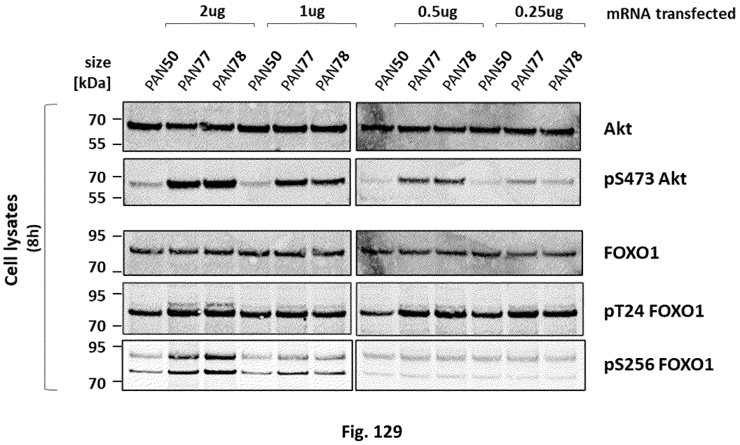

FIG. 129 shows the result of an immunoblot analysis of the dose-dependent expression of mRNAs encoding Nluc (PAN50) as an unrelated control and the truncated, hypermorphic Tie-2 variants with heterologous N-terminal domains (construct PAN77 and PAN78) in Tie-2-positive human pulmonary microvascular endothelial cells (HPMEC). Downstream Tie-2-pathway activation was evaluated using Akt-specific (Akt) and phospho-Akt-specific (pS473Akt) antibodies. Moreover, a FOXO1-specific antibody was used to assess the amount of total FOXO1 and phospho-FOXO1-specific antibodies pT24-FOXO1 and pS256-FOXO1 were used to evaluate FOXO1-phosphorylation status.

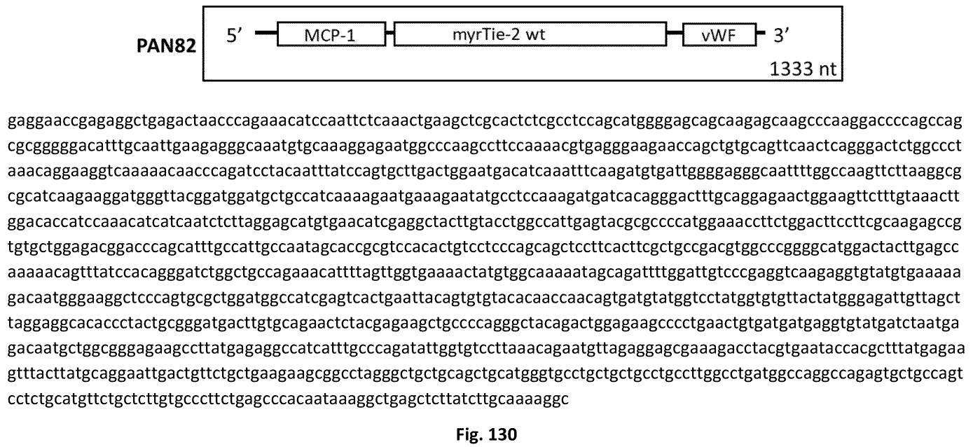

FIG. 130 shows the basic structure and nucleotide sequence of recombinant nucleic acid construct PAN 82.

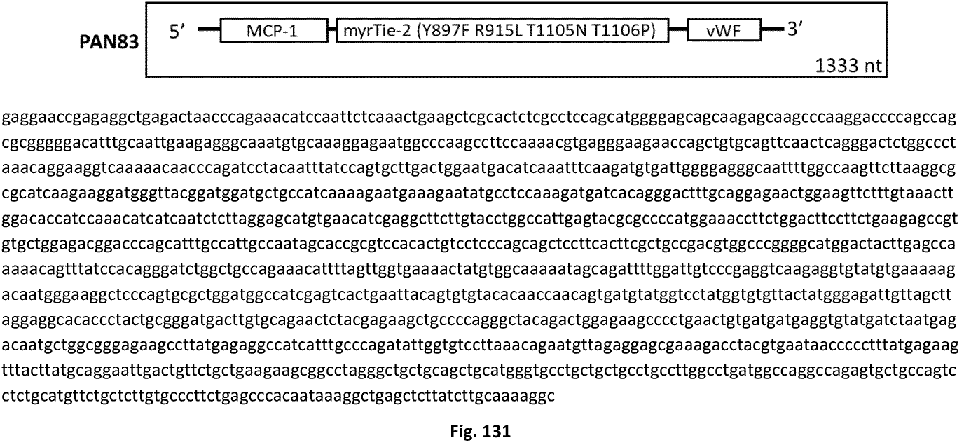

FIG. 131 shows the basic structure and nucleotide sequence of recombinant nucleic acid construct PAN 83.

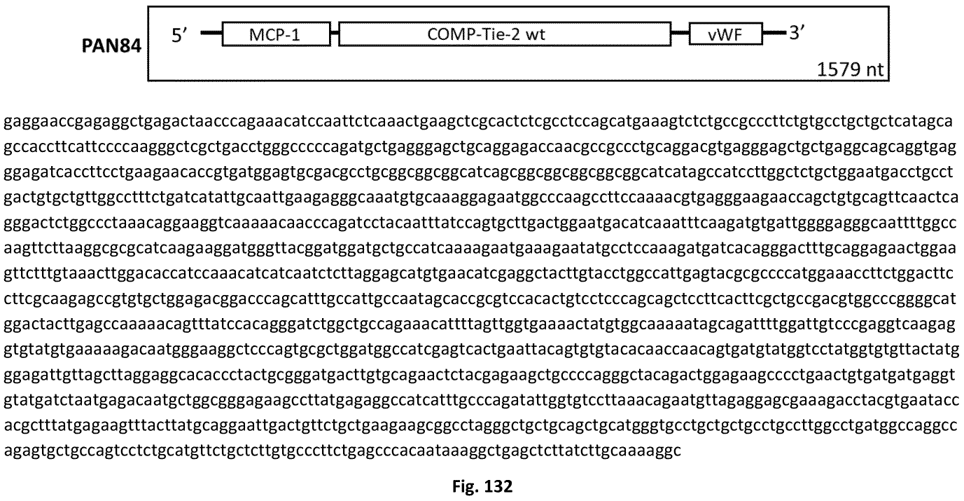

FIG. 132 shows the basic structure and nucleotide sequence of recombinant nucleic acid construct PAN 84.

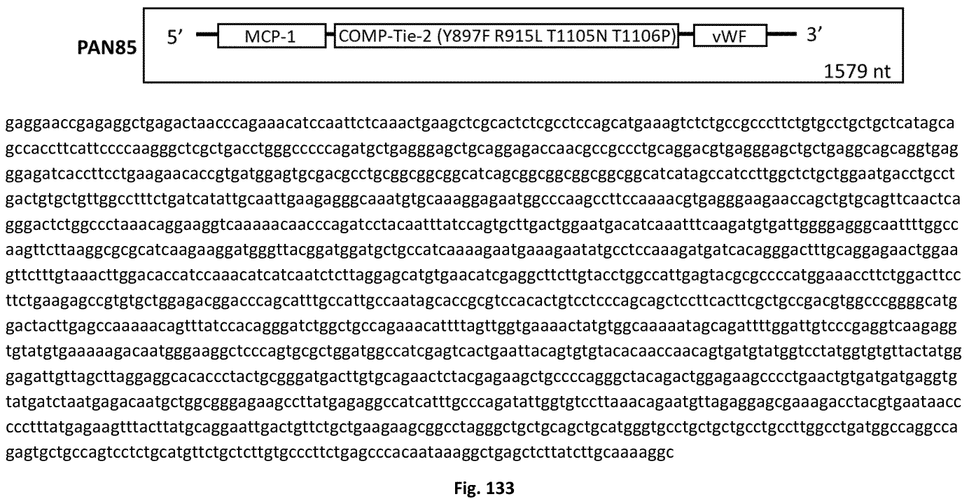

FIG. 133 shows the basic structure and nucleotide sequence of recombinant nucleic acid construct PAN 85.

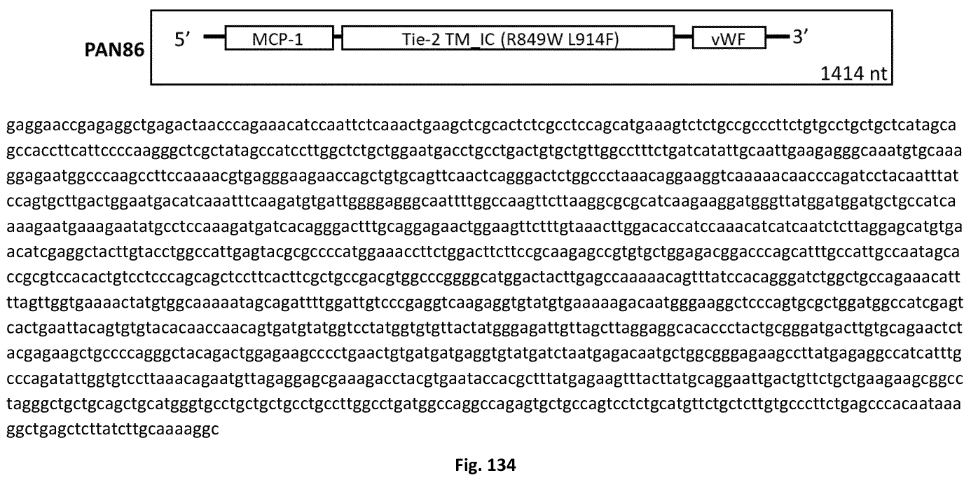

FIG. 134 shows the basic structure and nucleotide sequence of recombinant nucleic acid construct PAN 86.

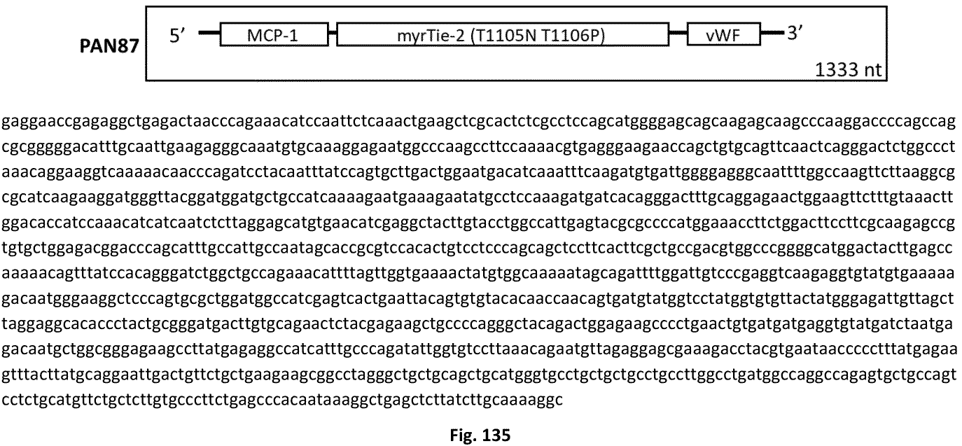

FIG. 135 shows the basic structure and nucleotide sequence of recombinant nucleic acid construct PAN 87.

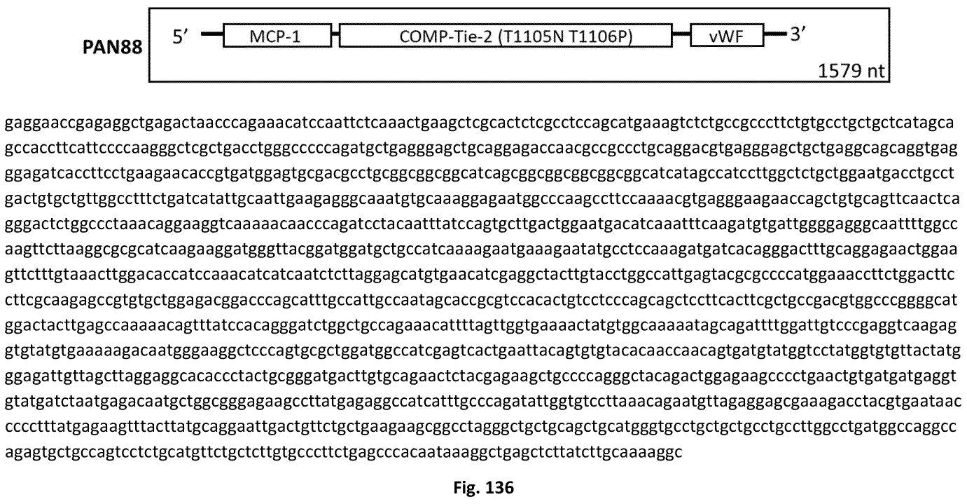

FIG. 136 shows the basic structure and nucleotide sequence of recombinant nucleic acid construct PAN 88.

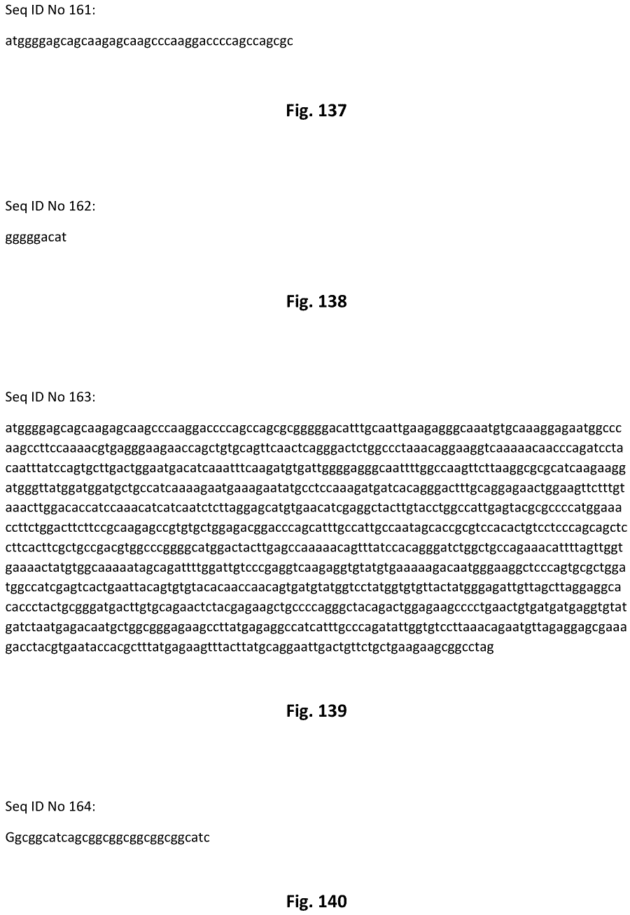

FIG. 137 shows the nucleotide sequence formyristylation signal of Scr-kinase.

FIG. 138 shows the nucleotide sequence for a GGH linker.

FIG. 139 shows the nucleotide sequence of the effector molecule in PAN77.

FIG. 140 shows the nucleotide sequence of the GGISGGGGGI-linker.

FIG. 141 shows the nucleotide sequence of the effector molecule in PAN78.

FIG. 142 shows the nucleotide sequence of the effector molecule in PAN82.

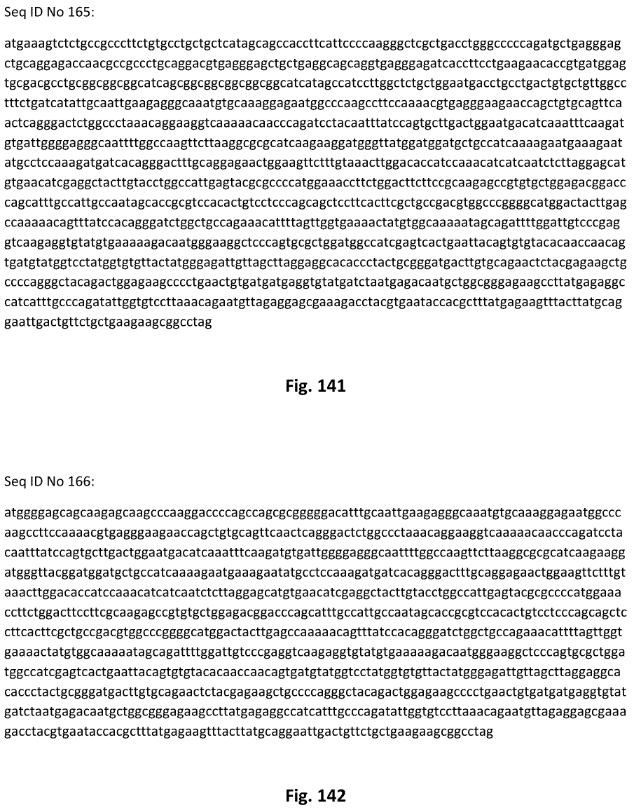

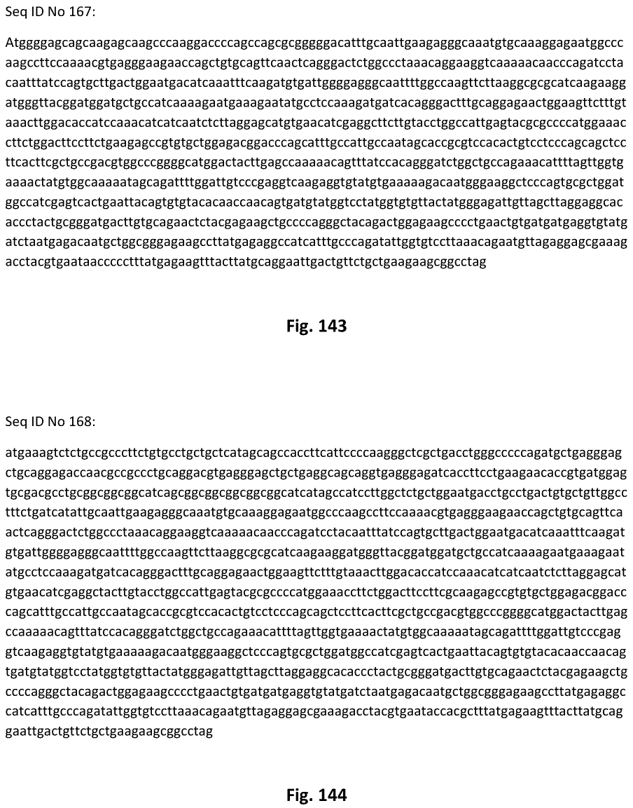

FIG. 143 shows the nucleotide sequence of the effector molecule in PAN83.

FIG. 144 shows the nucleotide sequence of the effector molecule in PAN84.

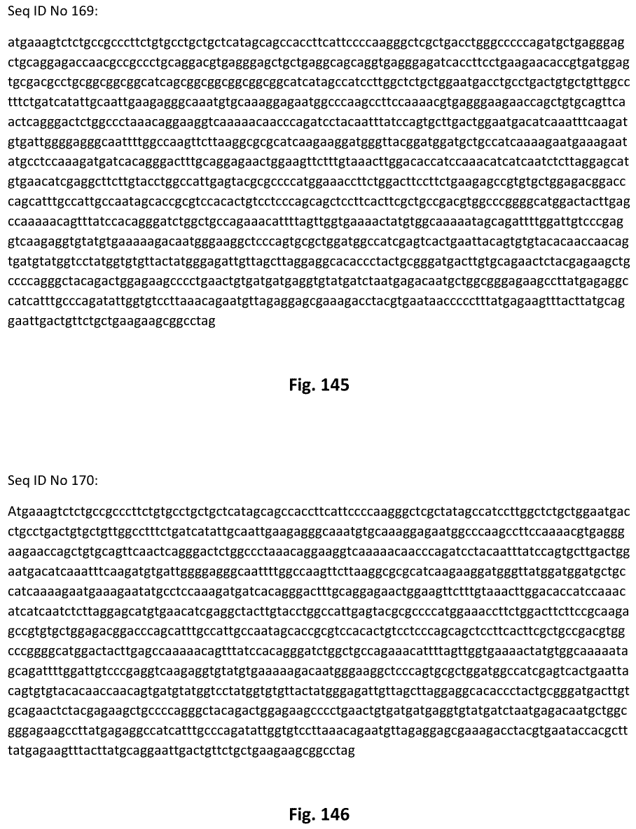

FIG. 145 shows the nucleotide sequence of the effector molecule in PAN85.

FIG. 146 shows the nucleotide sequence of the effector molecule in PAN86.

FIG. 147 shows the nucleotide sequence of the effector molecule in PAN87.

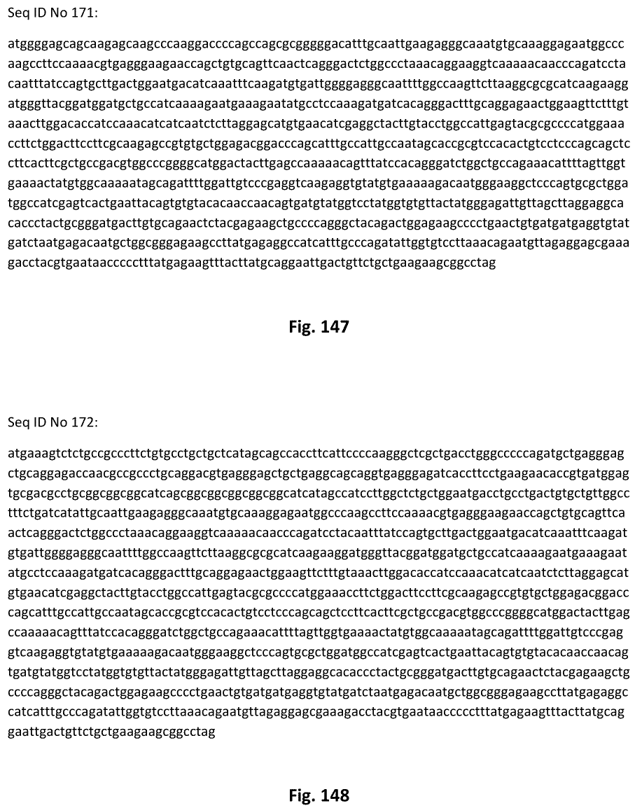

FIG. 148 shows the nucleotide sequence of the effector molecule in PAN88.

FIG. 1 is a schematic representation of conventional mRNA molecule structure. Eukaryotic including mammalian mRNAs consist of a cap region, a 5′ untranslated region (UTR), the coding sequence (CDS) with an open reading frame (ORF) starting with a consensus Kozak sequence for optimal translation initiation and in case of secreted proteins followed by an signal peptide leader sequences, a 3′ UTR, and a poly A-tail (>120 nt) at the 3′ end.

FIG. 2 is a schematic representation of selected examples for mRNA constructs with the coding sequence with an open reading frame being the one of Nano-luciferase. It is within the present invention that for each and any of the indicated mRNA constructs the coding region comprising a sequence with an open reading frame for Nano-luciferase may be replaced by a coding regions comprising a sequence coding for an effector molecule, particularly by a coding region comprising a sequence coding for an effector molecule disclosed herein; in a preferred embodiment thereof, the effector molecule is Ang1 or COMP-Ang1. In accordance with the present invention, the recombinant nucleic acid constructs are designed as follows:

Recombinant nucleic acid construct PAN02 comprises as the 5′ non-translated region the 5′ UTR of Ang1, as the nucleic acid sequence coding for a signal peptide the nucleic acid sequence coding for a signal peptide of Ang1, as the coding region coding for an effector molecule the coding region coding for Nano luciferase, and as the 3′ non-translated region the 3′ UTR of Ang1.

Recombinant nucleic acid construct PAN05 comprises as the 5′ non-translated region the 5′ UTR of Ang2, as the nucleic acid sequence coding for a signal peptide the nucleic acid sequence coding for a signal peptide of Ang2, as the coding region coding for an effector molecule the coding region coding for Nano luciferase, and as the 3′ non-translated region the 3′ UTR of von Willebrand Factor (vWF).

Recombinant nucleic acid construct PAN07 comprises as the 5′ non-translated region the 5′ UTR of Galectin-9, as the nucleic acid sequence coding for a signal peptide the nucleic acid sequence coding for a signal peptide of Ang2, as the coding region coding for an effector molecule the coding region coding for Nano luciferase, and as the 3′ non-translated region the 3′ UTR of von Willebrand Factor (vWF).

Recombinant nucleic acid construct PAN08 comprises as the 5′ non-translated region the 5′ UTR of Hsp70, as the nucleic acid sequence coding for a signal peptide the nucleic acid sequence coding for a signal peptide of Ang2, as the coding region coding for an effector molecule the coding region coding for Nano luciferase, and as the 3′ non-translated region the 3′ UTR of HSP70.

Recombinant nucleic acid construct PAN09 comprises as the 5′ non-translated region the 5′ UTR of H3.3, as the nucleic acid sequence coding for a signal peptide the nucleic acid sequence coding for a signal peptide of Ang2, as the coding region coding for an effector molecule the coding region coding for Nano luciferase, and as the 3′ non-translated region the 3′ UTR of H3.3.

Recombinant nucleic acid construct PAN10 comprises as the 5′ non-translated region the 5′ UTR of RPL12s.c., as the nucleic acid sequence coding for a signal peptide the nucleic acid sequence coding for a signal peptide of Ang2, as the coding region coding for an effector molecule the coding region coding for Nano luciferase, and as the 3′ non-translated region the 3′ UTR of RPL12s.c. This construct additionally comprises another start codon preceding the start codon of the coding region for Nano-luciferase in-frame.

Recombinant nucleic acid construct PAN11 comprises as the 5′ non-translated region the 5′ UTR of GADD34, as the nucleic acid sequence coding for a signal peptide the nucleic acid sequence coding for a signal peptide of Ang2, as the coding region coding for an effector molecule the coding region coding for Nano luciferase, and as the 3′ non-translated region the 3′ UTR of GADD34.

Recombinant nucleic acid construct PAN12 comprises as the 5′ non-translated region the 5′ UTR of MCP-1 as the nucleic acid sequence coding for a signal peptide the nucleic acid sequence coding for a signal peptide of MCP-1, as the coding region coding for an effector molecule the coding region coding for Nano luciferase, and as the 3′ non-translated region the 3′ UTR of MCP-1.

Recombinant nucleic acid construct PAN13 comprises as the 5′ non-translated region the 5′ UTR of EDN1, as the nucleic acid sequence coding for a signal peptide the nucleic acid sequence coding for a signal peptide of EDN1, as the coding region coding for an effector molecule the coding region coding for Nano luciferase, and as the 3′ non-translated region the 3′ UTR of EDN1.

Recombinant nucleic acid construct PAN28 comprises as the 5′ non-translated region the 5′ UTR of Ang2, as the nucleic acid sequence coding for a signal peptide the nucleic acid sequence coding for a signal peptide of IL-6, as the coding region coding for an effector molecule the coding region coding for Nano luciferase, and as the 3′ non-translated region the 3′ UTR of vWF.

Recombinant nucleic acid construct PAN29 comprises as the 5′ non-translated region the 5′ UTR of MCP-1, as the nucleic acid sequence coding for a signal peptide the nucleic acid sequence coding for a signal peptide of MCP-1, as the coding region coding for an effector molecule the coding region coding for Nano luciferase, and as the 3′ non-translated region the 3′ UTR of von Willebrand Factor (vWF).

Recombinant nucleic acid construct PAN30 comprises as the 5′ non-translated region the 5′ UTR of MCP-1, as the nucleic acid sequence coding for a signal peptide the nucleic acid sequence coding for a signal peptide of MCP-1, as the coding region coding for an effector molecule the coding region coding for Nano luciferase, and as the 3′ non-translated region the 3′ UTR of HSP70.

Recombinant nucleic acid construct PAN31 comprises as the 5′ non-translated region the 5′ UTR of MCP-1, as the nucleic acid sequence coding for a signal peptide the nucleic acid sequence coding for a signal peptide of IL-6, as the coding region coding for an effector molecule the coding region coding for Nano luciferase, and as the 3′ non-translated region the 3′ UTR of MCP-1.

Recombinant nucleic acid construct PAN32 comprises as the 5′ non-translated region the 5′ UTR of MCP-1, as the nucleic acid sequence coding for a signal peptide the nucleic acid sequence coding for a signal peptide of Ang2, as the coding region coding for an effector molecule the coding region coding for Nano luciferase, and as the 3′ non-translated region the 3′ UTR of MCP-1.

Recombinant nucleic acid construct PAN33 comprises as the 5′ non-translated region the 5′ UTR of MCP-1, no nucleic acid sequence coding for a signal peptide, as the coding region coding for an effector molecule the coding region coding for Nano luciferase, and as the 3′ non-translated region the 3′ UTR of MCP-1.

Recombinant nucleic acid construct PAN34 comprises as the 5′ non-translated region the 5′ UTR of Hsp70, as the nucleic acid sequence coding for a signal peptide the nucleic acid sequence coding for a signal peptide of MCP-1, as the coding region coding for an effector 3′ UTR of MCP-1.

Recombinant nucleic acid construct PAN35 comprises as the 5′ non-translated region the 5′ UTR of RPL 12 s.c. (spinach chloroplast), as the nucleic acid sequence coding for a signal peptide the nucleic acid sequence coding for a signal peptide of MCP-1, as the coding region coding for an effector molecule the coding region coding for Nano luciferase, and as the 3′ non-translated region the 3′ UTR of MCP-1.

Recombinant nucleic acid construct PAN36 comprises as the 5′ non-translated region the 5′ UTR of MCP-1, as the nucleic acid sequence coding for a signal peptide the nucleic acid sequence coding for a signal peptide of MCP-1, as the coding region coding for an effector molecule the coding region coding for Ang1, and as the 3′ non-translated region the 3′ UTR of MCP-1.

Recombinant nucleic acid construct PAN37 comprises as the 5′ non-translated region the 5′ UTR of MCP-1, as the nucleic acid sequence coding for a signal peptide the nucleic acid sequence coding for a signal peptide of MCP-1, as the coding region coding for an effector molecule the coding region coding for hCOMP-Ang1, and as the 3′ non-translated region the 3′ UTR of MCP-1.

Recombinant nucleic acid construct PAN38 comprises as the 5′ non-translated region the 5′ UTR of Ang2, as the nucleic acid sequence coding for a signal peptide the nucleic acid sequence coding for a signal peptide of MCP-1, as the coding region coding for an effector molecule the coding region coding for Nano luciferase, and as the 3′ non-translated region the 3′ UTR of von Willebrand Factor (vWF).

Recombinant nucleic acid construct PAN39 comprises as the 5′ non-translated region the 5′ UTR of Ang2* (Ang2 5′-UTR with deleted upstream ATGs), as the nucleic acid sequence coding for a signal peptide the nucleic acid sequence coding for a signal peptide of IL-6, as the coding region coding for an effector molecule the coding region coding for Nano luciferase, and as the 3′ non-translated region the 3′ UTR of von Willebrand Factor (vWF).

Recombinant nucleic acid construct PAN40 comprises as the 5′ non-translated region the 5′ UTR of Ang2, as the nucleic acid sequence coding for a signal peptide the nucleic acid sequence coding for a signal peptide of Gaussia luciferase, as the coding region coding for an effector molecule the coding region coding for Nano luciferase, and as the 3′ non-translated region the 3′ UTR of von Willebrand Factor (vWF).

Recombinant nucleic acid construct PAN41 comprises as the 5′ non-translated region the 5′ UTR of RPL12 s.c., as the nucleic acid sequence coding for a signal peptide the nucleic acid sequence coding for a signal peptide of MCP-1 with an additional upstream ATG for translational start, as the coding region coding for an effector molecule the coding region coding for Nano luciferase, and as the 3′ non-translated region the 3′ UTR ofRPL12 s.c.. This construct additionally comprises another start codon preceding the start codon of the coding region for Nano-luciferase in-frame.

Recombinant nucleic acid construct PAN42 comprises as the 5′ non-translated region the 5′ UTR of RPL12 s.c., as the nucleic acid sequence coding for a signal peptide the nucleic acid sequence coding for a signal peptide of MCP-1, as the coding region coding for an effector molecule the coding region coding for Nano luciferase, and as the 3′ non-translated region the 3′ UTR of RPL12 s.c..

Recombinant nucleic acid construct PAN43 comprises as the 5′ non-translated region the 5′ UTR of RPL12 s.c., as the nucleic acid sequence coding for a signal peptide the nucleic acid sequence coding for a signal peptide of Ang2, as the coding region coding for an effector molecule the coding region coding for Nano luciferase, and as the 3′ non-translated region the 3′ UTR of RPL12 s.c..

Recombinant nucleic acid construct PAN44 comprises as the 5′ non-translated region the 5′ UTR of Hsp70, as the nucleic acid sequence coding for a signal peptide the nucleic acid sequence coding for a signal peptide of MCP-1, as the coding region coding for an effector molecule the coding region coding for Nano luciferase, and as the 3′ non-translated region the 3′ UTR of Hsp70.

Recombinant nucleic acid construct PAN45 comprises as the 5′ non-translated region the 5′ UTR of Hsp70m5, as the nucleic acid sequence coding for a signal peptide the nucleic acid sequence coding for a signal peptide of MCP-1, as the coding region coding for an effector molecule the coding region coding for Nano luciferase, and as the 3′ non-translated region the 3′ UTR of von Willebrand Factor (vWF).

Recombinant nucleic acid construct PAN46 comprises as the 5′ non-translated region the 5′ UTR of E-selectin, as the nucleic acid sequence coding for a signal peptide the nucleic acid sequence coding for a signal peptide of IL-6, as the coding region coding for an effector molecule the coding region coding for Nano luciferase, and as the 3′ non-translated region the 3′ UTR of von Willebrand Factor (vWF).

Recombinant nucleic acid construct PAN47 comprises as the 5′ non-translated region the 5′ UTR of ICAM1, as the nucleic acid sequence coding for a signal peptide the nucleic acid sequence coding for a signal peptide of IL-6, as the coding region coding for an effector molecule the coding region coding for Nano luciferase, and as the 3′ non-translated region the 3′ UTR of von Willebrand Factor (vWF).

Recombinant nucleic acid construct PAN48 comprises as the 5′ non-translated region the 5′ UTR of IL-6, as the nucleic acid sequence coding for a signal peptide the nucleic acid sequence coding for a signal peptide of IL-6, as the coding region coding for an effector molecule the coding region coding for Nano luciferase, and as the 3′ non-translated region the 3′ UTR of IL-6).

Recombinant nucleic acid construct PAN49 comprises as the 5′ non-translated region the 5′ UTR of von Willebrand Factor (vWF), as the nucleic acid sequence coding for a signal peptide the nucleic acid sequence coding for a signal peptide of von Willebrand Factor (vWF), as the coding region coding for an effector molecule the coding region coding for Nano luciferase, and as the 3′ non-translated region the 3′ UTR of von Willebrand Factor (vWF).

FIG. 3 shows a restriction map of a plasmid pcDNA3.1 (-) used as an illustrative example for a plasmid for the expression of an exemplary recombinant nucleic acid construct of the present invention, whereby such exemplary recombinant nucleic acid construct is construct PAN 11; restriction sites XhoI/HindIII are shown.

FIG. 4 shows a restriction digestion map of the PAN11 expressing pcDNA3.1- plasmid of FIG. 3; the insert represents PAN11 sequence with engineered 5′ and 3′ UTR, signal sequence and the coding region coding for Nano-Luciferase.

FIG. 5 is an 1% agarose gel stained with EtBr showing the non-linearized (uncut, supercoiled, right lanes) and linearization product (left lanes) of pcDNA3.1(-) plasmids upon BamHI restriction of the constructs PAN28 (“28”), PAN13 (“13”), PAN12 (“12”), PAN11 (“11”), PAN10 (“10”), PAN09 (“09”), PAN08 (“08”), PAN07 (“07”) and PAN05 (“05”).

FIG. 6 shows 1% agarose gels stained with EtBr showing PCR products of Poly-A tailing PCRs for the addition of 120 nt of Poly-A tail by 120 nt long poly-T 3′ primer flanking the different recombinant nucleic acid constructs. On top are the optimized PCR conditions for the indicated recombinant nucleic acid constructs. For example, for Pan02 the conditions are as follows: denaturation for 2 minutes at 94° C. 33 cycles of 30 seconds at 96° C., 15 seconds at 55° C. and 4 minutes at 72° C. Final extension for 8 minutes at 72° C.

FIG. 8 is an image of a 1% non-denaturing agarose gel stained with EtBr showing the in vitro transcribed mRNAs of constructs PAN28 (“28”), PAN13 (“13”), PAN12 (“12”), PAN11 (“11”), PAN10 (“10”), PAN09 (“09”), PAN08 (“08”), PAN07 (“07”) and PAN05 (“05”). M: is a lane with a high range RiboRuler RNA ladder. From this FIG. 8 may be taken that the transcribed mRNAs were intact and, more specifically, were not degraded.

FIG. 24 shows the basic structure and nucleotide sequence of recombinant nucleic acid construct PAN01. PAN1 is an embodiment of the present invention comprising as the 5′ non-translated region the 5′ UTR of Ang1, as the nucleic acid sequence coding for a signal peptide the nucleic acid sequence coding for a signal peptide of Ang1, as the coding region coding for an effector molecule the coding region coding for Ang1, and as the 3′ non-translated region the 3′ UTR of Ang1.

FIG. 25 shows the basic structure and nucleotide sequence of recombinant nucleic acid construct PAN03. PAN03 is an embodiment of the present invention comprising as the 5′ non-translated region the 5′ UTR of Ang1, as the nucleic acid sequence coding for a signal peptide the nucleic acid sequence coding for a signal peptide of Ang1, as the coding region coding for an effector molecule the coding region coding for COMP-Ang1, and as the 3′ non-translated region the 3′ UTR of Ang1.

FIG. 26 shows the basic structure and nucleotide sequence of recombinant nucleic acid construct PAN04. PAN04 is an embodiment of the present invention comprising as the 5′ non-translated region the 5′ UTR of Ang2, as the nucleic acid sequence coding for a signal peptide the nucleic acid sequence coding for a signal peptide of Ang2, as the coding region coding for an effector molecule the coding region coding for Ang1, and as the 3′ non-translated region the 3′ UTR of von Willebrand Factor (vWF).

FIG. 27 shows the basic structure and nucleotide sequence of recombinant nucleic acid construct PAN06. PAN06 is an embodiment of the present invention comprising as the 5′ non-translated region the 5′ UTR of Ang2, as the nucleic acid sequence coding for a signal peptide the nucleic acid sequence coding for a signal peptide of Ang2, as the coding region coding for an effector molecule the coding region coding for COMP-Ang1, and as the 3′ non-translated region the 3′ UTR of von Willebrand Factor (vWF).

FIG. 28 shows the basic structure and nucleotide sequence of recombinant nucleic acid construct PAN36. PAN36 is an embodiment of the present invention comprising as the 5′ non-translated region the 5′ UTR of MCP-1, as the nucleic acid sequence coding for a signal peptide the nucleic acid sequence coding for a signal peptide of MCP-1, as the coding region coding for an effector molecule the coding region coding for Ang1, and as the 3′ non-translated region the 3′ UTR of MCP-1.

FIG. 67 shows recombinant nucleic acid construct PAN50 which is another embodiment of an mRNA of the present invention comprising as the 5′ non-translated region the 5′ UTR of MCP-1, as the nucleic acid sequence coding for a signal peptide the nucleic acid sequence coding for a signal peptide of IL-6, as the coding region coding for an effector molecule the coding region coding for Nano luciferase, and as the 3′ non-translated region the 3′ UTR of von Willebrand Factor (vWF).

FIG. 68 shows recombinant nucleic acid construct PAN51 which is another embodiment of an mRNA of the present invention comprising as the 5′ non-translated region the 5′ UTR of HSP70, as the nucleic acid sequence coding for a signal peptide the nucleic acid sequence coding for a signal peptide of IL-6, as the coding region coding for an effector molecule the coding region coding for Nano luciferase (NLuc), and as the 3′ non-translated region the 3′ UTR of von Willebrand Factor (vWF).

FIG. 69 shows recombinant nucleic acid construct PAN52 which is another embodiment of an mRNA of the present invention comprising as the 5′ non-translated region the 5′ UTR of MCP-1, as the nucleic acid sequence coding for a signal peptide the nucleic acid sequence coding for a signal peptide of MCP-1, as the coding region coding for an effector molecule the coding region coding for Ang1, and as the 3′ non-translated region the 3′ UTR of von Willebrand Factor (vWF).

FIG. 70 shows recombinant nucleic acid construct PAN53 which is another embodiment of an mRNA of the present invention comprising as the 5′ non-translated region the 5′ UTR of MCP-1, as the nucleic acid sequence coding for a signal peptide the nucleic acid sequence coding for a signal peptide of MCP-1, as the coding region coding for an effector molecule the coding region coding for hCOMP-Ang1, and as the 3′ non-translated region the 3′ UTR of von Willebrand Factor (vWF).

FIG. 71A is an image of a 1% non-denaturing agarose gel stained with EtBr showing the tail-PCR products of constructs PAN35 (“35”), PA34 (“34”), PAN33 (“33”), PAN32 (“32”), PAN31 (“31”), PAN30 (“30”), PAN29 (“29”) and PAN12 (“12”). M: is a lane with a high range RiboRuler RNA ladder. From this FIG. 71A may be taken that the transcribed mRNAs were intact and, more specifically, were not degraded.

FIG. 71B is an image of a 1% non-denaturing agarose gel stained with EtBr showing the in vitro mRNA transcripts of constructs PAN35 (“35”), PA34 (“34”), PAN33 (“33”), PAN32 (“32”), PAN31 (“31”), PAN30 (“30”), PAN29 (“29”) and PAN12 (“12”). M: is a lane with a high range RiboRuler RNA ladder. From this FIG. 71B may be taken that the transcribed mRNAs were intact and, more specifically, were not degraded.

FIG. 86 shows recombinant nucleic acid construct PAN54 which is another embodiment of an mRNA of the present invention comprising as the 5′ non-translated region the 5′ UTR of RPL12s.c. as the nucleic acid sequence coding for a signal peptide the nucleic acid sequence coding for a signal peptide of MCP-1, as the coding region coding for an effector molecule the coding region coding for Nano-Luciferase (NLuc), and as the 3′ non-translated region the 3′ UTR of von Willebrand Factor (vWF).

FIG. 87 shows recombinant nucleic acid construct PAN55 which is another embodiment of an mRNA of the present invention comprising as the 5′ non-translated region the 5′ UTR of vWF, as the nucleic acid sequence coding for a signal peptide the nucleic acid sequence coding for a signal peptide of MCP-1, as the coding region coding for an effector molecule the coding region coding for Nano Luciferase (NLuc), and as the 3′ non-translated region the 3′ UTR of von Willebrand Factor (vWF).

FIG. 88 shows recombinant nucleic acid construct PAN56 which is another embodiment of an mRNA of the present invention comprising as the 5′ non-translated region the 5′ UTR of vWF, as the nucleic acid sequence coding for a signal peptide the nucleic acid sequence coding for a signal peptide of vWF, as the coding region coding for an effector molecule the coding region coding for Nano Luciferase (NLuc), and as the 3′ non-translated region the 3′ UTR of von Willebrand Factor (vWF).

FIG. 89 shows recombinant nucleic acid construct PAN57 which is another embodiment of an mRNA of the present invention comprising as the 5′ non-translated region a synthetic nucleic acid sequence as described in Jiang, Lei et al. (2018). Systemic messenger RNA as an etiological treatment for acute intermittent porphyria. Nature Medicine. 24, 1899-2909 (2018), as the nucleic acid sequence coding for a signal peptide the nucleic acid sequence coding for a signal peptide of MCP-1, as the coding region coding for an effector molecule the coding region coding for Nano Luciferase (NLuc), and as the 3′ non-translated region the 3′ UTR a nucleic acid sequence as described in Jiang, Lei et al. (2018). Systemic messenger RNA as an etiological treatment for acute intermittent porphyria. Nature Medicine. 24; 1899-1909 (2018). The 5′-and 3′ sequences are shown underlined and in bold.

FIG. 90 shows recombinant nucleic acid construct PAN58 which is another embodiment of an mRNA of the present invention comprising as the 5′ non-translated region the 5′ UTR of MCP-1, as the nucleic acid sequence coding for a signal peptide the nucleic acid sequence coding for a signal peptide of MCP-1, as the coding region coding for an effector molecule the coding region coding for Nano Luciferase (NLuc), and as the 3′ non-translated region the 3′ UTR of von Willebrand Factor (vWF). In addition, PAN58 comprises a consensus Kozak sequence (GCCACC) in the six bases upstream of the start AUG.

FIG. 91 shows recombinant nucleic acid construct PAN59 which is another embodiment of an mRNA of the present invention comprising as the 5′ non-translated region the 5′ UTR of MCP-1, as the nucleic acid sequence coding for a signal peptide the nucleic acid sequence coding for a signal peptide of MCP-1, as the coding region coding for an effector molecule the coding region coding for CMP-Ang1, and as the 3′ non-translated region the 3′ UTR of von Willebrand Factor (vWF).

FIG. 95 shows recombinant nucleic acid construct PAN60 which is another embodiment of an mRNA of the present invention comprising as the 5′ non-translated region the 5′ UTR of MCP-1, as the coding region coding for an effector molecule the coding region coding for wild type (wt) Tie-2, and as the 3′ non-translated region the 3′ UTR of von Willebrand Factor (vWF).

FIG. 96 shows recombinant nucleic acid construct PAN61 which is another embodiment of an mRNA of the present invention comprising as the 5′ non-translated region the 5′ UTR of MCP-1, as the coding region coding for an effector molecule the coding region coding for Tie-2 mutant R849W, and as the 3′ non-translated region the 3′ UTR of von Willebrand Factor (vWF).

FIG. 97 shows recombinant nucleic acid construct PAN66 which is another embodiment of an mRNA of the present invention comprising as the 5′ non-translated region the 5′ UTR of MCP-1, as the coding region coding for an effector molecule the coding region coding for wild type PIK3CA which is, according to GenBank Homo sapiens phosphatidylinositol-4,5-bisphosphate 3-kinase catalytic subunit alpha (PIK3CA) with the respective reference number being 006218.4., and as the 3′ non-translated region the 3′ UTR of von Willebrand Factor (vWF).

FIG. 101 shows the basic structure and length of recombinant nucleic acid constructs PAN60, PAN61, PAN62, PAN63, PAN64 and PAN65.

Recombinant nucleic acid construct PAN60 comprises as the 5′ non-translated region the 5′ UTR of MCP-1, as the coding region coding for an effector molecule the coding region coding for wild type of Tie-2, and as the 3′ non-translated region the 3′ UTR of vWF.

Recombinant nucleic acid construct PAN61 comprises as the 5′ non-translated region the 5′ UTR of MCP-1, as the coding region coding for an effector molecule the coding region coding for mutant Tie-2 Tie-2 R849W, and as the 3′ non-translated region the 3′ UTR of vWF.

Recombinant nucleic acid construct PAN62 comprises as the 5′ non-translated region the 5′ UTR of MCP-1, as the coding region coding for an effector molecule the coding region coding for mutant Tie-2 Tie-2 L914R, and as the 3′ non-translated region the 3′ UTR of vWF.

Recombinant nucleic acid construct PAN63 comprises as the 5′ non-translated region the 5′ UTR of MCP-1, as the coding region coding for an effector molecule the coding region coding for mutant Tie-2 Tie-2 R849W L914F, and as the 3′ non-translated region the 3′ UTR of vWF.

Recombinant nucleic acid construct PAN64 comprises as the 5′ non-translated region the 5′ UTR of MCP-1, as the coding region coding for an effector molecule the coding region coding for mutant Tie-2 Tie-2 Y897F R915L, and as the 3′ non-translated region the 3′ UTR of vWF.

Recombinant nucleic acid construct PAN65 comprises as the 5′ non-translated region the 5′ UTR of MCP-1, as the coding region coding for an effector molecule the coding region coding for mutant Tie-2 Tie-2 Y897F R915L T1105N T1106P, and as the 3′ non-translated region the 3′ UTR of vWF.

FIG. 102 shows the basic structure and length of recombinant nucleic acid constructs PAN66, PAN67, PAN68, and PAN69.

Recombinant nucleic acid construct PAN66 comprises as the 5′ non-translated region the 5′ UTR of MCP-1, as the coding region coding for an effector molecule the coding region coding for wild type of PIK3CA, and as the 3′ non-translated region the 3′ UTR of vWF.

Recombinant nucleic acid construct PAN67 comprises as the 5′ non-translated region the 5′ UTR of MCP-1, as the coding region coding for an effector molecule the coding region coding for mutant PIK3CA PIK3CA E542K, and as the 3′ non-translated region the 3′ UTR of vWF.

Recombinant nucleic acid construct PAN68 comprises as the 5′ non-translated region the 5′ UTR of MCP-1, as the coding region coding for an effector molecule the coding region coding for mutant PIK3CA PIK3CA E545K, and as the 3′ non-translated region the 3′ UTR of vWF.

Recombinant nucleic acid construct PAN69 comprises as the 5′ non-translated region the 5′ UTR of MCP-1, as the coding region coding for an effector molecule the coding region coding for mutant PIK3CA PIK3CA H1047R, and as the 3′ non-translated region the 3′ UTR of vWF.

FIG. 103 shows the basic structure and length of recombinant nucleic acid construct PAN77. Recombinant nucleic acid construct PAN77 comprises as the 5′ non-translated region the 5′ UTR of MCP-1, as the coding region coding for an effector molecule the sequence coding for mutant Tie-2, wherein said mutant Tie-2 is a truncated form of Tie-2 which comprises an N-terminal myristylation signal (MGSSKSKPKDPSQR (SEQ ID NO: 161)) of Src kinase fused via a short GGH-linker (SEQ ID NO: 162) to the C-terminal intracellular (cytoplasmic) kinase domain of Tie-2 (amino acids 770-1124 of Tie-2; UniProtKB - Q02763; NCBI Refseq NP_000450.2) comprising the R849W_L914F double mutation, preferably the truncated form of Tie-2 comprises a nucleotide sequence of SEQ ID NO: 163, and as the 3′ non-translated region the 3′ UTR of vWF.

FIG. 104 shows the basic structure and length of recombinant nucleic acid construct PAN78. Recombinant nucleic acid construct PAN78 comprises as the 5′ non-translated region the 5′ UTR of MCP-1, as the coding region coding for an effector molecule the sequence coding for mutant Tie-2, wherein said mutant Tie-2 is a COMP-Tie-2 fusion in which an N-terminal coiled-coil (multimerization) domain of human COMP (amino acids 29-73 of cartilage oligomeric matrix protein; UniProtKB P49747 (NCBI Refseq: NP_000086.2; NM_000095.3)) is fused via a GGISGGGGGI-linker (SEQ ID NO: 164) to the C-terminal transmembrane and intracellular kinase domain of Tie-2 (amino acids 749-1124 of Tie-2; UniProtKB - Q02763; NCBI Refseq NP_000450.2) comprising the R849W_L914F double mutation, preferably the truncated form of Tie-2 comprises a nucleotide sequence of SEQ ID NO: 165, and as the 3′ non-translated region the 3′ UTR of vWF. The entire construct comprises 1579 nt.

FIG. 105 shows the basic structure, the length of recombinant nucleic acid and the nucleotide sequence of construct PAN70. Recombinant nucleic acid construct PAN70 comprises as the 5′ non-translated region the 5′ UTR of MCP-1, as the coding region coding for an effector molecule the coding sequence for myrP1 10*, and as the 3′ non-translated region the 3′ UTR of vWF.

FIG. 130 shows the basic structure and nucleotide sequence of recombinant nucleic acid construct PAN 82. Recombinant nucleic acid construct PAN82 comprises as the 5′ non-translated region the 5′ UTR of MCP-1, as the coding region coding for an effector molecule the sequence coding for myrTie-2 wt (SEQ ID NO: 166), i.e. the wildtype-homolog of PAN77 having the wildtype amino acid sequence in the cytoplasmic Tie-2 kinase domain, and as the 3′ non-translated region the 3′ UTR of vWF. The entire construct comprises 1333 nt.

FIG. 131 shows the basic structure and nucleotide sequence of recombinant nucleic acid construct PAN 83. Recombinant nucleic acid construct PAN83 comprises as the 5′ non-translated region the 5′ UTR of MCP-1, as the coding region coding for an effector molecule the sequence coding for myrTie-2 (Y897F R915L T1105N T1106) (SEQ ID NO: 167), i.e. a homolog of PAN77 but having the amino acid exchanges Y897F, R915L, T1105N, T1106 instead of the R849W_L914F exchange in the cytoplasmic Tie-2 kinase domain, and as the 3′ non-translated region the 3′ UTR of vWF. The entire construct comprises 1333 nt.

FIG. 132 shows the basic structure and nucleotide sequence of recombinant nucleic acid construct PAN 84. Recombinant nucleic acid construct PAN84 comprises as the 5′ non-translated region the 5′ UTR of MCP-1, as the coding region coding for an effector molecule the sequence coding for wild type COMP-Tie-2 (SEQ ID NO: 168), i.e. a wildtype-homolog of PAN78 having the wildtype amino acid sequence in the Tie-2 kinase domain, and as the 3′ non-translated region the 3′ UTR of vWF. The entire construct comprises 1579 nt.

FIG. 133 shows the basic structure and nucleotide sequence of recombinant nucleic acid construct PAN 85. Recombinant nucleic acid construct PAN85 comprises as the 5′ non-translated region the 5′ UTR of MCP-1, as the coding region coding for an effector molecule the sequence coding for a mutant COMP-Tie-2 (Y897F R915L T1105N T1106) (SEQ ID NO: 169), i.e. a homolog of PAN78 but having the amino acid exchanges Y897F, R915L, T1105N, T1106 instead of the R849W _L914F exchange in the cytoplasmic Tie-2 kinase domain, and as the 3′ non-translated region the 3′ UTR of vWF. The entire construct comprises 1579 nt.

FIG. 134 shows the basic structure and nucleotide sequence of recombinant nucleic acid construct PAN 86. Recombinant nucleic acid construct PAN86 comprises as the 5′ non-translated region the 5′ UTR of MCP-1, as the coding region coding for an effector molecule the sequence coding for Tie-2 TM_IC (R849W L914F) (SEQ ID NO: 170) which is the transmembrane and intracellular domain of Tie-2 (amino acids 749-1124 of human Tie-2; UniProtKB Q02763; NCBI Refseq NP_000450.2) carrying a R849W and L914F amino acid exchange, and as the 3′ non-translated region the 3′ UTR of vWF. The entire construct comprises 1579 nt.

FIG. 135 shows the basic structure and nucleotide sequence of recombinant nucleic acid construct PAN 87. Recombinant nucleic acid construct PAN87 comprises as the 5′ non-translated region the 5′ UTR of MCP-1, as the coding region coding for an effector molecule the sequence coding for myrTie-2 (T1105N T1106P) (SEQ ID NO: 171), i.e. a homolog of PAN77 having the amino acid exchanges T1105N T1106P instead of the R849W_L914F exchange in the cytoplasmic Tie-2 kinase domain, and as the 3′ non-translated region the 3′ UTR of vWF. The entire construct comprises 1333 nt.

FIG. 136 shows the basic structure and nucleotide sequence of recombinant nucleic acid construct PAN 88. Recombinant nucleic acid construct PAN88 comprises as the 5′ non-translated region the 5′ UTR of MCP-1, as the coding region coding for an effector molecule the sequence coding for a mutant COMP-Tie-2 (T1105N and T1106) (SEQ ID NO: 172), i.e. a homolog of PAN78 but having the amino acid exchanges T1105N and T1106 instead of the R849W_L914F exchange in the cytoplasmic Tie-2 kinase domain, and as the 3′ non-translated region the 3′ UTR of vWF. The entire construct comprises 1579 nt.

The SEQ ID NOs: of the sequence listing are related to the instant disclosure as summarized in Table 1.

TABLE 1

| Seq ID No | Subject to Fig | Further information |
|---|---|---|
| 1 | 7A | 5‘Primer |
| 2 | 7A | 5′Primer |
| 3 | 7A | 5′Primer |
| 4 | 7A | 5′Primer |
| 5 | 7A | 5′Primer |
| 6 | 7A | 5′Primer |
| 7 | 7A | 5′Primer |
| 8 | 7A | 5′Primer |
| 9 | 7A | 5′Primer |
| 10 | 7A | 5′Primer |
| 11 | 7B | 3 ‘Primer |
| 12 | 7B | 3′Primer |
| 13 | 7B | 3′Primer |
| 14 | 7B | 3′Primer |
| 15 | 7B | 3′Primer |
| 16 | 7B | 3′Primer |
| 17 | 7B | 3′Primer |
| 18 | 7B | 3′Primer |
| 19 | 7B | 3′Primer |
| 20 | 15A | 5 ‘UTR MCP 1 |
| 21 | 15B | SP (signal peptide) nucleotide sequence of MCP1 |
| 22 | 15B | SP amino acid sequence of MCP1 |
| 23 | 15C | 3′UTR of MCP1 |
| 24 | 16A | 5′UTR of RPL12 |
| 25 | 16B | 3′UTR of RPL12 |
| 26 | 17A | 5′UTR of Ang2 |
| 27 | 17B | SP (signal peptide) nucleotide sequence of Ang2 |
| 28 | 17B | SP (signal peptide) amino acid sequence of Ang2 |
| 29 | 17C | 3′UTR of Ang2 |
| 30 | 18A | 5′UTR ofIL6 |
| 31 | 18B | SP (signal peptide) nucleotide sequence of IL6 |
| 32 | 18B | SP (signal peptide) amino acid sequence of IL6 |
| 33 | 18C | 3′UTR of IL6 |
| 34 | 19A | 5′UTR of vWF |
| 35 | 19B | SP(signal peptide) nucleotide sequence of vWF |
| 36 | 19B | SP (signal peptide) amino aicd sequence of vWF |
| 37 | 19C | 3′UTR of vWF |
| 38 | 20A | 5′UTR of HSP70 A1 |
| 39 | 20B | 3′UTR of HSP70 A1 |
| 40 | 21A | 5′UTR of HSP70 A5 |
| 41 | 21B | SP (signal peptide) nucleotide sequence of HSP70 A5 |
| 42 | 21B | SP (signal peptide) amino acid sequence of HSP70 A5 |
| 43 | 21C | 3′UTR of HSP70 A5 |
| 44 | 22A | 5′UTR of H3.3 |
| 45 | 22B | 3′UTR of H3.3 |
| 46 | 23A | 5′UTR of LGALS9 |
| 47 | 23B | 3′UTR of LGALS9 |
| 48 | 24 | Construct PAN01 |
| 49 | 25 | Construct PAN03 |
| 50 | 26 | Construct PAN04 |
| 51 | 27 | Construct PAN06 |
| 52 | 28 | Construct PAN36 |
| 53 | 29 | Construct PAN37 |
| 54 | 30 | Construct PAN02 |
| 55 | 31 | Construct PAN05 |
| 56 | 32 | Construct PAN07 |
| 57 | 33 | Construct PAN08 |
| 58 | 34 | Construct PAN09 |
| 59 | 35 | Construct PAN10 |
| 60 | 36 | Construct PAN11 |
| 61 | 37 | Construct PAN12 |
| 62 | 38 | Construct PAN13 |
| 63 | 39 | Construct PAN28 |
| 64 | 40 | Construct PAN29 |
| 65 | 41 | Construct PAN30 |
| 66 | 42 | Construct PAN31 |
| 67 | 43 | Construct PAN32 |
| 68 | 44 | Construct PAN33 |
| 69 | 45 | Construct PAN34 |
| 70 | 46 | Construct PAN35 |
| 71 | 47 | Construct PAN38 |
| 72 | 48 | Construct PAN39 |
| 73 | 49 | Construct PAN40 |
| 74 | 50 | Construct PAN41 |
| 75 | 51 | Construct PAN42 |
| 76 | 52 | Construct PAN43 |
| 77 | 53 | Construct PAN44 |
| 78 | 54 | Construct PAN45 |
| 79 | 55 | Construct PAN46 |
| 80 | 56 | Construct PAN47 |
| 81 | 57 | Construct PAN48 |
| 82 | 58 | Construct PAN49 |
| 83 | 59A | 5′UTR of Ang1 |
| 84 | 59B | SP (signal peptide) nucleotide sequence of Ang1 |
| 85 | 59B | SP (signal peptide) amino acid sequence of Ang1 |
| 86 | 59C | CDS of Ang1(mature peptide+stop codon) |
| 87 | – | Amino acid sequence derived from SEQ ID No. 86 |
| 88 | 59D | 3′UTR of Ang1 |
| 89 | 60A | 5′UTR of Ang4 |
| 90 | 60B | SP (signal peptide) nucleotide sequence of Ang4 |
| 91 | 60B | SP (signal peptide) amino acid sequecne of Ang4 |
| 92 | 60C | CDS of Ang4 (mature peptide+stop codon) |
| 93 | – | Amino acid sequence derived from SEQ ID No.92 |
| 94 | 60D | 3′UTR of Ang4 |
| 95 | 61 | COMP-Ang1 CDS (mature pep+stop codon) (rat) |
| 96 | – | Amino acid sequence derived from SEQ ID No.95 |
| 97 | 62 | hCOMP-Ang1 CDS (mature pep+stop codon) (human) |
| 98 | – | Amino acid sequence derived from SEQ ID No.97 |
| 99 | 63 | CMP-Ang1 CDS (mat peptide+stop codon) |
| 100 | 64 | COMP-Ang2 CDS (mat peptide+stop codon) |
| 101 | 65 | Tie-2 CDS (start+mat pep+stop codon) |
| 102 | - | Amino acid sequence derived from SEQ ID No. 101 |
| 103 | 66 | Tie-2* CDS (R849W)(start+mat pep+stop codon) |
| 104 | - | Amino acid sequence derived from SEQ ID No. 103 |
| 105 | 67 | Construct PAN50 |
| 106 | 68 | Construct PAN51 |
| 107 | 69 | Construct PAN52 |
| 108 | 70 | Construct PAN53 |
| 109 | 86 | Construct PAN54 |
| 110 | 87 | Construct PAN55 |
| 111 | 88 | Construct PAN56 |
| 112 | 89 | Construct PAN57 (Mod) |
| 113 | 90 | Construct PAN58 |
| 114 | 91 | Construct PAN59 |
| 115 | 95 | Construct PAN60 (Tie-2 wt) |
| 116 | 96 | Construct PAN61 (Tie-2 R849W) |
| 117 | 97 | Construct PAN66 (PIK3CA wt) |
| 118 | | Construct CDS (start+mat pep+stop codon) PIK3CA |
| 119 | | CDS (mature peptide+stop cod) Nluc |
| 120 | 100 a | 5′UTR of (PAN57) |
| 121 | 100 b | 3′UTR of (PAN57) |
| 122 | 7B | 3′Primer for constructs PAN01-PAN03 |
| 123 | 100 c | 5′Primer for construct PAN57 |
| 124 | 100 d | 3′Primer for construct PAN57 |
| 125 | 100 e | 5′Primer for construct PAN55 (FIG. 87 ) and PAN56 (FIG. 88 ) |
| 126 | 114 | nucleotide sequence of human wild type Tie-2 |
| 127 | 114 | amino acid sequence of human wild type Tie-2 |
| 128 | 107 | PAN62 |
| 129 | 108 | PAN63 |
| 130 | 109 | PAN64 |
| 131 | 110 | PAN65 |
| 132 | 115 | nucleotide sequence of human wild type PIK3CA |
| 133 | 115 | amino acid sequence of human wild type PIK3CA |
| 134 | 111 | PAN67 |
| 135 | 112 | PAN68 |
| 136 | 113 | PAN69 |
| 137 | 116 | nucleotide sequence of human wild type Akt1 |
| 138 | 116 | amino acid sequence of human wild type Akt1 |
| 139 | 117 | nucleotide sequence of human wild type mTor |
| 140 | 117 | amino acid sequence of human wild type mTor |
| 141 | 118 | nucleotide sequence of human wild type GNA11 |
| 142 | 118 | amino acid sequence of human wild type GNA11 |
| 143 | 119 | nucleotide sequence of human wild type GNAQ |
| 144 | 119 | amino acid sequence of human wild type GNAQ |
| 145 | 120 | nucleotide sequence of human wild type KRAS |
| 146 | 120 | amino acid sequence of human wild type KRAS |
| 147 | 121 | nucleotide sequence of human wild type NRAS |
| 148 | 121 | amino acid sequence of human wild type NRAS |
| 149 | 122 | nucleotide sequence of human wild type IDH1 |
| 150 | 122 | amino acid sequence of human wild type IDH1 |
| 151 | 105 | nucleotide sequence of PAN70 |
| 152 | 103 | nucleotide sequence of PAN77 |
| 153 | 104 | nucleotide sequence of PAN78 |
| 154 | 130 | nucleotide sequence of PAN82 |
| 155 | 131 | nucleotide sequence of PAN83 |
| 156 | 132 | nucleotide sequence of PAN84 |
| 157 | 133 | nucleotide sequence of PAN85 |
| 158 | 134 | nucleotide sequence of PAN86 |
| 159 | 135 | nucleotide sequence of PAN87 |
| 160 | 136 | nucleotide sequence of PAN88 |
| 161 | 137 | nucleotide sequence formyristylation signal of Scr-kinase |
| 162 | 138 | nucleotide sequence for a GGH linker |
| 163 | 139 | nucleotide sequence of effector molecule in PAN77 |
| 164 | 140 | nucleotide sequence of GGISGGGGGI-linker |
| 165 | 141 | nucleotide sequence of effector molecule in PAN78 |
| 166 | 142 | nucleotide sequence of effector molecule in PAN82 |
| 167 | 143 | nucleotide sequence of effector molecule in PAN83 |
| 168 | 144 | nucleotide sequence of effector molecule in PAN84 |
| 169 | 145 | nucleotide sequence of effector molecule in PAN85 |
| 170 | 146 | nucleotide sequence of effector molecule in PAN86 |
| 171 | 147 | nucleotide sequence of effector molecule in PAN87 |
| 172 | 148 | nucleotide sequence of effector molecule in PAN88 |

### Example 1: Materials and Methods

### Plasmid Template Generation by Gene Synthesis and Cloning

Sequences encoding Nluc reporter protein, Ang-1 protein and derivatives thereof (e.g. COMP-Ang-1, CMP-Ang-1) with different signal peptides were flanked by different heterologous 5′ and 3′ UTRs and were designed and produced by gene synthesis by BioCat (Heidelberg) and cloned (Xhol-BamHI) into pcDNA3.1- (Thermo Fisher).

Sequences encoding wt Tie-2 and sequences with the specified Tie-2 mutation in the coding region flanked by different heterologous 5′ and 3′ UTRs were designed (see FIGS. 95 and 96) and produced by gene synthesis by BioCat (Heidelberg) and cloned (Xhol-BamHI) into pcDNA3.1- (Thermo Fisher).

### In Vitro Transcription From PCR Products to Generate Polyadenylated mRNAs

In vitro transcription is the synthesis of RNA transcripts by RNA polymerase from a linear DNA template containing the corresponding promoter sequence (T7, T3, SP6) and the gene to be transcribed. A typical transcription reaction consists of the template DNA, RNA polymerase, ribonucleotide triphosphates, RNase inhibitor and buffer containing Mg2⁺ ions. Linearized plasmid DNA, PCR products and synthetic DNA oligonucleotides can be used as templates for transcription as long as they have the T7 promoter sequence upstream of the gene to be transcribed. In order to generate DNA templates with a poly-(A) tail a tail-PCR using 5′ primers containing T7 RNA polymerase sequences and 3′ primers with a 120 nt Poly-T sequence were synthesized and purified (BioSpring, Frankfurt).

The Linear DNA templates for in vitro transcription were generated by PCR from linearized plasmid. Plasmids were digested with BamHI that cut once 3-terminal in the vector backbone to produce a linearized vector that can be used as the template for the poly-(A) tail PCR. 5 µg plasmid are digested for 2 h at 37° C. with 5U BamHI restriction enzyme in 50 µl and 1x HF Buffer. Small Aliquots of digested mix were analyzed by gel electrophoresis to check for complete digestion of the plasmid (see, for example, FIG. 5 for constructs PAN28 (“28”), PAN13 (“13”), PAN12 (“12”), PAN11 (“11”), PAN10 (“10”), PAN09 (“09”), PAN08 (“08”), PAN07 (“07”) and PAN05 (“05”)). The restriction enzyme is heat-inactivated by incubating at 80° C. for 20 min and the digested plasmid is purified by a PCR purification column following the manufacturer’s protocol (NucleoSpin Gel and PCR clean-up, Macherey-Nagel). The linearized plasmid can be stored at -20° C. for several months and can be used for PCR template generation. The purpose of linearization was to eliminate circular templates that could potentially generate run-on transcripts during the IVT reaction.

Addition of poly-(A) tail by PCR was performed by using Hot Start HiFidelity polymerase (Qiagen) or Taq DNA Polymerase (Roche) following the manufacturer’s protocol. For this purpose, Adapter primers containing T7 promoter sequences and 3′ Poly A tail sequences (120nt) were used to amplify linear DNA-templates. Each of the adapter primers had overlap sequences which fused regulatory sequences necessary for translation and transcription to the gene of interest (see, for example, FIGS. 7a and 7b). A typical 50 µl PCR reaction contained final concentrations of 200 µM (of each) dNTP, 0.5 µM for each primer, 50 ng linearized plasmid DNA, 1x PCR reaction buffer (1.5 mM MgC12) and 1.25 U Taq DNA Polymerase per reaction (0.25 µl of 5U/µl). The specific PCR cycle programs or thermal profiles were adjusted according to the Tm of the primer pairs, according to the expected length of the PCR product and according to the used thermal cycler. The quality of the PCR products was checked by analyzing an aliquot by gel electrophoresis and the reactions are purified by commercial PCR purification kits ((NucleoSpin Gel and PCR clean-up, Macherey-Nagel).

### In Vitro Transcription Reaction

Addition of a 5′ end cap structure to the RNA is an important process in eukaryotes. It is essential for RNA stability, efficient translation, nuclear transport and splicing. The process involved addition of a 7-methylguanosine cap at the 5′ triphosphate end of the RNA. RNA capping can be carried out post-transcriptionally using capping enzymes or co-transcriptionally using cap analogs such as ARCA (Jena Bioscience) or CleanCap (Trilink Biotechnologies) or EZ-Cap (ApexBio). In the enzymatic method, the mRNA may be capped using the vaccinia virus mRNA capping enzyme (NEB) as per manufacturer’s protocol. The enzyme adds on a 7-methylguanosine cap at the 5′ end of the RNA using GTP and S-adenosyl methionine as donors (cap-0 structure). Both methods yield functionally active capped RNA suitable for transfection or other applications. In addition, a 2′-O-Methyltransferase can be used to introduce cap-1 and cap-2 structures.

In the example described below, the T7 High Yield Transcription Kit (Thermo Scientific) was used to synthesize capped RNA transcripts (PAN 01-88) using cap analogs co-transriptionally.

The DNA template for the transcription reaction was a linearized plasmid or a PCR product. For the capped RNA the reaction at room temperature was set up in the following order:

| Component | Volume (µl) | Final |
|---|---|---|
| Nuclease free water* | To 20 | |
| 5X Reaction Buffer | 4 | 1X |
| ATP (100 mM) | 2 | 10 mM |
| CTP (100 mM) or 5′-Methylcytidine-5′-Triphophate (100 mM) | 2 | 10 mM |
| UTP (100 mM) or | | |
| Pseudouridine-5′-Triphosphate (100 mM) or 5-Methoxyuridine-5′-Triphosphate (100 mM) N1-Methyl-pseudouridine (100 mM) | 2 | 10 mM |
| GTP (30 mM) | 2 | 3 mM |
| 3′-0-Me-m⁷G(5′)ppp(5′)Gcap analog (ARCA;Anti Reverse Cap Analog) (100 mM) | 2 | 10 mM |
| Template DNA | X | 1 µg |
| T7 RNA Polymerase Mix | 2 | |
| Total | 20 | |

1 µg DNA was added and the total reaction volume to 20 µl was obtained by adding nuclease free water. The amount of water to be added varied based on the concentration of the template DNA. The reactions at were well mixed by vortexing and incubated at 37° C. for 2 hours in a dry air incubator. The transcription reactions were treated with DNase I to remove the DNA template before proceeding with purification as follows:
1. 70 µl nuclease free water was added to the transcription reactions followed by 10 µl of DNase I reaction buffer.
2. 2 µl DNase I were added to the reactions.
3. The reaction was incubated at 37° C. for 15 minutes.

Column Purification of Capped mRNA
1. The capped RNA was purified using the MEGAclearKit (ThermoFisher, Waltham, MA, USA) as per the manufacturer’s instructions (comparable RNA purification kits from other vendors may be used).
2. The RNA was quantified using a NanoDrop Spectrophotometer.
3. As per the manufacturer’s instructions, RNA sample quality was assessed using the Agilent RNA 6000 Nano Kit and Agilent 2100 Bioanalyzer or by native 1% agarose gel electrophoresis and ethidium bromide staining (see, FIG. 8).

### mRNA Transfection of HeLa Cells, HEK293 Cells, HPMEC Cells, HPAEC Cells and HUVEC Cells

HeLa cells, were grown in DMEM complete medium. The human embryonic kidney 293 cell line (HEK293) is grown in EMEM (EBSS) + 2 mM Glutamine + 1% Non Essential Amino Acids (NEAA) + 10% FCS culture medium.

The human embryonic kidney 293 cell line (HEK293) is grown in EMEM (EBSS) + 2 mM Glutamine + 1% Non Essential Amino Acids (NEAA) + 10% FCS culture medium. Subculture Routine is to split sub-confluent cultures (70-80%) 1:2 to 1:6 i.e. seeding at 2-5×10,000 cells/cm² using 0.25% trypsin or trypsin/EDTA; 5% CO₂; 37° C. mRNA transfection are performed with the Lipofectamine™ MessengerMAX™ Transfection Reagent (Invitrogen™) according to the manufactures protocol.

Primary Human Pulmonary Microvascular Endothelial Cells (HPMEC) and Primary Human Pulmonary Artery Endothelial Cells (HPAEC) are isolated from human pulmonary arteries are most appropriate for studying human lung diseases and are isolated from the lung from a single donor. Since lung tissue contains blood and lymphatic capillaries, HPMEC comprise Blood and Lymphatic Microvascular Endothelial Cells.

The cell type of HPMEC’s are characterized by immunofluorescent staining. They stain positive for CD31 and von Willebrand factor and negative for smooth muscle alpha-actin.

HUVEC, HPAEC and HPMEC cells were grown in Endothelial Cell Growth Medium as recommended from PromoCell (Heidelberg, Germany) respectively. The Endothelial Cell Growth Medium is basal Medium supplemented with Fetal Calf Serum (0.05 ml/ml), Endothelial Cell Growth Supplement (bovine hypothalamic extract; 0.004 ml ml), Heparin 90 µg/ml and Hydrocortisone 1 µg/ml. The experiments with HUVEC, HPAEC and HPMEC cells are performed on cells with passage numbers below 8.

RNA transfections and more mRNA transfections were carried out using Lipofectamin MessengerMAX (Invitrogen, Carlsbad, CA; Thermo Fisher Scientific).. For mRNA transfection in 24-well plate format 1×10⁵ cells/well were seeded 24 h before transfection to reach 70-90 confluence immediately before transfection. For one single well 1.5 µl Lipofectamine MessengerMAX Reagent was added to 25 µl OptMEM (Invitrogen, Carlsbad, CA; Thermo Fisher Scientific), vortexed and incubated for 10 min at RT. In a second well 500 ng or 1 µg mRNA were added to 25 µl OptiMEM, and vortexed. After incubation diluted mRNA was added to diluted Lipofectamin, mixed well, incubated for 5 min at RT and added dropwise to cells in the presence of 0.5 ml of complete medium for Hela and HEK293. For HUVEC, HPAEC and HPMEC the Endothelial Cell Growth medium was replaced and the cells washed with OptiMEM and then the Lipofectamin MessengerMAxX mRNA complex solution added to the well with 0.5 ml OptiMEM prewarmed to 37° C. . Cells were incubated at 37° C. for 2h and the OptiMEM medium replaced with complete Endothelial Cell Growth Medium (PromoCell, Heidelberg Germany).

Cell Lysates from Transfected cells or serum were analyzed at the indicated time points. Alternatively, mRNA-LNPs based on the cationic lipids L-Arginyl-β-alanine-N-palmityl-N-oleyl-amide or β-(L-Arginyl)-L-2,3-diamino propionic acid-N-palmityl-N-oleyl-amide in combination with neutral and PEGylated co-lipids were used for in vitro transfection. The co-lipids were Diphytanoyl-PE and the PEGylated lipid is methoxyPEG2000-DSPE

Successful introduction of mRNA into host cells was monitored using various known methods, such as a fluorescent marker, such as Green Fluorescent Protein (GFP), such as reporter enzymes (e.g. luciferase derivatives). Alternatively, transfection of a modified mRNA could also be determined by measuring the protein expression level of the target polypeptide by e.g., Western Blotting or immunocytochemistry or ELISA.

Fully modified mRNAs with 5-methylcytidine and/or pseudouridine (or 5-methoxyuridine or N1-methylpseudouridine) are transfected into HUVEC or HPMEC (Human Pulmonary Microvascular Endothelial Cells, PromoCell, Heidelberg) using Lipofectamin MessengerMAX (Invitrogen, Carlsbad, CA; Thermo Fisher Scientific). The cell lysates are harvested and run by ELISA (or on immunoblot assays (western) at different time points (30″, 1 h, 2 h, 4 h, 6 h hours) after transfection to determine the protein expression.

### Nano-Luc Luciferase Activity Reporter Measurement

A time dependent quantitative detection of Nano-Glo-Luciferase expression (Promega) was performed for secreted versions by analyzing samples from the tissue culture supernatant according to the reporter assay technical manual Nano-Glo Luciferase Assay System (Promega). In brief, 5-20 µl of serum or lysate were diluted in 100 µl H₂O final volume and equal volume of reconstituted Nano-Glo luciferase assay reagent was combined in a 96 well GloMax 96 Microplate. After at least 3 min incubation at RT luminescence were measured in an appropriate luminometer.

### Immunoblot Detection of Tie-2 and Derivates

For immunoblot detection of Tie-2 and derivatives protein lysates are loaded on NuPage SDS-PAGE system (chambers and power supply) with 1.5 mm ready-to-use Bis-Tris gels and 4-12% acrylamide gradient with MOPS-buffer as running aid (all Life Technologies, Grand Island, NY). Each lysate sample is prepared to 40 µl final volume. This sample contains 25 µg protein lysate in variable volume, RIPA buffer to make up volume to 26 µl, 4 µl of 10x reducing agent and 10 µl 4x SDS loading buffer (both from Life Technologies, Grand Island, NY). Samples are heated at 95° C. for 5 min and loaded on the gel. Standard settings are chosen by the manufacturer, 200 V, 120 mA and max. 25 W. Run time is 60 min, but no longer than running dye reaching the lower end of the gel.

After the run is terminated, the plastic case is cracked and the encased gel transferred to a ready-to-use nitrocellulose membrane kit and power supply (iBLOT; LifeTechnologies, Grand Island, NY). Using default settings, the protein lysate is transferred by high Ampere electricity from the gel to the membrane.

After the transfer, the membranes are incubated in 5% BSA in IX TBS for 15 minutes then in 5% BSA in IX TBS + 0.1 % Tween for another 15 minutes. Primary antibodies against human Tie-2 proteins or Phospho-specific Antibodies (P-Tie-2, P-Akt, see below) are applied in 3 ml of 5% BSA in IX TBS solution at a 1:500 to 1:2000 dilution for 3 hours at room temperature and gentle agitation on an orbital shaker. Membranes are washed 3 times with IX TBS/0.1 % Tween, 5 minutes each time with gentle agitation. The secondary antibody (Goat anti-rabbit HRP conjugate; Abeam, Cambridge, MA) is conjugated to horse radish peroxidase and binds to the primary antibody antibodies. The secondary antibody is diluted from 1:1000 to 1:5000 in 5% BSA in IX TBS and incubated for 3 hrs at RT. At the end of incubation time, the membranes are washed 3 times with IX TBS/0.1% Tween, 5 minutes each time with gentle agitation. The membranes are developed in 5 ml Pierce WestPico Chemiluminescent Substrate (Thermo Fisher, Rockford, IL) as directed.

### Example 2: Expression of Luciferase, Ang-1 or Ang-1-Derivatives Expressing Recombinant Nucleic Acid Constructs in HPMEC, HUVEC, HPAEC and HeLa Cells

### mRNA Transfection of HeLa, HUVEC, HPAEC and HPMEC Cells

Primary Human Pulmonary Microvascular Endothelial Cells (HPMEC) are most appropriate for studying human lung diseases and are isolated from the lung from a single donor. Since lung tissue contains blood and lymphatic capillaries, HPMEC comprise Blood and Lymphatic Microvascular Endothelial Cells. The cells were routinely analyzed by immunofluorescent staining: they stain positive for CD31 and von Willebrand factor and negative for smooth muscle alpha-actin. HeLa cells, HUVEC, HPMEC and HPAEC (human pulmonary artery endothelial cells) cells, respectively, were grown in DMEM complete medium or Endothelial Cell Growth Medium (PromoCell, Heidelberg Germany, CatNo.: C-22020) respectively. The Endothelial Cell Growth Medium was basal Medium supplemented with Fetal Calf Serum (0.05 ml/ml), Endothelial Cell Growth Supplement (bovine hypothalamic extract; 0.004 ml ml), Heparin 90 µg/ml and Hydrocortisone 1pg/ml. The experiments with HUVEC and HPMEC cells were performed on cells with passage numbers below 8.

mRNA transfections were carried out using Lipofectamin MessengerMAX (Invitrogen, Carlsbad, CA; Thermo Fisher Scientific). For mRNA transfection in 24-wellplate format 1x10⁵ cells/well were seeded 24h before transfection to reach 70-90 confluence immediate before transfection. For one single well 1.5 µl Lipofectamine MessengerMAX Reagent was added to 25 µl OptiMEM, vortex and incubated for 10 min at RT. In a second well add 500 ng or 1 µg mRNA were added to 25 µl OptiMEM, vortex. After incubation diluted mRNA was added to diluted Lipofectamin, mixed well, incubated for 5 min at RT and added dropwise to cells in the presence of 0.5 ml of DMEM complete medium for HeLa. For HUVEC, HPMEC and HPAEC the Endothelial Cell Growth medium was replaced and the cells washed with OptiMEM and then added the Lipofectamin MessengerMAX mRNA complex solution to the well with 0.5 ml OptiMEM prewarmed to 37° C. Cells were incubated at 37° C. for 2 h and the OptiMEM medium was replaced with complete Endothelial Cell Growth Medium (PromoCell, Heidelberg Germany) and transfected cells or serum were analyzed at the indicated time points. Alternatively, mRNA-LNPs based on the cationic lipids L-Arginyl-β-alanine-N-palmityl-N-oleyl-amide or β-(L-Arginyl)-L-2,3-diamino propionic acid-N-palmityl-N-oleyl-amide in combination with neutral and PEGylated co-lipids can be used for in vitro transfection.

Successful introduction of mRNA into host cells can be monitored using various known methods, such as a fluorescent marker, such as Green Fluorescent Protein (GFP), such as reporter enzymes (e.g. luciferase derivatives) or transfection of a modified mRNA can also be determined by measuring the protein expression level of the target polypeptide by e.g., Western Blotting or immunocytochemistry or ELISA.

### Nano-Luc Luciferase Activity Reporter Measurement

A time dependent quantitative detection of Nano-Glo-Luciferase expression (Promega) was performed for secreted versions by analyzing samples from the tissue culture supernatant according to the reporter assay technical manual Nano-Glo Luciferase Assay System (Promega). In brief, 5-20 µl of serum or lysate were diluted in 100 µl H₂O final volume and equal volume of reconstituted Nano-Glo luciferase assay reagent was combined in a 96 well GloMax 96 Microplate. After at least 3 min incubation at RT luminescence could be measured in an appropriate luminometer.

### Detection of Ang-1, COM-Ang-1, CMP-Ang-1 Protein in Cell Lysates and Supernatant

A time dependent quantitative detection of secreted Ang-1 and secreted COMP-Ang-1 or COMP-Ang-2 expression is performed by analyzing samples from the supernatant tissue culture by ELISA (Human Angiopoietin-1 Quantikine ELISA Kit (R&D systems, Minneapolis, MN, USA) or by Western blot according to the technical manual.

500 ng of COMP-Ang-1 (mRNA sequence shown in SEQ ID NO: 108) with poly A tail of approximately 120 nucleotides not shown in sequence; 5 ‘cap,) fully modified with 5-methylcytidine and pseudouridine (COMP-Ang-1, 5 mC/pU), fully modified with 5-methylcytidine and Nl-methyl-pseudouridine (COMP-Ang-1,5mC/NlmpU) or unmodified (COMP-Ang-, unmod) is transfected into HUVEC or HPMEC (Human Pulmonary Microvascular Endothelial Cells, PromoCell, Heidelberg) using Lipofectamin MessengerMAX (Invitrogen, Carlsbad, CA; Thermo Fisher Scientific). The supernatant is harvested and run by ELISA 4 hours after transfection to determine the protein expression and cytokine induction.

For immunoblot detection of Ang-1 and derivatives protein lysates were loaded on NuPage SDS-PAGE system (chambers and power supply) with 1.5 mm ready-to-use Bis-Tris gels and 4-12% acrylamide gradient with MOPS-buffer as running aid (all Life Technologies, Grand Island, NY). Each lysate sample was prepared to 40 ul final volume. This sample contained 25 ug protein lysate in variable volume, RIPA buffer to make up volume to 26 ul, 4 ul of 10x reducing agent and 10 µl 4x SDS loading buffer (both from Life Technologies, Grand Island, NY). Samples were heated at 95oC for 5 min and loaded on the gel. Standard settings were chosen by the manufacturer, 200 V, 120 mA and max. 25 W. Run time was 60 min, but no longer than running dye reaching the lower end of the gel.

After the run is terminated, the plastic case is cracked and the encased gel transferred to a ready-to-use nitrocellulose membrane kit and power supply (iBLOT; LifeTechnologies, Grand Island, NY). Using default settings, the protein lysate is transferred by high Ampere electricity from the gel to the membrane.

After the transfer, the membranes were incubated in 5% BSA in IX TBS for 15 minutes then in 5% BSA in IX TBS + 0.1% Tween for another 15 minutes. Primary antibodies (Ang-1 ab183701 Abcam, Cambridge, UK) against human Ang-1 proteins are applied in 3 ml of 5% BSA in IX TBS solution at a 1:5000 dilution overnight at 4° C. and gentle agitation on an orbital shaker. Membranes are washed 3 times with 1X TBS/0.1% Tween, 5 minutes each time with gentle agitation. The secondary antibody (Goat anti-rabbit HRP conjugate; Abeam, Cambridge, MA) is conjugated to horse radish peroxidase and binds to the primary antibody antibodies. The secondary antibody is diluted of 1:10000 in 5% BSA in IX TBS and incubated for 1 hr at RT. At the end of incubation time, the membranes are washed 3 times with IX TBS/0.1% Tween, 5 minutes each time with gentle agitation. The membranes are developed in 5 ml Pierce WestPico Chemiluminescent Subtrate (Thermo Fisher, Rockford, IL) as directed. The Western Blot detects protein around the expected size of 70 kd for human Ang-1, and of 37 kDa for COMP-Ang-1 and CMP-Ang1. For reference, recombinant human Ang-1 protein was purchased from R&D Systems (biotechne).

### Results

Protein expression data presented in the FIGS. 10 to 14, 72 to 85 and 92 to 94 underlines that combinations of different regulatory nucleotide sequences flanking a coding region can significantly change the protein expression levels. Such change is further impacted by modification of the nucleotides forming the constructs as exemplified by replacing 100% of uridine and 100% cytidine nucleotides by pseudo-uridine and 5-methyl-cytidine (the constructs marked with “*” in FIG. 74 and FIGS. 77 to 85. Changes are not only observed in a particular cell type but also across different cell types. For example, PAN02 (wild-type Ang-1 mRNA) shows the lowest Luciferase activity in all cell lines (FIGS. 10 to 14). All other constructs show enhanced protein expressions throughout all cell types. Therefore, the approach of using regulatory sequences from different genes can increase protein expression activity. To this end, replacing the endogenous Ang-1 sequences have shown superior effects on the protein expression of the reporter.

Worth noting, even the same regulatory sequences showed distinct protein expression activity in different cell types (FIGS. 75 to 81. These data further support our approach that the use of specific regulatory sequences can influence the activity of protein expression in a context-specific, i.e. cell-type specific cellular environment.

Furthermore, we have shown that our data are also concerning a different aspect i.e. secretion. For example, the regulatory sequences used in construct PAN12 show cell-type specific (HPMEC) expression in whole cell lysates but also in medium (supernatant) (FIGS. 10-14). This indicated that these sequences are not only optimal for protein expression (translation) but also secretion. In contrast, construct PAN28 shows expression in all cell types more or less equally. The regulatory sequences used in construct PAN29 (and PAN58), namely the 5′-UTR and the signal peptide sequence of MCP-1 in combination with the 3′-UTR of vWF, were found to be particularly useful for highly efficient protein expression in human (microvascular) pulmonary endothelial cells (see e.g. FIGS. 72 and 75). The regulatory sequences used in construct PAN54, namely the 5′-UTR of spinach chloroplast RPL12, the signal peptide sequence of MCP-1 and the 3′-UTR of vWF, were found to be equally efficient for protein expression and secretion as shown in FIG. 92. As shown in FIG. 98 the regulatory sequences of PAN29 are also functional using three different ORFs, namely wt Ang-1 in PAN52; hCOMP-Ang-1 in PAN53 and CMP-Ang-1 in PAN59 in directing expression in primary human pulmonary microendothelial cells (HPMEC). In contrast the mRNA construct containing the endogenous 5′-and 3′-UTRs of human Ang-1 (PAN01) is not translated (FIG. 98). FIG. 99 shows that all four mRNA constructs display comparable quality (integrity and purity).

### Example 3: Tie-2 Pathway Activation After Transfection of Modified mRNAs Encoding the Wildtype Tie-2 (PAN60) or the Activating Tie-2 Mutations (e.g. R849W (PAN61))

Modified mRNAs containing heterologous 5′ and 3′ UTRs are generated by in vitro transcription of PCR templates as described previously in example 1 above. Different concentration of this mRNA encoding the activating Tie-2 mutations (e.g. R849W) are transfected in different cell lines (HeLa, primary endothelial cells such as HUVECs or HPMEC or HPAEC) next to mRNAs encoding the wild-type Tie-2 using Lipofectamin MessengerMAX (Invitrogen, Carlsbad, CA; Thermo Fisher Scientific.). For qualitative Western blot cell lysates at different time points post transfection of mRNAs (1-24 h) are probed with anti-phosphotyrosine antibody (R&D systems) to evaluate Tie-2 phosphorylation (pTyr, 140 kD,). Blots are stripped and re-probed with anti-Tie-2 antibody (R&D systems) to detect total Tie-2. For quantitative measurement two different ELISAs are performed measuring human Tie-2 or Phospho-Tie-2 using the human Tie-2 DuoSet ELISA kit or human-Phospho-Tie-2 DuoSet IC ELISA according to the manufactures (e.g. both ELISA-Kits from R&D systems) protocol.

Transfection of Tie-2 mutation (PAN61, R849W) encoding mRNAs increase the presence of Phospho-Tie-2 in comparison to cell lysates derived from cells transfected with mRNAs encoding wildtype Tie-2 mRNA or in comparison to non-transfected cells. This increase in Tie-2 phosphorylation is observed in the presence or absence of co-stimulation with recombinant human Ang-1 ligand and can be observed in different cell lines. To confirm the activation of the Tie-2 signaling pathway and to demonstrate a biological relevant functional downstream signal transduction by expressing the Tie-2 (R849W) derivate a phosphorylation of Akt is demonstrated in a time course experiment. These experiments indicate that mRNAs encoding for the Tie-2 activating mutation (R849W) are functional and stronger in activating the Tie-2 signaling pathway than wt Tie-2 encoding mRNAs. These hyperactivation of the pathway can be demonstrated in the absence or presence of Tie-2 ligands (e.g. Ang-1) in human endothelial and/or non-endothelial cells.

### Example 4: mRNA Formulation in Cationic LNPs for in Vivo Applications By Intravenous Administration

For in vivo experiments mRNA-LNPs are prepared in a formulation process with β-(L-Arginyl)- L-2,3-diamino propionic acid-N-palmityl-N-oleyl-amide as cationic lipid. Alternatively, the cationic lipid L-Arginyl-β-alanine-N-palmityl-N-oleyl-amide can be used in an identical procedure to prepare mRNA-LNPs.

mRNA-LNP formulations are prepared using a modified procedure of a method described for siRNA (Chen, S., Tam, Y.Y., Lin, P.J., Sung, M.M., Tam, Y.K., and Cullis, P.R. (2016), Influence of particle size on the in vivo potency of lipid nanoparticle formulations of siRNA. J. Control. Release 235, 236-244. & (ii) patent application US20170121712).

Briefly, lipids are dissolved in ethanol at appropriate molar ratios (e.g. 50:49:1 β-(L-arginyl)-L-2,3-diamino propionic acid-N-palmityl-N-oleyl-amide: DPyPE: mPEG2000-DSPE). The lipid mixture is combined with an isotonic Sucrose solution of mRNA at a volume ratio of 2:1 (aqueous:ethanol) using a microfluidic mixer (NanoAssemblr®; Precision Nanosystems, Vancouver, BC) and flow rates of 18 ml/min. Similarly, LNP formulations can be obtained using citrate or acetate buffered mRNA solutions (pH 3-4) and a slightly differing mixing ratio of 3:1 (v/v; aqueous:ethanol) and flow rates of 12 ml/min.

After the mixing process, the formulations are dialyzed against 10 mM HEPES or TRIS buffered isotonic Sucrose solution using 3.5 K MWCO Slide-A-Lyzer Dialysis Cassettes (Thermo Fisher Scientific) for at least 18 hours at 4° C. Instead of Sucrose, other sugars like Trehalose or Glucose can be equally used within the formulation process.

Subsequently, the formulations are tested for particle size (Zetasizer Nano ZS instrument (Malvern Instruments Ltd, Malvern, UK), RNA encapsulation (Quant-iT RiboGreen RNA Assay Kit following manufacturer’s (Thermo Fisher Scientific) protocol), and endotoxin and are found to be between 30 to 100 nm in size with a Zeta-potential of >25 mV, display greater than 90% mRNA encapsulation and < 1 EU/ml of endotoxin.

mRNA-LNP formulations are stored at -80° C. at a concentration of RNA of ~ 0.3 µg/µl and an RNA to total lipid ratio of ~ 0.03-0.05 (wt/wt) until further in vitro or in vivo use.

### Example 5: Material and Methods - 2

### Plasmid Template Generation by Gene Synthesis and Cloning

Sequences encoding wt Tie-2 and sequences with the specified Tie-2 mutations in the coding region flanked by different heterologous 5′ and 3′ UTRs were designed and produced by gene synthesis by BioCat (Heidelberg) and cloned (Xhol—BamHI) into pcDNA3.1-(Thermo Fisher).

### In Vitro Transcription From PCR Products to Generate Polyadenylated mRNAs

In vitro transcription is the synthesis of RNA transcripts by RNA polymerase from a linear DNA template containing the corresponding promoter sequence (T7, T3, SP6) and the gene to be transcribed. A typical transcription reaction consists of the template DNA, RNA polymerase, ribonucleotide triphosphates, RNase inhibitor and buffer containing Mg2+ ions. Linearized plasmid DNA, PCR products and synthetic DNA oligonucleotides can be used as templates for transcription as long as they have the T7 promoter sequence upstream of the gene to be transcribed. In order to generate DNA templates with a poly-(A) tail a tail-PCR using 5′ primers containing T7 RNA polymerase sequences and 3′ primers with a 120 nt Poly-T sequence are synthesized and purified (BioSpring, Frankfurt).

The Linear DNA templates for in vitro transcription are generated by PCR from linearized plasmid. Plasmids are digested with BamHI that cut once 3-terminal in the vector backbone to produce a linearized vector that can be used as the template for the poly-(A) tail PCR. 5 µg plasmid are digested for 2 h at 37oC with 5U BamHI restriction enzyme in 50µl and 1x HF Buffer. Small Aliquots of digested mix are analyzed by gel electrophoresis to check for complete digestion of the plasmid. The restriction enzyme is heat-inactivated by incubating at 80oC for 20 min and the digested plasmid is purified by a PCR purification column following the manufacturer’s protocol (NucleoSpin Gel and PCR clean-up, Macherey-Nagel). The linearized plasmid can be stored at -20OC for several months and can be used for PCR template generation. The purpose of linearization is to eliminate circular templates that could potentially generate run-on transcripts during the IVT reaction.

Addition of poly-(A) tail by PCR is performed by using Hot StarHiFidelity polymerase (Qiagen) or Taq DNA Polymerase (Roche) following the manufacturer’s protocol. For this purpose, Adapter primers containing T7 promoter sequences and 3′ Poly A tail sequences (120 nt) are used to amplify linear DNA-templates. Each of the adapter primers has overlap sequences which fuse regulatory sequences necessary for translation and transcription to the gene of interest (see, for example, FIGS. 7a and 7b). A typical 50 µl PCR reaction contains final concentrations of 200 µM (of each) dNTP, 0.5 µM for each primer, 50 ng linearized plasmid DNA, 1x PCR reaction buffer (1.5 mM MgCl2) and 1.25 U Taq DNA Polymerase per reaction (0.25 µl of /5U/µl). The specific PCR cycle programs or thermal profiles are adjusted according to the Tm of the primer pairs, according to the expected length of the PCR product and according to the used thermal cycler. The quality of the PCR products are checked by analyzing an aliquot by gel electrophoresis and the reactions are purified by commercial PCR purification kits ((NucleoSpin Gel and PCR clean-up, Macherey-Nagel).

### In Vitro Transcription Reaction

Addition of a 5′ end cap structure to the RNA is an important process in eukaryotes. It is essential for RNA stability, efficient translation nuclear transport and splicing. The process involves addition of a 7-methylguanosine cap at the 5′ triphosphate end of the RNA. RNA capping can be carried out post-transcriptionally using capping enzymes or co-transcriptionally using cap analogs such as ARCA (Jena Bioscience). In the enzymatic method, the mRNA may be capped using the Vaccinia virus capping enzyme12,13. The enzyme adds on a 7-methylguanosine cap at the 5′ end of the RNA using GTP and S-adenosyl methionine as donors (cap 0 structure). Both methods yield functionally active capped RNA suitable for transfection or other applications.

In the example 6 described below, the T7 High Yield Transcription Kit (Thermo Scientific) has been used to synthesize capped RNA transcripts (PAN 01-88) using cap analogs co-transcriptionally.

The DNA template for the transcription reaction is a linearized plasmid or a PCR product. For the capped RNA the reaction at room temperature is set up in the following order:

| Component | Volume (µl) | Final |
|---|---|---|
| Nuclease free water* | To 20 | |
| 5X Reaction Buffer | 4 | 1X |
| ATP (100 mM) | 2 | 10 mM |
| CTP (100 mM) or 5-Methylcytidine-5′-Triphophate (100 mM) | 2 | 10 mM |
| UTP (100 mM) or Pseudouridine-5′-Triphosphate (100 mM) or 5-Methoxyuridine-5′- Triphosphate (100 mM) | 2 | 10 mM |
| GTP (30 mM) | 2 | 3 mM |
| 3′-0-Me-m⁷G(5′)ppp(5′)G cap analog (ARCA; Anti Reverse Cap Analog) (100 mM) | 2 | 10 mM |
| Template DNA* | X | 1 µg |
| T7 RNA Polymerase Mix | 2 | |
| Total | 20 | |

Add 1 µg DNA and make up the total reaction volume to 20 µl with nuclease free water. The amount of water to be added will vary based on the concentration of the template DNA. Mix well by vortexing and incubate the reactions at 37° C. for 2 hours in a dry air incubator. The transcription reactions are treated with DNase I to remove the DNA template before proceeding with purification.
1. Add 70 µl nuclease free water to the transcription reactions followed by 10 µl of DNase I reaction buffer.
2. Add 2 µl DNase I to the reactions.
3. Incubate at 37° C. for 15 minutes.

Column Purification of Capped mRNA
1. Purify the capped RNA using the MEGAclearKit as per the manufacturer’s instructions (any spin column based-RNA purification kit may be used).
2. Quantify the RNA using a NanoDrop Spectrophotometer.
3. As per the manufacturer’s instructions, assess RNA sample quality using the Agilent RNA 6000 Nano Kit and Agilent 2100 Bioanalyzer or by native 1% agarose gelelectrophoresis and ethidium bromide staining.

### mRNA Transfection of HeLa, HEK293, HUVEC and HPMEC Cells

Primary Human Pulmonary Microvascular Endothelial Cells (HPMEC) are most appropriate for studying human lung diseases and are isolated from the lung from a single donor. Since lung tissue contains blood and lymphatic capillaries, HPMEC comprise Blood and Lymphatic Microvascular Endothelial Cells. The cells are routinely analyzed by immunofluorescent staining: they stain positive for CD31 and von Willebrand factor and negative for smooth muscle alpha-actin. HeLa cells, HUVEC, HPMEC cells were grown in DMEM complete medium or Endothelial Cell Growth Medium (PromoCell, Heidelberg Germany, CatNo.: C-22020) respectively. The Endothelial Cell Growth Medium is basal Medium supplemented with Fetal Calf Serum (0.05 ml/ml), Endothelial Cell Growth Supplement (bovine hypothalamic extract; 0.004 ml ml), Heparin 90 µg/ml and Hydrocortisone 1 µg/ml. The experiments with HUVEC and HPMEC cells are performed on cells with passage numbers below 8.

mRNA transfections are carried out using Lipofectamin MessengerMAX (Invitrogen, Carlsbad, CA; Thermo Fisher Scientific). For mRNA transfection in 24-well plate format 1×105 cells/well were seeded 24h before transfection to reach 70-90 confluence immediate before transfection. For one single well add 1.5 µl Lipofectamine MessengerMAX Reagent to 25 µl OpitMEM, vortex and incubate for 10 min at RT. In a second well add 500 ng or 1 µg mRNA to 25 µl OptiMEM, vortex. After incubation add diluted mRNA to diluted Lipofectamin, mix well, incubate for 5 min at RT and add dropwise to cells in the presence of 0.5 ml of DMEM complete medium for HeLa. For HUVEC and HPMEC replace the Endothelial Cell Growth medium and wash the cells with OptiMEM and then add the Lipofectamin MessengerMAxX mRNA complex solution to the well with 0.5 ml OptiMEM prewarmed to 37 oC . Incubate cells at 37oC for 2 h and replace the OptiMEM medium with complete Endothelial Cell Growth Medium (PromoCell, Heidelberg Germany) and analyze transfected cells or serum at the indicated time points. Alternatively mRNA-LNPs based on the cationic lipids L-Arginyl-β-alanine-N-palmityl-N-oleyl-amide or β-(L-Arginyl)-L-2,3-diamino propionic acid-N-palmityl-N-oleyl-amide in combination with neutral and PEGylated co-lipids can be used for in vitro transfection.

Successful introduction of mRNA into host cells can be monitored using various known methods, such as measuring the protein expression level of the target polypeptide by e.g., Western Blotting or immunocytochemistry or ELISA.

Fully modified mRNAs with 5-methylcytosine and/or pseudouridine (or 5-methoxyuridine) are transfected into HUVEC or HPMEC (Human Pulmonary Microvascular Endothelial Cells, PromoCell, Heidelberg) using Lipofectamin MessengerMAX (Invitrogen, Carlsbad, CA; Thermo Fisher Scientific). The cell lysates are harvested and run by ELISA at different time points (30″, 1 h, 2 h, 4 h, 6 h hours) after transfection to determine the protein expression.

For immunoblot detection of Tie-2 and derivatives protein lysates were loaded on NuPage SDS-PAGE system (chambers and power supply) with 1.5 mm ready-to-use Bis-Tris gels and 4-12% acrylamide gradient with MOPS-buffer as running aid (all Life Technologies, Grand Island, NY). Each lysate sample was prepared to 40 ul final volume. This sample contained 25ug protein lysate in variable volume, RIPA buffer to make up volume to 26 ul, 4 ul of 1Ox reducing agent and 10 ul 4x SDS loading buffer (both from Life Technologies, Grand Island, NY). Samples were heated at 95oC for 5 min and loaded on the gel. Standard settings were chosen by the manufacturer, 200 V, 120 mA and max. 25 W. Run time was 60 min, but no longer than running dye reaching the lower end of the gel.

After the run was terminated, the plastic case was cracked and the encased gel transferred to a ready-to-use nitrocellulose membrane kit and power supply (iBLOT; LifeTechnologies, Grand Island, NY). Using default settings, the protein lysate was transferred by high Ampere electricity from the gel to the membrane.

After the transfer, the membranes were incubated in 5% BSA in IX TBS for 15 minutes then in 5% BSA in IX TBS + 0.1 % Tween for another 15 minutes. Primary antibodies against human Tie-2 proteins or Phospho-specific Antibodies (P-TIE2, P-AKT, see below) were applied in 3 ml of 5% BSA in IX TBS solution at a 1:500 to 1:2000 dilution for 3 hours at room temperature and gentle agitation on an orbital shaker. Membranes are washed 3 times with IX TBS/0.1% Tween, 5 minutes each time with gentle agitation. The secondary antibody (Goat anti-rabbit HRP conjugate; Abeam, Cambridge, MA) was conjugated to horse radish peroxidase and binds to the primary antibody antibodies. The secondary antibody was diluted of 1:1000 to 1:5000 in 5% BSA in IX TBS and incubated for 3 hrs at RT. At the end of incubation time, the membranes were washed 3 times with IX TBS/0.1 % Tween, 5 minutes each time with gentle agitation. The membranes were developed in 5ml Pierce WestPico Chemiluminescent Subtrate (Thermo Fisher, Rockford, IL) as directed.

### Example 6: Tie-2 Pathway Activation After Transfection of Modified mRNAs Encoding the Wildtype Tie-2 (PAN60) or Activating Tie-2 Mutations (PAN61-65)

Different mRNA construct encoding for Tie-2 wt or activating Tie-2 point-mutation are prepared by in vitro transcription and transfected into mammalian cells including primary microvascular endothelial cells. Tie-2 expression and Tie-2 phosphorylation status are analyzed to demonstrate expression and activation. Examples for Tie-2 activation are, for example, increased Phopho-Tie-2, Phospho-Akt or downstream effector protein expression such as VE-cadherin.

Modified mRNAs containing heterologous 5′ and 3′ UTRs are generated by in vitro transcription of PCR templates as described previously in example 5. Different concentration of this mRNA encoding the activating Tie-2 mutations are transfected in different cell lines (HeLa, primary endothelial cells such as HUVECs or HPMEC) next to mRNAs encoding the wild-typeTie-2 using Lipofectamin MessengerMAX (Invitrogen, Carlsbad, CA; Thermo Fisher Scientific.). For qualitative Western blot cell lysates at different time points post transfection of mRNAs (1-24 h) are probed with anti-phosphotyrosine antibody to evaluate Tie-2 phosphorylation (pY992 Tie-2, RnD Systems, Minneapolis, MN; Bio-Techne), 140 kD,). Blots are stripped and reprobed with anti-Tie-2 antibody to detect total Tie-2.

### Results

Interestingly transfection of Tie-2 mutations (R849W) encoding mRNAs do increase the presence of Phospho-Tie-2 in comparison to cell lysates derived from cells transfected with mRNAs encoding wildtype Tie-2 mRNA or in comparison to untransfected cells. This increase in Tie-2 phosphorylation is observed in the presence or absence of co-stimulation with recombinant human Ang-1 ligand and can be observed in different cell lines. To confirm the activation of the Tie-2 signaling pathway and to demonstrate a biological relevant functional downstream signal transduction by expressing the Tie-2 mutants derivate a phosphorylation of Akt is demonstrated in a time course experiment. These experiments indicate that mRNAs encoding for the Tie-2 activating mutations is functional and stronger in activating the Tie-2 signalling pathway than wt Tie-2 encoding mRNAs. These hyperactivation of the pathway is demonstrated in the absence or presence of Tie-2 agonist (e.g. Ang-1) in human endothelial and/or non-endothelial cells.

More specifically, FIG. 123 shows 1% agarose gels stained with EtBr showing PCR products of Poly-A tailing PCRs for the addition of 120 nt of Poly-A tail and mRNAs derived from these templates. Upper panel shows a quality control of PCR generated DNA templates of the indicated constructs (PAN 60 encoding for TIE2 wt, or for the mutated constructs PAN 61 encoding for TIE2 R849W, PAN 62 encoding for TIE2 L914F, PAN 63 encoding for TIE2 R849W L914F, PAN 64 encoding for Y897F R915L and PAN 65 encoding for TIE2 Y897F R915L T1105N T1106P. Lower Panel show a quality control of IVT generated mRNA of the indicated constructs. 1 µg of the DNA or mRNA were loaded on a non-denaturing 1% agarose gel containing EtBr, the electrophorese was performed at 120 V for 30 min and the nucleic acid was visualized under UV light.

Furthermore, as indicated in the immunoblots shown in FIGS. 124 and 125 the constructs PAN62, PAN63 and PAN64, show a constitutive Tie-2 activation indicated by the phosphorylation of the Tie-2 receptor in the absence of Ang-1. Upper panel immunoblot on HeLa cell lysates 7 h after mRNA transfection of the different constructs. PAN 51 serves as a negative control. Lower panel immunoblot on HeLa cells lysates transfected for 24 h with the indicated mRNA constructs and stimulated with recombinant ANG-1 for 15 or 30 min. A reduction of Tie-2 protein level in comparison to the wt (PAN60) is a result of the increased internalization and degradation of the Tie-2 receptor itself after the increased Tie-2 activation of the constitutive active mutants. Both experiments show a Tie-2 phosphorylation in the absence of Tie-2 agonist with mRNAs derived from constructs PAN62, PAN63 and PAN64.

### Example 7: Tie-2 Pathway Activation After Transfection of Human Pulmonary Microvascular Endothelial Cells (HPMEC) With mRNAs Encoding Full-Length Wildtype Tie-2 (PAN60) or Mutated Full-Length Tie-2 Constructs (PAN61-63)

Modified mRNAs containing heterologous 5′ and 3′ UTRs and coding sequences for wildtype (wt) Tie-2 (PAN60) or activating Tie-2 mutations (PAN61-63) were transfected in Tie-2-positive HPMEC. Subsequently, Tie-2 expression and phosphorylation as well as downstream signaling activation was analyzed by Western blot analysis.

Therefore, cells were grown at 90% confluency in 6well plates. After starvation of the cells in 1%-serum ECM (endothelial cell medium, Promocell, Heidelberg, Germany) for 24 h, the cells were transfected in serum-free ECM with 2 µg of the respective mRNA (PAN61-63) using Lipofectamin MessengerMAX (Invitrogen, Carlsbad, CA; Thermo Fisher Scientific). The Nluc encoding mRNA PAN51 was used as control. After 3h, serum-free ECM media was replaced with 1%-serum ECM. 9 hours after mRNA transfection, cells were washed with ice-cold PBS, lysed in RIPA buffer and protein lysates were separated using SDS-PAGE and protein levels were assessed by immunoblot analysis using anti-phospho-Tie-2 antibody (pY992 Tie-2, RnD Systems, Minneapolis, MN; Bio-Techne), anti-phospho-Akt antibody (pS473 Akt, Cell Signaling Technology, Danvers, MA), anti-Tie-2 (Cell Signaling Technology, Danvers, MA) and anti-Akt antibody (Cell Signaling Technology, Danvers, MA).

The results are shown in FIG. 126. For mRNA encoding the wildtype Tie-2 (PAN60) and the TIE 2_R849W mutation (PAN61) almost no Tie-2 autophosphorylation was detectable. However, cells transfected with mRNA encoding the Tie-2_L914F mutation (PAN62) showed moderate ligand-(Ang-1)-independent autophosphorylation. Surprisingly, cells transfected with mRNA encoding the double-mutation variant Tie-2-R849W_L914F (PAN63) showed very strong Tie-2 autophosphorylation. The remarkably strong ligand-independent Tie-2 pathway activation for PAN63 was further corroborated by a strong downstream phospho-Akt signal. These data indicate that PAN63 is a suitable mRNA construct for expression of constitutive active Tie-2 derivatives and can be used for therapeutic Tie-2 activation in endothelial cells even under conditions where endogenous Tie-2 protein expression has been down regulated.

### Example 8: Time-Dependent Expression of mRNAs Encoding Hypermorphic Full-Length Tie-2 (PAN63) or Hypermorphic Truncated Tie-2 Variants, with Heterologous N-Terminal Domains (PAN77, PAN78) in Tie-2-Positive Human Pulmonary Microvascular Endothelial Cells (HPMEC)

Modified mRNAs containing heterologous 5′ and 3′ UTRs and coding sequences for hypermorphic (R849W _L914F double mutation) full-length Tie-2 (PAN63;) or hypermorphic (R849W_L914F double mutation) truncated Tie-2 mutations (PAN77, PAN78) were transfected in Tie-2-positive HPMEC. As a negative control Nluc encoding mRNA was used whereas an Ang1 ligand was used as a positive control.

The novel truncated hypermorphic Tie-2 variants with heterologous N-terminal domains were designed as follows: For PAN77 an N-terminal myristylation signal (MGSSKSKPKDPSQR) of Src kinase was fused via a short GGH-linker to the C-terminal intracellular kinase domain (KD) of Tie-2 (amino acids 770-1124 of Tie-2; UniProtKB -Q02763) comprising the Tie-2 R849W_L914F double mutation. For PAN78 an N-terminal coiled-coil (multimerization) domain of human COMP (amino acids 29-73 of cartilage oligomeric matrix protein; UniProtKB P49747 (Refseq: NP_000086.2; NM_000095.3)) was fused via a GGISGGGGGI-linker to the C-terminal transmembrane and kinase domain of Tie-2 (amino acids 749-1124 of Tie-2; UniProtKB - Q02763) comprising the Tie-2 R849W_L914F double mutation. The fusion proteins were thus termed myrTie-2 (encoded by PAN77) or COMP-Tie-2, respectively.

After transfection into HPMEC the Tie-2 expression and phosphorylation as well as downstream signaling activation was analyzed by Western blot analysis.

For transfection, cells were grown at 90% confluency in 6well plates. After starvation of the cells in 1%-serum ECM (endothelial cell medium, Promocell, Heidelberg, Germany) for 24 h, the cells were transfected in serum-free ECM with 2 µg of the respective mRNA using Lipofectamin MessengerMAX (Invitrogen, Carlsbad, CA; Thermo Fisher Scientific). After 3 h, serum-free ECM media was replaced with 1%-serum ECM. 2, 4, 6 and 8 hours after mRNA transfection, cells were washed with ice-cold PBS, lysed in RIPA buffer and protein lysates were separated using SDS-PAGE and protein levels were assessed by immunoblot analysis using anti-phospho-Tie-2 antibody (pY992 Tie-2, RnD Systems, Minneapolis, MN; Bio-Techne), anti-phospho-Akt antibody (pS473 Akt, Cell Signaling Technology, Danvers, MA), N-terminal anti-Tie-2 (ab33, MerckMillipore, Darmstadt, Germany), C-terminal anti-Tie-2 antibody (SAB4502942, MerckMillipore, Darmstadt, Germany), and anti-Akt antibody (Cell Signaling Technology, Danvers, MA).

The results are shown in FIG. 127. 6 and 8 hours after transfection with mRNA encoding the hypermorphic full-length Tie-2 (PAN63) the cells showed strong Tie-2 auto-phosphorylation which is significantly stronger than Ang-1 ligand mediated Tie-2 activation. Concomitantly, downstream Tie-2 pathway activation was shown by increased Akt phosphorylation.

Surprisingly, compared to the already high auto-phosphorylation activity of PAN63, cells transfected with mRNAs encoding the truncated hypermorphic Tie-2 variants with heterologous N-terminal domains (PAN77, PAN78) showed massively increased Tie-2 phosphorylation as well as downstream Akt activation.

Even more surprisingly, the Tie-2 phosphorylation and downstream pathway activation was already present just 2 hours after mRNA transfection compared to 6-8 hours for PAN63.

### Example 9: Dose-dependent Expression of mRNAs Encoding Hypermorphic Full-Length Tie-2 (PAN63) or Hypermorphic Truncated Tie-2 Variants with Heterologous N-Terminal Domains (PAN77, PAN78) in Tie-2-Positive Human Pulmonary Microvascular Endothelial Cells (HPMEC).

Modified mRNAs containing heterologous 5′ and 3′ UTRs and coding sequences for hypermorphic (R849W_L914F double mutation) full-length Tie-2 (PAN63;) or hypermorphic (R849W_L914F double mutation) truncated Tie-2 mutations (PAN77, PAN78) were transfected in Tie-2-positive HPMEC. As a negative control Nluc encoding mRNA (PAN50) was used.

The truncated hypermorphic Tie-2 variants with heterologous N-terminal domains were designed as outlined in Example 8.

For transfection, cells were grown at 90% confluency in 6well plates. After starvation of the cells in 1%-serum ECM (endothelial cell medium, Promocell, Heidelberg, Germany) for 24 h, the cells were transfected in serum-free ECM with 2, 1, 0.5, 0.25 µg/well of the respective mRNA using Lipofectamin MessengerMAX (Invitrogen, Carlsbad, CA; Thermo Fisher Scientific). After 3h, serum-free ECM media was replaced with 1%-serum ECM. 8 hours after mRNA transfection, cells were washed with ice-cold PBS, lysed in RIPA buffer and protein lysates were separated using SDS-PAGE and protein levels were assessed by immunoblot analysis using anti-phospho Y992 antibody (RnD Systems, Minneapolis, MN; Bio-Techne) and anti-phospho Y1102 Tie-2 antibodies (RnD Systems, Minneapolis, MN; Bio-Techne), anti-phospho S1119 Tie-2 antibody (Cell Signaling Technology, Danvers, MA), anti-phospho pS473 Akt antibody (Cell Signaling Technology, Danvers, MA), N-terminal anti-Tie-2 (ab33, MerckMillipore, Darmstadt, Germany), C-terminal anti-Tie-2 antibody (SAB4502942, MerckMillipore, Darmstadt, Germany), and anti-Akt antibody (Cell Signaling Technology, Danvers, MA).

The results are shown in FIG. 128. 8 hours after transfection a clear dose dependent expression of the mRNAs encoding the hypermorphic truncated Tie-2 variants (PAN77, PAN78) was detectable. Furthermore, PAN77 as well as PAN78 showed very strong Tie-2 phosphorylation as assessed with pY992 Tie-2 antibody. Due to this very high activity, also phosphorylation in different Tie-2 segments using antibodies pY1102 Tie-2 and pS1119 Tie-2 with lower specificity became detectable.

Concomitantly, a dose dependent Tie-2 pathway downstream signaling activity was evident for PAN77 and PAN78 as assessed by phosphorylation of Akt.

### Example 10: Tie-2 Receptor Pathway Activation and Downstream Signaling Analysis After Expression of Hypermorphic Truncated Tie-2 Variants With Heterologous N-Terminal Domains (PAN77, PAN78) in Tie-2-Positive Human Pulmonary Microvascular Endothelial Cells (HPMEC)

Modified mRNAs containing heterologous 5′ and 3′ UTRs and coding sequences for hypermorphic (R849W_L914F double mutation) truncated TIE¬ 2 mutations with heterologous N-terminal domains (PAN77, PAN78) were transfected in Tie-2-positive HPMEC. As a negative control Nluc encoding mRNA (PAN50) was used. The truncated hypermorphic Tie-2 variants were designed as outlined in Example 8. For transfection, cells were grown at 90% confluency in 6well plates. After starvation of the cells in 1%-serum ECM (endothelial cell medium, Promocell, Heidelberg, Germany) for 24 h, the cells were transfected in serum-free ECM with 2, 1, 0.5, 0.25 µg/well of the respective mRNA using Lipofectamin MessengerMAX (Invitrogen, Carlsbad, CA; Thermo Fisher Scientific). After 3 h, serum-free ECM media was replaced with 1%-serum ECM. 8 hours after mRNA transfection, cells were washed with ice-cold PBS, lysed in RIPA buffer and protein lysates were separated using BisTRIS-PAGE and protein levels were assessed by immunoblot analysis using anti-Akt (Cell Signaling Technology, Danvers, MA) and anti-FOXO1 (Cell Signaling Technology, Danvers, MA) antibodies or anti-phospho-Akt (pS473 Akt, Cell Signaling Technology, Danvers, MA) and anti-phospho-FOXO1 (pT24 FOXO1, Cell Signaling Technology, Danvers, MA; pS256 FOXO1, Cell Signaling Technology, Danvers, MA) antibodies, respectively.

The immunoblot results are shown in FIG. 129. 8 hours after transfecting the cells with mRNAs PAN77 (encoding myrTie-2) and PAN78 (encoding COMP-Tie-2) a dose dependent strong increase in phospho-Akt and downstream phospho-FOXO1 was detectable, reflecting Tie-2 receptor pathway activation.

The features of the present invention disclosed in the specification, the claims and/or the drawings may both separately and in any combination thereof be material for realizing the invention in various forms thereof.

1. A recombinant nucleic acid construct comprising in 5′ -> 3′ direction
a 5′ UTR,
a coding region coding for an effector molecule, and
a 3′ UTR,
wherein the 5′ UTR is selected from the group consisting of a 5′ UTR of a gene coding for MCP-1 or a derivative thereof having a nucleotide identity of at least 85%, a 5′ UTR of a gene coding for RPL12s.c. or a derivative thereof having a nucleotide identity of at least 85 %, a 5′ UTR of a gene coding for Ang-2 or a derivative thereof having a nucleotide identity of at least 85%, a 5′ UTR of a gene coding for HSP70 or a derivative thereof having a nucleotide identity of at least 85%, a 5′ UTR of a gene coding for H3.3. or a derivative thereof having a nucleotide identity of at least 85%, a 5′ UTR of a gene coding for Galectin-9 or a derivative thereof having a nucleotide identity of at least 85%, a 5′ UTR of a gene coding for GADD34 or a derivative thereof having a nucleotide identity of at least 85%, a 5′ UTR of a gene coding for EDN1 or a derivative thereof having a nucleotide identity of at least 85%, a 5′ UTR of a gene coding for HSP70m5 or a derivative thereof having a nucleotide identity of at least 85%, a 5′ UTR of a gene coding for E-selectin or a derivative thereof having a nucleotide identity of at least 85% a 5′ UTR of a gene coding for ICAM-1 or a derivative thereof having a nucleotide identity of at least 85%, a 5′ UTR of a gene coding for IL-6 or a derivative thereof having a nucleotide identity of at least 85% and a 5′ UTR of a gene coding for vWF or a derivative thereof having a nucleotide identity of at least 85%;
wherein the 3′ UTR is selected from the group consisting of a 3′ UTR of a gene coding for vWF or a derivative thereof having a nucleotide identity of at least 85%, a 3′ UTR of a gene coding for MCP-1 or a derivative thereof having a nucleotide identity of at least 85%,, a 3′ UTR of a gene coding for RPL12s.c. or a derivative thereof having a nucleotide identity of at least 85%, a 3′ UTR of a gene coding for HSP70 or a derivative thereof having a nucleotide identity of at least 85%, a 3′ UTR of a gene coding for H3.3. or a derivative thereof having a nucleotide identity of at least 85%, a 3′ UTR of a gene coding for GADD34 or a derivative thereof having a nucleotide identity of at least 85%, a 3′ UTR of a gene coding for EDN1 or a derivative thereof having a nucleotide identity of at least 85%, and a 3′ UTR of a gene coding for IL-6 or a derivative thereof having a nucleotide identity of at least 85%,
wherein the effector molecule is effective in restoring a cellular function of a cell or is effective in exercising a therapeutic effect in or on a cell,
wherein the effector molecule is a mutant effector molecule, wherein the mutant effector molecule is a gain of function mutant effector molecule and/or a hypermorphic mutant effector molecule,
wherein the 5′ UTR and the 3′ UTR of the recombinant construct are of different origin, and wherein the recombinant nucleic acid construct is different from a wild type mRNA coding for the effector molecule.

2. The recombinant nucleic acid construct of claim 1, wherein the mutant effector molecule is a mutant Tie-2, and wherein the mutant Tie-2 optionally is selected from the group comprising Tie-2 L914 F, Tie-2 R849E L914F, Tie-2 Y897F R915L and Tie-2 T1105N T1106P.

3. The recombinant nucleic acid construct of claim 1, wherein the mutant effector molecule is a mutant PIK3, preferably and wherein the mutant PIK3CA optionally is selected from the group comprising PIK3CA E542K, PIK2CA E545K and PIK3CA H1047R.

4. The recombinant nucleic acid construct of claim 1, wherein the mutant effector molecule is a mutant P110, and wherein the mutant P110 optionally is an engineered myristoylated form of the catalytic subunit p110alpha.

5. The recombinant nucleic acid construct of claim 1, wherein
a) the 5′ UTR is a 5′ UTR of a gene coding for MCP-1 or a derivative thereof having a nucleotide identity of at least 85%, and wherein the 3′ UTR is selected from the group comprising a 3′ UTR of a gene coding for vWF or a derivative thereof having a nucleotide identity of at least 85%, a 3′ UTR of a gene coding for HSP70 or a derivative thereof having a nucleotide identity of at least 85%, a 3′ UTR of a gene coding for RPL12s.c. or a derivative thereof having a nucleotide identity of at least 85% and a 3′ UTR of a gene coding for MCP-1 or a derivative thereof having a nucleotide identity of at least 85%, or
b) the 5′ UTR is a 5′ UTR of a gene coding for RPL12s.c. or a derivative thereof having a nucleotide identity of at least 85%, and wherein the 3′ UTR is selected from the group comprising a 3′ UTR of a gene coding for vWF or a derivative thereof having a nucleotide identity of at least 85%, a 3′ UTR of a gene coding for HSP70 or a derivative thereof having a nucleotide identity of at least 85%, a 3′ UTR of a gene coding for RPL12s.c. or a derivative thereof having a nucleotide identity of at least 85%, and a 3′ UTR of a gene coding for MCP-1 or a derivative thereof having a nucleotide identity of at least 85%, or
c) the 5′ UTR is a 5′ UTR of a gene coding for HSP70 or a derivative thereof having a nucleotide identity of at least 85%, and wherein the 3′ UTR is selected from the group comprising a 3′ UTR of a gene coding for HSP70 or a derivative thereof having a nucleotide identity of at least 85%, a 3′ UTR of a gene coding for RPL12s.c. or a derivative thereof having a nucleotide identity of at least 85%, a 3′ UTR of a gene coding for vWF or a derivative thereof having a nucleotide identity of at least 85% and a 3′ UTR of a gene coding for MCP-1 or a derivative thereof having a nucleotide identity of at least 85%, or
d) the 5′ UTR is a 5′ UTR of a gene coding for ANG-2 or a derivative thereof having a nucleotide identity of at least 85%, and wherein the 3′ UTR is selected from the group comprising a 3′ UTR of a gene coding for HSP70 or a derivative thereof having a nucleotide identity of at least 85%, a 3′ UTR of a gene coding for RPL12s.c. or a derivative thereof having a nucleotide identity of at least 85%, a 3′ UTR of a gene coding for vWF or a derivative thereof having a nucleotide identity of at least 85% and a 3′ UTR of a gene coding for MCP-1 or a derivative thereof having a nucleotide identity of at least 85%.

6. The recombinant nucleic acid construct of claim 1, wherein the
a) 3′UTR is a 3′ UTR of a gene coding for vWF or a derivative thereof having a nucleotide identity of at least 85% and the 5′ UTR is selected from the group comprising a 5′ UTR of a gene coding for MCP-1 or a derivative thereof having a nucleotide identity of at least 85%, a 5′ UTR of a gene coding for RPL12s.c. or a derivative thereof having a nucleotide identity of at least 85%, a 5′ UTR of a gene coding for HSP70 or a derivative thereof having a nucleotide identity of at least 85% and a 5′ UTR of a gene coding for ANG-2 of a derivative thereof having a nucleotide identity of at least 85%, or
b) 3′UTR is a 3′ UTR of a gene coding for HSP70 or a derivative thereof having a nucleotide identity of at least 85% and the 5′ UTR is selected from the group comprising a 5′ UTR of a gene coding for HSP70 or a derivative thereof having a nucleotide identity of at least 85% a 5′ UTR of a gene coding for RPL12s.c. or a derivative thereof having a nucleotide identity of at least 85%, a 5′ UTR of a gene coding for ANG-2 of a derivative thereof having a nucleotide identity of at least 85%, and a 5′ UTR of a gene coding for MCP-1 or a derivative thereof having a nucleotide identity of at least 85%, or
c) 3′UTR is a 3′ UTR of a gene coding for RPL12s.c. or a derivative thereof having a nucleotide identity of at least 85% and the 5′ UTR is selected from the group comprising a 5′ UTR of a gene coding for HSP70 or a derivative thereof having a nucleotide identity of at least 85% a 5′ UTR of a gene coding for RPL12s.c. or a derivative thereof having a nucleotide identity of at least 85%, a 5′ UTR of a gene coding for ANG-2 of a derivative thereof having a nucleotide identity of at least 85%, and a 5′ UTR of a gene coding for MCP-1 or a derivative thereof having a nucleotide identity of at least 85%, or
d) 3′UTR is a 3′ UTR of a gene coding for MCP-1 or a derivative thereof having a nucleotide identity of at least 85 % and the 5′ UTR is selected from the group comprising a 5′ UTR of a gene coding for HSP70 or a derivative thereof having a nucleotide identity of at least 85%, a 5′ UTR of a gene coding for RPL12s.c. or a derivative thereof having a nucleotide identity of at least 85%, a 5′ UTR of a gene coding for ANG-2 of a derivative thereof having a nucleotide identity of at least 85%, and a 5′ UTR of a gene coding for MCP-1 or a derivative thereof having a nucleotide identity of at least 85%.

7. The recombinant nucleic acid construct of claim 1, wherein the construct is selected from the group consisting of:
a construct, wherein the 5′ UTR is a 5′ UTR of a gene coding for MCP-1 or a derivative thereof having a nucleotide identity of at least 85%, and the 3′ UTR is a 3′ UTR of a gene coding for vWF or a derivative thereof having a nucleotide identity of at least 85%,
a construct, wherein the 5′ UTR is a 5′ UTR of a gene coding for RPL12s.c. or a derivative thereof having a nucleotide identity of at least 85%, and the 3′ UTR is a 3′ UTR of a gene coding for vWF or a derivative thereof having a nucleotide identity of at least 85%,
a construct, wherein the 5′ UTR is a 5′ UTR of a gene coding for MCP-1 or a derivative thereof having a nucleotide identity of at least 85%, and the 3′ UTR is a 3′ UTR of a gene coding for MCP-1 or a derivative thereof having a nucleotide identity of at least 85%,
a construct, wherein the 5′ UTR is a 5′ UTR of a gene coding for ANG-2 or a derivative thereof having a nucleotide identity of at least 85%, and the 3′ UTR is a 3′ UTR of a gene coding for vWF or a derivative thereof having a nucleotide identity of at least 85%,
a construct, wherein the 5′ UTR is a 5′ UTR of a gene coding for RPL12s.c. or a derivative thereof having a nucleotide identity of at least 85%, and the 3′ UTR is a 3′ UTR of a gene coding for RPL12s.c. or a derivative thereof having a nucleotide identity of at least 85%,
a construct, wherein the 5′ UTR is a 5′ UTR of a gene coding for HSP70 or a derivative thereof having a nucleotide identity of at least 85%, and the 3′ UTR is a 3′ UTR of a gene coding for HSP70 or a derivative thereof having a nucleotide identity of at least 85%,
a construct, wherein the 5′ UTR is a 5′ UTR of a gene coding for H3.3. or a derivative thereof having a nucleotide identity of at least 85%, and the 3′ UTR is a 3′ UTR of a gene coding for H3.3. or a derivative thereof having a nucleotide identity of at least 85%, and
a construct, wherein the 5′ UTR is a 5′ UTR of a gene coding for Galectin 9 or a derivative thereof having a nucleotide identity of at least 85%, and the 3′ UTR is a 3′ UTR of a gene coding for Galectin 9 or a derivative thereof having a nucleotide identity of at least 85%,
wherein optionally the construct is
a construct, wherein the 5′ UTR is a 5′ UTR of a gene coding for MCP-1 or a derivative thereof having a nucleotide identity of at least 85%, and the 3′ UTR is a 3′ UTR of a gene coding for vWF or a derivative thereof having a nucleotide identity of at least 85%, or a construct, wherein the 5′ UTR is a 5′ UTR of a gene coding for RPL12s.c. or a derivative thereof having a nucleotide identity of at least 85%, and the 3′ UTR is a 3′ UTR of a gene coding for vWF or a derivative thereof having a nucleotide identity of at least 85%.

8. The recombinant nucleic acid construct of claim 1, wherein the construct comprises a poly-A tail, wherein said poly-A tail optionally is at the 3′ terminal end of the recombinant nucleic acid construct.

9. The recombinant nucleic acid construct of claim 1, wherein the construct comprises a CAP structure, wherein said CAP structure optionally is at the 5′ terminal end of the recombinant nucleic acid construct.

10. The recombinant nucleic acid construct of claim 1, wherein the construct comprises a IRES (internal ribosomal entry site) sequence, wherein optionally said IRES is at the 5′ terminal end of the recombinant nucleic acid construct.

11. The recombinant nucleic acid construct of claim 1, wherein the construct comprises nucleic acid sequence coding for a signal peptide, the wherein optionally said signal peptide is in-frame with nucleic acid sequence coding for a signal peptide and is arranged between the 5′ UTR and the coding region coding for an effector molecule.

12. The recombinant nucleic acid construct of claim 11, wherein the nucleotide sequence coding for a signal peptide is selected from the group consisting of a nucleotide sequence coding for a signal peptide of MCP-1 or a derivative thereof having a nucleotide identity of at least 85%, a nucleotide sequence coding for a signal peptide of IL-6 or a derivative thereof having a nucleotide identity of at least 85%,, a nucleotide sequence coding for a signal peptide of Ang-2 or a derivative thereof having a nucleotide identity of at least 85%, and a nucleotide sequence coding for a signal peptide of Ang-1 or a derivative thereof having a nucleotide identity of at least 85%.

13. The recombinant nucleic acid construct of claim 1, wherein the cell a cellular function of which is restored and/or the cell in or on which a therapeutic effect is exercised is an endothelial cell, wherein said endotheial cell optionally is a vascular endothelial cell, wherein said endotheial cell optionally is a vascular endothelial cell, or a or a microvascular endothelial cell.

14. The recombinant nucleic acid construct of claim 1, wherein the cellular function is one which can be restored by an effector molecule having anti-permeability effect of endothelial cells, an anti-vascular leakage effect, an anti-apoptotic effect of endothelial cells or an anti-inflammatory effect of endothelial cells or an anti-stress response effect, and wherein optionally the effect is linked to or associated with the Tie-2 signalling pathway, VEGF-receptor pathway, NOTCH signalling pathway, PI3-kinase pathway, eNOS signalling pathway, sirtuin-dependent metabolic and energy homeostasis pathway, oxidative stress pathway, shear stress response pathway, ET-1 signal transduction pathway, NO-mediated signal transduction pathway, or mechanochemical transduction pathway.

15. A vector comprising a nucleic acid construct according to claim 1.

16. A cell comprising a nucleic acid construct according to claim 1.

17. A delivery vehicle comprising a nucleic acid construct according to claim 1, wherein the delivery vehicle is a cationic lipid delivery particle, wherein optionally said the particle is a nanoparticle, and wherein optionally the average size of the nanoparticle is from about 30 nm to about 200 nm, from about 30 nm to about 140 nm or from about 30 nm to about 60 nm.

18. A pharmaceutical composition comprising a nucleic acid construct according to claim 1, and a pharmaceutically acceptable diluent.

19. A method for the treatment and/or prevention of a disease comprising administering to a subject suffering from the disease and effective amount of a construct according to claim 1.

20. A method for restoring a cellular function of a cell, comprising contacting the cell with a recombinant nucleic acid construct according to claim 1.


SEQUENCE LISTING (171 sequences)

SEQ ID NO: 1 - DNA, 38 bp, synthetic construct
  source 1..38: mol_type = other DNA
taatacgactcactatagggccctaagccatcagcaat

SEQ ID NO: 2 - DNA, 38 bp, synthetic construct
  source 1..38: mol_type = other DNA
taatacgactcactataggaaagtgattgattcggata

SEQ ID NO: 3 - DNA, 38 bp, synthetic construct
  source 1..38: mol_type = other DNA
taatacgactcactataggtccccattgaataacagcc

SEQ ID NO: 4 - DNA, 38 bp, synthetic construct
  source 1..38: mol_type = other DNA
taatacgactcactataggataaaagcccaggggcaag

SEQ ID NO: 5 - DNA, 38 bp, synthetic construct
  source 1..38: mol_type = other DNA
taatacgactcactatagggtcagccatctttcaattg

SEQ ID NO: 6 - DNA, 38 bp, synthetic construct
  source 1..38: mol_type = other DNA
taatacgactcactataggcatatcttcattcttcact

SEQ ID NO: 7 - DNA, 38 bp, synthetic construct
  source 1..38: mol_type = other DNA
taatacgactcactataggataaaagcctagtggccat

SEQ ID NO: 8 - DNA, 38 bp, synthetic construct
  source 1..38: mol_type = other DNA
taatacgactcactatagggaggaaccgagaggctgag

SEQ ID NO: 9 - DNA, 38 bp, synthetic construct
  source 1..38: mol_type = other DNA
taatacgactcactataggggagctgtttacccccact

SEQ ID NO: 10 - DNA, 38 bp, synthetic construct; identical to SEQ ID NO: 2
  source 1..38: mol_type = other DNA

SEQ ID NO: 11 - DNA, 139 bp, synthetic construct
  source 1..139: mol_type = other DNA
ttttttttttttttttttttttttttttttttttttttttttttttttttttttttttttttttttttttttttttttttttttttttttttttttttttttttttttttttttttttttgccttttgcaagataagag

SEQ ID NO: 12 - DNA, 139 bp, synthetic construct; identical to SEQ ID NO: 11
  source 1..139: mol_type = other DNA

SEQ ID NO: 13 - DNA, 139 bp, synthetic construct
  source 1..139: mol_type = other DNA
ttttttttttttttttttttttttttttttttttttttttttttttttttttttttttttttttttttttttttttttttttttttttttttttttttttttttttttttttttttttttgaattttaaagtttattta

SEQ ID NO: 14 - DNA, 139 bp, synthetic construct
  source 1..139: mol_type = other DNA
ttttttttttttttttttttttttttttttttttttttttttttttttttttttttttttttttttttttttttttttttttttttttttttttttttttttttttttttttttttttttatagtttaatgtattttaa

SEQ ID NO: 15 - DNA, 139 bp, synthetic construct
  source 1..139: mol_type = other DNA
ttttttttttttttttttttttttttttttttttttttttttttttttttttttttttttttttttttttttttttttttttttttttttttttttttttttttttttttttttttttttttaactacatctaaaccca

SEQ ID NO: 16 - DNA, 139 bp, synthetic construct
  source 1..139: mol_type = other DNA
tttttttttttttttttttttttttttttttttttttttttttttttttttttttttttttttttttttttttttttttttttttttttttttttttttttttttttttttttttttttttcgctacaaatcaaaaggc

SEQ ID NO: 17 - DNA, 139 bp, synthetic construct
  source 1..139: mol_type = other DNA
tttttttttttttttttttttttttttttttttttttttttttttttttttttttttttttttttttttttttttttttttttttttttttttttttttttttttttttttttttttttttgtacaaaaatatatttat

SEQ ID NO: 18 - DNA, 144 bp, synthetic construct
  source 1..144: mol_type = other DNA
tttttttttttttttttttttttttttttttttttttttttttttttttttttttttttttttttttttttttttttttttttttttttttttttttttttttttttttttttttttttttttacagtaaggaaaaaaatattt

SEQ ID NO: 19 - DNA, 139 bp, synthetic construct; identical to SEQ ID NO: 11
  source 1..139: mol_type = other DNA

SEQ ID NO: 20 - DNA, 73 bp, Homo sapiens
  source 1..73: mol_type = genomic DNA
gaggaaccgagaggctgagactaacccagaaacatccaattctcaaactgaagctcgcactctcgcctccagc

SEQ ID NO: 21 - DNA, 69 bp, Homo sapiens
  source 1..69: mol_type = genomic DNA
atgaaagtctctgccgcccttctgtgcctgctgctcatagcagccaccttcattccccaagggctcgct

SEQ ID NO: 22 - protein, 23 aa, Homo sapiens
MKVSAALLCLLLIAATFIPQGLA

SEQ ID NO: 23 - DNA, 373 bp, Homo sapiens
  source 1..373: mol_type = genomic DNA
acactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtaca

SEQ ID NO: 24 - DNA, 94 bp, Spinacia oleracea
  source 1..94: mol_type = genomic DNA
catatcttcattcttcactctttccccccccccccttatcctcctatctaatcctctcatctctctcctcctcttcctccaacaacaactaaca

SEQ ID NO: 25 - DNA, 148 bp, Spinacia oleracea
  source 1..148: mol_type = genomic DNA
ttttgagtaattttcaattttgtttttgtttttgtttttgttgttgggagttgttgtaatttggtaaatgttggggtcactaaaatcttttgttgcaattttagcacctttttattagatatataaaattgggtttagatgtagttaa

SEQ ID NO: 26 - DNA, 329 bp, Homo sapiens
  source 1..329: mol_type = genomic DNA
aaagtgattgattcggatactgacactgtaggatctggggagagaggaacaaaggaccgtgaaagctgctctgtaaaagctgacacagccctcccaagtgagcaggactgttcttcccactgcaatctgacagtttactgcatgcctggagagaacacagcagtaaaaaccaggtttgctactggaaaaagaggaaagagaagactttcattgacggacccagccatggcagcgtagcagccctgcgttttagacggcagcagctcgggactctggacgtgtgtttgccctcaagtttgctaagctgctggtttattactgaagaaaga

SEQ ID NO: 27 - DNA, 54 bp, Homo sapiens
  source 1..54: mol_type = genomic DNA
atgtggcagattgttttctttactctgagctgtgatcttgtcttggccgcagcc

SEQ ID NO: 28 - protein, 18 aa, Homo sapiens
MWQIVFFTLSCDLVLAAA

SEQ ID NO: 29 - DNA, 3450 bp, Homo sapiens
  source 1..3450: mol_type = genomic DNA
acatcccagtccacctgaggaactgtctcgaactattttcaaagacttaagcccagtgcactgaaagtcacggctgcgcactgtgtcctcttccaccacagagggcgtgtgctcggtgctgacgggacccacatgctccagattagagcctgtaaactttatcacttaaacttgcatcacttaacggaccaaagcaagaccctaaacatccataattgtgattagacagaacacctatgcaaagatgaacccgaggctgagaatcagactgacagtttacagacgctgctgtcacaaccaagaatgttatgtgcaagtttatcagtaaataactggaaaacagaacacttatgttatacaatacagatcatcttggaactgcattcttctgagcactgtttatacactgtgtaaatacccatatgtcctgaattcaccatcactatcacaattaaaaggaagaaaaaaactctctaagccataaaaagacatattcagggatattctgagaaggggttactagaagtttaatatttggaaaaacagttagtgcatttttactccatctcttaggtgctttaaatttttatttcaaaaacagcgtatttacatttatgttgacagcttagttataagttaatgctcaaatacgtatttcaaatttatatggtagaaacttccagaatctctgaaattatcaacagaaacgtgccattttagtttatatgcagaccgtactatttttttctgcctgattgttaaatatgaaggtatttttagtaattaaatataacttattaggggatatgcctatgtttaacttttatgataatatttacaattttataatttgtttccaaaagacctaattgtgccttgtgataaggaaacttcttacttttaatgatgaggaaaattatacatttcattctatgacaaagaaactttactatcttctcactattctaaaacagaggtctgttttctttcctagtaagatatatttttatagaactagactacaatttaatttctggttgagaaaagccttctatttaagaaatttacaaagctatatgtctcaagattcacccttaaatttacttaaggaaaaaaataattgacactagtaagtttttttatgtcaatcagcaaactgaaaaaaaaaaaagggtttcaaagtgcaaaaacaaaatctgatgttcataatatatttaaatatttaccaaaaatttgagaacacagggctgggcgcagtggctcacacctataatcccagtacattggtaggcaaggtgggcagatcacctgaggtcaggagttcaagaccagcctggacaacatggtgaaaccctgtctctactaaataatacaaaaattagccaggcgtgctggcgggcacctgtaatcccagctactcgggaggctgaggcagggagaattgcttgcaccagggaggtagaggttgcagtgagccaagatcgcaccactgcactccagccggggcaacagagcaagactccatctcaaaaaaaaaaaaaaaaaaagaaagaaaagaaaatttgagaacacagctttatactcgggactacaaaaccataaactcctggagttttaactccttttgaaattttcatagtacaattaatactaatgaacatttgtgtaaagctttataatttaaaggcaatttctcatatattcttttctgaatcatttgcaaggaagttcagagtccagtctgtaactagcatctactatatgtctgtcttcaccttacagtgttctaccattattttttctttattccatttcaaaatctaatttattttaccccaacttctccccaccacttgacgtagttttagaacacacaggtgttgctacatatttggagtcaatgatggactctggcaaagtcaaggctctgttttatttccaccaaggtgcacttttccaacaactatttaactagttaagaacctccctatcttagaactgtatctactttatatttaagaaggttttatgaattcaacaacggtatcatggccttgtatcaagttgaaaaacaactgaaaataagaaaatttcacagcctcgaaagacaacaacaagtttctaggatatctcaatgacaagagtgatggatacttaggtagggaaacgctaatgcaggaaaaactggcaacaacacaatttatatcaattctctttgtaggcaggtgataaaaaattcaaggacaaatctcattatgtcattgtgcatcatatataatctcttatgagcgagaatggggggaatttgtgtttttactttacacttcaattccttacacggtatttcaaacaaacagttttgctgagaggagcttttgtctctccttaagaaaatgtttataaagctgaaaggaaatcaaacagtaatcttaaaaatgaaaacaaaacaacccaacaacctagataactacagtgatcagggagcacagttcaactccttgttatgttttagtcatatggcctactcaaacagctaaataacaacaccagtggcagataaaaatcaccatttatctttcagctattaatcttttgaatgaataaactgtgacaaacaaattaacatttttgaacatgaaaggcaacttctgcacaatcctgtatccaagcaaactttaaattatccacttaattattacttaatcttaaaaaaaattagaacccagaacttttcaatgaagcatttgaaagttgaagtggaatttaggaaagccataaaaatataaatactgttatcacagcaccagcaagccataatctttatacctatcagttctatttctattaacagtaaaaacattaagcaagatataagactacctgcccaagaattcagtcttttttcatttttgtttttctcagttctgaggatgttaatcgtcaaattttctttggactgcattcctcactactttttgcacaatggtctcacgttctcacatttgttctcgcgaataaattgataaaaggtgttaagttctgtgaatgtctttttaattatgggcataattgtgcttgactggataaaaacttaagtccacccttatgtttataataatttcttgagaacagcaaactgcatttaccatcgtaaaacaacatctgacttacgggagctgcagggaagtggtgagacagttcgaacggctcctcagaaatccagtgacccaattctaaagaccatagcacctgcaagtgacacaacaagcagatttattatacatttattagccttagcaggcaataaaccaagaatcactttgaagacacagcaaaaagtgatacactccgcagatctgaaatagatgtgttctcagacaacaaagtcccttcagaatcttcatgttgcataaatgttatgaatattaataaaaagttgattgagaaaaa

SEQ ID NO: 30 - DNA, 121 bp, Homo sapiens
  source 1..121: mol_type = genomic DNA
gtctcaatattagagtctcaacccccaataaatataggactggagatgtctgaggctcattctgccctcgagcccaccgggaacgaaagagaagctctatctcccctccaggagcccagct

SEQ ID NO: 31 - DNA, 87 bp, Homo sapiens
  source 1..87: mol_type = genomic DNA
atgaactccttctccacaagcgccttcggtccagttgccttctccctggggctgctcctggtgttgcctgctgccttccctgcccca

SEQ ID NO: 32 - protein, 29 aa, Homo sapiens
MNSFSTSAFGPVAFSLGLLLVLPAAFPAP

SEQ ID NO: 33 - DNA, 429 bp, Homo sapiens
  source 1..429: mol_type = genomic DNA
catgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaa

SEQ ID NO: 34 - DNA, 255 bp, Homo sapiens
  source 1..255: mol_type = genomic DNA
gtggcagctcacagctattgtggtgggaaagggagggtggttggtggatgtcacagcttgggctttatctcccccagcagtggggactccacagcccctgggctacataacagcaagacagtccggagctgtagcagacctgattgagcctttgcagcagctgagagcatggcctagggtgggcggcaccattgtccagcagctgagtttcccagggaccttggagatagccgcagccctcatttgcaggggaag

SEQ ID NO: 35 - DNA, 66 bp, Homo sapiens
  source 1..66: mol_type = genomic DNA
atgattcctgccagatttgccggggtgctgcttgctctggccctcattttgccagggaccctttgt

SEQ ID NO: 36 - protein, 22 aa, Homo sapiens
MIPARFAGVLLALALILPGTLC

SEQ ID NO: 37 - DNA, 141 bp, Homo sapiens
  source 1..141: mol_type = genomic DNA
ggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 38 - DNA, 243 bp, Homo sapiens
  source 1..243: mol_type = genomic DNA
ataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccggc

SEQ ID NO: 39 - DNA, 258 bp, Homo sapiens
  source 1..258: mol_type = genomic DNA
gggcctttccaagattgctgtttttgttttggagcttcaagactttgcatttcctagtatttctgtttgtcagttctcaatttcctgtgtttgcaatgttgaaattttttggtgaagtactgaacttgctttttttccggtttctacatgcagagatgaatttatactgccatcttacgactatttcttctttttaatacacttaactcaggccattttttaagttggttacttcaaagtaaataaactttaaaattc

SEQ ID NO: 40 - DNA, 261 bp, Homo sapiens
  source 1..261: mol_type = genomic DNA
gggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctggcaag

SEQ ID NO: 41 - DNA, 54 bp, Homo sapiens
  source 1..54: mol_type = genomic DNA
atgaagctctccctggtggccgcgatgctgctgctgctcagcgcggcgcgggcc

SEQ ID NO: 42 - protein, 18 aa, Homo sapiens
MKLSLVAAMLLLLSAARA

SEQ ID NO: 43 - DNA, 1744 bp, Homo sapiens
  source 1..1744: mol_type = genomic DNA
acactgatctgctagtgctgtaatattgtaaatactggactcaggaacttttgttaggaaaaaattgaaagaacttaagtctcgaatgtaattggaatcttcacctcagagtggagttgaaactgctatagcctaagcggctgtttactgcttttcattagcagttgctcacatgtctttgggtgggggggagaagaagaattggccatcttaaaaagcgggtaaaaaacctgggttagggtgtgtgttcaccttcaaaatgttctatttaacaactgggtcatgtgcatctggtgtaggaagttttttctaccataagtgacaccaataaatgtttgttatttacactggtctaatgtttgtgagaagcttctaattagatcaattacttattttaggaaatttaagactagatactcgtgtgtggggtgaggggagggagtatttggtatgttgggataaggaaacacttctatttaatgcttccagggatttttttttttttttttaaccctcctgggcccaagtgatccttccacctcagtctcccagctaattgagaccacaggcttgttaccaccatgctcggcttttgcattaatctaagaaaaggggagagaagttaatccacatctttactcaggcaaggggcatttcacagtgcccaagagtggggttttcttgaacatacttggtttcctatttccccttatctttctaaaactgcctttctggtggctttttttaaaattattactaatgatgcttttatagctgcttggattctctgagaaatgatggggagtgagtgatcactggtattaactttatacacttggatttcatttgtaactttaggatgtaaaggtatattgtgaaccctagctgtgtcagaatctccatccctgaaatttctcattagtggtactggggtgggatcttggatggtgacattgaaactacactaaatcccctcactatgaatgggttgttaaaggcaatggtttgtgtcaaaactggtttaggattacttagattgtgttcctgaagaaaagagtccaggtaaatggtatgatcaataaaggacaggctggtgctaacataaaatccaatattgtaatcctagcactttgggaggccaaggcgggtggatcacaaggtcaagagatagagaccatctttgccaacatggtgaaactccatctctactgaaaatacaaaaattagctgggcgtggtagtgcaagctgaaggctgaggcaggagaatcactcgaacccgggaggcagaggttgcagtgagccgagatcacaccactgtactccagcccggcactccagcctggcgacaagagtgagactccacctcaaaaaaaaaaaaaagaatccaatactgcccaaggataggtattttatagatgggcaactggctgaaaggttaattctctagggctagtagaactggatcccaacaccaaactcttaattagacctaggcctcagctgcactgcccgaaaagcatttgggcagaccctgagcagaatactggtctcaggccaagcccaatacagccattaaagatgacctacagtgctgtgtaccctggggcaatagggttaaatggtagttagcaactagggctagtcttcccttacctcaaaggctctcactaccgtggaccacctagtctgtaactctttctgaggagctgttactgaatattaaaaagatagacttcaactatg

SEQ ID NO: 44 - DNA, 128 bp, Homo sapiens
  source 1..128: mol_type = genomic DNA
gtcagccatctttcaattgtgttcgcagccgccgccgcgccgccgtcgctctccaacgccagcgccgcctctcgctcgccgagctccagccgaaggagaaggggggtaagtaaggaggtctctgtacc

SEQ ID NO: 45 - DNA, 521 bp, Homo sapiens
  source 1..521: mol_type = genomic DNA
gaatccactatgatgggaaacatttcattctcaaaaaaaaaaaaaaaaatttctcttcttcctgttattggtagttctgaacgttagatattttttttccatggggtcaaaaggtacctaagtatatgattgcgagtggaaaaataggggacagaaatcaggtattggcagtttttccattttcatttgtgtgtgaatttttaatataaatgcggagacgtaaagcattaatgcaagttaaaatgtttcagtgaacaagtttcagcggttcaactttataataattataaataaacctgttaaatttttctggacaatgccagcatttggatttttttaaaacaagtaaatttcttattgatggcaactaaatggtgtttgtagcatttttatcatacagtagattccatccattcactatacttttctaactgagttgtcctacatgcaagtacatgtttttaatgttgtctgtcttctgtgctgttcctgtaagtttgctattaaaatacattaaacta

SEQ ID NO: 46 - DNA, 118 bp, Homo sapiens
  source 1..118: mol_type = genomic DNA
tccccattgaataacagccaagttgctttggtttctatttctttgttaagtcgttccctctacaaaggacttcctagtgggtgtgaaaggcagcggtggccacagaggcggcggagag

SEQ ID NO: 47 - DNA, 578 bp, Homo sapiens
  source 1..578: mol_type = genomic DNA
gcggcttcctggccctggggccgggggctggggtgtggggcagtctgggtcctctcatcatccccacttcccaggcccagcctttccaaccctgcctgggatctgggctttaatgcagaggccatgtccttgtctggtcctgcttctggctacagccaccctggaacggagaaggcagctgacggggattgccttcctcagccgcagcagcacctggggctccagctgctggaatcctaccatcccaggaggcaggcacagccagggagaggggaggagtgggcagtgaagatgaagccccatgctcagtcccctcccatcccccacgcagctccaccccagtcccaagccaccagctgtctgctcctggtgggaggtggcctcctcagcccctcctctctgacctttaacctcactctcaccttgcaccgtgcaccaacccttcacccctcctggaaagcaggcctgatggcttcccactggcctccaccacctgaccagagtgttctcttcagaggactggctcctttcccagtgtccttaaaataaagaaatgaaaatgcttgttggcacattca

SEQ ID NO: 48 - DNA, 4367 bp, synthetic construct
  source 1..4367: mol_type = other DNA
gccctaagccatcagcaatccttagtataggggcacactcatgcattcctgtcaagtcatcttgtgaaaggctgcctgcttccagcttggcttggatgtgcaaccttaataaaactcactgaggtctgggagaaaatagcagatctgcagcagatagggtagaggaaagggtctagaatatgtacacgcagctgactcaggcaggctccatgctgaacggtcacacagagaggaaacaataaatctcagctactatgcaataaatatctcaagttttaacgaagaaaaacatcattgcagtgaaataaaaaattttaaaattttagaacaaagctaacaaatggctagttttctatgattcttcttcaaacgctttctttgagggggaaagagtcaaacaaacaagcagttttacctgaaataaagaactagttttagaggtcagaagaaaggagcaagttttgcgagaggcacggaaggagtgtgctggcagtacaatgacagttttcctttcctttgctttcctcgctgccattctgactcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaaagcgcaatgtcagaagcgattatgaaagcaacaaagaaatccggagaagctgccaggtgagaaactgtttgaaaacttcagaagcaaacaatattgtctcccttccagcaataagtggtagttatgtgaagtcaccaaggttcttgaccgtgaatctggagccgtttgagttcacaagagtctctacttggggtgacagtgctcacgtggctcgactatagaaaactccactgactgtcgggctttaaaaagggaagaaactgctgagcttgctgtgcttcaaactactactggaccttattttggaactatggtagccagatgataaatatggttaatttcatgtaaaacagaaaaaaagagtgaaaaagagaatatacatgaagaatagaaacaagcctgccataatcctttggaaaagatgtattataccagtgaaaaggtgttatatctatgcaaacctactaacaaattatactgttgcacaattttgataaaaatttagaacagcattgtcctctgagttggttaaatgttaatggatttcagaagcctaattccagtatcatacttactagttgatttctgcttacccatcttcaaatgaaaattccatttttgtaagccataatgaactgtagtacatggacaataagtgtgtggtagaaacaaactccattactctgatttttgatacagttttcagaaaaagaaatgaacataatcaagtaaggatgtatgtggtgaaaacttaccacccccatactatggttttcatttactctaaaaactgattgaatgatatataaatatatttatagcctgagtaaagttaaaagaatgtaaaatatatcatcaagttcttaaaataatatacatgcatttaatatttcctttgatattatacaggaaagcaatattttggagtatgttaagttgaagtaaaagcaagtactctggagcagttcattttacagtatctacttgcatgtgtatacatacatgtaacttcattattttaaaaatatttttagaactccaatactcaccctgttatgtcttgctaatttaaattttgctaattaactgaaacatgcttaccagattcacactgttccagtgtctataaaagaaacactttgaagtctataaaaaataaaataattataaatatcattgtacatagcatgtttatatctgcaaaaaacctaatagctaattaatctggaatatgcaacattgtccttaattgatgcaaataacacaaatgctcaaagaaatctactatatcccttaatgaaatacatcattcttcatatatttctccttcagtccattcccttaggcaatttttaatttttaaaaattattatcaggggagaaaaattggcaaaactattatatgtaagggaaatatatacaaaaagaaaattaatcatagtcacctgactaagaaattctgactgctagttgccataaataactcaatggaaatattcctatgggataatgtattttaagtgaatttttggggtgcttgaagttactgcattattttatcaagaagtcttctctgcctgtaagtgtccaaggttatgacagtaaacagtttttattaaaacatgagtcactatgggatgagaaaattgaaataaagctactgggcctcctctcataaaagagacagttgttggcaaggtagcaataccagtttcaaacttggtgacttgatccactatgccttaatggtttcctccatttgagaaaataaagctattcacattgttaagaaaaatactttttaaagtttaccatcaagtcttttttatatttatgtgtctgtattctacccctttttgccttacaagtgatatttgcaggtattataccatttttctattcttggtggcttcttcatagcaggtaagcctctccttctaaaaacttctcaactgttttcatttaagggaaagaaaatgagtattttgtccttttgtgttcctacagacactttcttaaaccagtttttggataaagaatactatttccaaactcatattacaaaaacaaaataaaataataaaaaaagaaagcatgatatttactgttttgttgtctgggtttgagaaatgaaatattgtttccaattatttataataaatcagtataaaatgttttatgattgttatgtgtattatgtaatacgtacatgtttatggcaatttaacatgtgtattcttttaattgtttcagaataggataattaggtattcgaattttgtctttaaaattcatgtggtttctatgcaaagttcttcatatcatcacaacattatttgatttaaataaaattgaaagtaatatttgtgcaa

SEQ ID NO: 49 - DNA, 3782 bp, synthetic construct
  source 1..3782: mol_type = other DNA
gccctaagccatcagcaatccttagtataggggcacactcatgcattcctgtcaagtcatcttgtgaaaggctgcctgcttccagcttggcttggatgtgcaaccttaataaaactcactgaggtctgggagaaaatagcagatctgcagcagatagggtagaggaaagggtctagaatatgtacacgcagctgactcaggcaggctccatgctgaacggtcacacagagaggaaacaataaatctcagctactatgcaataaatatctcaagttttaacgaagaaaaacatcattgcagtgaaataaaaaattttaaaattttagaacaaagctaacaaatggctagttttctatgattcttcttcaaacgctttctttgagggggaaagagtcaaacaaacaagcagttttacctgaaataaagaactagttttagaggtcagaagaaaggagcaagttttgcgagaggcacggaaggagtgtgctggcagtacaatgacagttttcctttcctttgctttcctcgctgccattctgactgggatcttagacctagccccacagatgcttcgagaactccaggagactaatgcggcgctgcaagacgtgagagagctcttgcgacagcaggtcaaggagatcaccttcctgaagaatacggtgatggaatgtgacgcttgcggaggctcccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaaagcgcaatgtcagaagcgattatgaaagcaacaaagaaatccggagaagctgccaggtgagaaactgtttgaaaacttcagaagcaaacaatattgtctcccttccagcaataagtggtagttatgtgaagtcaccaaggttcttgaccgtgaatctggagccgtttgagttcacaagagtctctacttggggtgacagtgctcacgtggctcgactatagaaaactccactgactgtcgggctttaaaaagggaagaaactgctgagcttgctgtgcttcaaactactactggaccttattttggaactatggtagccagatgataaatatggttaatttcatgtaaaacagaaaaaaagagtgaaaaagagaatatacatgaagaatagaaacaagcctgccataatcctttggaaaagatgtattataccagtgaaaaggtgttatatctatgcaaacctactaacaaattatactgttgcacaattttgataaaaatttagaacagcattgtcctctgagttggttaaatgttaatggatttcagaagcctaattccagtatcatacttactagttgatttctgcttacccatcttcaaatgaaaattccatttttgtaagccataatgaactgtagtacatggacaataagtgtgtggtagaaacaaactccattactctgatttttgatacagttttcagaaaaagaaatgaacataatcaagtaaggatgtatgtggtgaaaacttaccacccccatactatggttttcatttactctaaaaactgattgaatgatatataaatatatttatagcctgagtaaagttaaaagaatgtaaaatatatcatcaagttcttaaaataatatacatgcatttaatatttcctttgatattatacaggaaagcaatattttggagtatgttaagttgaagtaaaagcaagtactctggagcagttcattttacagtatctacttgcatgtgtatacatacatgtaacttcattattttaaaaatatttttagaactccaatactcaccctgttatgtcttgctaatttaaattttgctaattaactgaaacatgcttaccagattcacactgttccagtgtctataaaagaaacactttgaagtctataaaaaataaaataattataaatatcattgtacatagcatgtttatatctgcaaaaaacctaatagctaattaatctggaatatgcaacattgtccttaattgatgcaaataacacaaatgctcaaagaaatctactatatcccttaatgaaatacatcattcttcatatatttctccttcagtccattcccttaggcaatttttaatttttaaaaattattatcaggggagaaaaattggcaaaactattatatgtaagggaaatatatacaaaaagaaaattaatcatagtcacctgactaagaaattctgactgctagttgccataaataactcaatggaaatattcctatgggataatgtattttaagtgaatttttggggtgcttgaagttactgcattattttatcaagaagtcttctctgcctgtaagtgtccaaggttatgacagtaaacagtttttattaaaacatgagtcactatgggatgagaaaattgaaataaagctactgggcctcctctcataaaagagacagttgttggcaaggtagcaataccagtttcaaacttggtgacttgatccactatgccttaatggtttcctccatttgagaaaataaagctattcacattgttaagaaaaatactttttaaagtttaccatcaagtcttttttatatttatgtgtctgtattctacccctttttgccttacaagtgatatttgcaggtattataccatttttctattcttggtggcttcttcatagcaggtaagcctctccttctaaaaacttctcaactgttttcatttaagggaaagaaaatgagtattttgtccttttgtgttcctacagacactttcttaaaccagtttttggataaagaatactatttccaaactcatattacaaaaacaaaataaaataataaaaaaagaaagcatgatatttactgttttgttgtctgggtttgagaaatgaaatattgtttccaattatttataataaatcagtataaaatgttttatgattgttatgtgtattatgtaatacgtacatgtttatggcaatttaacatgtgtattcttttaattgtttcagaataggataattaggtattcgaattttgtctttaaaattcatgtggtttctatgcaaagttcttcatatcatcacaacattatttgatttaaataaaattgaaagtaatatttgtgcaa

SEQ ID NO: 50 - DNA, 1976 bp, synthetic construct
  source 1..1976: mol_type = other DNA
aaagtgattgattcggatactgacactgtaggatctggggagagaggaacaaaggaccgtgaaagctgctctgtaaaagctgacacagccctcccaagtgagcaggactgttcttcccactgcaatctgacagtttactgcatgcctggagagaacacagcagtaaaaaccaggtttgctactggaaaaagaggaaagagaagactttcattgacggacccagccatggcagcgtagcagccctgcgttttagacggcagcagctcgggactctggacgtgtgtttgccctcaagtttgctaagctgctggtttattactgaagaaagaatgtggcagattgttttctttactctgagctgtgatcttgtcttggccgcagcccacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 51 - DNA, 1391 bp, synthetic construct
  source 1..1391: mol_type = other DNA
aaagtgattgattcggatactgacactgtaggatctggggagagaggaacaaaggaccgtgaaagctgctctgtaaaagctgacacagccctcccaagtgagcaggactgttcttcccactgcaatctgacagtttactgcatgcctggagagaacacagcagtaaaaaccaggtttgctactggaaaaagaggaaagagaagactttcattgacggacccagccatggcagcgtagcagccctgcgttttagacggcagcagctcgggactctggacgtgtgtttgccctcaagtttgctaagctgctggtttattactgaagaaagaatgtggcagattgttttctttactctgagctgtgatcttgtcttggccgcagccgggatcttagacctagccccacagatgcttcgagaactccaggagactaatgcggcgctgcaagacgtgagagagctcttgcgacagcaggtcaaggagatcaccttcctgaagaatacggtgatggaatgtgacgcttgcggaggctcccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 52 - DNA, 1967 bp, synthetic construct
  source 1..1967: mol_type = other DNA
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtaca

SEQ ID NO: 53 - DNA, 1382 bp, synthetic construct
  source 1..1382: mol_type = other DNA
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctgggatcttagacttgggcccgcagatgcttcgggaactgcaggaaaccaacgcggcgctgcaggacgtgcgggagctgctgcggcagcaggtcagggagatcacgttcctgaaaaacacggtgatggagtgtgacgcgtgcggaggctcccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtaca

SEQ ID NO: 54 - DNA, 3428 bp, synthetic construct
  source 1..3428: mol_type = other DNA
gccctaagccatcagcaatccttagtataggggcacactcatgcattcctgtcaagtcatcttgtgaaaggctgcctgcttccagcttggcttggatgtgcaaccttaataaaactcactgaggtctgggagaaaatagcagatctgcagcagatagggtagaggaaagggtctagaatatgtacacgcagctgactcaggcaggctccatgctgaacggtcacacagagaggaaacaataaatctcagctactatgcaataaatatctcaagttttaacgaagaaaaacatcattgcagtgaaataaaaaattttaaaattttagaacaaagctaacaaatggctagttttctatgattcttcttcaaacgctttctttgagggggaaagagtcaaacaaacaagcagttttacctgaaataaagaactagttttagaggtcagaagaaaggagcaagttttgcgagaggcacggaaggagtgtgctggcagtacaatgacagttttcctttcctttgctttcctcgctgccattctgactgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaaagcgcaatgtcagaagcgattatgaaagcaacaaagaaatccggagaagctgccaggtgagaaactgtttgaaaacttcagaagcaaacaatattgtctcccttccagcaataagtggtagttatgtgaagtcaccaaggttcttgaccgtgaatctggagccgtttgagttcacaagagtctctacttggggtgacagtgctcacgtggctcgactatagaaaactccactgactgtcgggctttaaaaagggaagaaactgctgagcttgctgtgcttcaaactactactggaccttattttggaactatggtagccagatgataaatatggttaatttcatgtaaaacagaaaaaaagagtgaaaaagagaatatacatgaagaatagaaacaagcctgccataatcctttggaaaagatgtattataccagtgaaaaggtgttatatctatgcaaacctactaacaaattatactgttgcacaattttgataaaaatttagaacagcattgtcctctgagttggttaaatgttaatggatttcagaagcctaattccagtatcatacttactagttgatttctgcttacccatcttcaaatgaaaattccatttttgtaagccataatgaactgtagtacatggacaataagtgtgtggtagaaacaaactccattactctgatttttgatacagttttcagaaaaagaaatgaacataatcaagtaaggatgtatgtggtgaaaacttaccacccccatactatggttttcatttactctaaaaactgattgaatgatatataaatatatttatagcctgagtaaagttaaaagaatgtaaaatatatcatcaagttcttaaaataatatacatgcatttaatatttcctttgatattatacaggaaagcaatattttggagtatgttaagttgaagtaaaagcaagtactctggagcagttcattttacagtatctacttgcatgtgtatacatacatgtaacttcattattttaaaaatatttttagaactccaatactcaccctgttatgtcttgctaatttaaattttgctaattaactgaaacatgcttaccagattcacactgttccagtgtctataaaagaaacactttgaagtctataaaaaataaaataattataaatatcattgtacatagcatgtttatatctgcaaaaaacctaatagctaattaatctggaatatgcaacattgtccttaattgatgcaaataacacaaatgctcaaagaaatctactatatcccttaatgaaatacatcattcttcatatatttctccttcagtccattcccttaggcaatttttaatttttaaaaattattatcaggggagaaaaattggcaaaactattatatgtaagggaaatatatacaaaaagaaaattaatcatagtcacctgactaagaaattctgactgctagttgccataaataactcaatggaaatattcctatgggataatgtattttaagtgaatttttggggtgcttgaagttactgcattattttatcaagaagtcttctctgcctgtaagtgtccaaggttatgacagtaaacagtttttattaaaacatgagtcactatgggatgagaaaattgaaataaagctactgggcctcctctcataaaagagacagttgttggcaaggtagcaataccagtttcaaacttggtgacttgatccactatgccttaatggtttcctccatttgagaaaataaagctattcacattgttaagaaaaatactttttaaagtttaccatcaagtcttttttatatttatgtgtctgtattctacccctttttgccttacaagtgatatttgcaggtattataccatttttctattcttggtggcttcttcatagcaggtaagcctctccttctaaaaacttctcaactgttttcatttaagggaaagaaaatgagtattttgtccttttgtgttcctacagacactttcttaaaccagtttttggataaagaatactatttccaaactcatattacaaaaacaaaataaaataataaaaaaagaaagcatgatatttactgttttgttgtctgggtttgagaaatgaaatattgtttccaattatttataataaatcagtataaaatgttttatgattgttatgtgtattatgtaatacgtacatgtttatggcaatttaacatgtgtattcttttaattgtttcagaataggataattaggtattcgaattttgtctttaaaattcatgtggtttctatgcaaagttcttcatatcatcacaacattatttgatttaaataaaattgaaagtaatatttgtgcaa

SEQ ID NO: 55 - DNA, 1037 bp, synthetic construct
  source 1..1037: mol_type = other DNA
aaagtgattgattcggatactgacactgtaggatctggggagagaggaacaaaggaccgtgaaagctgctctgtaaaagctgacacagccctcccaagtgagcaggactgttcttcccactgcaatctgacagtttactgcatgcctggagagaacacagcagtaaaaaccaggtttgctactggaaaaagaggaaagagaagactttcattgacggacccagccatggcagcgtagcagccctgcgttttagacggcagcagctcgggactctggacgtgtgtttgccctcaagtttgctaagctgctggtttattactgaagaaagaatgtggcagattgttttctttactctgagctgtgatcttgtcttggccgcagccgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 56 - DNA, 826 bp, synthetic construct
  source 1..826: mol_type = other DNA
tccccattgaataacagccaagttgctttggtttctatttctttgttaagtcgttccctctacaaaggacttcctagtgggtgtgaaaggcagcggtggccacagaggcggcggagagatgtggcagattgttttctttactctgagctgtgatcttgtcttggccgcagccgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 57 - DNA, 1068 bp, synthetic construct
  source 1..1068: mol_type = other DNA
ataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcgaatccagtgttccgtttccagcccccaatctcagagcggagccgacagagagcagggaaccggcatgtggcagattgttttctttactctgagctgtgatcttgtcttggccgcagccgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaagggcctttccaagattgctgtttttgttttggagcttcaagactttgcatttcctagtatttctgtttgtcagttctcaatttcctgtgtttgcaatgttgaaattttttggtgaagtactgaacttgctttttttccggtttctacatgcagagatgaatttatactgccatcttacgactatttcttctttttaatacacttaactcaggccattttttaagttggttacttcaaagtaaataaactttaaaattc

SEQ ID NO: 58 - DNA, 1217 bp, synthetic construct
  source 1..1217: mol_type = other DNA
gtcagccatctttcaattgtgttcgcagccgccgccgcgccgccgtcgctctccaacgccagcgccgcctctcgctcgccgagctccagccgaaggagaaggggggtaagtaaggaggtctctgtaccatgtggcagattgttttctttactctgagctgtgatcttgtcttggccgcagccgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaagaatccactatgatgggaaacatttcattctcaaaaaaaaaaaaaaaaatttctcttcttcctgttattggtagttctgaacgttagatattttttttccatggggtcaaaaggtacctaagtatatgattgcgagtggaaaaataggggacagaaatcaggtattggcagtttttccattttcatttgtgtgtgaatttttaatataaatgcggagacgtaaagcattaatgcaagttaaaatgtttcagtgaacaagtttcagcggttcaactttataataattataaataaacctgttaaatttttctggacaatgccagcatttggatttttttaaaacaagtaaatttcttattgatggcaactaaatggtgtttgtagcatttttatcatacagtagattccatccattcactatacttttctaactgagttgtcctacatgcaagtacatgtttttaatgttgtctgtcttctgtgctgttcctgtaagtttgctattaaaatacattaaactat

SEQ ID NO: 59 - DNA, 827 bp, synthetic construct
  source 1..827: mol_type = other DNA
catatcttcattcttcactctttccccccccccccttatcctcctatctaatcctctcatctctctcctcctcttcctccaacaacaactaacaatggcagcaactacaacaatggcacagattgttttctttactctgagctgtgatcttgtcttggccgcagccgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaattttgagtaattttcaattttgtttttgtttttgtttttgttgttgggagttgttgtaatttggtaaatgttggggtcactaaaatcttttgttgcaattttagcacctttttattagatatataaaattgggtttagatgtagttaa

SEQ ID NO: 60 - DNA, 925 bp, synthetic construct
  source 1..925: mol_type = other DNA
ataaaagcctagtggccattgtgttcgttgctcttatcggttcccatcccagttgttgatcttatgcaagacgctgcacgaccccgcgcccgcttgtcgccacggcacttgaggcagccggagatactctgagttactcggagcccgacgcctgagggtgagatgaacgcgctggcctccctaaccgtccggacctgtgatcgcttctggcagaccgaaccggcgctcctgcccccggggtgacgcgcagctcccagccgcccagacacatgtggcagattgttttctttactctgagctgtgatcttgtcttggccgcagccgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaagaccaactggtttgcctataatttattaactatttattttttctaagtgtgggtttatataaggaataaagccttttgatttgtagcga

SEQ ID NO: 61 - DNA, 1028 bp, synthetic construct
  source 1..1028: mol_type = other DNA
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtaca

SEQ ID NO: 62 - DNA, 2027 bp, synthetic construct
  source 1..2027: mol_type = other DNA
ggagctgtttacccccactctaataggggttcaatataaaaagccggcagagagctgtccaagtcagacgcgcctctgcatctgcgccaggcgaacgggtcctgcgcctcctgcagtcccagctctccaccgccgcgtgcgcctgcagacgctccgctcgctgccttctctcctggcaggcgctgccttttctccccgttaaaagggcacttgggctgaaggatcgctttgagatctgaggaacccgcagcgctttgagggacctgaagctgtttttcttcgttttcctttgggttcagtttgaacgggaggtttttgatccctttttttcagaatggattatttgctcatgattttctctctgctgtttgtggcttgccaaggagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaacagaccttcggggcctgtctgaagccatagcctccacggagagccctgtggccgactctgcactctccaccctggctgggatcagagcaggagcatcctctgctggttcctgactggcaaaggaccagcgtcctcgttcaaaacattccaagaaaggttaaggagttcccccaaccatcttcactggcttccatcagtggtaactgctttggtctcttctttcatctggggatgacaatggacctctcagcagaaacacacagtcacattcgaattcgggtggcatcctccggagagagagagaggaaggagattccacacaggggtggagtttctgacgaaggtcctaagggagtgtttgtgtctgactcaggcgcctggcacatttcagggagaaactccaaagtccacacaaagattttctaaggaatgcacaaattgaaaacacactcaaaagacaaacatgcaagtaaagaaaaaaaaaagaaagacttttgtttaaatttgtaaaatgcaaaactgaatgaaactgttactaccataaatcaggatatgtttcatgaatatgagtctacctcacctatattgcactctggcagaagtatttcccacatttaattattgcctccccaaactcttcccacccctgctgccccttcctccatcccccatactaaatcctagcctcgtagaagtctggtctaatgtgtcagcagtagatataatattttcatggtaatctactagctctgatccataagaaaaaaaagatcattaaatcaggagattccctgtccttgatttttggagacacaatggtatagggttgtttatgaaatatattgaaaagtaagtgtttgttacgctttaaagcagtaaaattattttcctttatataaccggctaatgaaagaggttggattgaattttgatgtacttatttttttatagatatttatattcaaacaatttattccttatatttaccatgttaaatatctgtttgggcaggccatattggtctatgtatttttaaaatatgtatttctaaatgaaattgagaacatgctttgttttgcctgtcaaggtaatgactttagaaaataaatatttttttccttactgtaaa

SEQ ID NO: 63 - DNA, 1070 bp, synthetic construct
  source 1..1070: mol_type = other DNA
aaagtgattgattcggatactgacactgtaggatctggggagagaggaacaaaggaccgtgaaagctgctctgtaaaagctgacacagccctcccaagtgagcaggactgttcttcccactgcaatctgacagtttactgcatgcctggagagaacacagcagtaaaaaccaggtttgctactggaaaaagaggaaagagaagactttcattgacggacccagccatggcagcgtagcagccctgcgttttagacggcagcagctcgggactctggacgtgtgtttgccctcaagtttgctaagctgctggtttattactgaagaaagaatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 64 - DNA, 796 bp, synthetic construct
  source 1..796: mol_type = other DNA
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 65 - DNA, 913 bp, synthetic construct
  source 1..913: mol_type = other DNA
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaagggcctttccaagattgctgtttttgttttggagcttcaagactttgcatttcctagtatttctgtttgtcagttctcaatttcctgtgtttgcaatgttgaaattttttggtgaagtactgaacttgctttttttccggtttctacatgcagagatgaatttatactgccatcttacgactatttcttctttttaatacacttaactcaggccattttttaagttggttacttcaaagtaaataaactttaaaattc

SEQ ID NO: 66 - DNA, 1046 bp, synthetic construct
  source 1..1046: mol_type = other DNA
gaggaaccgagaggctgagactaacccagaaacatccaattctcaaactgaagctcgcactctcgcctccagcatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtaca

SEQ ID NO: 67 - DNA, 1013 bp, synthetic construct
  source 1..1013: mol_type = other DNA
gaggaaccgagaggctgagactaacccagaaacatccaattctcaaactgaagctcgcactctcgcctccagcatgtggcagattgttttctttactctgagctgtgatcttgtcttggccgcagccgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtaca

SEQ ID NO: 68 - DNA, 962 bp, synthetic construct
  source 1..962: mol_type = other DNA
gaggaaccgagaggctgagactaacccagaaacatccaattctcaaactgaagctcgcactctcgcctccagcatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtaca

SEQ ID NO: 69 - DNA, 1198 bp, synthetic construct
  source 1..1198: mol_type = other DNA
ataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcgaatccagtgttccgtttccagcccccaatctcagagcggagccgacagagagcagggaaccggcatgaaagtctctgccgcccttctgtgcctgctgctcatagcagccaccttcattccccaagggctcgctgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtaca

SEQ ID NO: 70 - DNA, 1067 bp, synthetic construct
  source 1..1067: mol_type = other DNA
catatcttcattcttcactctttccccccccccccttatcctcctatctaatcctctcatctctctcctcctcttcctccaacaacaactaacaatggcagcaactacaacaatgaaagtctctgccgcccttctgtgcctgctgctcatagcagccaccttcattccccaagggctcgctgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtaca

SEQ ID NO: 71 - DNA, 1052 bp, synthetic construct
  source 1..1052: mol_type = other DNA
aaagtgattgattcggatactgacactgtaggatctggggagagaggaacaaaggaccgtgaaagctgctctgtaaaagctgacacagccctcccaagtgagcaggactgttcttcccactgcaatctgacagtttactgcatgcctggagagaacacagcagtaaaaaccaggtttgctactggaaaaagaggaaagagaagactttcattgacggacccagccatggcagcgtagcagccctgcgttttagacggcagcagctcgggactctggacgtgtgtttgccctcaagtttgctaagctgctggtttattactgaagaaagaatgaaagtctctgccgcccttctgtgcctgctgctcatagcagccaccttcattccccaagggctcgctgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 72 - DNA, 1064 bp, synthetic construct
  source 1..1064: mol_type = other DNA
aaagtgattgattcggatactgacactgtaggatctggggagagaggaacaaaggaccgtgaaagctgctctgtaaaagctgacacagccctcccaagtgagcaggactgttcttcccactgcaatctgacagtttactgccctggagagaacacagcagtaaaaaccaggtttgctactggaaaaagaggaaagagaagactttcattgacggacccagccgcagcgtagcagccctgcgttttagacggcagcagctcgggactctggacgtgtgtttgccctcaagtttgctaagctgctggtttattactgaagaaagaatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 73 - DNA, 1034 bp, synthetic construct
  source 1..1034: mol_type = other DNA
aaagtgattgattcggatactgacactgtaggatctggggagagaggaacaaaggaccgtgaaagctgctctgtaaaagctgacacagccctcccaagtgagcaggactgttcttcccactgcaatctgacagtttactgcatgcctggagagaacacagcagtaaaaaccaggtttgctactggaaaaagaggaaagagaagactttcattgacggacccagccatggcagcgtagcagccctgcgttttagacggcagcagctcgggactctggacgtgtgtttgccctcaagtttgctaagctgctggtttattactgaagaaagaatgggagtgaaagttctttttgcccttatttgtattgctgtggccgaggccgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 74 - DNA, 842 bp, synthetic construct
  source 1..842: mol_type = other DNA
catatcttcattcttcactctttccccccccccccttatcctcctatctaatcctctcatctctctcctcctcttcctccaacaacaactaacaatggcagcaactacaacaatgaaagtctctgccgcccttctgtgcctgctgctcatagcagccaccttcattccccaagggctcgctgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaattttgagtaattttcaattttgtttttgtttttgtttttgttgttgggagttgttgtaatttggtaaatgttggggtcactaaaatcttttgttgcaattttagcacctttttattagatatataaaattgggtttagatgtagttaa

SEQ ID NO: 75 - DNA, 824 bp, synthetic construct
  source 1..824: mol_type = other DNA
catatcttcattcttcactctttccccccccccccttatcctcctatctaatcctctcatctctctcctcctcttcctccaacaacaactaacaatgaaagtctctgccgcccttctgtgcctgctgctcatagcagccaccttcattccccaagggctcgctgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaattttgagtaattttcaattttgtttttgtttttgtttttgttgttgggagttgttgtaatttggtaaatgttggggtcactaaaatcttttgttgcaattttagcacctttttattagatatataaaattgggtttagatgtagttaa

SEQ ID NO: 76 - DNA, 809 bp, synthetic construct
  source 1..809: mol_type = other DNA
catatcttcattcttcactctttccccccccccccttatcctcctatctaatcctctcatctctctcctcctcttcctccaacaacaactaacaatgtggcagattgttttctttactctgagctgtgatcttgtcttggccgcagccgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaattttgagtaattttcaattttgtttttgtttttgtttttgttgttgggagttgttgtaatttggtaaatgttggggtcactaaaatcttttgttgcaattttagcacctttttattagatatataaaattgggtttagatgtagttaa

SEQ ID NO: 77 - DNA, 1083 bp, synthetic construct
  source 1..1083: mol_type = other DNA
ataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcgaatccagtgttccgtttccagcccccaatctcagagcggagccgacagagagcagggaaccggcatgaaagtctctgccgcccttctgtgcctgctgctcatagcagccaccttcattccccaagggctcgctgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaagggcctttccaagattgctgtttttgttttggagcttcaagactttgcatttcctagtatttctgtttgtcagttctcaatttcctgtgtttgcaatgttgaaattttttggtgaagtactgaacttgctttttttccggtttctacatgcagagatgaatttatactgccatcttacgactatttcttctttttaatacacttaactcaggccattttttaagttggttacttcaaagtaaataaactttaaaattc

SEQ ID NO: 78 - DNA, 984 bp, synthetic construct
  source 1..984: mol_type = other DNA
gggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctggcaagatgaaagtctctgccgcccttctgtgcctgctgctcatagcagccaccttcattccccaagggctcgctgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 79 - DNA, 898 bp, synthetic construct
  source 1..898: mol_type = other DNA
agctgttcttggctgacttcacatcaaaactcctatactgacctgagacagaggcagcagtgatacccacctgagagatcctgtgtttgaacaactgcttcccaaaacggaaagtatttcaagcctaaacctttgggtgaaaagaactcttgaagtcatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 80 - DNA, 1060 bp, synthetic construct
  source 1..1060: mol_type = other DNA
caagcttagcctggccgggaaacgggaggcgtggaggccgggagcagcccccggggtcatcgccctgccaccgccgcccgattgctttagcttggaaattccggagctgaagcggccagcgagggaggatgaccctctcggcccgggcaccctgtcagtccggaaataactgcagcatttgttccggaggggaaggcgcgaggtttccgggaaagcagcaccgccccttggcccccaggtggctagcgctataaaggatcacgcgccccagtcgacgctgagctcctctgctactcagagttgcaacctcagcctcgctatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 81 - DNA, 1150 bp, synthetic construct
  source 1..1150: mol_type = other DNA
gtctcaatattagagtctcaacccccaataaatataggactggagatgtctgaggctcattctgccctggagcccaccgggaacgaaagagaagctctatctcccctccaggagcccagctatgaactccttctccacaagcgccttcggtccagttgccttctccctggggctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaacatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaa

SEQ ID NO: 82 - DNA, 975 bp, synthetic construct
  source 1..975: mol_type = other DNA
gtggcagctcacagctattgtggtgggaaagggagggtggttggtggatgtcacagcttgggctttatctcccccagcagtggggactccacagcccctgggctacataacagcaagacagtccggagctgtagcagacctgattgagcctttgcagcagctgagagcatggcctagggtgggcggcaccattgtccagcagctgagtttcccagggaccttggagatagccgcagccctcatttgcaggggaagatgattcctgccagatttgccggggtgctgcttgctctggccctcattttgccagggaccctttgtgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 83 - DNA, 497 bp, Homo sapiens
  source 1..497: mol_type = genomic DNA
gccctaagccatcagcaatccttagtataggggcacactcatgcattcctgtcaagtcatcttgtgaaaggctgcctgcttccagcttggcttggatgtgcaaccttaataaaactcactgaggtctgggagaaaatagcagatctgcagcagatagggtagaggaaagggtctagaatatgtacacgcagctgactcaggcaggctccatgctgaacggtcacacagagaggaaacaataaatctcagctactatgcaataaatatctcaagttttaacgaagaaaaacatcattgcagtgaaataaaaaattttaaaattttagaacaaagctaacaaatggctagttttctatgattcttcttcaaacgctttctttgagggggaaagagtcaaacaaacaagcagttttacctgaaataaagaactagttttagaggtcagaagaaaggagcaagttttgcgagaggcacggaaggagtgtgctggcagtaca

SEQ ID NO: 84 - DNA, 45 bp, Homo sapiens
  source 1..45: mol_type = genomic DNA
atgacagttttcctttcctttgctttcctcgctgccattctgact

SEQ ID NO: 85 - protein, 15 aa, Homo sapiens
MTVFLSFAFLAAILT

SEQ ID NO: 86 - DNA, 1452 bp, Homo sapiens
  source 1..1452: mol_type = genomic DNA
cacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttga

SEQ ID NO: 87 - protein, 483 aa, Homo sapiens
HIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 88 - DNA, 2373 bp, Homo sapiens
  source 1..2373: mol_type = genomic DNA
aagcgcaatgtcagaagcgattatgaaagcaacaaagaaatccggagaagctgccaggtgagaaactgtttgaaaacttcagaagcaaacaatattgtctcccttccagcaataagtggtagttatgtgaagtcaccaaggttcttgaccgtgaatctggagccgtttgagttcacaagagtctctacttggggtgacagtgctcacgtggctcgactatagaaaactccactgactgtcgggctttaaaaagggaagaaactgctgagcttgctgtgcttcaaactactactggaccttattttggaactatggtagccagatgataaatatggttaatttcatgtaaaacagaaaaaaagagtgaaaaagagaatatacatgaagaatagaaacaagcctgccataatcctttggaaaagatgtattataccagtgaaaaggtgttatatctatgcaaacctactaacaaattatactgttgcacaattttgataaaaatttagaacagcattgtcctctgagttggttaaatgttaatggatttcagaagcctaattccagtatcatacttactagttgatttctgcttacccatcttcaaatgaaaattccatttttgtaagccataatgaactgtagtacatggacaataagtgtgtggtagaaacaaactccattactctgatttttgatacagttttcagaaaaagaaatgaacataatcaagtaaggatgtatgtggtgaaaacttaccacccccatactatggttttcatttactctaaaaactgattgaatgatatataaatatatttatagcctgagtaaagttaaaagaatgtaaaatatatcatcaagttcttaaaataatatacatgcatttaatatttcctttgatattatacaggaaagcaatattttggagtatgttaagttgaagtaaaagcaagtactctggagcagttcattttacagtatctacttgcatgtgtatacatacatgtaacttcattattttaaaaatatttttagaactccaatactcaccctgttatgtcttgctaatttaaattttgctaattaactgaaacatgcttaccagattcacactgttccagtgtctataaaagaaacactttgaagtctataaaaaataaaataattataaatatcattgtacatagcatgtttatatctgcaaaaaacctaatagctaattaatctggaatatgcaacattgtccttaattgatgcaaataacacaaatgctcaaagaaatctactatatcccttaatgaaatacatcattcttcatatatttctccttcagtccattcccttaggcaatttttaatttttaaaaattattatcaggggagaaaaattggcaaaactattatatgtaagggaaatatatacaaaaagaaaattaatcatagtcacctgactaagaaattctgactgctagttgccataaataactcaatggaaatattcctatgggataatgtattttaagtgaatttttggggtgcttgaagttactgcattattttatcaagaagtcttctctgcctgtaagtgtccaaggttatgacagtaaacagtttttattaaaacatgagtcactatgggatgagaaaattgaaataaagctactgggcctcctctcataaaagagacagttgttggcaaggtagcaataccagtttcaaacttggtgacttgatccactatgccttaatggtttcctccatttgagaaaataaagctattcacattgttaagaaaaatactttttaaagtttaccatcaagtcttttttatatttatgtgtctgtattctacccctttttgccttacaagtgatatttgcaggtattataccatttttctattcttggtggcttcttcatagcaggtaagcctctccttctaaaaacttctcaactgttttcatttaagggaaagaaaatgagtattttgtccttttgtgttcctacagacactttcttaaaccagtttttggataaagaatactatttccaaactcatattacaaaaacaaaataaaataataaaaaaagaaagcatgatatttactgttttgttgtctgggtttgagaaatgaaatattgtttccaattatttataataaatcagtataaaatgttttatgattgttatgtgtattatgtaatacgtacatgtttatggcaatttaacatgtgtattcttttaattgtttcagaataggataattaggtattcgaattttgtctttaaaattcatgtggtttctatgcaaagttcttcatatcatcacaacattatttgatttaaataaaattgaaagtaatatttgtgcaa

SEQ ID NO: 89 - DNA, 120 bp, Homo sapiens
  source 1..120: mol_type = genomic DNA
agacagaggtttgtagctgcagctgcaggcaagcctggccactgttggctgcagcaggacatcccaggcacagcccctagggctctgagcagacatccctcgccattgacacatcttcag

SEQ ID NO: 90 - DNA, 66 bp, Homo sapiens
  source 1..66: mol_type = genomic DNA
atgctctcccagctagccatgctgcagggcagcctcctccttgtggttgccaccatgtctgtggct

SEQ ID NO: 91 - protein, 22 aa, Homo sapiens
MLSQLAMLQGSLLLVVATMSVA

SEQ ID NO: 92 - DNA, 1446 bp, Homo sapiens
  source 1..1446: mol_type = genomic DNA
caacagacaaggcaggaggcggataggggctgcgagacacttgtagtccagcacggccactgtagctacaccttcttgctgcccaagtctgagccctgccctccggggcctgaggtctccagggactccaacaccctccagagagaatcactggccaacccactgcacctggggaagttgcccacccagcaggtgaaacagctggagcaggcactgcagaacaacacgcagtggctgaagaagctagagagggccatcaagacgatcttgaggtcgaagctggagcaggtccagcagcaaatggcccagaatcagacggcccccatgctagagctgggcaccagcctcctgaaccagaccactgcccagatccgcaagctgaccgacatggaggctcagctcctgaaccagacatcaagaatggatgcccagatgccagagacctttctgtccaccaacaagctggagaaccagctgctgctacagaggcagaagctccagcagcttcagggccaaaacagcgcgctcgagaagcggttgcaggccctggagaccaagcagcaggaggagctggccagcatcctcagcaagaaggcgaagctgctgaacacgctgagccgccagagcgccgccctcaccaacatcgagcgcggcctgcgcggtgtcaggcacaactccagcctcctgcaggaccagcagcacagcctgcgccagctgctggtgttgttgcggcacctggtgcaagaaagggctaacgcctcggccccggccttcataatggcaggtgagcaggtgttccaggactgtgcagagatccagcgctctggggccagtgccagtggtgtctacaccatccaggtgtccaatgcaacgaagcccaggaaggtgttctgtgacctgcagagcagtggaggcaggtggaccctcatccagcgccgtgagaatggcaccgtgaattttcagcggaactggaaggattacaaacagggcttcggagacccagctggggagcactggctgggcaatgaagtggtgcaccagctcaccagaagggcagcctactctctgcgtgtggagctgcaagactgggaaggccacgaggcctatgcccagtacgaacatttccacctgggcagtgagaaccagctatacaggctttctgtggtcgggtacagcggctcagcagggcgccagagcagcctggtcctgcagaacaccagctttagcacccttgactcagacaacgaccactgtctctgcaagtgtgcccaagtgatgtctggagggtggtggtttgacgcctgtggcctgtcaaacctcaacggcgtctactaccacgctcccgacaacaagtacaagatggacggcatccgctggcactacttcaagggccccagctactcactgcgtgcctctcgcatgatgatacggcctttggacatctaa

SEQ ID NO: 93 - protein, 481 aa, Homo sapiens
QQTRQEADRGCETLVVQHGHCSYTFLLPKSEPCPPGPEVSRDSNTLQRESLANPLHLGKLPTQQVKQLEQALQNNTQWLKKLERAIKTILRSKLEQVQQQMAQNQTAPMLELGTSLLNQTTAQIRKLTDMEAQLLNQTSRMDAQMPETFLSTNKLENQLLLQRQKLQQLQGQNSALEKRLQALETKQQEELASILSKKAKLLNTLSRQSAALTNIERGLRGVRHNSSLLQDQQHSLRQLLVLLRHLVQERANASAPAFIMAGEQVFQDCAEIQRSGASASGVYTIQVSNATKPRKVFCDLQSSGGRWTLIQRRENGTVNFQRNWKDYKQGFGDPAGEHWLGNEVVHQLTRRAAYSLRVELQDWEGHEAYAQYEHFHLGSENQLYRLSVVGYSGSAGRQSSLVLQNTSFSTLDSDNDHCLCKCAQVMSGGWWFDACGLSNLNGVYYHAPDNKYKMDGIRWHYFKGPSYSLRASRMMIRPLDI

SEQ ID NO: 94 - DNA, 3061 bp, Homo sapiens
  source 1..3061: mol_type = genomic DNA
cgagcagctgtgccagaggctggaccacacaggagaagctcggacttggcactcctggacaacctggacccagatgcaagacactgtgccaccgccttccctgacaccctgggcttcctgagccagccctccttgacccagaagtccagaagggtcatctgccccccaactcccctccgtctgtgacatggagggtgttcggggcccatccctctgatgtagtcctcgcccctcttctctccctcccccttcaggggctccctgcctgagggtcacagtaccttgaatgggctgagaacagaccaaacttgattcccatgaccaatggtggggttgcaggcaggtgggaatgtatttgcacatcggaagctgcccagatggcccaggttctctcccttggattggcaagaaggccatctcccattctaagctcctgttccaagattttctagtcttgagatgtccttgaactttcttttcaagtctgaaggggctgcatccaccccttagtgggtgggttaatcattatttccccttcacacttcaccacttctaggttctaatgaccctagatctcagggtctttagacttcaccacttctaggctttaccacttcaccacttctaggctccaatgtttggagctcagggtctttaggagacccaaaaggacatgctccttcacctccagcatgtcctagaggatgtgtcacagggaataactatggcttgtctctaaaagtacctatgagcaatgagaaaaggaaacagcaggttaagtcaaagtgaacaggcactcttcactgcaggactgatcagagcctttaatatggccaagtgccttgtgactacccatgaaggggctagagtgggcagctttctccaaatttacttatttgaaaatgggctcggtttgtcccagagcatctcacaggactgtagatgctcttggacaaagctagtgctcccctggcataaggaggagccctacgaccccatccccaccccagctatactcaccctttttggctacaagggccacagtgacagcctcaaacaacctctaaaaacaactggaaataacctttcagttaaaacagataccatccctgaagaagggtctagaactaggtccctgtctgtgttataggctcatgtcctccaaggctccttcaagtcccaggaagctgatctctacctgggtggcttcccttaggactccctgtaacctcaactcccccaggctcaattacagggactgttaggcaggacatctgtctccaagtccagatcctctctgcctccaagccctaacccctagcctccctcccttccccatccagcagtgatgctgcctctgtggtggtaggtggggagctgcaggggaggagataaggcctctgcctgagtttgggagaccagggccctcatagcttctttcagaggatggagtcagaaaggatccacagctactctgtcacctgcccccatcactgtgtcatgctgtctgccctgttgtcatcagccaacacccaggcatagccaggagcccacctgccctaccgccaggatacacctctgtcctcagaaggttttctcctggatgagactgagccaatgggaatgggaccccttcatccccctggctcgccccagccctgagtcccactctcagccgatccctgagtaaacccagcacagactgactttgatctcattcctgggaattagcactcttccccttcaagactcaaaggacatggttgctaatggtggcatttcaggcatgatgggaaatctttaggggcagattgctgcccagagagctcaaatcgccttaagcagcatttgcccagcagacctttatttagcctctactgtgtgcagtgtggtgtggtgggcagggctttggagtcggacaaacctgctccagctctgacactttggtccagtggctcagcctctcaaggcaccagttatcttcacatcatcaaagcctcagttttcccatctgtaaaatggagatgataatattccttcctggctgggctatggcaaggaggaaatgagaccatgtatgtcatcttcttaatagagcctggcatgaagcaggtgcctaataaatgtttgtcctcaaagaggagaatggggtgaggaaggcattccccagcacatgccgccccttctcctgcactcaggtgaggaaaaggcattttatttttgtatccacatcatttatttttctattgtagtttctaggctgactgcaagctagagaggagacagggcaaagctgtgaggcccagggacagaactcctctgggtgggttgaaggcccaagtccctctctactcccattttataagggggcaggaagctgatttgagttatcctcagacacctgttctttatgtaattttattttatttttttgagacagagtctcactctgtcacccaggctggagtgcagtggcatgatctcagatcactgcaatctctgcctcctggttcaagtgattctcctacctcagcctcctgagtaatgggattacagacgcctaccaccacgcccggaaaacttttgtatttttagtagaaacgggttttcaccatgttggccaggctggtctcaaactcctggcctcatgtgatccacctgcctcagcctcccaaagtgctgggattacaggcatgagccaccatacccagcctcagacacctgttcttaaatattcatccttctttcttaccttccttcctcttccatgccaggactcaggtataagggatagaaattctagccctaaggaataaattgactcacataactggaaagtctaagggtaaaggcaagtgaggttagatccagaggctcaaatgatgtcagctccacctctcagcccctccatctgccccgttgacttcattctcagccaggatctttcctcacaagaaggctctggcagccccaggctcatgtcctcccagctcagcatccctgacccggggagctccctcgtctccatgattccagtaaaggaatgattttctgcagccag

SEQ ID NO: 95 - DNA, 867 bp, synthetic construct
  source 1..867: mol_type = other DNA
gggatcttagacctagccccacagatgcttcgagaactccaggagactaatgcggcgctgcaagacgtgagagagctcttgcgacagcaggtcaaggagatcaccttcctgaagaatacggtgatggaatgtgacgcttgcggaggctcccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttga

SEQ ID NO: 96 - protein, 288 aa, synthetic construct
  REGION 1..288: note = Synthetic Construct
GILDLAPQMLRELQETNAALQDVRELLRQQVKEITFLKNTVMECDACGGSLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 97 - DNA, 867 bp, synthetic construct
  source 1..867: mol_type = other DNA
gggatcttagacttgggcccgcagatgcttcgggaactgcaggaaaccaacgcggcgctgcaggacgtgcgggagctgctgcggcagcaggtcagggagatcacgttcctgaaaaacacggtgatggagtgtgacgcgtgcggaggctcccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttga

SEQ ID NO: 98 - protein, 288 aa, synthetic construct
  REGION 1..288: note = Synthetic Construct
GILDLGPQMLRELQETNAALQDVRELLRQQVREITFLKNTVMECDACGGSLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 99 - DNA, 861 bp, synthetic construct
  source 1..861: mol_type = other DNA
gggatcttagaggaagacccggccgcctgcgagtccctggtgaaattccaagccaaagtggaggggctgctgcaggccctgaccaggaaactggaagctgtgagtaagcggctggccatcctggagaacacagttgtcggatcccttgtcaatcttgccactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttga

SEQ ID NO: 100 - DNA, 870 bp, synthetic construct
  source 1..870: mol_type = other DNA
gggatcttagacctagccccacagatgcttcgagaactccaggagactaatgcggcgctgcaagacgtgagagagctcttgcgacagcaggtcaaggagatcaccttcctgaagaatacggtgatggaatgtgacgcttgcggaggctccttactgactatgatgtccacatcaaactcagctaaggaccccactgttgctaaagaagaacaaatcagcttcagagactgtgctgaagtattcaaatcaggacacaccacgaatggcatctacacgttaacattccctaattctacagaagagatcaaggcctactgtgacatggaagctggaggaggcgggtggacaattattcagcgacgtgaggatggcagcgttgattttcagaggacttggaaagaatataaagtgggatttggtaacccttcaggagaatattggctgggaaatgagtttgtttcgcaactgactaatcagcaacgctatgtgcttaaaatacaccttaaagactgggaagggaatgaggcttactcattgtatgaacatttctatctctcaagtgaagaactcaattataggattcaccttaaaggacttacagggacagccggcaaaataagcagcatcagccaaccaggaaatgattttagcacaaaggatggagacaacgacaaatgtatttgcaaatgttcacaaatgctaacaggaggctggtggtttgatgcatgtggtccttccaacttgaacggaatgtactatccacagaggcagaacacaaataagttcaacggcattaaatggtactactggaaaggctcaggctattcgctcaaggccacaaccatgatgatccgaccagcagatttctaa

SEQ ID NO: 101 - DNA, 3375 bp, Homo sapiens
  source 1..3375: mol_type = genomic DNA
atggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggatccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccaccggatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagcttctatacaaacccgttaatcactatgaggcttggcaacatattcaagtgacaaatgagattgttacactcaactatttggaacctcggacagaatatgaactctgtgtgcaactggtccgtcgtggagagggtggggaagggcatcctggacctgtgagacgcttcacaacagcttctatcggactccctcctccaagaggtctaaatctcctgcctaaaagtcagaccactctaaatttgacctggcaaccaatatttccaagctcggaagatgacttttatgttgaagtggagagaaggtctgtgcaaaaaagtgatcagcagaatattaaagttccaggcaacttgacttcggtgctacttaacaacttacatcccagggagcagtacgtggtccgagctagagtcaacaccaaggcccagggggaatggagtgaagatctcactgcttggacccttagtgacattcttcctcctcaaccagaaaacatcaagatttccaacattacacactcctcagctgtgatttcttggacaatattggatggctattctatttcttctattactatccgttacaaggttcaaggcaagaatgaagaccagcacgttgatgtgaagataaagaatgccaccatcactcagtatcagctcaagggcctagagcctgaaacagcataccaggtggacatttttgcagagaacaacatagggtcaagcaacccagccttttctcatgaactggtgaccctcccagaatctcaagcaccagcggacctcggaggggggaagatgctgcttatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctag

SEQ ID NO: 102 - protein, 1124 aa, Homo sapiens
MDSLASLVLCGVSLLLSGTVEGAMDLILINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIPRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPIFPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATITQYQLKGLEPETAYQVDIFAENNIGSSNPAFSHELVTLPESQAPADLGGGKMLLIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

SEQ ID NO: 103 - DNA, 3375 bp, Homo sapiens
  source 1..3375: mol_type = genomic DNA
atggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggatccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccaccggatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagcttctatacaaacccgttaatcactatgaggcttggcaacatattcaagtgacaaatgagattgttacactcaactatttggaacctcggacagaatatgaactctgtgtgcaactggtccgtcgtggagagggtggggaagggcatcctggacctgtgagacgcttcacaacagcttctatcggactccctcctccaagaggtctaaatctcctgcctaaaagtcagaccactctaaatttgacctggcaaccaatatttccaagctcggaagatgacttttatgttgaagtggagagaaggtctgtgcaaaaaagtgatcagcagaatattaaagttccaggcaacttgacttcggtgctacttaacaacttacatcccagggagcagtacgtggtccgagctagagtcaacaccaaggcccagggggaatggagtgaagatctcactgcttggacccttagtgacattcttcctcctcaaccagaaaacatcaagatttccaacattacacactcctcagctgtgatttcttggacaatattggatggctattctatttcttctattactatccgttacaaggttcaaggcaagaatgaagaccagcacgttgatgtgaagataaagaatgccaccatcactcagtatcagctcaagggcctagagcctgaaacagcataccaggtggacatttttgcagagaacaacatagggtcaagcaacccagccttttctcatgaactggtgaccctcccagaatctcaagcaccagcggacctcggaggggggaagatgctgcttatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttatggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctag

SEQ ID NO: 104 - protein, 1124 aa, Homo sapiens
MDSLASLVLCGVSLLLSGTVEGAMDLILINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIPRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPIFPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATITQYQLKGLEPETAYQVDIFAENNIGSSNPAFSHELVTLPESQAPADLGGGKMLLIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLWMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

SEQ ID NO: 105 - DNA, 814 bp, synthetic construct
  source 1..814: mol_type = other DNA
gaggaaccgagaggctgagactaacccagaaacatccaattctcaaactgaagctcgcactctcgcctccagcatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 106 - DNA, 984 bp, synthetic construct
  source 1..984: mol_type = other DNA
ataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcgaatccagtgttccgtttccagcccccaatctcagagcggagccgacagagagcagggaaccggcatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 107 - DNA, 1735 bp, synthetic construct
  source 1..1735: mol_type = other DNA
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 108 - DNA, 1150 bp, synthetic construct
  source 1..1150: mol_type = other DNA
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctgggatcttagacttgggcccgcagatgcttcgggaactgcaggaaaccaacgcggcgctgcaggacgtgcgggagctgctgcggcagcaggtcagggagatcacgttcctgaaaaacacggtgatggagtgtgacgcgtgcggaggctcccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 109 - DNA, 817 bp, synthetic construct
  source 1..817: mol_type = other DNA
catatcttcattcttcactctttccccccccccccttatcctcctatctaatcctctcatctctctcctcctcttcctccaacaacaactaacaatgaaagtctctgccgcccttctgtgcctgctgctcatagcagccaccttcattccccaagggctcgctgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 110 - DNA, 978 bp, synthetic construct
  source 1..978: mol_type = other DNA
gtggcagctcacagctattgtggtgggaaagggagggtggttggtggatgtcacagcttgggctttatctcccccagcagtggggactccacagcccctgggctacataacagcaagacagtccggagctgtagcagacctgattgagcctttgcagcagctgagagcatggcctagggtgggcggcaccattgtccagcagctgagtttcccagggaccttggagatagccgcagccctcatttgcaggggaagatgaaagtctctgccgcccttctgtgcctgctgctcatagcagccaccttcattccccaagggctcgctgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 111 - DNA, 975 bp, synthetic construct; identical to SEQ ID NO: 82
  source 1..975: mol_type = other DNA

SEQ ID NO: 112 - DNA, 778 bp, synthetic construct
  source 1..778: mol_type = other DNA
gggaaataagagagaaaagaagagtaagaagaaatataagaccccggcgccgccaccatgaaagtctctgccgcccttctgtgcctgctgctcatagcagccaccttcattccccaagggctcgctgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataataggctggagcctcggtggcctagcttcttgccccttgggcctccccccagcccctcctccccttcctgcacccgtaccccctccataaagtaggaaacactacagtggtctttgaataaagtctgagtgggcggc

SEQ ID NO: 113 - DNA, 796 bp, synthetic construct
  source 1..796: mol_type = other DNA
gaggaaccgagaggctgagactaacccagaaacatccaattctcaaactgaagctcgcactctcgccgccaccatgaaagtctctgccgcccttctgtgcctgctgctcatagcagccaccttcattccccaagggctcgctgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 114 - DNA, 1144 bp, synthetic construct
  source 1..1144: mol_type = other DNA
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctgggatcttagaggaagacccggccgcctgcgagtccctggtgaaattccaagccaaagtggaggggctgctgcaggccctgaccaggaaactggaagctgtgagtaagcggctggccatcctggagaacacagttgtcggctcccttgtcaatcttgccactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 115 - DNA, 3589 bp, synthetic construct
  source 1..3589: mol_type = other DNA
gaggaaccgagaggctgagactaacccagaaacatccaattctcaaactgaagctcgcactctcgcctccagcatggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggacccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccacagaatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagcttctatacaaacccgttaatcactatgaggcttggcaacatattcaagtgacaaatgagattgttacactcaactatttggaacctcggacagaatatgaactctgtgtgcaactggtccgtcgtggagagggtggggaagggcatcctggacctgtgagacgcttcacaacagcttctatcggactccctcctccaagaggtctaaatctcctgcctaaaagtcagaccactctaaatttgacctggcaaccaatatttccaagctcggaagatgacttttatgttgaagtggagagaaggtctgtgcaaaaaagtgatcagcagaatattaaagttccaggcaacttgacttcggtgctacttaacaacttacatcccagggagcagtacgtggtccgagctagagtcaacaccaaggcccagggggaatggagtgaagatctcactgcttggacccttagtgacattcttcctcctcaaccagaaaacatcaagatttccaacattacacactcctcagctgtgatttcttggacaatattggatggctattctatttcttctattactatccgttacaaggttcaaggcaagaatgaagaccagcacgttgatgtgaagataaagaatgccaccatcactcagtatcagctcaagggcctagagcctgaaacagcataccaggtggacatttttgcagagaacaacatagggtcaagcaacccagccttttctcatgaactggtgaccctcccagaatctcaagcaccagcggacctcggaggggggaagatgctgcttatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctagggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 116 - DNA, 3589 bp, synthetic construct
  source 1..3589: mol_type = other DNA
gaggaaccgagaggctgagactaacccagaaacatccaattctcaaactgaagctcgcactctcgcctccagcatggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggacccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccacagaatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagcttctatacaaacccgttaatcactatgaggcttggcaacatattcaagtgacaaatgagattgttacactcaactatttggaacctcggacagaatatgaactctgtgtgcaactggtccgtcgtggagagggtggggaagggcatcctggacctgtgagacgcttcacaacagcttctatcggactccctcctccaagaggtctaaatctcctgcctaaaagtcagaccactctaaatttgacctggcaaccaatatttccaagctcggaagatgacttttatgttgaagtggagagaaggtctgtgcaaaaaagtgatcagcagaatattaaagttccaggcaacttgacttcggtgctacttaacaacttacatcccagggagcagtacgtggtccgagctagagtcaacaccaaggcccagggggaatggagtgaagatctcactgcttggacccttagtgacattcttcctcctcaaccagaaaacatcaagatttccaacattacacactcctcagctgtgatttcttggacaatattggatggctattctatttcttctattactatccgttacaaggttcaaggcaagaatgaagaccagcacgttgatgtgaagataaagaatgccaccatcactcagtatcagctcaagggcctagagcctgaaacagcataccaggtggacatttttgcagagaacaacatagggtcaagcaacccagccttttctcatgaactggtgaccctcccagaatctcaagcaccagcggacctcggaggggggaagatgctgcttatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttatggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctagggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 117 - DNA, 3207 bp, Homo sapiens
  source 1..3207: mol_type = genomic DNA
atgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactga

SEQ ID NO: 118 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 119 - DNA, 513 bp, synthetic construct
  source 1..513: mol_type = other DNA
gtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 120 - DNA, 57 bp, synthetic construct
  source 1..57: mol_type = other DNA
gggaaataagagagaaaagaagagtaagaagaaatataagaccccggcgccgccacc

SEQ ID NO: 121 - DNA, 139 bp, synthetic construct
  source 1..139: mol_type = other DNA
taataggctggagcctcggtggcctagcttcttgccccttgggcctccccccagcccctcctccccttcctgcacccgtaccccctccataaagtaggaaacactacagtggtctttgaataaagtctgagtgggcggc

SEQ ID NO: 122 - DNA, 139 bp, synthetic construct
  source 1..139: mol_type = other DNA
ttttttttttttttttttttttttttttttttttttttttttttttttttttttttttttttttttttttttttttttttttttttttttttttttttttttttttttttttttttttttttgcacaaatattactttc

SEQ ID NO: 123 - DNA, 38 bp, synthetic construct
  source 1..38: mol_type = other DNA
taatacgactcactatagggaaataagagagaaaagaa

SEQ ID NO: 124 - DNA, 139 bp, synthetic construct
  source 1..139: mol_type = other DNA
ttttttttttttttttttttttttttttttttttttttttttttttttttttttttttttttttttttttttttttttttttttttttttttttttttttttttttttttttttttttttgccgcccactcagacttta

SEQ ID NO: 125 - DNA, 38 bp, synthetic construct
  source 1..38: mol_type = other DNA
taatacgactcactatagggtggcagctcacagctatt

SEQ ID NO: 126 - DNA, 4683 bp, Homo sapiens
  source 1..4683: mol_type = genomic DNA
ctctgcccctacagcagcagcaaaagcagcagcagaagcaacagcaacagataagtgttttgatgaattgcgagatggatagggcttgagtgcccccagccctgctgataccaaatgcctttaagatacagcctttcccatcctaatctacaaaggaaacaggaaaaaggaacttaaaactccctgtgctcagacagaaatgagactgttacagcctgcttctgtgctgttccttcttgcctctaacttgtaaacaagacgtagtaggacgatgctaatggaaagtcacaaaccgctgggtttttgaaaggatccttgggacctcatgcacatttgtggaaactggatggagagatttggggaagcatggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggatccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcatacagaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccaccggatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagcttctatacaaacccgttaatcactatgaggcttggcaacatattcaagtgacaaatgagattgttacactcaactatttggaacctcggacagaatatgaactctgtgtgcaactggtccgtcgtggagagggtggggaagggcatcctggacctgtgagacgcttcacaacagcttctatcggactccctcctccaagaggtctaaatctcctgcctaaaagtcagaccactctaaatttgacctggcaaccaatatttccaagctcggaagatgacttttatgttgaagtggagagaaggtctgtgcaaaaaagtgatcagcagaatattaaagttccaggcaacttgacttcggtgctacttaacaacttacatcccagggagcagtacgtggtccgagctagagtcaacaccaaggcccagggggaatggagtgaagatctcactgcttggacccttagtgacattcttcctcctcaaccagaaaacatcaagatttccaacattacacactcctcagctgtgatttcttggacaatattggatggctattctatttcttctattactatccgttacaaggttcaaggcaagaatgaagaccagcacgttgatgtgaagataaagaatgccaccatcactcagtatcagctcaagggcctagagcctgaaacagcataccaggtggacatttttgcagagaacaacatagggtcaagcaacccagccttttctcatgaactggtgaccctcccagaatctcaagcaccagcggacctcggaggggggaagatgctgcttatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctaggacagaacatctgtataccctctgtttccctttcactggcatgggagacccttgacacctgctgagaaaacatgcctctgccaaaggatgtgatatataagtgtacatatgtgctgtacacctgggaccttcaccactgtagatcccatgcatggatctatgtagtatgctctgactctaataggactgtatatactgttttaagaatgggctgaaatcagaatgcctgtttgtggtttcatatgcaataatatatttttttaaaaatgtggacttcataggaaggcgtgagtacaattagtataatgcataactcattgttgtcctagatattttgatatttacctttatgttgaatgctattaaatgttttcctgtgtcaaagtaaaatattgttaataaacctaacaatgaccctgatagtacaggttaagtgagagaactatatgaattctaacaagtcataggttaatatttaagacactgaaaaatctaagtgatataaatcagattcttctctctcaattttatccctcacctgtagcagccagtcccgtttcatttagtcatgtgaccactctgtcttgtgtttccacagcctgcaagtcagtccaggatgctaacatctaaaaatagacttaaatctcattgcttacaagcctaagaatctttagagaagtatacataagtttaggataaaataatgggattttcttttcttttctctggtaatattgacttgtatattttaagaaataacagaaagcctgggtgacatttgggagacatgtgacatttatatattgaattaatatccctacatgtattgcacattgtaaaaagttttagttttgatgagttgtgagtttaccttgtatactgtaggcacactttgcactgatatatcatgagtgaataaatgtcttgcctactcacgtctca

SEQ ID NO: 127 - protein, 1124 aa, Homo sapiens
  REGION 1..1124: note = MISC_FEATURE - Tie-2, wild type
MDSLASLVLCGVSLLLSGTVEGAMDLILINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIQRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPIFPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATITQYQLKGLEPETAYQVDIFAENNIGSSNPAFSHELVTLPESQAPADLGGGKMLLIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

SEQ ID NO: 128 - DNA, 3589 bp, synthetic construct
  source 1..3589: mol_type = other DNA
gaggaaccgagaggctgagactaacccagaaacatccaattctcaaactgaagctcgcactctcgcctccagcatggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggacccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccacagaatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagcttctatacaaacccgttaatcactatgaggcttggcaacatattcaagtgacaaatgagattgttacactcaactatttggaacctcggacagaatatgaactctgtgtgcaactggtccgtcgtggagagggtggggaagggcatcctggacctgtgagacgcttcacaacagcttctatcggactccctcctccaagaggtctaaatctcctgcctaaaagtcagaccactctaaatttgacctggcaaccaatatttccaagctcggaagatgacttttatgttgaagtggagagaaggtctgtgcaaaaaagtgatcagcagaatattaaagttccaggcaacttgacttcggtgctacttaacaacttacatcccagggagcagtacgtggtccgagctagagtcaacaccaaggcccagggggaatggagtgaagatctcactgcttggacccttagtgacattcttcctcctcaaccagaaaacatcaagatttccaacattacacactcctcagctgtgatttcttggacaatattggatggctattctatttcttctattactatccgttacaaggttcaaggcaagaatgaagaccagcacgttgatgtgaagataaagaatgccaccatcactcagtatcagctcaagggcctagagcctgaaacagcataccaggtggacatttttgcagagaacaacatagggtcaagcaacccagccttttctcatgaactggtgaccctcccagaatctcaagcaccagcggacctcggaggggggaagatgctgcttatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttcttccgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctagggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 129 - DNA, 3589 bp, synthetic construct
  source 1..3589: mol_type = other DNA
gaggaaccgagaggctgagactaacccagaaacatccaattctcaaactgaagctcgcactctcgcctccagcatggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggacccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccacagaatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagcttctatacaaacccgttaatcactatgaggcttggcaacatattcaagtgacaaatgagattgttacactcaactatttggaacctcggacagaatatgaactctgtgtgcaactggtccgtcgtggagagggtggggaagggcatcctggacctgtgagacgcttcacaacagcttctatcggactccctcctccaagaggtctaaatctcctgcctaaaagtcagaccactctaaatttgacctggcaaccaatatttccaagctcggaagatgacttttatgttgaagtggagagaaggtctgtgcaaaaaagtgatcagcagaatattaaagttccaggcaacttgacttcggtgctacttaacaacttacatcccagggagcagtacgtggtccgagctagagtcaacaccaaggcccagggggaatggagtgaagatctcactgcttggacccttagtgacattcttcctcctcaaccagaaaacatcaagatttccaacattacacactcctcagctgtgatttcttggacaatattggatggctattctatttcttctattactatccgttacaaggttcaaggcaagaatgaagaccagcacgttgatgtgaagataaagaatgccaccatcactcagtatcagctcaagggcctagagcctgaaacagcataccaggtggacatttttgcagagaacaacatagggtcaagcaacccagccttttctcatgaactggtgaccctcccagaatctcaagcaccagcggacctcggaggggggaagatgctgcttatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttatggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttcttccgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctagggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 130 - DNA, 3589 bp, synthetic construct
  source 1..3589: mol_type = other DNA
gaggaaccgagaggctgagactaacccagaaacatccaattctcaaactgaagctcgcactctcgcctccagcatggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggacccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccacagaatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagcttctatacaaacccgttaatcactatgaggcttggcaacatattcaagtgacaaatgagattgttacactcaactatttggaacctcggacagaatatgaactctgtgtgcaactggtccgtcgtggagagggtggggaagggcatcctggacctgtgagacgcttcacaacagcttctatcggactccctcctccaagaggtctaaatctcctgcctaaaagtcagaccactctaaatttgacctggcaaccaatatttccaagctcggaagatgacttttatgttgaagtggagagaaggtctgtgcaaaaaagtgatcagcagaatattaaagttccaggcaacttgacttcggtgctacttaacaacttacatcccagggagcagtacgtggtccgagctagagtcaacaccaaggcccagggggaatggagtgaagatctcactgcttggacccttagtgacattcttcctcctcaaccagaaaacatcaagatttccaacattacacactcctcagctgtgatttcttggacaatattggatggctattctatttcttctattactatccgttacaaggttcaaggcaagaatgaagaccagcacgttgatgtgaagataaagaatgccaccatcactcagtatcagctcaagggcctagagcctgaaacagcataccaggtggacatttttgcagagaacaacatagggtcaagcaacccagccttttctcatgaactggtgaccctcccagaatctcaagcaccagcggacctcggaggggggaagatgctgcttatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggcttcttgtacctggccattgagtacgcgccccatggaaaccttctggacttccttctgaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctagggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 131 - DNA, 3589 bp, synthetic construct
  source 1..3589: mol_type = other DNA
gaggaaccgagaggctgagactaacccagaaacatccaattctcaaactgaagctcgcactctcgcctccagcatggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggacccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccacagaatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagcttctatacaaacccgttaatcactatgaggcttggcaacatattcaagtgacaaatgagattgttacactcaactatttggaacctcggacagaatatgaactctgtgtgcaactggtccgtcgtggagagggtggggaagggcatcctggacctgtgagacgcttcacaacagcttctatcggactccctcctccaagaggtctaaatctcctgcctaaaagtcagaccactctaaatttgacctggcaaccaatatttccaagctcggaagatgacttttatgttgaagtggagagaaggtctgtgcaaaaaagtgatcagcagaatattaaagttccaggcaacttgacttcggtgctacttaacaacttacatcccagggagcagtacgtggtccgagctagagtcaacaccaaggcccagggggaatggagtgaagatctcactgcttggacccttagtgacattcttcctcctcaaccagaaaacatcaagatttccaacattacacactcctcagctgtgatttcttggacaatattggatggctattctatttcttctattactatccgttacaaggttcaaggcaagaatgaagaccagcacgttgatgtgaagataaagaatgccaccatcactcagtatcagctcaagggcctagagcctgaaacagcataccaggtggacatttttgcagagaacaacatagggtcaagcaacccagccttttctcatgaactggtgaccctcccagaatctcaagcaccagcggacctcggaggggggaagatgctgcttatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggcttcttgtacctggccattgagtacgcgccccatggaaaccttctggacttccttctgaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataaccccctttatgagaagtttacttatgcaggaattgactgttctgctgaagaagcggcctagggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 132 - DNA, 9259 bp, Homo sapiens
  source 1..9259: mol_type = genomic DNA
agttccggtgccgccgctgcggccgctgaggtgtcgggctgctgctgccgcggccgctgggactggggctggggccgccggcgaggcagggctcgggcccggccgggcagctccggagcggcgggggagaggggccgggaggcgggggccgtgccgcccgctctcctctccctcggcgccgccgccgccgcccgcggggctgggacccgatgcggttagagccgcggagcctggaagagccccgagcgtttctgctttgggacaaccatacatctaattccttaaagtagttttatatgtaaaacttgcaaagaatcagaacaatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaataaaatactatataatttaaataatgtaaacgcaaacagggtttgatagcacttaaactagttcatttcaaaattaagctttagaataatgcgcaatttcatgttatgccttaagtccaaaaaggtaaactttgaagattgtttgtatctttttttaaaaaacaaaacaaaacaaaaatccccaaaatatatagaaatgatggagaaggaaaaagtgatggttttttttgtcttgcaaatgttctatgttttgaaatgtggacacaacaaaggctgttattgcattaggtgtaagtaaactggagtttatgttaaattacattgattggaaaagaatgaaaatttcttatttttccattgctgttcaatttatagtttgaagtgggtttttgactgcttgtttaatgaagaaaaatgcttggggtggaagggactcttgagatttcaccagagactttttctttttaataaatcaaaccttttgatgatttgaggttttatctgcagttttggaagcagtcacaaatgagacctgttataaggtggtatttttttttttcttctggacagtatttaaaggatcttattcttatttcccagggaaattctgggctcccacaaagtaaaaaaaaaaaaaaatcatagaaaaagaatgagcaggaatagttcttattccagaattgtacagtattcaccttaagttgattttttttctccttctgcaattgaactgaatacatttttcatgcatgttttccagaaaatagaagtattaatgttattaaaaagattattttttttattaaaggctatttatattatagaaactatcattaatatatattctttatttacatgatctgtcccatagtcatgcattgttttgcaccccaaattttttattgttcatagcagcatggtcagctttcttcttgatctatagatgaggctcaggcactatcccatttataccaataaccagtgtataactacttaaggaaaacataaaaacttcatcttctttccttttatttcttatgtgaatctcccgtcttccattctcttttataattgagaatgtctcaatcatatgaaattagttaccagaattaacacaatttagactatcttcctgattccttaaacccctttactgaagtatactcatgaataatactttaaaatatgggggaatagaaaccatgaactttttacctttttaaactatttatccatatctccaaagtagaacattaaaccattttaagatatgtctcattcccaagtagtcagagctcactctccaactttattaaatactatttgagcacaggacacattcttaaacattttgaaaaacattaacccaagatgtagaggctactgctagtcgtcattctagaatctgatattttactctgtatttgaaatgaatgattaatgtcctaggaaattagctttagcagatgtccaggtgccacatcaaaaaagtgcaataattattgacagttttttagattaggcatattattggaaaacaactttataaagagtgaacattgtatactctagtaaaacagcatcactttaaaaatattcatttatgaaatctgttacctatagttgaagtcttgagtagtgaacaagggactctaataccaatactcttaatatctggctattttagatcccttaaagggcataattattggaaatttaggtatttcactaaagcatgtatataatattgccaacaagaaaagtaaatttgaagattaagggaacttacttctgcaaactgtcttgcgatagttaagcagaatttaaactctgttttaagcaggaaaccagaaagattattttgcagttgtagaagatttcataacttattaaaacttattaacattttgtgttgtttagatataggcagttgatacatactaacatcccagccttttcaatatcagggttaaattataggaaaactcagtaaaatggtacaaatctgaaagtttgatggtagaaactgaagatttaacagagaactgtgttttacccgagtgccaaaaatgctgtgagcctccttgcacaaaatttataccacttttgcatttttatctatcagtccagatagttgtctcccctccttctcccaggacctctccaccattaaaatgcacaaaccacatggccgatttcaccatttacatttattttcaaaagttactacaaccaaattaattctattagaagaaatgtagacaaattctataaagactatagattgtgacctaagaaagaaatgaggcaaagaaccaaacattgaattaaatgctacatgggtgactaagatctgtttcaagtcagtgataatatagccacttctgggtacttcagtatcagagatcagttctcgtggtttagacagttcctatctatagctgactatccttgtccttgaatatggtgtaactgactattggctctacagttttattgggccacttaagaaatatttccttgaataattattttgagaaaaagtctaaaagtaataaaaataattttaaacacactgtagtaagaaatgactgttggaaaattatgctttcactttctaccatattctcagctatacaaaaccatttattttgaagatttttagactactgttaatttgaaatctgttactcttattgtggaatttgtttttttaaaaaagatgtttctaattggatttttaaaagaagaatggaatttggttgctattttacaatagaacctaagctttttgtggttcttagtgtcctatgtaaaacttagtgtcaaagtaatcaactttgagattttcccttctattctgctttatattaaaagcccattagaaaatgggaacctggtgaatatataatgaattgtaaaatattttaatgtgtaactttttcaactgtgaaactgacttgattttttgatgaaaacagctgctgataaagtattttgtgtaaagtgtagttcttattaatcaggaaaatgatgacttgattagactgtatatgccctcttggattttattttaaatggattggtgactttcacataggtaaaacacagtccatctgtattcttttttccatcaaaaatcgagtgatttggaattataaaaaaattgtgagcagcctatttgaaaggcatcatggaaatttcacagcacaataacacggatttgttttttcttaatgatgtaaatccgtttaattcatactttgatcaatagcccatgcttgccaactctgaagaaatttaatttccagcagtattttaaagctagcctgttaactttttctgaatatttaaagttcctcttttttctatgtctgcacaaactgcagacctgggctggacccacatactcaagagtccaccttaagaaattattttgatgtccaagacatcactaaaatatttaagtttaaagataatatgtggtgttaatagattgtggtgcttttactatttaaagacaactttcatacttcagatgtttttgagaagaggggaatgtgaggggagggggcagaacagggaggagttgtttgaatgaattacattctttatatccatcctgctcatttggggcatgtctttaagagaaggctgaaagttgtgagagtatattgtataccgtaagagaatcaactcttcatcatggatgggattgtgaaggctgaactataaaattcagcattgacagcatcctcaattaataattcttggtgacagaataatacagctgggctgttttttaaaatataaacaataccatttttaattattacattaaaaattgtaaatatatctatgtgccatggcctgggaagcctgctttcttttttcataaaaattatttttactgtatgaaaagatcatggggtttagctcaaaatatctgtggtcctgataaaattggattggtaactctacctcagaaggaaaatgggaaaaaaaaatagatgagtcacaattcaatacttcaagctcagaaactgtgcagatcactgaattttagatttataaagtcagagttggcatgccttgtttttaatgatatggaagaccttaagaaaaaaacttggctgaagtttaatcgttggtccagccatttgaaaaaggcaatagtttgaggaggttcccgaattcggcatttgaaattcattttgttctctcttcttcattattagtgcatttggtgtgtgtatacttgcacacaattctgtttgtgtacacactgcttgcttagccctagtcaagaggcatcttttataaaaggtgtaaagaaatatcaaggttctaaaattcggaagagtttagaatttattaggagtttcccaagttgggatgttagtctttaaataaacttcatgcacctattccacttaaggttttgcacctcctttttattagtgcagtgccatttcttctgcttgattttaggtatgttaatattccagccttgctagttagcataaagtgacaggtgtgagccatgaggaaattttctgacttaatttgtacacaactacatataagagttttagtggaggaaaaaaattagtcccttgtgcgtatacagtagttaggtaaatgatttttctaccaacagtatactccattcctcatgtaggtaagtacagaaaaggtttttaaatgtatttttttagccagttaaagtctatgaatctatctgcaaccttatttaatctgtcactataataattttgtggttatgctaagaaccatgtatacttttaggtattcttatttttgtcaatttttctaggttggcaaggaggcagaaaaccttcattgtttcatattaaaatataattagactaaacttaattctagtatgaatttccaaaatcattatctatttatttcatttttatttaattttgtttttatttcatttttaaaagtcccttgttcaatttaacttatgttcctaagagaggttggagaacttggccttcatctgatttcaaaaatgttttgagtttcaaatgaagttaatggtttcagtgtgattcagtcctcagacctaattgggttgaataaaatctaaaagaatatacccttttggagcataacattttaataccttggggaatgtggcactaccaaaagaagactactaacacgtcagatgttcacctggaagctttatcaagaaattcgaaccacccttttggccccattaattgtagcaagtttatttctctatattttgtcattcagtgaattgaagtcctgtggtatactgcattcattagaagaaaaacgtttttaatgtccttttaatgatggcccagaaagcatttgacacagcaagatgcatgtgttactatattgagaatatagaataataacagtatcactaaatttaagacctcttcccagtcttgctgttcctagcaagaagtttggcctgtgactgcacttactgtttatgctcatcagaaactgtcaatgtctgcttttctttaactctgcagtctgtaacatcacgctgtttattaaaaaaaaaaagaaaaatta

SEQ ID NO: 133 - protein, 1068 aa, Homo sapiens; identical to SEQ ID NO: 118
  REGION 1..1068: note = MISC_FEATURE - PIK3CA, wild type

SEQ ID NO: 134 - DNA, 3421 bp, synthetic construct
  source 1..3421: mol_type = other DNA
gaggaaccgagaggctgagactaacccagaaacatccaattctcaaactgaagctcgcactctcgcctccagcatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctaaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 135 - DNA, 3421 bp, synthetic construct
  source 1..3421: mol_type = other DNA
gaggaaccgagaggctgagactaacccagaaacatccaattctcaaactgaagctcgcactctcgcctccagcatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactaagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 136 - DNA, 3421 bp, synthetic construct
  source 1..3421: mol_type = other DNA
gaggaaccgagaggctgagactaacccagaaacatccaattctcaaactgaagctcgcactctcgcctccagcatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacggcatggtggctggacaacaaaaatggattggatcttccacacaattaaacagcatgcattgaactgaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 137 - DNA, 2987 bp, synthetic construct
  source 1..2987: mol_type = other DNA
taattatgggtctgtaaccaccctggactgggtgctcctcactgacggacttgtctgaacctctctttgtctccagcgcccagcactgggcctggcaaaacctgagacgcccggtacatgttggccaaatgaatgaaccagattcagaccggcaggggcgctgtggtttaggaggggcctggggtttctcccaggaggtttttgggcttgcgctggagggctctggactcccgtttgcgccagtggcctgcatcctggtcctgtcttcctcatgtttgaatttctttgctttcctagtctggggagcagggaggagccctgtgccctgtcccaggatccatgggtaggaacaccatggacagggagagcaaacggggccatctgtcaccaggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttca

SEQ ID NO: 138 - protein, 480 aa, Homo sapiens [sequence omitted]

SEQ ID NO: 139 - DNA, 8721 bp, Homo sapiens
  source 1..8721: mol_type = genomic DNA
gctcccggcttagaggacagcggggaaggcgggcggtggggcagggggcctgaagcggcggtaccggtgctggcggcggcagctgaggccttggccgaagccgcgcgaacctcagggcaagatgcttggaaccggacctgccgccgccaccaccgctgccaccacatctagcaatgtgagcgtcctgcagcagtttgccagtggcctaaagagccggaatgaggaaaccagggccaaagccgccaaggagctccagcactatgtcaccatggaactccgagagatgagtcaagaggagtctactcgcttctatgaccaactgaaccatcacatttttgaattggtttccagctcagatgccaatgagaggaaaggtggcatcttggccatagctagcctcataggagtggaaggtgggaatgccacccgaattggcagatttgccaactatcttcggaacctcctcccctccaatgacccagttgtcatggaaatggcatccaaggccattggccgtcttgccatggcaggggacacttttaccgctgagtacgtggaatttgaggtgaagcgagccctggaatggctgggtgctgaccgcaatgagggccggagacatgcagctgtcctggttctccgtgagctggccatcagcgtccctaccttcttcttccagcaagtgcaacccttctttgacaacatttttgtggccgtgtgggaccccaaacaggccatccgtgagggagctgtagccgcccttcgtgcctgtctgattctcacaacccagcgtgagccgaaggagatgcagaagcctcagtggtacaggcacacatttgaagaagcagagaagggatttgatgagaccttggccaaagagaagggcatgaatcgggatgatcggatccatggagccttgttgatccttaacgagctggtccgaatcagcagcatggagggagagcgtctgagagaagaaatggaagaaatcacacagcagcagctggtacacgacaagtactgcaaagatctcatgggcttcggaacaaaacctcgtcacattacccccttcaccagtttccaggctgtacagccccagcagtcaaatgccttggtggggctgctggggtacagctctcaccaaggcctcatgggatttgggacctcccccagtccagctaagtccaccctggtggagagccggtgttgcagagacttgatggaggagaaatttgatcaggtgtgccagtgggtgctgaaatgcaggaatagcaagaactcgctgatccaaatgacaatccttaatttgttgccccgcttggctgcattccgaccttctgccttcacagatacccagtatctccaagataccatgaaccatgtcctaagctgtgtcaagaaggagaaggaacgtacagcggccttccaagccctggggctactttctgtggctgtgaggtctgagtttaaggtctatttgcctcgcgtgctggacatcatccgagcggccctgcccccaaaggacttcgcccataagaggcagaaggcaatgcaggtggatgccacagtcttcacttgcatcagcatgctggctcgagcaatggggccaggcatccagcaggatatcaaggagctgctggagcccatgctggcagtgggactaagccctgccctcactgcagtgctctacgacctgagccgtcagattccacagctaaagaaggacattcaagatgggctactgaaaatgctgtccctggtccttatgcacaaaccccttcgccacccaggcatgcccaagggcctggcccatcagctggcctctcctggcctcacgaccctccctgaggccagcgatgtgggcagcatcactcttgccctccgaacgcttggcagctttgaatttgaaggccactctctgacccaatttgttcgccactgtgcggatcatttcctgaacagtgagcacaaggagatccgcatggaggctgcccgcacctgctcccgcctgctcacaccctccatccacctcatcagtggccatgctcatgtggttagccagaccgcagtgcaagtggtggcagatgtgcttagcaaactgctcgtagttgggataacagatcctgaccctgacattcgctactgtgtcttggcgtccctggacgagcgctttgatgcacacctggcccaggcggagaacttgcaggccttgtttgtggctctgaatgaccaggtgtttgagatccgggagctggccatctgcactgtgggccgactcagtagcatgaaccctgcctttgtcatgcctttcctgcgcaagatgctcatccagattttgacagagttggagcacagtgggattggaagaatcaaagagcagagtgcccgcatgctggggcacctggtctccaatgccccccgactcatccgcccctacatggagcctattctgaaggcattaattttgaaactgaaagatccagaccctgatccaaacccaggtgtgatcaataatgtcctggcaacaataggagaattggcacaggttagtggcctggaaatgaggaaatgggttgatgaactttttattatcatcatggacatgctccaggattcctctttgttggccaaaaggcaggtggctctgtggaccctgggacagttggtggccagcactggctatgtagtagagccctacaggaagtaccctactttgcttgaggtgctactgaattttctgaagactgagcagaaccagggtacacgcagagaggccatccgtgtgttagggcttttaggggctttggatccttacaagcacaaagtgaacattggcatgatagaccagtcccgggatgcctctgctgtcagcctgtcagaatccaagtcaagtcaggattcctctgactatagcactagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaacgtcattcgagtctgtgatggggccatccgggaatttttgttccagcagctgggaatgttggtgtcctttgtgaagagccacatcagaccttatatggatgaaatagtcaccctcatgagagaattctgggtcatgaacacctcaattcagagcacgatcattcttctcattgagcaaattgtggtagctcttgggggtgaatttaagctctacctgccccagctgatcccacacatgctgcgtgtcttcatgcatgacaacagcccaggccgcattgtctctatcaagttactggctgcaatccagctgtttggcgccaacctggatgactacctgcatttactgctgcctcctattgttaagttgtttgatgcccctgaagctccactgccatctcgaaaggcagcgctagagactgtggaccgcctgacggagtccctggatttcactgactatgcctcccggatcattcaccctattgttcgaacactggaccagagcccagaactgcgctccacagccatggacacgctgtcttcacttgtttttcagctggggaagaagtaccaaattttcattccaatggtgaataaagttctggtgcgacaccgaatcaatcatcagcgctatgatgtgctcatctgcagaattgtcaagggatacacacttgctgatgaagaggaggatcctttgatttaccagcatcggatgcttaggagtggccaaggggatgcattggctagtggaccagtggaaacaggacccatgaagaaactgcacgtcagcaccatcaacctccaaaaggcctggggcgctgccaggagggtctccaaagatgactggctggaatggctgagacggctgagcctggagctgctgaaggactcatcatcgccctccctgcgctcctgctgggccctggcacaggcctacaacccgatggccagggatctcttcaatgctgcatttgtgtcctgctggtctgaactgaatgaagatcaacaggatgagctcatcagaagcatcgagttggccctcacctcacaagacatcgctgaagtcacacagaccctcttaaacttggctgaattcatggaacacagtgacaagggccccctgccactgagagatgacaatggcattgttctgctgggtgagagagctgccaagtgccgagcatatgccaaagcactacactacaaagaactggagttccagaaaggccccacccctgccattctagaatctctcatcagcattaataataagctacagcagccggaggcagcggccggagtgttagaatatgccatgaaacactttggagagctggagatccaggctacctggtatgagaaactgcacgagtgggaggatgcccttgtggcctatgacaagaaaatggacaccaacaaggacgacccagagctgatgctgggccgcatgcgctgcctcgaggccttgggggaatggggtcaactccaccagcagtgctgtgaaaagtggaccctggttaatgatgagacccaagccaagatggcccggatggctgctgcagctgcatggggtttaggtcagtgggacagcatggaagaatacacctgtatgatccctcgggacacccatgatggggcattttatagagctgtgctggcactgcatcaggacctcttctccttggcacaacagtgcattgacaaggccagggacctgctggatgctgaattaactgcgatggcaggagagagttacagtcgggcatatggggccatggtttcttgccacatgctgtccgagctggaggaggttatccagtacaaacttgtccccgagcgacgagagatcatccgccagatctggtgggagagactgcagggctgccagcgtatcgtagaggactggcagaaaatccttatggtgcggtcccttgtggtcagccctcatgaagacatgagaacctggctcaagtatgcaagcctgtgcggcaagagtggcaggctggctcttgctcataaaactttagtgttgctcctgggagttgatccgtctcggcaacttgaccatcctctgccaacagttcaccctcaggtgacctatgcctacatgaaaaacatgtggaagagtgcccgcaagatcgatgccttccagcacatgcagcattttgtccagaccatgcagcaacaggcccagcatgccatcgctactgaggaccagcagcataagcaggaactgcacaagctcatggcccgatgcttcctgaaacttggagagtggcagctgaatctacagggcatcaatgagagcacaatccccaaagtgctgcagtactacagcgccgccacagagcacgaccgcagctggtacaaggcctggcatgcgtgggcagtgatgaacttcgaagctgtgctacactacaaacatcagaaccaagcccgcgatgagaagaagaaactgcgtcatgccagcggggccaacatcaccaacgccaccactgccgccaccacggccgccactgccaccaccactgccagcaccgagggcagcaacagtgagagcgaggccgagagcaccgagaacagccccaccccatcgccgctgcagaagaaggtcactgaggatctgtccaaaaccctcctgatgtacacggtgcctgccgtccagggcttcttccgttccatctccttgtcacgaggcaacaacctccaggatacactcagagttctcaccttatggtttgattatggtcactggccagatgtcaatgaggccttagtggagggggtgaaagccatccagattgatacctggctacaggttatacctcagctcattgcaagaattgatacgcccagacccttggtgggacgtctcattcaccagcttctcacagacattggtcggtaccacccccaggccctcatctacccactgacagtggcttctaagtctaccacgacagcccggcacaatgcagccaacaagattctgaagaacatgtgtgagcacagcaacaccctggtccagcaggccatgatggtgagcgaggagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagctgcctcagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggcagcaacggacatgagtttgttttccttctaaaaggccatgaagatctgcgccaggatgagcgtgtgatgcagctcttcggcctggttaacacccttctggccaatgacccaacatctcttcggaaaaacctcagcatccagagatacgctgtcatccctttatcgaccaactcgggcctcattggctgggttccccactgtgacacactgcacgccctcatccgggactacagggagaagaagaagatccttctcaacatcgagcatcgcatcatgttgcggatggctccggactatgaccacttgactctgatgcagaaggtggaggtgtttgagcatgccgtcaataatacagctggggacgacctggccaagctgctgtggctgaaaagccccagctccgaggtgtggtttgaccgaagaaccaattatacccgttctttagcggtcatgtcaatggttgggtatattttaggcctgggagatagacacccatccaacctgatgctggaccgtctgagtgggaagatcctgcacattgactttggggactgctttgaggttgctatgacccgagagaagtttccagagaagattccatttagactaacaagaatgttgaccaatgctatggaggttacaggcctggatggcaactacagaatcacatgccacacagtgatggaggtgctgcgagagcacaaggacagtgtcatggccgtgctggaagcctttgtctatgaccccttgctgaactggaggctgatggacacaaataccaaaggcaacaagcgatcccgaacgaggacggattcctactctgctggccagtcagtcgaaattttggacggtgtggaacttggagagccagcccataagaaaacggggaccacagtgccagaatctattcattctttcattggagacggtttggtgaaaccagaggccctaaataagaaagctatccagattattaacagggttcgagataagctcactggtcgggacttctctcatgatgacactttggatgttccaacgcaagttgagctgctcatcaaacaagcgacatcccatgaaaacctctgccagtgctatattggctggtgccctttctggtaactggaggcccagatgtgcccatcacgttttttctgaggcttttgtactttagtaaatgcttccactaaactgaaaccatggtgagaaagtttgactttgttaaatattttgaaatgtaaatgaaaagaactactgtatattaaaagttggtttgaaccaactttctagctgctgttgaagaatatattgtcagaaacacaaggcttgatttggttcccaggacagtgaaacatagtaataccacgtaaatcaagccattcattttggggaacagaagatccataactttagaaatacgggttttgacttaactcacaagagaactcatcataagtacttgctgatggaagaatgacctagttgctcctctcaacatgggtacagcaaactcagcacagccaagaagcctcaggtcgtggagaacatggattaggatcctagactgtaaagacacagaagatgctgacctcacccctgccacctatcccaagacctcactggtctgtggacagcagcagaaatgtttgcaagataggccaaaatgagtacaaaaggtctgtcttccatcagacccagtgatgctgcgactcacacgcttcaattcaagacctgaccgctagtagggaggtttattcagatcgctggcagcctcggctgagcagatgcacagaggggatcactgtgcagtgggaccaccctcactggccttctgcagcagggttctgggatgttttcagtggtcaaaatactctgtttagagcaagggctcagaaaacagaaatactgtcatggaggtgctgaacacagggaaggtctggtacatattggaaattatgagcagaacaaatactcaactaaatgcacaaagtataaagtgtagccatgtctagacaccatgttgtatcagaataatttttgtgccaataaatgacatcagaattttaaacata

SEQ ID NO: 140 - protein, 2549 aa, Homo sapiens
  REGION 1..2549: note = MISC_FEATURE - mTor, wild type
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 141 - DNA, 4190 bp, Homo sapiens
  source 1..4190: mol_type = genomic DNA
aggttgtccggcgctgtcgctcggttgcggcggctgcggttggcggtggctgcggcggcggcgcgggctgagtgcggccgcgcgggagtccgcggctggcgcggcccgagcggggacccggcggctcgccaggcggcggccgaggcggggcgggccggcccggggccgagggccggtggccgaggccggagggccgcggcgggcggcggccgaggcggctccggccagggccgggccgggggccggggggcggcggcgggcaggcggccgcgtcggccggggccgggacgatgactctggagtccatgatggcgtgttgcctgagcgatgaggtgaaggagtccaagcggatcaacgccgagatcgagaagcagctgcggcgggacaagcgcgacgcccggcgcgagctcaagctgctgctgctcggcacgggcgagagcgggaagagcacgttcatcaagcagatgcgcatcatccacggcgccggctactcggaggaggacaagcgcggcttcaccaagctcgtctaccagaacatcttcaccgccatgcaggccatgatccgggccatggagacgctcaagatcctctacaagtacgagcagaacaaggccaatgcgctcctgatccgggaggtggacgtggagaaggtgaccaccttcgagcatcagtacgtcagtgccatcaagaccctgtgggaggacccgggcatccaggaatgctacgaccgcaggcgcgagtaccagctctccgactctgccaagtactacctgaccgacgttgaccgcatcgccaccttgggctacctgcccacccagcaggacgtgctgcgggtccgcgtgcccaccaccggcatcatcgagtaccctttcgacctggagaacatcatcttccggatggtggatgtggggggccagcggtcggagcggaggaagtggatccactgctttgagaacgtgacatccatcatgtttctcgtcgccctcagcgaatacgaccaagtcctggtggagtcggacaacgagaaccggatggaggagagcaaagccctgttccggaccatcatcacctacccctggttccagaactcctccgtcatcctcttcctcaacaagaaggacctgctggaggacaagatcctgtactcgcacctggtggactacttccccgagttcgatggtccccagcgggacgcccaggcggcgcgggagttcatcctgaagatgttcgtggacctgaaccccgacagcgacaagatcatctactcacacttcacgtgtgccaccgacacggagaacatccgcttcgtgttcgcggccgtgaaggacaccatcctgcagctcaacctcaaggagtacaacctggtctgagcgcccaggcccagggagacgggatggagacacggggcaggaccttccttccacggagcctgcggctgccgggcgggtggcgctgccgagtccgggccggggcctctgcccgcgggaggagatttttttttttcatatttttaacaaatggtttttatttcacagttatcaggggatgtacatctctccctccgtacacttcgcgcaccttctcaccttttgtcaacggcaaaggcagcctttttctggccttgacttatggctcgcttttttctaaaaaaaaaaaaaaaagaaagaaagaaaaaaagcaacgaaacataaaacacacaagcgccccgtgcccccagtgactctgggcctcacagagcccccgccagccagcatggggccccgccctgcagccagtcacgcgcccccacaccgcagccccccgtggctgtccttccaaccccacgtgctttttctttctcctgcccgcttcttttcttcatcacaaaaggcgtggagactcggagacggacgtttttccccttttttaagttattgacgcccagcgcgcctcgcctcttcacccatcaacgctgtgctttgcccactggactcctgaagagggggtggggggctccctcggtcgcccaccctgggaagtgcctaaccttttattttattttatttttttgaggaaaaagaacgcctgactcacaggttgaagaaacaccctgggccctctctcatggccgggttccccgtccctctgcagaggctgggaagggtccccgggctggagccacgggggcttctctgggctgtgcctccggggccaacactggctgcttggggctgcccggggactccagagggctgcacggccaccctgccctggctagagcgcaccccaccggagcccacgtgggctgggcggctggagggatggtcccccggtgacactgggagaaaggccacttggatgggggcgtttctgttttgttccgctttgtgatgtcaccaatttggaaacagcgagggtgggtggggacttttacagaatattctcaggtgtgtacccgagaggcagagagagggacgtggccggcagctctgtgcgtggccttgtcccaagcacttgcgcccgcccccgagcgccgcccccggggagcgggaagccagcactcgcactttggccaggggcgcgtggaaggtggtggcaggcaccggcctgggcagcttccaggcctggctggccacgaccacggcccgagggggagcccgccaggccacgccgcactgagccacagccccgggggccgcctcccggggccccttgaggcactgaggcaccgagactggttctccccgagagactcggaaggtggggaacgaggggactgtgtttggggaggtggctttttcgtctgctgttgactgaacactacagcgccctgtggttccgggcttcgcacagctgtcccagggatggatcgcctgtgctgccttcgcccgccgccacaccgggaccctgcacggctgcttctggcctcgacagatgacaaaagaaacagccccaaaatacgaccactccaaccagcagttcccgcctgcctgcccgccactgtcaggcctgccctggcctcctcgtccgcagggctgtctgctggcttctgggggcagaagagcggggagccccgtggaagggtcaggggagaccaggtcagggcagctacatttctggtgatcagccccatggggagacggggctggcgggataccccccccccggcttccccacaccacttctgtctcacccggaagcgtcctttttttgtgccaggtgtctacctaagagggttggtgccagaagccccccatggcgagtgctggggcccggcggtgccctgggggagcagatggggccacccctggcagggccgctacaaccttttccagcagcggagccctctggggggcctgtgcttgtggcatctctgagggcctagattgcacaaggtgacctggccgtggcctgagggtggagtcgcccagcacgcaggccggggcgctgcggggctaagtattaggccttcccagggagggggcgtgccaagcatcccagagccgggctgggaccgccaaaacgtcgtggcctggatcctctgggtctgagtgcctgatcccctgccccccaaaaaagcagaggtaggtgttgcaggcccagggcaggggtgcctgccccaggagagtcccaggcagtggttctcgtgccagtggcacccaggggcaaggacagccaacccccacccttgccacgtgtggggccacgtgggcatgtggggtgtgtgtttttaccttggtgaatctcacctgccaacgatttctcgtgagtgccgaccaccttctccgaccatgttacgcccgggcggcagcagcccccggccactgcaaacccatgccctgggtcccccggctcccccagggaggcatccccgtgccaatgtcccccagtggtggcagcagatcctgtggccggcctggcggacgggacccagtgatacttgtatattacacagtcctgatttcagacaatttcaaccttaatctatttaaaaaagaatattctatacaagctgtttttaagccttttaccatttgaaatgcatgtgttgtgcgcgttggggatgggaggaggggctgaggagcggctcagtgtcacctcccacagccaccggccctgacccttaatccagacaccgatggaagtcgacttttcatatctttctcctgaaatgaactctgttttaaattggaataaattttgttcctaaa

SEQ ID NO: 142 - protein, 359 aa, Homo sapiens
  REGION 1..359: note = MISC_FEATURE - GNA11, wild type
MTLESMMACCLSDEVKESKRINAEIEKQLRRDKRDARRELKLLLLGTGESGKSTFIKQMRIIHGAGYSEEDKRGFTKLVYQNIFTAMQAMIRAMETLKILYKYEQNKANALLIREVDVEKVTTFEHQYVSAIKTLWEDPGIQECYDRRREYQLSDSAKYYLTDVDRIATLGYLPTQQDVLRVRVPTTGIIEYPFDLENIIFRMVDVGGQRSERRKWIHCFENVTSIMFLVALSEYDQVLVESDNENRMEESKALFRTIITYPWFQNSSVILFLNKKDLLEDKILYSHLVDYFPEFDGPQRDAQAAREFILKMFVDLNPDSDKIIYSHFTCATDTENIRFVFAAVKDTILQLNLKEYNLV

SEQ ID NO: 143 - DNA, 6870 bp, Homo sapiens
  source 1..6870: mol_type = genomic DNA
gttgccgggcgggacgggaaccgggtctgcgcgcgagccgggcggtgggcgcggacagggccgagggcgcccgggagccgtgtcaggcggcggcgaggagcgggcgcgccgggcgcggggagccgtcggcggccgctcgctgcggggacaccccggtgaatgagctctcgccgccaggcgcacggcgtaggggagcctcgcaggcggcggcggcggcggcggctcggggggcgaggcgggccgcctgttcccggggaaggaggcgcgggcgtctgagcgaggccgggcgcggcggccttctctcacgcgccccccgagccagcgcccgcctcccgcgcccgcctctcttctcccgtcggcccagactatccgctcccaccgcgcccccggcccacctggtggccccggccctggccgccgcccccgcggcggttcccggagctcgtcccggacgcgcgcccgggcggcgggggctcggcggccaccgctgcctcgggggagcgagggcgggagggtgtgtgtgcgcgctgtgagcagggggtgccggcggggctgcagcggaggcactttggaagaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggagtacaatctggtctaattgtgcctcctagacacccgccctgcccttccctggtgggctattgaagatacacaagagggactgtatttctgtggaaaacaatttgcataatactaatttattgccgtcctggactctgtgtgagcgtgtccacagagtttgtagtaaatattatgattttatttaaactattcagaggaaaaacagaggatgctgaagtacagtcccagcacatttcctctctatcttttttttaggcaaaaccttgtgactcagtgtattttaaattctcagtcatgcactcacaaagataagacttgtttctttctgtctctctctctttttcttttctatggagcaaaacaaagctgatttcccttttttcttcccccgctaattcatacctccctcctgatgtttttcccaggttacaatggcctttatcctagttccattcttggtcaagtttttctctcaaatgatacagtcaggacacatcgttcgatttaagccatcatcagcttaatttaagtttgtagtttttgctgaaggattatatgtattaatacttacggttttaaatgtgttgctttggatacacacatagtttcttttttaatagaatatactgtcttgtctcactttggactgggacagtggatgcccatctaaaagttaagtgtcatttcttttagatgtttaccttcagccatagcttgattgctcagagaaatatgcagaaggcaggatcaaagacacacaggagtcctttcttttgaaatgccacgtgccattgtctttcctcccttctttgcttctttttcttaccctctctttcaattgcagatgccaaaaaagatgccaacagacactacattaccctaatggctgctacccagaacctttttataggttgttcttaatttttttgttgttgttgttcaagcttttcctttcttttttttcttggtgtttgggccacgattttaaaatgacttttattatgggtatgtgttgccaaagctggctttttgtcaaataaaatgaatacgaacttaaaaaataaaagctggtatcttaaaatgtaagagagtaagactgtgaagcctaaaatgactggctgagaatgaaccagaaatgccatttgccaaacagttgtaactagaaatttgattctcacggtccattcttttctttgtccttaagatgacattgttagtgttcacgtcccatgttcagtgtccaaaccggcaatgtaaaaagtatcctgtgtggtttaacaggaaatctgtttatgtctctttatttgaaaccagttttactctcagtggttctttaagttcaatgaagtctgccaggaacattggttggtagtattattccgacacctttaatttccaaaatctgaagttcctgctagtttaccaccttcatgatcttcttgaactggtaactgattaggttgaacttatggaagatttgtggacttaactcaaaagtaacctctcagtgttctatagaacatgtatttgtgtaactgaacctaccaggagaaatgtttggaattctatatgtgcaatttttcaacaaatgcaaaaaaaatacagcacatgtattgacaagcttctgtcaagcagcttgagttgaaatttgatttaagaaaataaatcatgattgttcaaagctgctgggacgttagaattaggccatgatactggtctcattttaactacagtggtatttggcactagtgtaaacttccatataaatcactcttttggaacaacaaagggggagggagaaaaatcacggcctgttaaatgagtaccaaagccgcccaacagtaatgagatgttctcatccttgattctcccagcctcaaacaacacagcttactttttttttcccttgctcagaaagtacctgtaatttaacaaacagactgcctgtaggtatagtgcaattacaaatgctctaatcattgtacatacatctctcttgatattgcagcatccatactggctttgtaatcattaattttttggcagattgaatgtgctgtattgatatgtatctatgtaattgtattgtatgtctatagctaattcacgttttgaataatgttattttatttacttttttaagagaggagaatgtaaatttgtcagtttatttctgactagggatattttctttccatttagaaaagaagaaaaaaaaaaaaccttactgtcatacagagcggtactagcgtcgtgctgtataaaatcatttgcacattcctgagtagaggtatactgattataagacccaaaggtaatttcatagcaaaatacataaaatcagtcggagcttttatacaaacatggaaaccaactttgtagaacttttgccatttgatctaggattggaatatgagcttttatacaattcatattcttatttggcaaatgcacagtttagtattacctctctgatggcctttactagaaaggcagttttagaagctattgtgatccactaaggaaatgttttaacagctagagaccactgcttgcctgaaagggcgttcttaaatttggtgcagcaaaaaaaaaaaaaaaaaaaaaaaaaattaaacaacaacatttgaaggcctacagtgtgtatagagaaaacctcatcacaagatcataagtgttacagttttagggaatcaagatattctatttaatagagctatagtaaatgtagtcaattaaacctgatctcaaagcttgaagaagctgagcaaaacagggaaagattgttatatttgtctttatgaaattgggatggaatttgctatgcagaattgaggtttgtggcttcgctgttcctgtagggtgcatgacaagatcccttctcttgagaaaggaaaaaattgatcaccctagcagcagtgatgcatagaaacctaattttagccacaccagtcaatcgaagctaaaggattttcttttttgtttcttcggggttttattgaaggggctaggggcgggacgggattcttttcagttttgtataaaaacaaagtttactcatgctttatattatattgtgattgcaagcgttataagcgtgtgccactggcctcctattgttgatgcttaggtaatggaggcctgtggtgagttttatggtgacttgggcatgtcttattcaaaaacaaaaacataaaacacagaaacctttcttcagcataccaaggcaagcagccatttcatgactcacttaacacattgcagtgtaccagtttacagatgatttttccctttttgcgtgacatggcagttctaacccccagagaattccttatttgtaaattggaagtttctactatgccttacagagcttaaattcagaagtttgtgcctcatatctgaaacaaagggaaataacacacccattcaaaagtaaataaatctcctagaagtttttgtttttaacatttccatataaagagctctgttgaatgtcatgaatagactggaaaaaaaaattttaagaacctgcatatgttgtttactagcagatgacaactacaaaaggaatctgaagaacacgtaaaacttgtatttttttttttttggtagattaactagcaggcctattttaaaaaggtaattcagctaaagggcaatttacttttttgtacttcagactatcttgattgtcaaagtgtacgaactgtaattttaaaatttatactgccacatgattgtaaattttagttgtcttaagttaggaattggtgaaaagctatttatgctggatttgggtcaaaatgacttatttgcaaaaaaataaataatgggaagaaagggctgtataatgaaatactgcaagactcacatattggttggaaatttccctcaaatcacctaccgattacccttgatttccctttgttttcagtttctcaaaacgaatgaaatgaaatatagcagaatgttaacccatataaaaataaagtgtacccaaatattgtaatgtatattgctgctcttcttcaaattaaataagggtttaaaaccacttaattggtaatcaacatctcaattgatacaaataaggtgtgcttggtatacattaatattttcttccaaagatatatctttggttagaaacacaaaaaaataaaactagtaatattgtatgtttatctatctctacatatttccagcatatgtagcgttaatagatctgtcctggtaactgtgtctttgggatttcattttggttccatcaaattaggaaaagaaatggcttagttgtatatgattagctagagatttttggagccagacacctgctgtttagtagataacttagtacagaccctaaacttgtcatttgtttttctcacagaatagccatttcctgctgtcttcccaatgatcactgccctttcaataacactcttgcctctagaatcatatgttcaaagtatgaatacacacctagcacatagtaggtgctcaaatattaatttcctccttgccttccttatctaccctgtgtcctccatttccccgtatgattccaacccaatatagcaaatgacatttacatgttatgaaaacatctattgggtaaaatcagatcttggataaagaaattctgacttttatataagcttttggtagacagaaaaaacagaaaggtattcgttggtagaacatttttaagttcaggaaagaaagctggaataatactacgtaactttgtccaggttactttgactgaaacacgtttttggtggatttcttttcctcaaagaactctctaaatgcaactccttgctggattcctcacccatcatcctgttggaaacccttactagacctatgtatttagggagttttgtcagaaaacatttttaacttgcagtatttaaaagaatatttactgttcctaaaatgtcattcaaatgcatgtactgtctattgtttggggatgggaactagttttgcaaaaaacacctaatgttgtataataatgccccaatgatcttgctggttaaaaatacagtatttttggccataattttgtactatagtattcttcattgttgagttttgcttccattaaagttgcccgagcccctctcagaatgttttgtattagtaggagtactgaaatacatctttccttccttaacacaccttgtgcatattgcattttccaaaataaacaagtttaatggtttca

SEQ ID NO: 144 - protein, 359 aa, Homo sapiens
  REGION 1..359: note = MISC_FEATURE - GNAQ
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKEYNLV

SEQ ID NO: 145 - DNA, 5430 bp, Homo sapiens
  source 1..5430: mol_type = genomic DNA
ctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggcttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccacacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgac

SEQ ID NO: 146 - protein, 189 aa, Homo sapiens [sequence omitted]

SEQ ID NO: 147 - DNA, 4326 bp, Homo sapiens
  source 1..4326: mol_type = genomic DNA
ggggccggaagtgccgctccttggtgggggctgttcatggcggttccggggtctccaacatttttcccggctgtggtcctaaatctgtccaaagcagaggcagtggagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaagttttgtcagaaaagagccactttcaagctgcactgacaccctggtcctgacttccctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctctgccaccccaggtttttcatctgaaaagcagttcatgtctgaaacagagaaccaaaccgcaaacgtgaaattctattgaaaacagtgtcttgagctctaaagtagcaactgctggtgattttttttttctttttactgttgaacttagaactatgctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaactttcacagtgaagtgcctttttcctagaagtggtttgtagacttcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcacatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaattacattttcactgtatatcatggtatcttaatgatgtatataattgccttcaatccccttctcaccccaccctctacagcttcccccacagcaataggggcttgattatttcagttgagtaaagcatggtgctaatggaccagggtcacagtttcaaaacttgaacaatccagttagcatcacagagaaagaaattcttctgcatttgctcattgcaccagtaactccagctagtaattttgctaggtagctgcagttagccctgcaaggaaagaagaggtcagttagcacaaaccctttaccatgactggaaaactcagtatcacgtatttaaacatttttttttcttttagccatgtagaaactctaaattaagccaatattctcatttgagaatgaggatgtctcagctgagaaacgttttaaattctctttattcataatgttctttgaagggtttaaaacaagatgttgataaatctaagctgatgagtttgctcaaaacaggaagttgaaattgttgagacaggaatggaaaatataattaattgatacctatgaggatttggaggcttggcattttaatttgcagataataccctggtaattctcatgaaaaatagacttggataacttttgataaaagactaattccaaaatggccactttgttcctgtctttaatatctaaatacttactgaggtcctccatcttctatattatgaattttcatttattaagcaaatgtcatattaccttgaaattcagaagagaagaaacatatactgtgtccagagtataatgaacctgcagagttgtgcttcttactgctaattctgggagctttcacagtactgtcatcatttgtaaatggaaattctgcttttctgtttctgctccttctggagcagtgctactctgtaattttcctgaggcttatcacctcagtcatttcttttttaaatgtctgtgactggcagtgattctttttcttaaaaatctattaaatttgatgtcaaattagggagaaagatagttactcatcttgggctcttgtgccaatagcccttgtatgtatgtacttagagttttccaagtatgttctaagcacagaagtttctaaatggggccaaaattcagacttgagtatgttctttgaataccttaagaagttacaattagccgggcatggtggcccgtgcctgtagtcccagctacttgagaggctgaggcaggagaatcacttcaacccaggaggtggaggttacagtgagcagagatcgtgccactgcactccagcctgggtgacaagagagacttgtctccaaaaaaaaagttacacctaggtgtgaattttggcacaaaggagtgacaaacttatagttaaaagctgaataacttcagtgtggtataaaacgtggtttttaggctatgtttgtgattgctgaaaagaattctagtttacctcaaaatccttctctttccccaaattaagtgcctggccagctgtcataaattacatattccttttggtttttttaaaggttacatgttcaagagtgaaaataagatgttctgtctgaaggctaccatgccggatctgtaaatgaacctgttaaatgctgtatttgctccaacggcttactatagaatgttacttaatacaatatcatacttattacaatttttactataggagtgtaataggtaaaattaatctctattttagtgggcccatgtttagtctttcaccatcctttaaactgctgtgaatttttttgtcatgacttgaaagcaaggatagagaaacactttagagatatgtggggtttttttaccattccagagcttgtgagcataatcatatttgctttatatttatagtcatgaactcctaagttggcagctacaaccaagaaccaaaaaatggtgcgttctgcttcttgtaattcatctctgctaataaattataagaagcaaggaaaattagggaaaatattttatttggatggtttctataaacaagggactataattcttgtacattatttttcatctttgctgtttctttgagcagtctaatgtgccacacaattatctaaggtatttgttttctataagaattgttttaaaagtattcttgttaccagagtagttgtattatatttcaaaacgtaagatgatttttaaaagcctgagtactgacctaagatggaattgtatgaactctgctctggagggaggggaggatgtccgtggaagttgtaagacttttatttttttgtgccatcaaatataggtaaaaataattgtgcaattctgctgtttaaacaggaactattggcctccttggccctaaatggaagggccgatattttaagttgattattttattgtaaattaatccaacctagttctttttaatttggttgaatgttttttcttgttaaatgatgtttaaaaaataaaaactggaagttcttggcttagtcataa

SEQ ID NO: 148 - protein, 189 aa, Homo sapiens
  REGION 1..189: note = MISC_FEATURE - NRAS, widl type
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 149 - DNA, 2393 bp, Homo sapiens
  source 1..2393: mol_type = genomic DNA
gggctgaggaggcggggcctgggaggggacaaagccgggaagaggaaaagctcggacctaccctgtggtcccgggtttctgcagagtctacttcagaagcggaggcactgggagtccggtttgggattgccaggctgtggttgtgagtctgagcttgtgagcggctgtggcgccccaactcttcgccagcatatcatcccggcaggcgataaactacattcagttgagtctgcaagactgggaggaactggggtgataagaaatctattcactgtcaaggtttattgaagtcaaaatgtccaaaaaaatcagtggcggttctgtggtagagatgcaaggagatgaaatgacacgaatcatttgggaattgattaaagagaaactcatttttccctacgtggaattggatctacatagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaatacagagcaactgattttgttgttcctgggcctggaaaagtagagataacctacacaccaagtgacggaacccaaaaggtgacatacctggtacataactttgaagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattgcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaatgtgcaacgttctgactacttgaatacatttgagttcatggataaacttggagaaaacttgaagatcaaactagctcaggccaaactttaagttcatacctgagctaagaaggataattgtcttttggtaactaggtctacaggtttacatttttctgtgttacactcaaggataaaggcaaaatcaattttgtaatttgtttagaagccagagtttatcttttctataagtttacagcctttttcttatatatacagttattgccacctttgtgaacatggcaagggacttttttacaatttttattttattttctagtaccagcctaggaattcggttagtactcatttgtattcactgtcactttttctcatgttctaattataaatgaccaaaatcaagattgctcaaaagggtaaatgatagccacagtattgctccctaaaatatgcataaagtagaaattcactgccttcccctcctgtccatgaccttgggcacagggaagttctggtgtcatagatatcccgttttgtgaggtagagctgtgcattaaacttgcacatgactggaacgaagtatgagtgcaactcaaatgtgttgaagatactgcagtcatttttgtaaagaccttgctgaatgtttccaatagactaaatactgtttaggccgcaggagagtttggaatccggaataaatactacctggaggtttgtcctctccatttttctctttctcctcctggcctggcctgaatattatactactctaaatagcatatttcatccaagtgcaataatgtaagctgaatcttttttggacttctgctggcctgttttatttcttttatataaatgtgatttctcagaaattgatattaaacactatcttatcttctcctgaactgttgattttaattaaaattaagtgctaattaccatta

SEQ ID NO: 150 - protein, 414 aa, Homo sapiens
  REGION 1..414: note = MISC_FEATURE - IDH1, wild type
MSKKISGGSVVEMQGDEMTRIIWELIKEKLIFPYVELDLHSYDLGIENRDATNDQVTKDAAEAIKKHNVGVKCATITPDEKRVEEFKLKQMWKSPNGTIRNILGGTVFREAIICKNIPRLVSGWVKPIIIGRHAYGDQYRATDFVVPGPGKVEITYTPSDGTQKVTYLVHNFEEGGGVAMGMYNQDKSIEDFAHSSFQMALSKGWPLYLSTKNTILKKYDGRFKDIFQEIYDKQYKSQFEAQKIWYEHRLIDDMVAQAMKSEGGFIWACKNYDGDVQSDSVAQGYGSLGMMTSVLVCPDGKTVEAEAAHGTVTRHYRMYQKGQETSTNPIASIFAWTRGLAHRAKLDNNKELAFFANALEEVSIETIEAGFMTKDLAACIKGLPNVQRSDYLNTFEFMDKLGENLKIKLAQAKL

SEQ ID NO: 151 - DNA, 3745 bp, synthetic construct
  source 1..3745: mol_type = other DNA
gaggaaccgagaggctgagactaacccagaaacatccaattctcaaactgaagctcgcactctcgccgccaccatggggagcagcaagagcaagcccaaggaccccagccagcgcgggggacatttatatgaagaatatacccgcacatcccaggaaatccaaatgaaaaggacagctattgaagcatttaatgaaaccataaaaatatttgaagaacagtgccagacccaagagcggtacagcaaagaatacatagaaaagtttaaacgtgaaggcaatgagaaagaaatacaaaggattatgcataattatgataagttgaagtctcgaatcagtgaaattattgacagtagaagaagattggaagaagacttgaagaagcaggcagctgagtatcgagaaattgacaaacgtatgaacagcattaaaggcggcatcagcggcggcggcggcggcatcgtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 152 - DNA, 1333 bp, synthetic construct
  source 1..1333: mol_type = other DNA
gaggaaccgagaggctgagactaacccagaaacatccaattctcaaactgaagctcgcactctcgcctccagcatggggagcagcaagagcaagcccaaggaccccagccagcgcgggggacatttgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttatggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttcttccgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctagggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 153 - DNA, 1579 bp, synthetic construct
  source 1..1579: mol_type = other DNA
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctgacctgggcccccagatgctgagggagctgcaggagaccaacgccgccctgcaggacgtgagggagctgctgaggcagcaggtgagggagatcaccttcctgaagaacaccgtgatggagtgcgacgcctgcggcggcggcatcagcggcggcggcggcggcatcatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttatggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttcttccgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctagggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 154 - DNA, 1333 bp, synthetic construct
  source 1..1333: mol_type = other DNA
gaggaaccgagaggctgagactaacccagaaacatccaattctcaaactgaagctcgcactctcgcctccagcatggggagcagcaagagcaagcccaaggaccccagccagcgcgggggacatttgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctagggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 155 - DNA, 1333 bp, synthetic construct
  source 1..1333: mol_type = other DNA
gaggaaccgagaggctgagactaacccagaaacatccaattctcaaactgaagctcgcactctcgcctccagcatggggagcagcaagagcaagcccaaggaccccagccagcgcgggggacatttgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggcttcttgtacctggccattgagtacgcgccccatggaaaccttctggacttccttctgaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataaccccctttatgagaagtttacttatgcaggaattgactgttctgctgaagaagcggcctagggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 156 - DNA, 1579 bp, synthetic construct
  source 1..1579: mol_type = other DNA
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctgacctgggcccccagatgctgagggagctgcaggagaccaacgccgccctgcaggacgtgagggagctgctgaggcagcaggtgagggagatcaccttcctgaagaacaccgtgatggagtgcgacgcctgcggcggcggcatcagcggcggcggcggcggcatcatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctagggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 157 - DNA, 1579 bp, synthetic construct
  source 1..1579: mol_type = other DNA
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctgacctgggcccccagatgctgagggagctgcaggagaccaacgccgccctgcaggacgtgagggagctgctgaggcagcaggtgagggagatcaccttcctgaagaacaccgtgatggagtgcgacgcctgcggcggcggcatcagcggcggcggcggcggcatcatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggcttcttgtacctggccattgagtacgcgccccatggaaaccttctggacttccttctgaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataaccccctttatgagaagtttacttatgcaggaattgactgttctgctgaagaagcggcctagggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 158 - DNA, 1414 bp, synthetic construct
  source 1..1414: mol_type = other DNA
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttatggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttcttccgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctagggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 159 - DNA, 1333 bp, synthetic construct
  source 1..1333: mol_type = other DNA
gaggaaccgagaggctgagactaacccagaaacatccaattctcaaactgaagctcgcactctcgcctccagcatggggagcagcaagagcaagcccaaggaccccagccagcgcgggggacatttgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataaccccctttatgagaagtttacttatgcaggaattgactgttctgctgaagaagcggcctagggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 160 - DNA, 1579 bp, synthetic construct
  source 1..1579: mol_type = other DNA
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctgacctgggcccccagatgctgagggagctgcaggagaccaacgccgccctgcaggacgtgagggagctgctgaggcagcaggtgagggagatcaccttcctgaagaacaccgtgatggagtgcgacgcctgcggcggcggcatcagcggcggcggcggcggcatcatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataaccccctttatgagaagtttacttatgcaggaattgactgttctgctgaagaagcggcctagggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 161 - DNA, 42 bp, synthetic construct
  source 1..42: mol_type = other DNA
atggggagcagcaagagcaagcccaaggaccccagccagcgc

SEQ ID NO: 162 - DNA, 1119 bp, Homo sapiens
  source 1..1119: mol_type = genomic DNA
atggggagcagcaagagcaagcccaaggaccccagccagcgcgggggacatttgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttatggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttcttccgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctag

SEQ ID NO: 163 - DNA, 30 bp, synthetic construct
  source 1..30: mol_type = other DNA
ggcggcatcagcggcggcggcggcggcatc

SEQ ID NO: 164 - DNA, 1365 bp, Homo sapiens
  source 1..1365: mol_type = genomic DNA
atgaaagtctctgccgcccttctgtgcctgctgctcatagcagccaccttcattccccaagggctcgctgacctgggcccccagatgctgagggagctgcaggagaccaacgccgccctgcaggacgtgagggagctgctgaggcagcaggtgagggagatcaccttcctgaagaacaccgtgatggagtgcgacgcctgcggcggcggcatcagcggcggcggcggcggcatcatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttatggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttcttccgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctag

SEQ ID NO: 165 - DNA, 1119 bp, Homo sapiens
  source 1..1119: mol_type = genomic DNA
atggggagcagcaagagcaagcccaaggaccccagccagcgcgggggacatttgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctag

SEQ ID NO: 166 - DNA, 1119 bp, Homo sapiens
  source 1..1119: mol_type = genomic DNA
atggggagcagcaagagcaagcccaaggaccccagccagcgcgggggacatttgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggcttcttgtacctggccattgagtacgcgccccatggaaaccttctggacttccttctgaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataaccccctttatgagaagtttacttatgcaggaattgactgttctgctgaagaagcggcctag

SEQ ID NO: 167 - DNA, 1365 bp, Homo sapiens
  source 1..1365: mol_type = genomic DNA
atgaaagtctctgccgcccttctgtgcctgctgctcatagcagccaccttcattccccaagggctcgctgacctgggcccccagatgctgagggagctgcaggagaccaacgccgccctgcaggacgtgagggagctgctgaggcagcaggtgagggagatcaccttcctgaagaacaccgtgatggagtgcgacgcctgcggcggcggcatcagcggcggcggcggcggcatcatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctag

SEQ ID NO: 168 - DNA, 1365 bp, Homo sapiens
  source 1..1365: mol_type = genomic DNA
atgaaagtctctgccgcccttctgtgcctgctgctcatagcagccaccttcattccccaagggctcgctgacctgggcccccagatgctgagggagctgcaggagaccaacgccgccctgcaggacgtgagggagctgctgaggcagcaggtgagggagatcaccttcctgaagaacaccgtgatggagtgcgacgcctgcggcggcggcatcagcggcggcggcggcggcatcatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggcttcttgtacctggccattgagtacgcgccccatggaaaccttctggacttccttctgaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataaccccctttatgagaagtttacttatgcaggaattgactgttctgctgaagaagcggcctag

SEQ ID NO: 169 - DNA, 1200 bp, Homo sapiens
  source 1..1200: mol_type = genomic DNA
atgaaagtctctgccgcccttctgtgcctgctgctcatagcagccaccttcattccccaagggctcgctatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttatggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttcttccgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctag

SEQ ID NO: 170 - DNA, 1119 bp, Homo sapiens
  source 1..1119: mol_type = genomic DNA
atggggagcagcaagagcaagcccaaggaccccagccagcgcgggggacatttgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataaccccctttatgagaagtttacttatgcaggaattgactgttctgctgaagaagcggcctag

SEQ ID NO: 171 - DNA, 1365 bp, Homo sapiens
  source 1..1365: mol_type = genomic DNA
atgaaagtctctgccgcccttctgtgcctgctgctcatagcagccaccttcattccccaagggctcgctgacctgggcccccagatgctgagggagctgcaggagaccaacgccgccctgcaggacgtgagggagctgctgaggcagcaggtgagggagatcaccttcctgaagaacaccgtgatggagtgcgacgcctgcggcggcggcatcagcggcggcggcggcggcatcatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataaccccctttatgagaagtttacttatgcaggaattgactgttctgctgaagaagcggcctag